(19) United States
(12) Patent Application Publication
(10) Pub. No.: US 2026/0274867 A1
(43) Pub. Date: Sep. 17, 2026

(54) PYRIDO[4,3-D]PYRIMIDINE DERIVATIVES AS MUTANT KRAS G12C INHIBITORS FOR THE TREATMENT OF CANCER

(71) Applicant: Frontier Medicines Corporation, South San Francisco, CA (US)

(72) Inventors: Philip A. GERKEN, San Francisco, CA (US); Snahel PATEL, Foster City, CA (US); Monika J. WILLIAMS, South San Francisco, CA (US)

(21) Appl. No.: 19/480,293

(22) PCT Filed: May 3, 2024
(86) PCT No.: PCT/US2024/027882

§ 371 Date: Oct. 30, 2025

Related U.S. Application Data
(60) Provisional application No. 63/464,170, filed on May 4, 2023.

(51) Int. Cl. C07D 519/00; A61K 31/519; A61K 31/5377; C07D 471/04

(52) U.S. Cl. CPC C07D 519/00; A61K 31/519; A61K 31/5377; C07D 471/04

(57) ABSTRACT

The present disclosure provides compounds of the formulae (I) to (VII) for use in the treatment and suppression of cancer, for example, useful for treating or suppressing cancers KRAS G12C. Also provided are pharmaceutical formulations containing such compounds and processes for preparing such compounds.

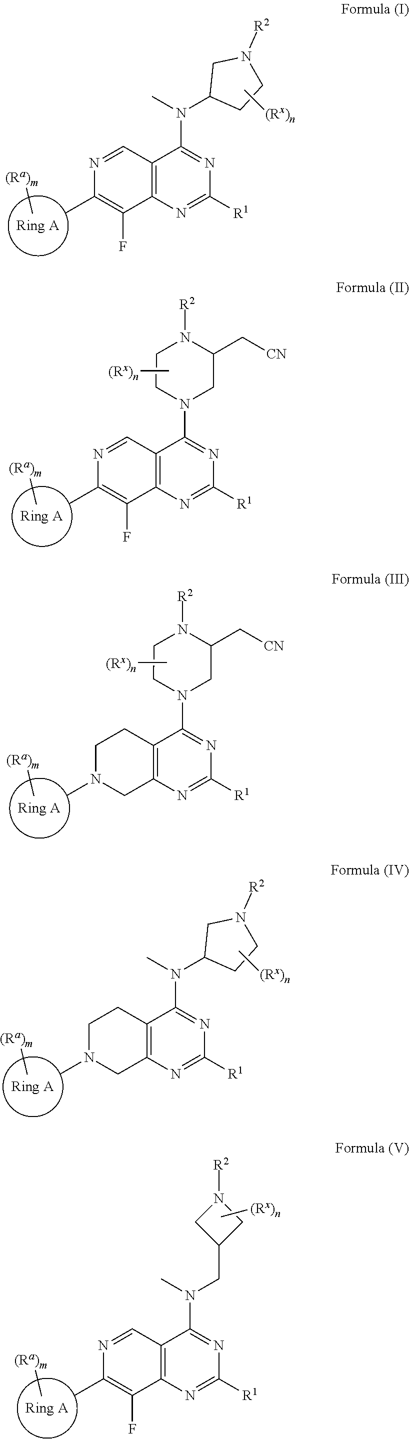

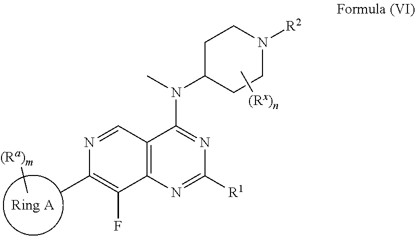

### CROSS-REFERENCE TO RELATED APPLICATIONS

This application claims priority to and benefit of U.S. Provisional Patent Application No. 63/464,170, filed on May 4, 2023, the disclosure of which is hereby incorporated herein by reference in its entirety.

### FIELD OF THE DISCLOSURE

The present disclosure provides compounds useful in treating or suppressing cancer, and in particular, useful in treating or suppressing cancers characterized by the KRAS G12C mutant. Also provided are pharmaceutical formulations containing such compounds, processes for preparing such compounds, and methods of using such compounds in the treatment or suppression of cancers.

### BACKGROUND

KRAS is a molecular switch. Under normal physiological conditions, the protein is bound to guanosine diphosphate (GDP) in the “off state.” In response to signaling through receptor tyrosine kinases (RTKs) such as EGFR, the GDP is exchanged to guanosine triphosphate (GTP) in a process facilitated by guanine nucleotide exchange factors (GEFs) such as SOS. The GTP-bound form of KRAS is in the “on state,” and interacts with proteins such as RAF and PI3K to promote downstream signaling that leads to cell proliferation and survival. KRAS can slowly hydrolyze GTP back to GDP, thus returning to the off-state, in a process facilitated by GAPs (GTPase-activating Proteins).

KRAS mutations are found in approximately 30% of all human cancers, and are highly prevalent among three of the deadliest forms of cancer: pancreatic (95%), colorectal (45%), and lung (35%). Together, these cancers occur in more than 200,000 patients annually in the US alone. One particular mutation, a glycine to cysteine substitution at position 12 (G12C), occurs in more than 40,000 patients per year. The KRAS G12C mutation impairs hydrolysis of GTP to GDP, thus trapping KRAS in the on-state and promoting cancer cell proliferation.

The cysteine residue of G12C provides an opportunity to develop targeted covalent drugs for this mutant KRAS. Early clinical trial results for KRAS G12C inhibitors AMG 510 and MRTX849 have shown encouraging results for non-small cell lung cancer (NSCLC), but the data are less compelling for colorectal cancer (CRC). Moreover, even in cases where patients respond to initial treatment, there are signs that the response may be limited in duration and that resistance could arise rapidly.

Most inhibitors of KRAS mutants bind preferentially to the GDP-bound form of the protein. For example, Amgen KRAS inhibitor AMG 510 and Mirati KRAS inhibitor MRTX849 react with the GDP-bound form of KRAS G12C at least 1000-fold more rapidly than with the GTP-bound form of the protein. One form of resistance that has been observed is for cancer cells to increase signaling through RTKs, thus increasing the amount of GTP-bound KRAS, which is less affected by current inhibitors. Thus, creating a molecule that could bind to and inhibit both the GDP- and GTP-bound forms of KRAS could have substantial utility.

What is needed are compounds useful in the treatment of cancer, such as cancers characterized by KRAS G12C. What is further needed are compounds useful in the treatment of cancers characterized by KRAS G12C, wherein the compounds bind to and inhibit both the inactive GDP- and activated GTP-bound forms of KRAS. What is further needed are compounds useful in the treatment of cancers characterized by KRAS G12C, wherein the compound has improved inhibition of the GTP-bound form of KRAS G12C.

### SUMMARY

In one aspect, the invention provides a compound of Formula (I), Formula (II) Formula (III), Formula (IV), Formula (V) or Formula (VI), wherein:

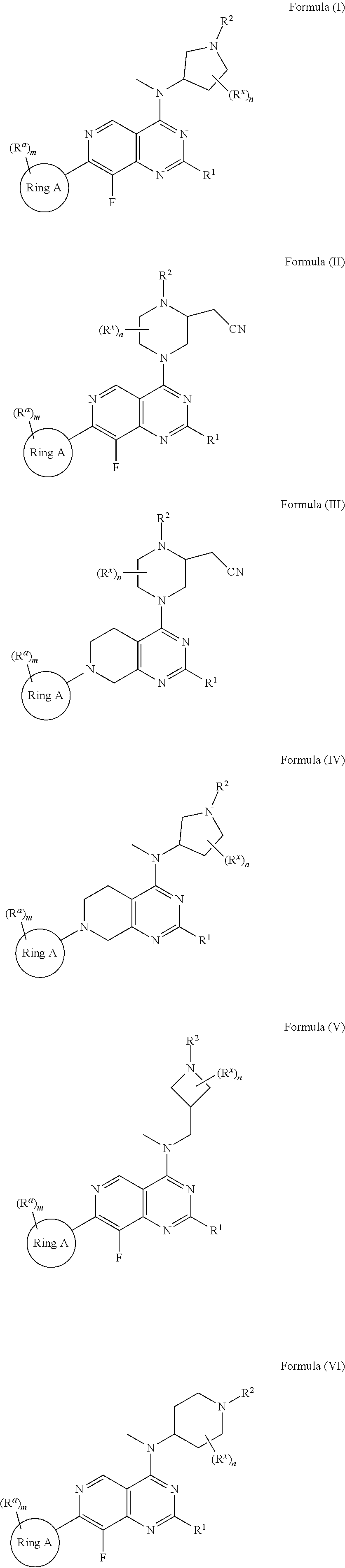

or a salt thereof; and/or an isotopologue thereof; wherein:
Ring A is a 6-10 membered aryl or a 5-10 membered heteroaryl;
each R^{a} is independently selected from the group consisting of halo, —OH, —NH₂, C₁-C₄ alkyl, C₃-C₄ cycloalkyl, C₁-C₄ alkoxy, C₁-C₄ haloalkyl, C₁-C₄ haloalkoxy, and C₂-C₃ alkynyl;
m is 0, 1, 2, or 3;
R¹ is selected from the group consisting of H, —OCH₂R^{1A}, —OC₁-C₄ alkyl, —C₁-C₄ alkyl, and

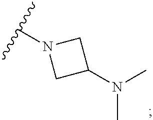 [SMILES: *N1CC(N(C)C)C1]

R^{1A} is selected from the group consisting of

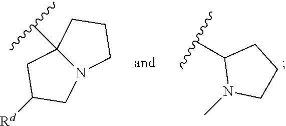

R^{d} is H or F;
R² is

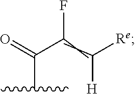

R^{e} is selected from the group consisting of —COOH, —C(O)O—C₁-C₄ alkyl, —C(O)O—C₁-C₄ haloalkyl, —C(O)—C₁-C₄ alkyl, —C(O)—C₁-C₄ haloalkyl, —C(O)N(C₁-C₄ alkyl)₂, —(C₁-C₂ alkyl)-(C₁-C₂ alkoxy), —S(O)₂—C₁-C₄ alkyl, —S(O)₂—C₁-C₄ haloalkyl, R^{e1}, —C(R^{Y1})(R^{Y2})R^{e}, R^{e2}, and —C(R^{Y1})(R^{Y2})R^{e3};
R^{Y1} and R^{Y2} in each occurrence are independently —H or —CH₃;
R^{e1} is a 4-10 membered heterocycle which is substituted with 0, 1, 2, 3, or 4 substituents independently selected from the group consisting of halo, hydroxy, C₁-C₄ alkyl, C₁-C₆ alkoxy, C₁-C₆alkoxyalkoxy, C₁-C₄ haloalkyl, and C₁-C₄ haloalkoxy;
R^{e2} is a 5-6 membered heteroaryl group substituted with 0, 1, 2, or 3 substituents independently selected from the group consisting of halo, hydroxy, C₁-C₄ alkyl, C₁-C₄ hydroxyalkyl, C₁-C₄ alkoxy, C₁-C₄ haloalkyl, C₁-C₄ haloalkoxy, C₃-C₆ heterocyclyl substituted with 0, 1, or 2 substituents independently selected from the group consisting of halo, hydroxy, and methyl, and C₃-C₆ cycloalkyl substituted with 0, 1, or 2 substituents independently selected from the group consisting of halo, hydroxy, and methyl;
R^{e3} is —NR³¹R³²;
R³¹ and R³² are independently selected from the group consisting of hydrogen, C₁-C₄ alkyl, C₁-C₄ alkoxy, C₁-C₄ haloalkoxy, and C₁-C₄ alkyl substituted with a 3-6 membered heterocycle;
each R^{x} is independently selected from the group consisting of —OH, halo, and C₁-C₄ alkyl; and
n is 0, 1, or 2.

In some embodiments, if R^{e} of Formula (II) is R^{e2}, then the 5-6 membered heteroaryl group of R^{e2} is substituted with 1, 2, or 3 substituents; and if R¹ of Formula (III) is

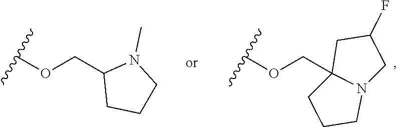

then

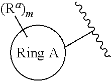

is not

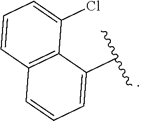 [SMILES: *c1cccc2cccc(Cl)c12]

In some embodiments, including any of the embodiments in the preceding paragraphs, the compound is selected from the group consisting of the compounds of Table 1; and all salts and isotopologues thereof.

In another aspect provided is a pharmaceutical formulation comprising a compound as described herein, including but not limited to a compound described in the preceding paragraphs, and a pharmaceutically acceptable carrier, wherein when the compound is a salt, the salt is a pharmaceutically acceptable salt.

In another aspect provided is a method of treating or suppressing cancer comprising: administering a therapeutically effective amount of a compound as described herein, including but not limited to a compound described in the preceding paragraphs, or a pharmaceutical formulation, including but not limited to the pharmaceutical formulation described in the preceding paragraphs, to a subject in need thereof, wherein when the compound is a salt, the salt is a pharmaceutically acceptable salt. In some embodiments, the cancer is selected from the group consisting of: lung, colorectal, pancreatic, bile duct, thyroid, gall bladder, uterine, mesothelioma, cervical, and bladder cancers. In some embodiments, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, non-small cell lung cancer (NSCLC), and melanoma. In some embodiments, including any of the foregoing embodiments, the method is for treating the cancer. In some embodiments, including any of the foregoing embodiments, the method is for suppressing the cancer. In some embodiments, including any of the foregoing embodiments, the cancer is a KRAS G12C mediated cancer. In some embodiments, including any of the foregoing embodiments, the subject has been diagnosed as having a KRAS G12C mediated cancer. In some embodiments, the method further comprises administering to the subject a therapeutically effective amount of an additional chemotherapeutic agent.

In another aspect provided is the use of a compound as described herein, including but not limited to any of the foregoing embodiments, as a medicament. In another aspect is the use of a compound as described herein, including but not limited to any of the foregoing embodiments, for treating or suppressing cancer. In another aspect is the use of a compound as described herein, including but not limited to any of the foregoing embodiments, in the manufacture of a medicament for use in treating or suppressing cancer. In some embodiments, including any of the foregoing embodiments, the use is for treating the cancer. In some embodiments, including any of the foregoing embodiments, the use is for suppressing the cancer.

In another aspect provided is a compound as described herein, including but not limited to any of the foregoing embodiments for use in the manufacturing of a medicament for treating or suppressing cancer. In another aspect is a compound as described herein, including but not limited to any of the foregoing embodiments, for use in treating or suppressing cancer. In another aspect is the compound as described herein, including but not limited to any of the foregoing embodiments, for use in the manufacture of a medicament for treating or suppressing cancer. In some embodiments, including any of the foregoing embodiments, the use is for treating the cancer. In some embodiments, including any of the foregoing embodiments, the use is for suppressing the cancer.

It is to be understood that the description of compounds, compositions, formulations, and methods of treatment described herein include “comprising”, “consisting of”, and “consisting essentially of” embodiments. In some embodiments, for all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

Additional embodiments, features, and advantages of the present disclosure will be apparent from the following detailed description and through practice of the present disclosure.

### DETAILED DESCRIPTION

Provided herein are compounds useful in treating cancer, and methods of using such compounds for treating cancer. In some embodiments, the compounds are useful in treating cancers characterized by KRAS G12C. In some embodiments, the compounds advantageously inhibit both the inactive GDP- and activated GTP-bound forms of KRAS G12C. In some embodiments, the compounds advantageously have improved inhibition of the GTP-bound form of KRAS G12C.

### Definitions

The abbreviations used herein have their conventional meaning within the chemical and biological arts, unless otherwise specified.

It is to be understood that descriptions of compound structures, including possible substitutions, are limited to those which are chemically possible.

Unless otherwise indicated, the absolute stereochemistry of all chiral atoms is as depicted. Compounds that have a stereogenic center where the configuration is not indicated in the structure as depicted are mixtures of enantiomers at that center.

A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

Reference to “about” a value or parameter herein includes (and describes) variations that are directed to that value or parameter per se. For example, description referring to “about X” includes description of “X”. As used herein, and unless otherwise specified, the terms “about” and “approximately,” when used in connection with temperatures, doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 15%, within 10%, within 5%, within 4%, within 3%, within 2%, within 1%, or within 0.5% of the specified dose, amount, or weight percent.

The terms “a” and “an,” as used in herein mean one or more, unless context clearly dictates otherwise.

The terms “subject,” “individual,” and “patient” mean an individual organism, preferably a vertebrate, more preferably a mammal, most preferably a human. Examples of patients include humans, livestock such as cows, goats, sheep, pigs, and rabbits, and companion animals such as dogs, cats, and horses. In some embodiments, the subject has been identified or diagnosed as having a cancer or tumor having a KRAS G12C mutation (e.g., as determined using a regulatory agency-approved, e.g., FDA-approved, assay or kit).

“Treating” a disorder with the compounds and methods discussed herein is defined as administering one or more of the compounds discussed herein, with or without additional therapeutic agents, in order to reduce or eliminate either the disorder or one or more symptoms of the disorder, or to retard the progression of the disorder or of one or more symptoms of the disorder, or to reduce the severity of the disorder or of one or more symptoms of the disorder.

“Suppression” of a disorder with the compounds and methods discussed herein is defined as administering one or more of the compounds discussed herein, with or without additional therapeutic agents, in order to suppress the clinical manifestation of the disorder, or to suppress the manifestation of adverse symptoms of the disorder. The distinction between treatment and suppression is that treatment occurs after adverse symptoms of the disorder are manifest in a subject, while suppression occurs before adverse symptoms of the disorder are manifest in a subject. Suppression may be partial, substantially total, or total. In some embodiments, genetic screening can be used to identify patients at risk of the disorder. The compounds and methods disclosed herein can then be administered to asymptomatic patients at risk of developing the clinical symptoms of the disorder, in order to suppress the appearance of any adverse symptoms.

“Therapeutic use” of the compounds discussed herein is defined as using one or more of the compounds discussed herein to treat or suppress a disorder, as defined herein. A “therapeutically effective amount” of a compound is an amount of the compound, which, when administered to a subject, is sufficient to reduce or eliminate either the disorder or one or more symptoms of the disorder, or to retard the progression of the disorder or of one or more symptoms of the disorder, or to reduce the severity of the disorder or of one or more symptoms of the disorder, or to suppress the clinical manifestation of a disorder, or to suppress the manifestation of adverse symptoms of a disorder. A therapeutically effective amount can be given in one or more administrations.

A “KRAS G12C mediated cancer” is used interchangeably herein with a “cancer characterized by KRAS G12C”, and indicates that the cancer comprises cells which contain the KRAS G12C mutant.

While the compounds described herein can occur and can be used as the neutral (non-salt) compound, the description is intended to embrace all salts of the compounds described herein, as well as methods of using such salts of the compounds. In some embodiments, the salts of the compounds comprise pharmaceutically acceptable salts.

A “pharmaceutically acceptable salt” of a compound means a salt that is pharmaceutically acceptable to humans and/or animals, and which, upon administration, retains at least some of the desired pharmacological activity of the parent compound. Such salts include: (a) acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (b) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. Additional information on suitable pharmaceutically acceptable salts can be found in Remington's Pharmaceutical Sciences, 17th ed., Mack Publishing Company, Easton, PA, 1985, which is incorporated herein by reference in its entirety.

Included herein, when chemically relevant, are all stereoisomers of the compounds, including diastereomers and enantiomers. Also included are mixtures of possible stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

“Isotopologue” refers herein to a compound which differs in its isotopic composition from its “natural” isotopic composition. “Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition. The description of compounds herein also includes all isotopologues, in some embodiments, partially deuterated or perdeuterated analogs, of all compounds herein. “Isotopically enriched” may also refer to a compound containing at least one atom having an isotopic composition other than the natural isotopic composition of that atom. “Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Alkyl” means a linear, branched, or a combination thereof, saturated monovalent hydrocarbon radical having the defined number of carbons. For example, C₁-C₄ alkyl includes e.g., methyl, ethyl, propyl, 2-propyl, butyl, and the like.

“Alkylene” means a linear, branched, or a combination thereof, saturated divalent hydrocarbon radical having the defined number of carbons. For example, C₁-C₄ alkylene includes e.g., methylene, ethylene, propylene, 1-methylpropylene, 2-methylpropylene, butylene, and the like.

“Alkenyl” means a linear or branched monovalent hydrocarbon radical containing one or more double bonds and having the defined number of carbons. For example, C₂-C₄ alkenyl includes e.g., vinyl, prop-1-en-2-yl, prop-1-en-1-yl, allyl and the like.

“Alkynyl” means a linear or branched monovalent hydrocarbon radical containing one or more triple bonds and having the defined number of carbons. For example, C₂-C₄ alkyne includes e.g., ethynyl, propynyl, 2-propynyl, butynyl, and the like.

“Alkoxy” means an —OR^{x}′ radical where R^{x}′ is alkyl as defined above, or a —R^{x}″OR^{x}′″ radical where R^{x}″ is an alkylene and R^{x}′″ is an alkyl group as defined above where the defined number of alkyl carbons in the alkoxy group are equal to the total number of carbons in R^{x}″ and R^{x}′″. For example, C₁-C₄ alkoxy indicates e.g., methoxy, ethoxy, propoxy, 2-propoxy, n-, iso-, tert-butoxy, methoxymethyl, ethoxymethyl, propoxymethyl, isopropoxymethyl, and the like. In some embodiments, alkoxy is a —OR^{x}′ radical. In some embodiments, alkoxy is a —R^{x}″OR^{x}′″ radical. In some embodiments, when a nitrogen is substituted with an alkoxy group, the alkoxy group is not linked to the nitrogen via the oxygen or a carbon that is immediately adjacent to the oxygen in the alkoxy group. For example, the alkoxy-substituted nitrogen is not N—OR^{x}′ or N—CH₂—O—R^{x}′″.

“Alkoxyalkoxy” means an —OR^{y} radical where R^{y} is alkoxy as defined above, provided that the attachment point of R is not an oxygen atom, or a —R^{Y}′OR^{Y}″ radical where R^{Y}′ is an alkylene and R^{Y}″ is an alkoxy group as defined above, provided that the attachment point of R^{Y}″ is not an oxygen atom, where the defined number of alkyl carbons in the alkoxyalkoxy group are equal to the total number of carbons in R^{Y}′ and R^{y}″. For example, C₁-C₆ alkoxyalkoxy indicates e.g., —OCH₂OCH₃, —OCH₂CH₂OCH₃, —OCH₂CH₂OCH₃, —CH₂OCH₂OCH₃, —CH₂OCH₂CH₂OCH₃, —CH₂OCH₂CH₂OCH₂CH₃, —CH₂CH₂OCH₂CH₂OCH₂CH₃ and the like. In some embodiments, alkoxyalkoxy is a —OR^{y} radical. In some embodiments, alkoxyalkoxy is a —R^{Y}′OR^{y}″ radical. In some embodiments, when a nitrogen is substituted with an alkoxyalkoxy group, the alkoxyalkoxy group is not linked to the nitrogen via the oxygen or a carbon that is immediately adjacent to the oxygen in the alkoxyalkoxy group. For example, the alkoxyalkoxy-substituted nitrogen is not N—OR^{y} or N—CH₂—O—R^{y}″.

“Aminoalkyl” means an —NHR^{z} radical where R^{z} is alkyl as defined above, or a —NR^{z}R^{z}′ radical where R^{z} and R^{z}′ are alkyl groups as defined above, or an —R^{z}″NH₂ radical where R^{z}″ is an alkylene group as defined above, or an —R^{z}″NHR^{z} radical where R^{z}″ is an alkylene group as defined above and R^{z}′ is an alkyl group as defined above, or a —R^{z}″NR^{z}Rfz′ radical where R^{z}″ is an alkylene group as defined above and R^{z} and R^{z}′ are alkyl groups as defined above, where the defined number of alkyl carbons in the aminoalkyl group is equal to the total number of carbons in R^{z}, R^{z}′ and R^{z}″ as applicable. For example, C₁-C₆ aminoalkyl indicates e.g., —NHCH₃, —NHCH₂CH₃, —NHCH₂(CH₃)₂, —N(CH₃)₂, —N(CH₃)CH₂CH₃, —N(CH₂CH₃)₂, —CH₂NH₂, —CH₂CH₂NH₂, —CH₂NHCH₃, —CH₂N(CH₃)₂, —CH₂CH₂NHCH₃, —CH₂CH₂N(CH₃)₂ and the like. In some embodiments, aminoalkyl is an —NHR^{z} radical. In some embodiments, aminoalkyl is an —NR^{z}R^{z}′ radical. In some embodiments, an aminoalkyl is an —R^{z}″NH₂ radical. In some embodiments, aminoalkyl is a —R^{z}″NHR^{z} radical. In some embodiments, aminoalkyl is a —R^{z}″NR^{z}R^{z}′ radical. In some embodiments, when an oxygen is substituted with an aminoalkyl group, the aminoalkyl group is not linked to the oxygen via the nitrogen or a carbon that is immediately adjacent to the nitrogen in the aminoalkyl group. For example, the aminoalkyl-substituted oxygen is not O—NR^{z} or O—CH₂—NHR^{z}.

“Aryl” refers to a radical of a monocyclic or polycyclic (e.g., bicyclic or tricyclic) aromatic ring system having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). In some embodiments, “aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryl groups include phenyl and naphthyl, wherein the attachment point can be on any carbon atom. Exemplary aryl groups also include indenyl, tetrahydronaphthyl, indolinyl, benzodihydrofuranyl, tetrahydroquinolinyl, tetrahydroisoquinolinyl and the like, wherein the attachment point is on the phenyl group. In some embodiments, “aryl” excludes ring systems wherein the aryl ring, as defined above, is fused with one or more carbocyclyl or heterocyclyl groups.

“Cycloalkyl” means a monocyclic saturated monovalent hydrocarbon radical having the defined number of carbon atoms. For example, C₃-C₆ cycloalkyl includes cyclopropyl, cyclobutyl, cyclopentyl, and cyclohexyl.

“Cycloalkylene” means a monocyclic saturated divalent hydrocarbon radical having the defined number of carbon atoms. For example, C₃-C₆ cycloalkylene includes cyclopropylene, cyclobutylene, cyclopentylene, and cyclohexylene.

“Cyanoalkyl” means an alkyl radical as defined above, which is substituted with a cyano group (—CN). A cyanoalkyl can also be referred to as an alkylnitrile.

“Halo” means fluoro, chloro, bromo, or iodo. In some embodiments, halo is fluoro or chloro.

“Haloalkyl” means an alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means an —OR^{a′} radical where R^{a′} is haloalkyl as defined above, or a —R^{b′}OR^{c′} radical where R^{b′} and R^{c′} are alkyl or haloalkyl groups as defined above where the defined number of alkyl carbons in the haloalkoxy group are equal to the total number of carbons in R^{b′} and R^{c′}. Halo atom(s) may be present in R^{b′}, or R^{c′}, or both, provided that at least one of R^{b′} and R^{c′} comprises a halo atom. For example, C₁-C₄ haloalkoxy indicates e.g., —OCF₃, —OCHF₂, —CH₂OCF₃, —CH₂CH(F)CH₂OCH₃, —CH₂CH(F)CH₂OCHF₂, and the like. In some embodiments, haloalkoxy is a —OR^{a′} radical. In some embodiments, haloalkoxy is a —R^{b′}OR^{c′} radical. When all of the halo atom(s) in the haloalkoxy group are fluoro, it can be referred to in this Application as fluoroalkoxy. In some embodiments, when a nitrogen is substituted with a haloalkoxy group, the haloalkoxy group is not linked to the nitrogen via the oxygen or a carbon that is immediately adjacent to the oxygen in the haloalkoxy group. For example, the haloalkoxy-substituted nitrogen is not N—OR^{a′} or N—C(H)ₙ(X)ₘO—R″ (wherein X is a halogen and n and m are integers, provided that m+n=2).

“Hydroxyalkyl” means an alkyl radical as defined above, which is substituted with one or more hydroxyl (—OH) groups, e.g., one to three hydroxyl groups, e.g., —CH₂OH, —CH₂CH₂OH, —C(OH)(CH₃)₂, —CH(OH)CH₃ and the like.

A “heterocyclic group” or “heterocycle”, unless otherwise specified, means a saturated or partially unsaturated cyclic group comprising 3-12 ring atoms, in which 1-4 ring atoms are heteroatoms independently selected from the group consisting of nitrogen, oxygen, and sulfur, the remaining rings being C. The sulfur group may be present either as —S— or as —S(O)₂—. Unless otherwise specified, the heterocyclic group includes single as well as multiple ring systems including fused, bridged, and spiro ring systems. “Heterocyclic group” or “heterocycle” also includes ring systems wherein the heterocyclic group, as defined above, is fused with one or more carbocyclic groups wherein the point of attachment is either on the carbocycle or heterocycle ring In some embodiments, “heterocyclic group” or “heterocycle” also includes ring systems wherein the heterocyclic group, as defined above, is fused with one or more aryl or heteroaryl groups, wherein the point of attachment is on the heterocyclyl ring, and in such instances, the number of ring members continue to designate the number of ring members in the heterocyclyl ring system. In some embodiments, the heterocyclic group is a single ring. In some embodiments, the heterocyclic group comprises two fused rings. In some embodiments, the heterocyclic group comprises two spiro rings. In some embodiments, the heterocyclic group comprises a bridged ring system.

A “carbocyclic group” or “carbocycle”, unless otherwise specified, means a saturated or partially unsaturated cyclic group comprising 3-12 ring atoms, in which the ring atoms are C. Unless otherwise specified, the carbocyclic group includes single as well as multiple ring systems including fused, bridged, and spiro ring systems. In some embodiments, the carbocyclic group is a single ring. In some embodiments, the carbocyclic group comprises two fused rings. In some embodiments, the carbocyclic group comprises two spiro rings. In some embodiments, the carbocyclic group comprises a bridged ring system.

“Heteroaryl” means a monovalent monocyclic or bicyclic aromatic radical of 5 to 10 ring atoms, unless otherwise stated, where one or more (in some embodiments, one, two, or three) ring atoms are heteroatom(s) independently selected from N, O, or S, the remaining ring atoms being carbon. In some embodiments, “heteroaryl” includes ring systems wherein the heteroaryl ring, as defined above, is fused with one or more carbocyclyl or heterocyclyl groups wherein the point of attachment is on the heteroaryl ring, In such instances, unless otherwise specified, the number of ring members continue to designate the number of ring members in the heteroaryl ring system. In some embodiments, “heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). In some embodiments, “heteroaryl” excludes ring systems wherein the heteroaryl ring is fused with a carbocyclyl or heterocyclyl group. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

A “spiro” cycloalkyl group indicates that the cycloalkyl group is linked to the remaining portion of the compound through a spiro linkage. A “spiro” cycloalkyl substituent has two attachments that connect to the same carbon of the moiety that is substituted, forming a spiro connection. For example, a cyclohexyl group that is substituted with a “spiro C₃-C₄ cycloalkyl” group indicates:

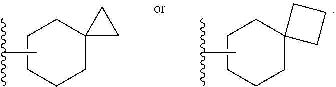

“In need of treatment” as used herein means the patient is being treated by a physician or other caregiver after diagnoses of the disease, or a determination that the patient is at risk for developing the disease. In some embodiments, the patient has been diagnosed as having a KRAS G12C mediated cancer. In some embodiments, the patient has been determined to be at risk of developing a KRAS G12C mediated cancer.

“Administration”, “administer” and the like, as they apply to, for example, a patient, cell, tissue, organ, or biological fluid, refer to contact of, for example, a compound of Formula (I), Formula (II), Formula (III), Formula (IV), Formula (V), Formula (VI), or a pharmaceutically acceptable salt and/or isotopologue thereof, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Optional” or “optionally” means that the subsequently described event or circumstance may but need not occur, and that the description includes instances where the event or circumstance occurs and instances in which it does not.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule or a tablet having a fixed ratio of active ingredients or in multiple, separate capsules or tablets for each active ingredient. In addition, such administration also encompasses use of each type of therapeutic agent in a sequential manner. In either case, the treatment regimen will provide beneficial effects of the drug combination in treating the conditions or disorders described herein.

### Compounds

Provided herein are compounds of Formula (I), Formula (II), Formula (III), Formula (IV), Formula (V) and Formula (VI). Unless the context requires otherwise, reference throughout this specification to “a compound of Formula (I), Formula (II), Formula (III), Formula (IV), Formula (V) or Formula (VI)” or “compounds of Formula (I), Formula (II), Formula (III), Formula (IV), Formula (V) or Formula (VI)” refers to all embodiments of Formula (I), Formula (II), Formula (III), Formula (IV), Formula (V) and Formula (VI) including, for example, compounds of Formulas (I-a), (I-b), (II-a), (II-b), (III-a), (III-b), (IV-a), (IV-b), (V-a), (V-b), (VI-a), (VI-b) as well as the compounds of Table 1. In some embodiments, provided are compounds of Formula (I), Formula (II), Formula (III), Formula (IV), Formula (V), Formula (VI) or pharmaceutically acceptable salts thereof. In some embodiments, the compounds of Formula (I), Formula (II), Formula (III), Formula (IV), Formula (V), Formula (VI) are provided as pharmaceutically acceptable salts. In some embodiments, the compounds of Formula (I), Formula (II), Formula (III), Formula (IV), Formula (V), Formula (VI) are provided as the corresponding free base (i.e., are not salts).

In an embodiment, provided is a compound of Formula (I), Formula (II), Formula (III), Formula (IV), Formula (V) or Formula (VI)

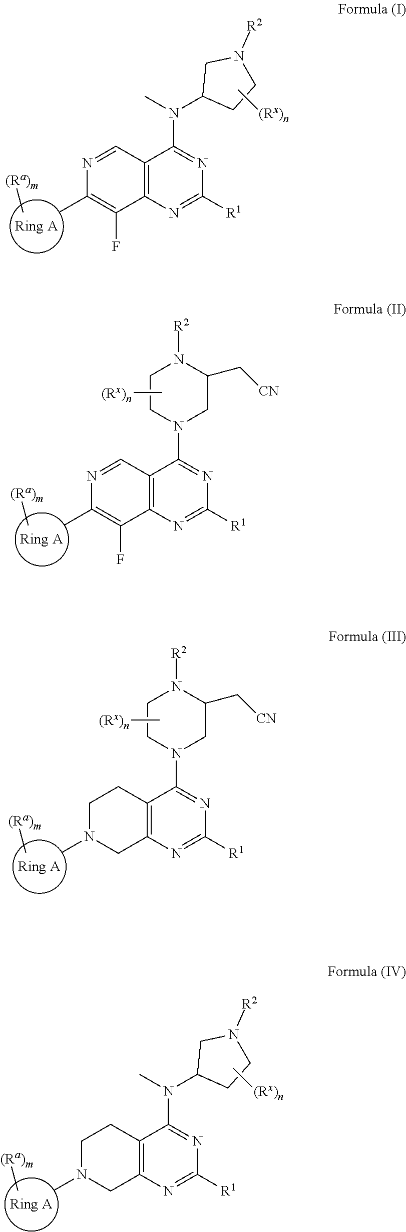

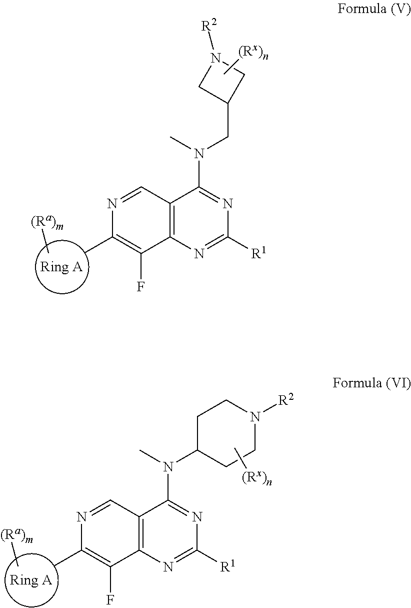

or a salt thereof; and/or an isotopologue thereof; wherein:
Ring A is a 6-10 membered aryl or a 5-10 membered heteroaryl;
each R^{a} is independently selected from the group consisting of halo, —OH, —NH₂, C₁-C₄ alkyl, C₃-C₄ cycloalkyl, C₁-C₄ alkoxy, C₁-C₄ haloalkyl, C₁-C₄ haloalkoxy, and C₂-C₃ alkynyl;
m is 0, 1, 2, or 3;
R¹ is selected from the group consisting of H, —OCH₂R^{1A}, —OC₁-C₄ alkyl, —C₁-C₄ alkyl, and

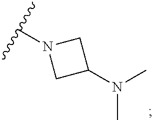 [SMILES: *N1CC(N(C)C)C1]

R^{1A} is selected from the group consisting of

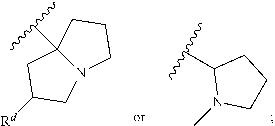

R^{d} is H or F;
R² is

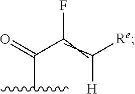

R^{e} is selected from the group consisting of —COOH, —C(O)O—C₁-C₄ alkyl, —C(O)O—C₁-C₄ haloalkyl, —C(O)—C₁-C₄ alkyl, —C(O)—C₁-C₄ haloalkyl, —C(O)N(C₁-C₄ alkyl)₂, —(C₁-C₂ alkyl)-(C₁-C₂ alkoxy), —S(O)₂—C₁-C₄ alkyl, —S(O)₂—C₁-C₄ haloalkyl, R^{e1}, —C(R^{Y1})(R^{Y2})R^{e}, R^{e2}, and —C(R^{Y1})(R^{Y2})R^{e3};
R^{Y1} and R^{Y2} in each occurrence are independently —H or —CH₃;
R^{e1} is a 4-10 membered heterocycle which is substituted with 0, 1, 2, 3, or 4 substituents independently selected from the group consisting of halo, hydroxy, C₁-C₄ alkyl, C₁-C₆ alkoxy, C₁-C₆alkoxyalkoxy, C₁-C₄ haloalkyl, and C₁-C₄ haloalkoxy; R^{e2} is a 5-6 membered heteroaryl group substituted with 0, 1, 2, or 3 substituents independently selected from the group consisting of halo, hydroxy, C₁-C₄ alkyl, C₁-C₄ hydroxyalkyl, C₁-C₄ alkoxy, C₁-C₄ haloalkyl, C₁-C₄ haloalkoxy, C₃-C₆ heterocyclyl substituted with 0, 1, or 2 substituents independently selected from the group consisting of halo, hydroxy, and methyl, and C₃-C₆ cycloalkyl substituted with 0, 1, or 2 substituents independently selected from the group consisting of halo, hydroxy, and methyl;
R^{e3} is —NR³¹R³²;
R³¹ and R³² are independently selected from the group consisting of hydrogen, C₁-C₄ alkyl, C₁-C₄ alkoxy, C₁-C₄ haloalkoxy, and C₁-C₄ alkyl substituted with a 3-6 membered heterocycle;
each R^{x} is independently selected from the group consisting of —OH, halo, and C₁-C₄ alkyl; and
n is 0, 1, or 2.

In some embodiments, if R^{e} of Formula (II) is R^{e2}, then the 5-6 membered heteroaryl group of R^{e2} is substituted with 1, 2, or 3 substituents. In some embodiments, if R¹ of Formula (II) is

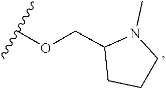 [SMILES: *OCC1CCCN1C]

then

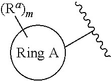

is not

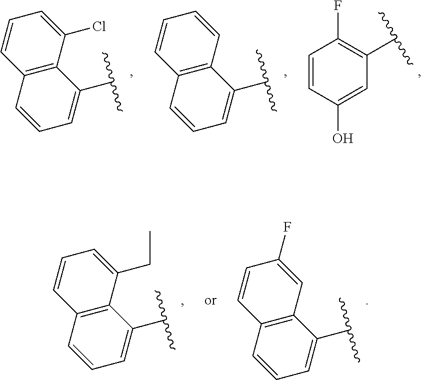

In some embodiments,

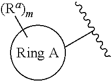

### of Formula (II) is not

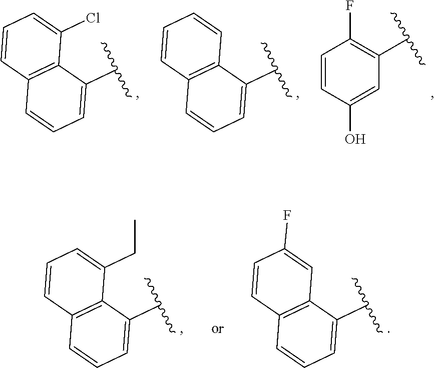

In some embodiments, if R¹ of Formula (II) is

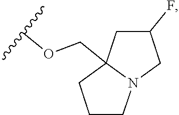 [SMILES: *OCC12CCCN1CC(F)C2]

then

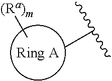

is not

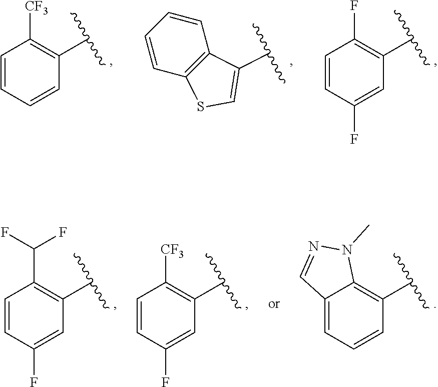 [SMILES: *c1ccccc1C(F)(F)F ; *c1csc2ccccc12 ; *c1cc(F)ccc1F ; *c1cc(F)ccc1C(F)F ; *c1cc(F)ccc1C(F)(F)F ; *c1cccc2cnn(C)c12]

In some embodiments,

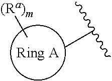

### of Formula (II) is not

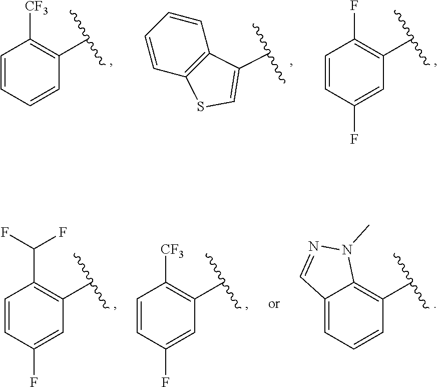 [SMILES: *c1ccccc1C(F)(F)F ; *c1csc2ccccc12 ; *c1cc(F)ccc1F ; *c1cc(F)ccc1C(F)F ; *c1cc(F)ccc1C(F)(F)F ; *c1cccc2cnn(C)c12]

In some embodiments, if R¹ of Formula (III) is or

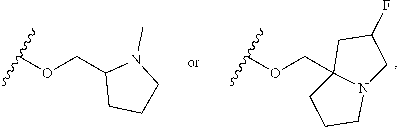

then

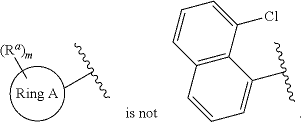

In some embodiments, if R^{e} of Formula (III) is R^{e2}, then the 5-6 membered heteroaryl group of R^{e2} is substituted with 1, 2, or 3 substituents. In some embodiments, if R^{e} of Formula (III) is R^{e2}, and R¹ is

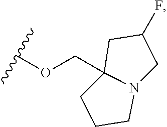 [SMILES: *OCC12CCCN1CC(F)C2]

then the 5-6 membered heteroaryl group of R^{e2} is substituted with 1, 2, or 3 substituents. In some embodiments, R¹ of Formula (III) is not

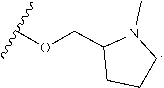 [SMILES: *OCC1CCCN1C]

In some embodiments, R¹ of Formula (III) is not

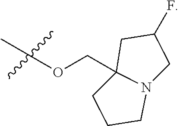 [SMILES: *OCC12CCCN1CC(F)C2]

In some embodiments,

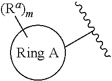

### of Formula (III) is not

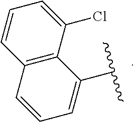 [SMILES: *c1cccc2cccc(Cl)c12]

In some embodiments, if R^{e} is R^{e2}, then the 5-6 membered heteroaryl group of R^{e2} is substituted with 1, 2, or 3 substituents. In some embodiments, if R¹ is

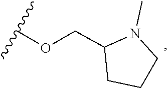 [SMILES: *OCC1CCCN1C]

then

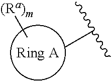

is not

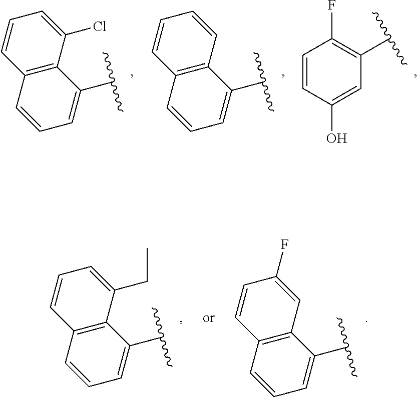

In some embodiments, if R¹ is

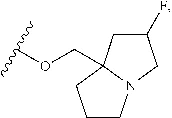 [SMILES: *OCC12CCCN1CC(F)C2]

then

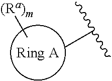

is not

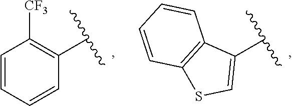 [SMILES: *c1ccccc1C(F)(F)F ; *c1csc2ccccc12]

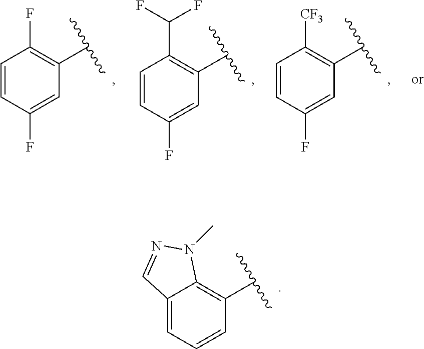 [SMILES: *c1cc(F)ccc1F ; *c1cc(F)ccc1C(F)F ; *c1cc(F)ccc1C(F)(F)F ; *c1cccc2cnn(C)c12]

In some embodiments,

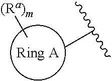

is not

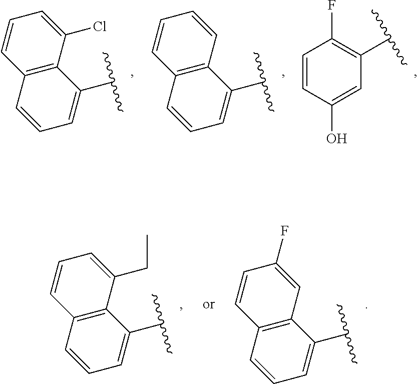

In some embodiments,

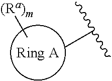

is not

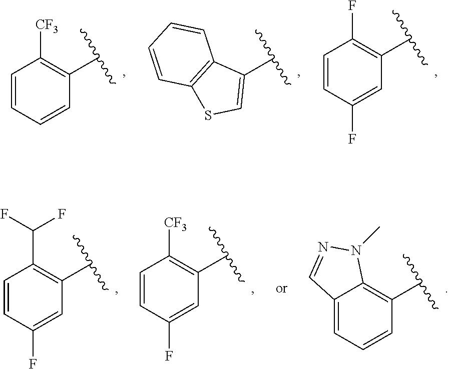 [SMILES: *c1ccccc1C(F)(F)F ; *c1csc2ccccc12 ; *c1cc(F)ccc1F ; *c1cc(F)ccc1C(F)F ; *c1cc(F)ccc1C(F)(F)F ; *c1cccc2cnn(C)c12]

In some embodiments, if R¹ is

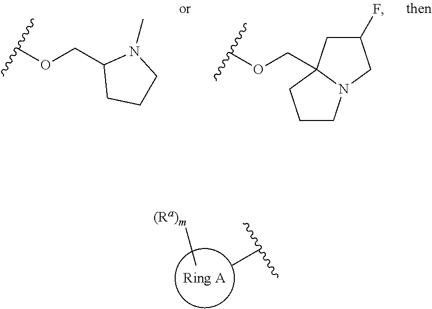

is not

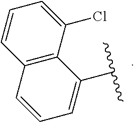 [SMILES: *c1cccc2cccc(Cl)c12]

In some embodiments, if R^{e} is R^{e2}, and R¹ is

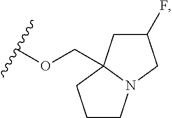 [SMILES: *OCC12CCCN1CC(F)C2]

then the 5-6 membered heteroaryl group of R^{e2} is substituted with 1, 2, or 3 substituents. In some embodiments, R¹ is not

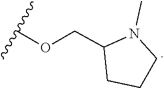 [SMILES: *OCC1CCCN1C]

In some embodiments, R¹ is not

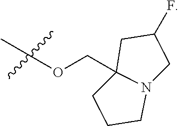 [SMILES: *OCC12CCCN1CC(F)C2]

In some embodiments,

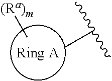

is not

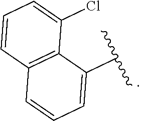 [SMILES: *c1cccc2cccc(Cl)c12]

In an embodiment, the compound is of Formula (I).

In an embodiment, the compound is of Formula (II).

In an embodiment, the compound is of formula (III).

In an embodiment, the compound is of formula (IV).

In an embodiment, the compound is of formula (V).

In an embodiment, the compound is of formula (VI).

In an embodiment, the stereochemistry of the pyrrolidine is (R) (i.e., the moiety represented as

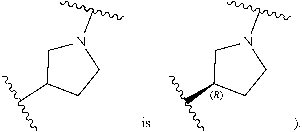

In an embodiment, the stereochemistry of the cyanomethyl groups is (S) (i.e., the moiety represented as

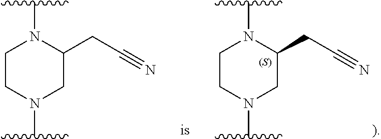

As generally defined herein, Ring A is a 6-10 membered aryl or a 5-10 membered heteroaryl.

In an embodiment, Ring A is selected from a 6-10 membered aryl and a 9-10 membered bicyclic heteroaryl having 1, 2 or 3 heteroatoms independently selected from N, O and S.

In an embodiment, Ring A is selected from the group consisting of naphthalenyl, phenyl, isoquinolinyl, indazolyl and pyridinyl. In an embodiment, Ring A is selected from the group consisting of naphthalenyl and phenyl.

In an embodiment, Ring A is naphthalenyl. In an embodiment, Ring A is phenyl.

In an embodiment, Ring A is selected from the group consisting of naphthalen-1-yl, phenyl, isoquinolin-1-yl, indazol-4-yl and pyridin-1-yl. In an embodiment, Ring A is selected from the group consisting of naphthalen-1-yl and phenyl. In an embodiment, Ring A is naphthalen-1-yl.

In an embodiment, Ring A is selected from the group consisting of:

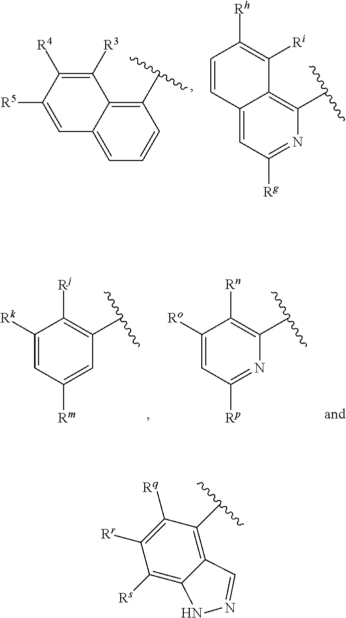

wherein each R³, R⁴, R⁵, R^{h}, Rⁱ, R^{j}, R^{k}, Rⁿ, R^{o}, R^{q}, R^{s}, R^{g}, R^{m} and R^{p} is as defined in any of the embodiments described herein.

In an embodiment, Ring A is selected from the group consisting of:

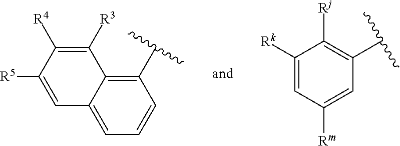

wherein each R³, R⁴, R^{h}, Rⁱ, R^{j}, R^{k}, and R^{m} is as defined in any of the embodiments described herein. In an embodiment, Ring A is

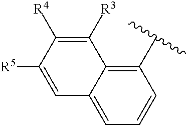

wherein each R³, R⁴ and R⁵ is as defined in any of the embodiments described herein. In an embodiment, Ring A is

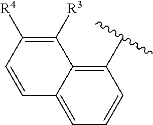

wherein each R³ and R⁴ is as defined in any of the embodiments described herein. In an embodiment, Ring A is

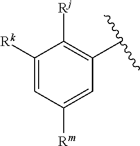

wherein each Rⁱ, R^{k}, and R^{m} is as defined in any of the embodiments described herein. In an embodiment, Ring A is selected from:

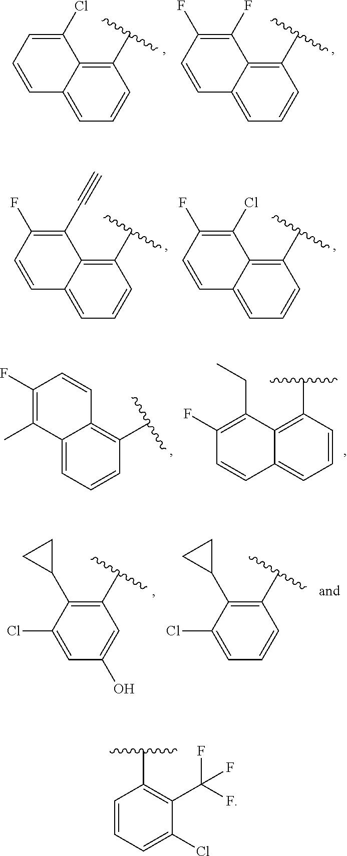

In an embodiment, Ring A is

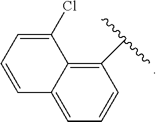 [SMILES: *c1cccc2cccc(Cl)c12]

In an embodiment, Ring A is

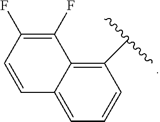 [SMILES: *c1cccc2ccc(F)c(F)c12]

an embodiment, Ring A is

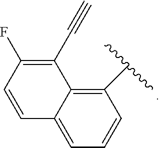 [SMILES: *c1cccc2ccc(F)c(C#C)c12]

In an embodiment, Ring A is

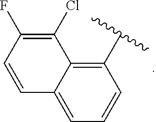 [SMILES: *c1cccc2ccc(F)c(Cl)c12]

In an embodiment, Ring A is

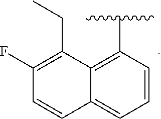 [SMILES: *c1cccc2ccc(F)c(CC)c12 ; "."]

In an embodiment, Ring A is

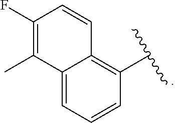 [SMILES: *c1cccc2c(C)c(F)ccc12]

In an embodiment, Ring A is

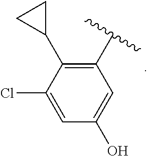 [SMILES: *c1cc(O)cc(Cl)c1C1CC1]

In an embodiment, Ring A is

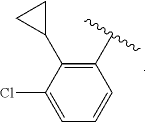 [SMILES: *c1cccc(Cl)c1C1CC1]

In an embodiment, Ring A is

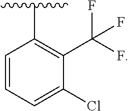 [SMILES: *c1cccc(Cl)c1C(F)(F)F]

As generally defined herein, each R^{a} is independently selected from halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{a} is independently selected from halo, —OH, C₁-C₄ alkyl, C₃-C₄ cycloalkyl, C₁-C₄ haloalkyl and C₂-C₃ alkynyl. In an embodiment each R^{a} is independently selected from halo, C₁-C₄ alkyl, C₃-C₄ cycloalkyl, C₁-C₄ haloalkyl and C₂-C₃ alkynyl.

In an embodiment, each R^{a} is independently selected from halo and C₁-C₄ alkyl. In an embodiment, each R^{a} is independently selected from halo and C₃-C₄ cycloalkyl. In an embodiment, each R^{a} is independently selected from halo and C₁-C₄ haloalkyl. In an embodiment, each R^{a} is independently selected from halo and C₂-C₃ alkynyl. In an embodiment, each R^{a} is independently halo.

In an embodiment, each R^{a} is independently selected from —F, —Cl, —OH, -Me, -Et, -cyclopropyl, —CF₃ and —C≡CH. In an embodiment, each R^{a} is independently selected from —F, —Cl, -Me, -Et, -cyclopropyl, —CF₃ and —C≡CH. In an embodiment, each R^{a} is independently selected from —F, —Cl, -Me and -Et. In an embodiment, each R^{a} is independently selected from —F, —Cl and -cyclopropyl. In an embodiment, each R^{a} is independently selected from —F, —Cl and —CF₃. In an embodiment, each R^{a} is independently selected from —F, —Cl and —C≡CH. In an embodiment, each R^{a} is independently selected from —F and —Cl. In an embodiment, each R^{a} is —F. In an embodiment, each R^{a} is —Cl. In an embodiment, each R^{a} is —OH. In an embodiment, each R^{a} is -Me. In an embodiment, each R^{a} is -Et. In an embodiment, each R^{a} is -cyclopropyl. In an embodiment, each R^{a} is —CF₃. In an embodiment, each R^{a} is —C≡CH.

As generally defined herein, m is 0, 1, 2 or 3. In an embodiment, m is 1, 2 or 3. In an embodiment, m is 1 or 2. In an embodiment, m is 2 or 3. In an embodiment, m is 0. In an embodiment, m is 1. In an embodiment, m is 2. In an embodiment, m is 3.

As generally defined herein, each R³ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R³ is independently selected from the group consisting of hydrogen, halo, C₁-C₄ alkyl, C₃-C₄ cycloalkyl, C₁-C₄ haloalkyl and C₂-C₃ alkynyl. In an embodiment, each R³ is independently selected from the group consisting of hydrogen, halo and C₁-C₄ alkyl. In an embodiment, each R³ is independently selected from the group consisting of hydrogen, halo and C₃-C₄ cycloalkyl. In an embodiment, each R³ is independently selected from the group consisting of hydrogen, halo and C₁-C₄ haloalkyl. In an embodiment, each R³ is independently selected from the group consisting of hydrogen, halo and C₂-C₃ alkynyl. In an embodiment, R³ is selected from halo and C₁-C₄ alkyl. In an embodiment, R³ is selected from halo and C₂-C₃ alkynyl. In an embodiment, each R³ is independently selected from the group consisting of hydrogen and halo. In an embodiment, each R³ is independently halo. In an embodiment, each R³ is independently selected from the group consisting of —H, —F, —Cl, -Me, -Et, -cyclopropyl, —CF₃ and —C≡CH. In an embodiment, each R³ is independently selected from the group consisting of —H, —F, —Cl, -Me and -Et. In an embodiment, each R³ is independently selected from the group consisting of —H, —F, —Cl and -cyclopropyl. In an embodiment, each R³ is independently selected from the group consisting of —H, —F, —Cl and —CF₃. In an embodiment, each R³ is independently selected from the group consisting of —H, —F, —Cl and —C≡CH. In an embodiment, each R³ is independently selected from the group consisting of —H, —F and —Cl. In an embodiment, R³ is selected from —F, —Cl, -Et, and —C≡CH. In an embodiment, R³ is selected from —F, —Cl, and -Et. In an embodiment, R³ is selected from —F, —Cl and —C≡CH. In an embodiment, each R³ is independently selected from the group consisting of —F and —Cl. In an embodiment, R³ is —F. In an embodiment, R³ is —Cl. In an embodiment, R³ is -Et. In an embodiment, R³ is —C≡CH.

As generally defined herein, each R⁴ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R⁴ is independently selected from the group consisting of hydrogen, halo, C₁-C₄ alkyl, C₃-C₄ cycloalkyl, C₁-C₄ haloalkyl and C₂-C₃ alkynyl. In an embodiment, each R⁴ is independently selected from the group consisting of hydrogen, halo and C₁-C₄ alkyl. In an embodiment, each R⁴ is independently selected from the group consisting of hydrogen, halo and C₃-C₄ cycloalkyl. In an embodiment, each R⁴ is independently selected from the group consisting of hydrogen, halo and C₁-C₄ haloalkyl. In an embodiment, each R⁴ is independently selected from the group consisting of hydrogen, halo and C₂-C₃ alkynyl. In an embodiment, each R⁴ is independently selected from the group consisting of hydrogen and halo. In an embodiment, each R⁴ is independently halo. In an embodiment, each R⁴ is independently selected from the group consisting of —H, —F, —Cl, -Me, -Et, -cyclopropyl, —CF₃ and —C≡CH. In an embodiment, each R⁴ is independently selected from the group consisting of —H, —F, —Cl, -Me and -Et. In an embodiment, each R⁴ is independently selected from the group consisting of —H, —F, —Cl and -cyclopropyl. In an embodiment, each R⁴ is independently selected from the group consisting of —H, —F, —Cl and —CF₃. In an embodiment, each R⁴ is independently selected from the group consisting of —H, —F, —Cl and —C≡CH. In an embodiment, each R⁴ is independently selected from the group consisting of —H, —F and —Cl. In an embodiment, each R⁴ is independently selected from the group consisting of —F and —Cl. In an embodiment, R⁴ is selected from —H and —F. In an embodiment, R⁴ is —H. In an embodiment, R⁴ is —F.

As generally defined herein, each R⁵ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R⁵ is independently selected from the group consisting of hydrogen, halo, C₁-C₄ alkyl, C₃-C₄ cycloalkyl, C₁-C₄ haloalkyl and C₂-C₃ alkynyl. In an embodiment, each R⁵ is independently selected from the group consisting of hydrogen, halo and C₁-C₄ alkyl. In an embodiment, each R⁵ is independently selected from the group consisting of hydrogen and C₁-C₄ alkyl. In an embodiment, each R⁵ is independently selected from the group consisting of hydrogen, halo and C₃-C₄ cycloalkyl. In an embodiment, each R⁵ is independently selected from the group consisting of hydrogen, halo and C₁-C₄ haloalkyl. In an embodiment, each R⁵ is independently selected from the group consisting of hydrogen, halo and C₂-C₃ alkynyl. In an embodiment, each R⁵ is independently selected from the group consisting of hydrogen and halo. In an embodiment, each R⁵ is independently halo. In an embodiment, each R⁵ is independently selected from the group consisting of —H, —F, —Cl, -Me, -Et, -cyclopropyl, —CF₃ and —C≡CH. In an embodiment, each R⁵ is independently selected from the group consisting of —H, —F, —Cl, -Me and -Et. In an embodiment, each R⁵ is independently selected from the group consisting of —H and -Me. In an embodiment, each R⁵ is independently selected from the group consisting of —H, —F, —Cl and -cyclopropyl. In an embodiment, each R^{s} is independently selected from the group consisting of —H, —F, —Cl and —CF₃. In an embodiment, each R⁵ is independently selected from the group consisting of —H, —F, —Cl and —C≡CH. In an embodiment, each R⁵ is independently selected from the group consisting of —H, —F and —Cl. In an embodiment, each R⁵ is independently selected from the group consisting of —F and —Cl. In an embodiment, each R⁵ is independently —H. In an embodiment, each R⁵ is independently -Me.

As generally defined herein, each R^{h} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{h} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{h} is independently selected from the group consisting of hydrogen, halo and C₁-C₄ alkyl. In an embodiment, each R^{h} is independently selected from the group consisting of hydrogen, halo and C₃-C₄ cycloalkyl. In an embodiment, each R^{h} is independently selected from the group consisting of hydrogen, halo and C₁-C₄ haloalkyl. In an embodiment, each R^{h} is independently selected from the group consisting of hydrogen, halo and C₂-C₃ alkynyl. In an embodiment, each R^{h} is independently selected from the group consisting of hydrogen and halo. In an embodiment, each R^{h} is independently halo. In an embodiment, each R^{h} is independently selected from the group consisting of —H, —F, —Cl, -Me, -Et, -cyclopropyl, —CF₃ and —C≡CH. In an embodiment, each R^{h} is independently selected from the group consisting of —H, —F, —Cl, -Me and -Et. In an embodiment, each R^{h} is independently selected from the group consisting of —H, —F, —Cl and -cyclopropyl. In an embodiment, each R^{h} is independently selected from the group consisting of —H, —F, —Cl and —CF₃. In an embodiment, each R^{h} is independently selected from the group consisting of —H, —F, —Cl and —C≡CH. In an embodiment, each R^{h} is independently selected from the group consisting of —H, —F and —Cl. In an embodiment, each R^{h} is independently selected from the group consisting of —F and —Cl.

As generally defined herein, each Rⁱ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each Rⁱ is independently selected from the group consisting of hydrogen, halo, C₁-C₄ alkyl, C₃-C₄ cycloalkyl, C₁-C₄ haloalkyl and C₂-C₃ alkynyl. In an embodiment, each Rⁱ is independently selected from the group consisting of hydrogen, halo and C₁-C₄ alkyl. In an embodiment, each Rⁱ is independently selected from the group consisting of hydrogen, halo and C₃-C₄ cycloalkyl. In an embodiment, each Rⁱ is independently selected from the group consisting of hydrogen, halo and C₁-C₄ haloalkyl. In an embodiment, each Rⁱ is independently selected from the group consisting of hydrogen, halo and C₂-C₃ alkynyl. In an embodiment, each Rⁱ is independently selected from the group consisting of hydrogen and halo. In an embodiment, each Rⁱ is independently halo. In an embodiment, each Rⁱ is independently selected from the group consisting of —H, —F, —Cl, -Me, -Et, -cyclopropyl, —CF₃ and —C≡CH. In an embodiment, each Rⁱ is independently selected from the group consisting of —H, —F, —Cl, -Me and -Et. In an embodiment, each Rⁱ is independently selected from the group consisting of —H, —F, —Cl and -cyclopropyl. In an embodiment, each Rⁱ is independently selected from the group consisting of —H, —F, —Cl and —CF₃. In an embodiment, each Rⁱ is independently selected from the group consisting of —H, —F, —Cl and —C≡CH. In an embodiment, each Rⁱ is independently selected from the group consisting of —H, —F and —Cl. In an embodiment, each Rⁱ is independently selected from the group consisting of —F and —Cl.

As generally defined herein, each Rⁱ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each Rⁱ is independently selected from the group consisting of hydrogen, halo, C₁-C₄ alkyl, C₃-C₄ cycloalkyl, C₁-C₄ haloalkyl and C₂-C₃ alkynyl. In an embodiment, each Rⁱ is independently selected from the group consisting of hydrogen, halo and C₁-C₄ alkyl. In an embodiment, each Rⁱ is independently selected from the group consisting of hydrogen, halo and C₃-C₄ cycloalkyl. In an embodiment, each Rⁱ is independently selected from the group consisting of hydrogen, halo and C₁-C₄ haloalkyl. In an embodiment, each Rⁱ is independently selected from the group consisting of hydrogen, halo and C₂-C₃ alkynyl. In an embodiment, each Rⁱ is independently selected from the group consisting of hydrogen and halo. In an embodiment, Rⁱ is selected from C₃-C₄ cycloalkyl and C₁-C₄ haloalkyl. In an embodiment, each R^{j} is independently halo. In an embodiment, each Rⁱ is independently selected from the group consisting of —H, —F, —Cl, -Me, -Et, -cyclopropyl, —CF₃ and —C≡CH. In an embodiment, each Rⁱ is independently selected from the group consisting of —H, —F, —Cl, -Me and -Et. In an embodiment, each Rⁱ is independently selected from the group consisting of —H, —F, —Cl and -cyclopropyl. In an embodiment, each Rⁱ is independently selected from the group consisting of —H, —F, —Cl and —CF₃. In an embodiment, each Rⁱ is independently selected from the group consisting of —H, —F, —Cl and —C≡CH. In an embodiment, each Rⁱ is independently selected from the group consisting of —H, —F and —Cl. In an embodiment, each Rⁱ is independently selected from the group consisting of —F and —Cl. In an embodiment, Rⁱ is selected from cyclopropyl, —CHF₂ and —CF₃. In an embodiment, Rⁱ is cyclopropyl. In an embodiment, Rⁱ is —CHF₂. In an embodiment, Rⁱ is —CF₃.

As generally defined herein, each R^{k} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{k} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{k} is independently selected from the group consisting of hydrogen, halo and C₁-C₄ alkyl. In an embodiment, each R^{k} is independently selected from the group consisting of hydrogen, halo and C₃-C₄ cycloalkyl. In an embodiment, each R^{k} is independently selected from the group consisting of hydrogen, halo and C₁-C₄ haloalkyl. In an embodiment, each R^{k} is independently selected from the group consisting of hydrogen, halo and C₂-C₃ alkynyl. In an embodiment, each R^{k} is independently selected from the group consisting of hydrogen and halo. In an embodiment, each R^{k} is independently halo. In an embodiment, each R^{k} is independently selected from the group consisting of —H, —F, —Cl, -Me, -Et, -cyclopropyl, —CF₃ and —C≡CH. In an embodiment, each R^{k} is independently selected from the group consisting of —H, —F, —Cl, -Me and -Et. In an embodiment, each R^{k} is independently selected from the group consisting of —H, —F, —Cl and -cyclopropyl. In an embodiment, each R^{k} is independently selected from the group consisting of —H, —F, —Cl and —CF₃. In an embodiment, each R^{k} is independently selected from the group consisting of —H, —F, —Cl and —C≡CH. In an embodiment, each R^{k} is independently selected from the group consisting of —H, —F and —Cl. In an embodiment, each R^{k} is independently selected from the group consisting of —F and —Cl. In an embodiment, R^{k} is selected from —H and —Cl. In an embodiment, R^{k} is —H. In an embodiment, R^{k} is —Cl.

As generally defined herein, each Rⁿ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each Rⁿ is independently selected from the group consisting of hydrogen, halo, C₁-C₄ alkyl, C₃-C₄ cycloalkyl, C₁-C₄ haloalkyl and C₂-C₃ alkynyl. In an embodiment, each Rⁿ is independently selected from the group consisting of hydrogen, halo and C₁-C₄ alkyl. In an embodiment, each Rⁿ is independently selected from the group consisting of hydrogen, halo and C₃-C₄ cycloalkyl. In an embodiment, each Rⁿ is independently selected from the group consisting of hydrogen, halo and C₁-C₄ haloalkyl. In an embodiment, each Rⁿ is independently selected from the group consisting of hydrogen, halo and C₂-C₃ alkynyl. In an embodiment, each Rⁿ is independently selected from the group consisting of hydrogen and halo. In an embodiment, each Rⁿ is independently halo. In an embodiment, each Rⁿ is independently selected from the group consisting of —H, —F, —Cl, -Me, -Et, -cyclopropyl, —CF₃ and —C≡CH. In an embodiment, each Rⁿ is independently selected from the group consisting of —H, —F, —Cl, -Me and -Et. In an embodiment, each Rⁿ is independently selected from the group consisting of —H, —F, —Cl and -cyclopropyl. In an embodiment, each Rⁿ is independently selected from the group consisting of —H, —F, —Cl and —CF₃. In an embodiment, each Rⁿ is independently selected from the group consisting of —H, —F, —Cl and —C≡CH. In an embodiment, each Rⁿ is independently selected from the group consisting of —H, —F and —Cl. In an embodiment, each Rⁿ is independently selected from the group consisting of —F and —Cl.

As generally defined herein, each R^{e} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{e} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{e} is independently selected from the group consisting of hydrogen, halo and C₁-C₄ alkyl. In an embodiment, each R^{e} is independently selected from the group consisting of hydrogen, halo and C₃-C₄ cycloalkyl. In an embodiment, each R^{e} is independently selected from the group consisting of hydrogen, halo and C₁-C₄ haloalkyl. In an embodiment, each R^{e} is independently selected from the group consisting of hydrogen, halo and C₂-C₃ alkynyl. In an embodiment, each R^{e} is independently selected from the group consisting of hydrogen and halo. In an embodiment, each R^{e} is independently halo. In an embodiment, each R^{o} is independently selected from the group consisting of —H, —F, —Cl, -Me, -Et, -cyclopropyl, —CF₃ and —C≡CH. In an embodiment, each R^{e} is independently selected from the group consisting of —H, —F, —Cl, -Me and -Et. In an embodiment, each R^{e} is independently selected from the group consisting of —H, —F, —Cl and -cyclopropyl. In an embodiment, each R^{e} is independently selected from the group consisting of —H, —F, —Cl and —CF₃. In an embodiment, each R^{e} is independently selected from the group consisting of —H, —F, —Cl and —C≡CH. In an embodiment, each R^{e} is independently selected from the group consisting of —H, —F and —Cl. In an embodiment, each R^{e} is independently selected from the group consisting of —F and —Cl.

As generally defined herein, each R^{q} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{q} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{q} is independently selected from the group consisting of hydrogen, halo and C₁-C₄ alkyl. In an embodiment, each R^{q} is independently selected from the group consisting of hydrogen, halo and C₃-C₄ cycloalkyl. In an embodiment, each R^{q} is independently selected from the group consisting of hydrogen, halo and C₁-C₄ haloalkyl. In an embodiment, each R^{q} is independently selected from the group consisting of hydrogen, halo and C₂-C₃ alkynyl. In an embodiment, each R^{q} is independently selected from the group consisting of hydrogen and halo. In an embodiment, R^{q} is selected from the group consisting of halo and C₁-C₄ alkyl.

In an embodiment, each R^{q} is independently halo. In an embodiment, each R^{q} is independently selected from the group consisting of —H, —F, —Cl, -Me, -Et, -cyclopropyl, —CF₃ and —C≡CH. In an embodiment, each R^{q} is independently selected from the group consisting of —H, —F, —Cl, -Me and -Et. In an embodiment, each R^{q} is independently selected from the group consisting of —H, —F, —Cl and -cyclopropyl. In an embodiment, each R^{q} is independently selected from the group consisting of —H, —F, —Cl and —CF₃. In an embodiment, each R^{q} is independently selected from the group consisting of —H, —F, —Cl and —C≡CH. In an embodiment, each R^{q} is independently selected from the group consisting of —H, —F and —Cl. In an embodiment, each R^{q} is independently selected from the group consisting of —F and —Cl. In an embodiment, each R^{q} is selected from the group consisting of hydrogen, —F, —Cl, and -Me. In an embodiment, each R^{q} is selected from the group consisting of —Cl, and -Me. In an embodiment, each R^{q} is —Cl. In an embodiment, each R^{q} is -Me.

As generally defined herein, each R^{r} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{r} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{r} is independently selected from the group consisting of hydrogen, halo and C₁-C₄ alkyl. In an embodiment, each R^{r} is independently selected from the group consisting of hydrogen, halo and C₃-C₄ cycloalkyl. In an embodiment, each R^{r} is independently selected from the group consisting of hydrogen, halo and C₁-C₄ haloalkyl. In an embodiment, each R^{r} is independently selected from the group consisting of hydrogen, halo and C₂-C₃ alkynyl. In an embodiment, each R^{r} is independently selected from the group consisting of hydrogen and halo. In an embodiment, R^{r} is selected from the group consisting of hydrogen and C₁-C₄ alkyl. In an embodiment, each R^{r} is independently halo. In an embodiment, each R^{r} is independently selected from the group consisting of —H, —F, —Cl, -Me, -Et, -cyclopropyl, —CF₃ and —C≡CH. In an embodiment, each R^{r} is independently selected from the group consisting of —H, —F, —Cl, -Me and -Et. In an embodiment, each R^{r} is independently selected from the group consisting of —H, —F, —Cl and -cyclopropyl. In an embodiment, each R^{r} is independently selected from the group consisting of —H, —F, —Cl and —CF₃. In an embodiment, each R^{r} is independently selected from the group consisting of —H, —F, —Cl and —C≡CH. In an embodiment, each R^{r} is independently selected from the group consisting of —H, —F and —Cl. In an embodiment, each R^{r} is independently selected from the group consisting of —F and —Cl. In an embodiment, R^{r} is selected from the group consisting of —H and -Me. In an embodiment, R^{r} is —H. In an embodiment, R^{r} is -Me.

As generally defined herein, each R^{s} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{s} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{s} is independently selected from the group consisting of hydrogen, halo and C₁-C₄ alkyl. In an embodiment, each R^{s} is independently selected from the group consisting of hydrogen, halo and C₃-C₄ cycloalkyl. In an embodiment, each R^{s} is independently selected from the group consisting of hydrogen, halo and C₁-C₄ haloalkyl. In an embodiment, each R^{s} is independently selected from the group consisting of hydrogen, halo and C₂-C₃ alkynyl. In an embodiment, each R^{s} is independently selected from the group consisting of hydrogen and halo. In an embodiment, each R^{s} is independently halo. In an embodiment, each R^{s} is independently selected from the group consisting of —H, —F, —Cl, -Me, -Et, -cyclopropyl, —CF₃ and —C≡CH. In an embodiment, each R^{s} is independently selected from the group consisting of —H, —F, —Cl, -Me and -Et. In an embodiment, each R^{s} is independently selected from the group consisting of —H, —F, —Cl and -cyclopropyl. In an embodiment, each R^{s} is independently selected from the group consisting of —H, —F, —Cl and —CF₃. In an embodiment, each R^{s} is independently selected from the group consisting of —H, —F, —Cl and —C≡CH. In an embodiment, each R^{s} is independently selected from the group consisting of —H, —F and —Cl. In an embodiment, each R^{s} is independently selected from the group consisting of —F and —Cl. In an embodiment, R^{s} is selected from the group consisting of —H and —F. In an embodiment, R^{s} is —H. In an embodiment, R^{s} is —F.

As generally defined herein, each R⁹ is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R⁹ is independently selected from the group consisting of —H and —OH. In an embodiment, each R⁹ is independently —H. In an embodiment, each R⁹ is independently —OH.

As generally defined herein, each R^{m} is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{m} is independently selected from the group consisting of —H and —OH. In an embodiment, each R^{m} is independently —H. In an embodiment, each R^{m} is independently —OH.

As generally defined herein, each R^{p} is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{p} is independently selected from the group consisting of —H and —OH. In an embodiment, each R^{p} is independently —H. In an embodiment, each R^{p} is independently —OH.

As generally defined herein, R¹ is selected from the group consisting of H, —OCH₂R¹OC₁-C₄ alkyl, —C₁-C₄ alkyl and

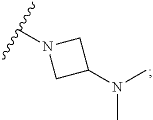

wherein R^{1A} is as defined in any of the embodiments described herein. In an embodiment, R¹ is selected from H, —OCH₂R^{1A}, —OMe, Me and

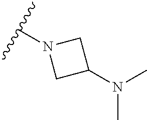

wherein R^{1A} is as defined in any of the embodiments described herein. In an embodiment, R¹ is H. In an embodiment, R¹ is —OMe. In an embodiment, R¹ is Me. In an embodiment, R¹ is

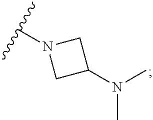 [SMILES: *N1CC(N(C)C)C1]

In an embodiment, R¹ is OCH₂R^{1A} wherein R^{1A} is as defined in any of the embodiments described herein.

In an embodiment, R¹ is selected from the group consisting of

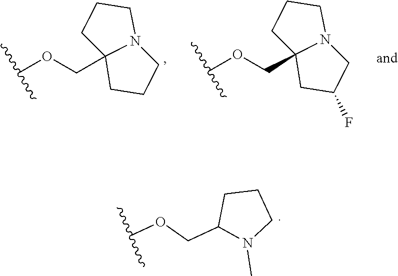

In an embodiment, R¹ is

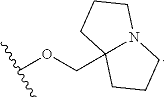 [SMILES: *OCC12CCCN1CCC2]

In an embodiment, R¹ is

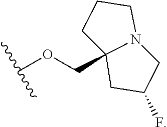 [SMILES: *OC[C@@]12CCCN1C[C@H](F)C2]

In an embodiment, R¹ is

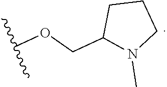 [SMILES: *OCC1CCCN1C]

As generally defined herein, R^{1A} is

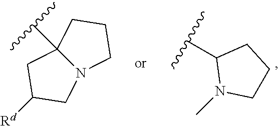

wherein R^{d} is as defined in any of the embodiments described herein. In an embodiment, R^{1A} is selected from

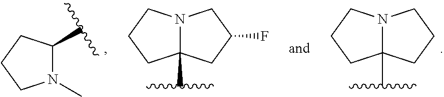

In an embodiment, R^{1A} is selected from and

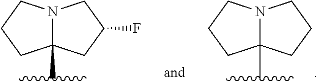 [SMILES: *[C@@]12CCCN1C[C@H](F)C2 ; *C12CCCN1CCC2]

In an embodiment, R^{1A} s

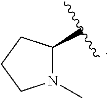 [SMILES: *[C@@H]1CCCN1C]

In an embodiment, R^{1A} is

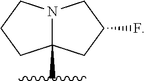 [SMILES: *[C@@]12CCCN1C[C@H](F)C2]

In an embodiment, R^{1A} is

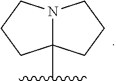 [SMILES: *C12CCCN1CCC2]

As generally defined herein, R^{d} is H or F. In an embodiment, R^{d} is H. In an embodiment, R^{d} is F.

As generally defined herein, R² is

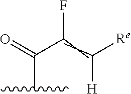

wherein R^{e} is as defined in any of the embodiments described herein. In an embodiment, R² is

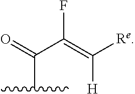

In an embodiment, R² is

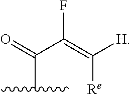

As generally defined herein, R^{e} is is selected from the group consisting of —COOH, —C(O)O—C₁-C₄ alkyl, —C(O)O—C₁-C₄ haloalkyl, —C(O)—C₁-C₄ alkyl, —C(O)—C₁-C₄ haloalkyl, —C(O)N(C₁-C₄ alkyl)₂, —(C₁-C₂ alkyl)-(C₁-C₂ alkoxy), —S(O)₂—C₁-C₄ alkyl, —S(O)₂—C₁-C₄ haloalkyl, R^{e1}, —C(R^{Y1})(R^{Y2})R^{e1}, R^{e2} and —C(R^{Y1})(R^{Y2})R^{e3}, wherein R^{o}e, R^{e2}, R^{e3}, R^{Y1}, and R^{Y2} are as defined in any of the embodiments described herein.

In an embodiment, R^{e} is selected from the group consisting of —C(O)O—C₁-C₄ alkyl, R^{e1}, —C(R^{Y1})(R^{Y2})R^{e1}, R^{e2} and —C(R^{Y1})(R^{Y2})R^{e3} wherein R^{o}e, R^{e2}, R^{e3}, R^{Y1}, and R^{Y2} are as defined in any of the embodiments described herein.

In an embodiment, R^{e} is selected from the group consisting of —C(O)OMe, R^{e1}, —CH₂R^{e1}, R^{e2} and —CH₂R^{e3} wherein R^{e1}, R^{e2} and R^{e3} are as defined in any of the embodiments described herein.

In an embodiment, R^{e} is selected from the group consisting of —C(O)OMe, R^{e1}, —CH₂R^{e1} and R^{e2} wherein R^{e1} and R^{e2} are as defined in any of the embodiments described herein.

In an embodiment, R^{e} is selected from the group consisting of R^{e1}, —CH₂R^{e1} and R^{e2} wherein R^{e1} and R^{e2} are as defined in any of the embodiments described herein.

In an embodiment, R^{e} is —R^{e1} wherein R^{e1} is as defined in any of the embodiments described herein. In an embodiment, R^{e} is C(R^{Y1})(R^{Y2})R^{e1} wherein R^{e1}, R^{Y1} and R^{Y2} are as defined in any of the embodiments described herein.

In an embodiment, R^{e} is R^{e2}, wherein R^{e2} is as defined in any of the embodiments described herein.

In an embodiment, R^{e} is —C(R^{Y1})(R^{Y2}), wherein R^{e3}, R^{Y1} and R^{Y2} are as defined in any of the embodiments described herein.

In an embodiment, R^{e} is selected from the group consisting of:

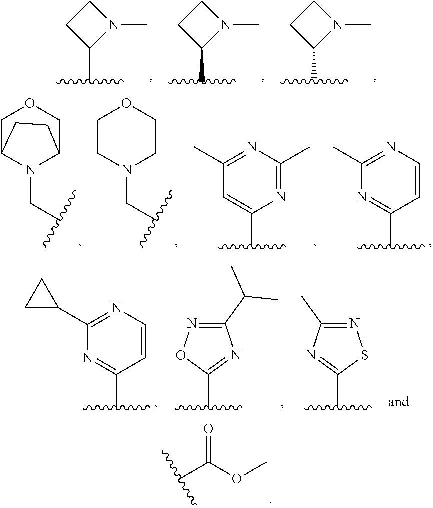

In an embodiment, R^{e} is

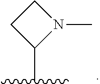 [SMILES: *C1CCN1C]

In an embodiment, R^{e} is

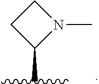 [SMILES: *[C@H]1CCN1C]

In an embodiment, R^{e} is

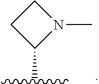 [SMILES: *[C@@H]1CCN1C]

In an embodiment, R^{e} is

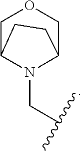 [SMILES: *CN1C2CCC1COC2]

In an embodiment, R^{e} is

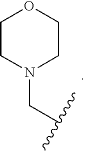 [SMILES: *CN1CCOCC1]

In an embodiment, R^{e} is

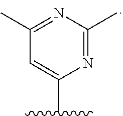

In an embodiment, R^{e} is

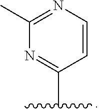 [SMILES: *c1ccnc(C)n1]

In an embodiment, R^{e} is

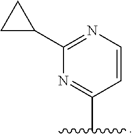 [SMILES: *c1ccnc(C2CC2)n1]

In an embodiment, R^{e} is

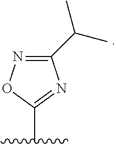 [SMILES: *c1nc(C(C)C)no1]

In an embodiment, R^{e} is

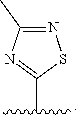 [SMILES: *c1nc(C)ns1]

In an embodiment, R^{e} is —C(O)OMe.

As generally defined herein, each R^{Y1} is independently selected from —H and —CH₃. In an embodiment, R^{Y1} is —H. In an embodiment, R^{Y1} is -Me.

As generally defined herein, each R^{Y2} is independently selected from —H and —CH₃. In an embodiment, R^{Y2} is —H. In an embodiment, R^{Y2} is -Me.

As generally defined herein, R^{e1} is a 4-10 membered heterocycle which is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl, and C₁-C₄ haloalkoxy.

In an embodiment, R^{e1} is a 4-7 membered monocyclic heterocycle containing a nitrogen atom as the only heteroatom or containing one nitrogen atom and one oxygen atom, wherein the monocyclic heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 4-7 membered monocyclic heterocycle containing a nitrogen atom as the only heteroatom, wherein the monocyclic heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, the monocyclic heterocycle of R^{e1} is substituted with 0 or 1 instance of methyl, ethyl, isopropyl or methoxyethyl.

In an embodiment, the monocyclic heterocycle of R^{e1} is substituted with 0 or 1 instance of C₁-C₄ alkyl. In an embodiment, the monocyclic heterocycle of R^{e1} is substituted with 0 or 1 instance of methyl.

In an embodiment, R^{e1} is selected from azetidinyl, pyrrolidinyl, tetrahydrofuranyl, and morpholinyl substituted with 0 or 1 instance of methyl, ethyl, isopropyl, or methoxyethyl.

In an embodiment, R^{e1} is selected from azetidinyl, pyrrolidinyl and morpholinyl substituted with 0 or 1 instance of methyl. In an embodiment, R^{e1} is azetidinyl substituted with 0 or 1 instance of methyl, ethyl, isopropyl or methoxyethyl.

In an embodiment, R^{e1} is azetidinyl substituted with 0 or 1 instance of methyl.

In an embodiment, R^{e1} is N-methyl azetidinyl.

In an embodiment, the attachment point for the monocyclic heterocycle is on a carbon atom.

In an embodiment, R^{e1} is selected from the group consisting of:

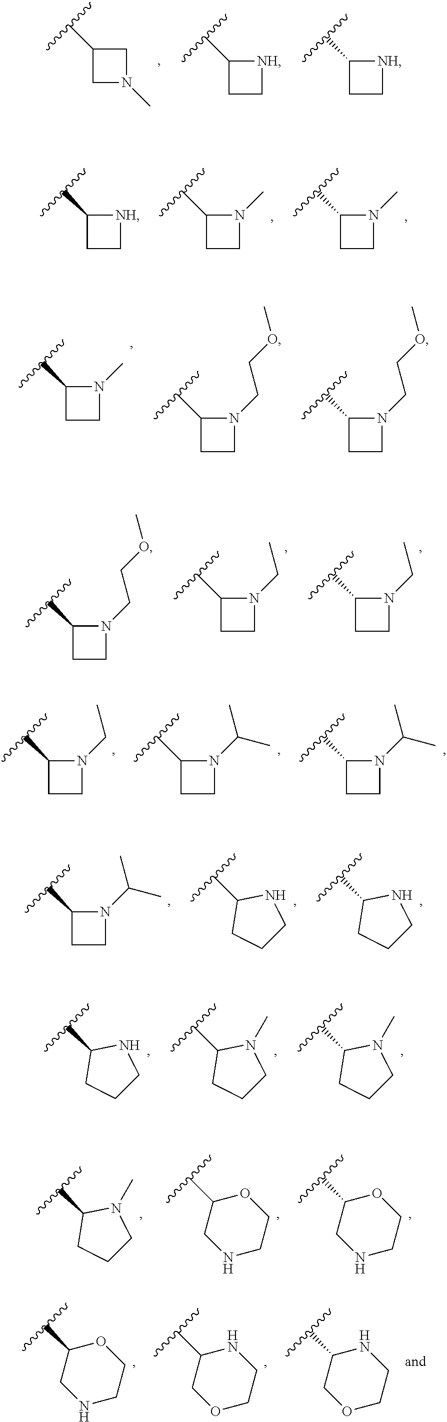

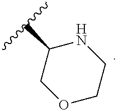 [SMILES: *[C@@H]1COCCN1]

In an embodiment, R^{e1} is

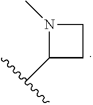 [SMILES: *C1CCN1C]

In an embodiment, R^{e1} is

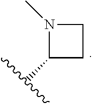 [SMILES: *[C@H]1CCN1C]

In an embodiment, R^{e1} is a 4-10 membered heterocycle containing a nitrogen atom and one or two additional heteroatoms independently selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 4-10 membered heterocycle containing a nitrogen atom and one or two additional heteroatoms independently selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 4-8 member monocyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 6-10 member fused bicyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 6-10 member bridged heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 6-10 member spiro heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is selected from azetidine, pyrrolidine, 2-azabicyclo [2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is morpholine substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is selected from the group consisting of:

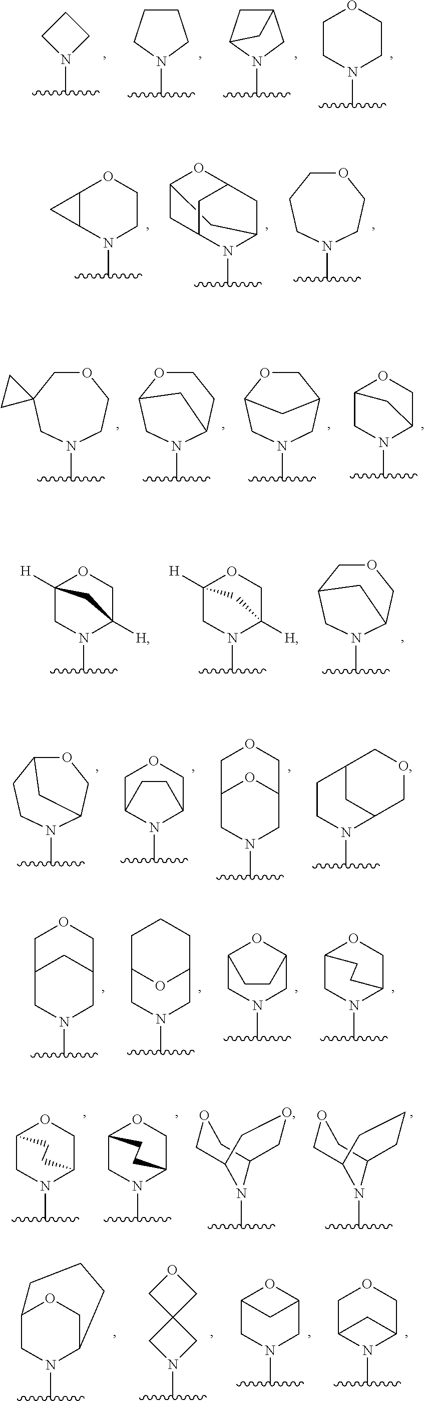

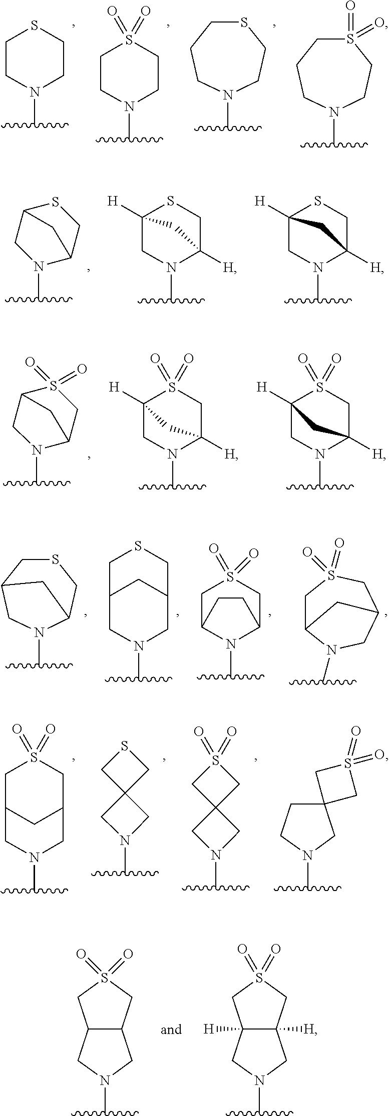

each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is selected from

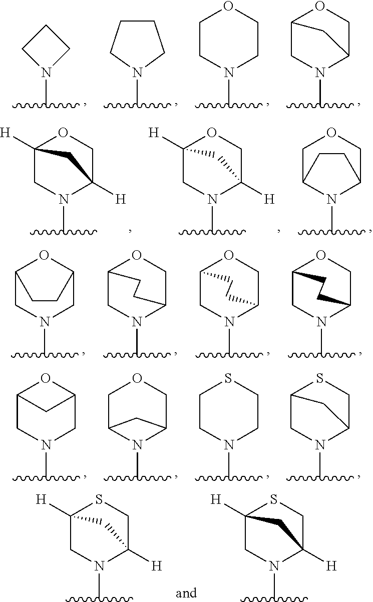

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is selected from

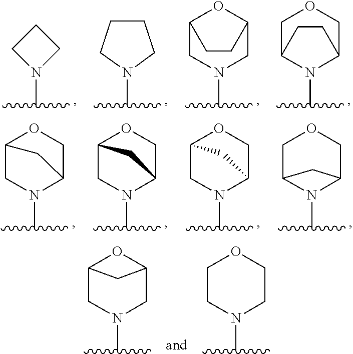

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1}

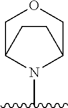 [SMILES: *N1C2CCC1COC2]

is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

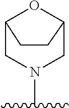 [SMILES: *N1CC2CCC(C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

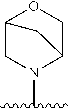 [SMILES: *N1CC2CC1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

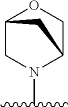 [SMILES: *N1C[C@H]2C[C@@H]1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

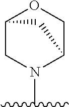 [SMILES: *N1C[C@@H]2C[C@H]1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

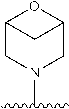 [SMILES: *N1CC2CC(C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

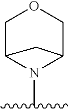 [SMILES: *N1C2COCC1C2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

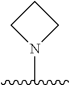 [SMILES: *N1CCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

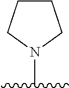 [SMILES: *N1CCCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

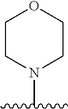 [SMILES: *N1CCOCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, the 4-10 membered heterocycle of R¹ is substituted with 0, 1 or 2 substituents independently selected from —F, —OMe and -Me.

In an embodiment, the 4-10 membered heterocycle of R¹ is substituted with 0, 1 or 2 substituents independently selected from —F and, —OMe.

In an embodiment, the 4-10 membered heterocycle of R¹ is substituted with 0, 1 or 2 instances of —F.

In an embodiment, the 4-10 membered heterocycle of R¹ is substituted with 0 or 1 instances of —OMe.

In an embodiment, the 4-10 membered heterocycle of R¹ is unsubstituted.

In an embodiment, R^{e1} is selected from the group consisting of:

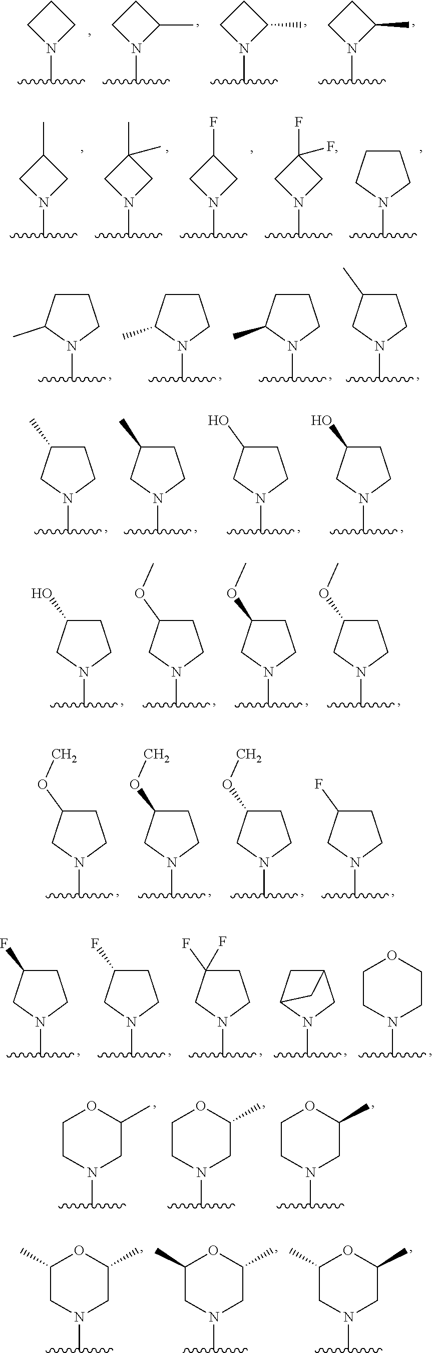

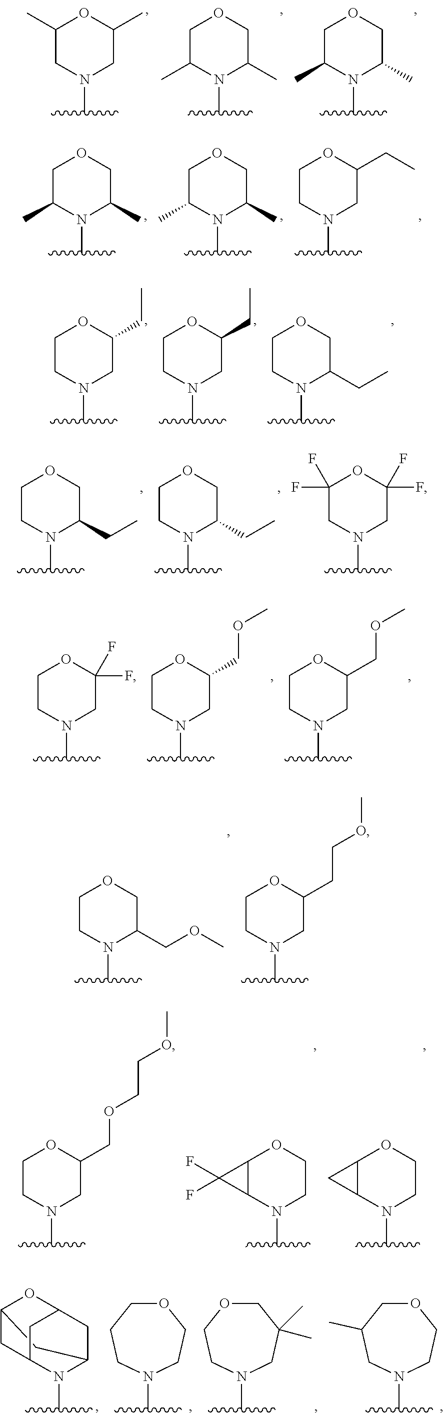

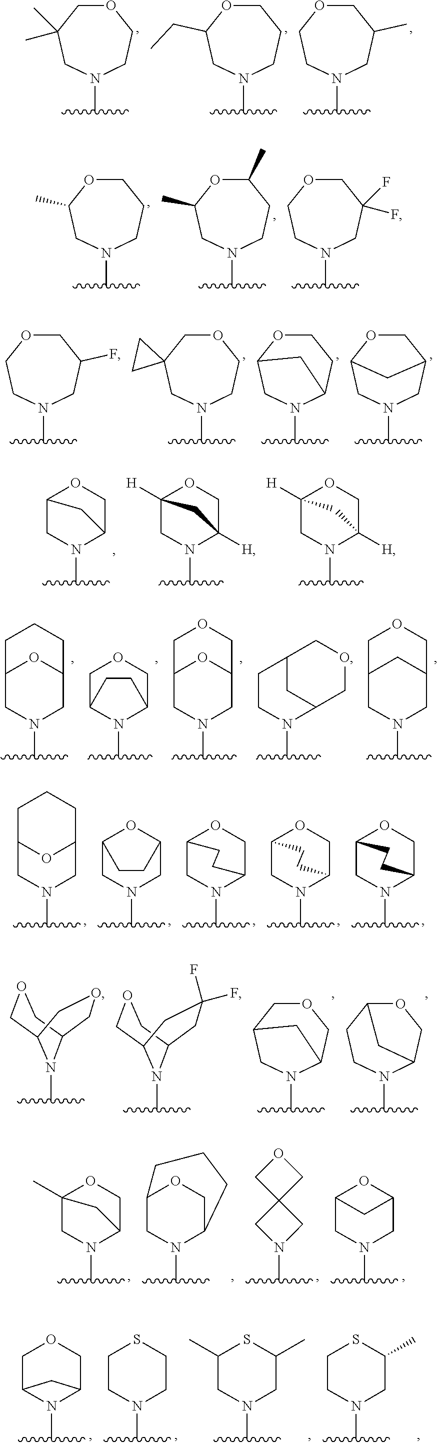

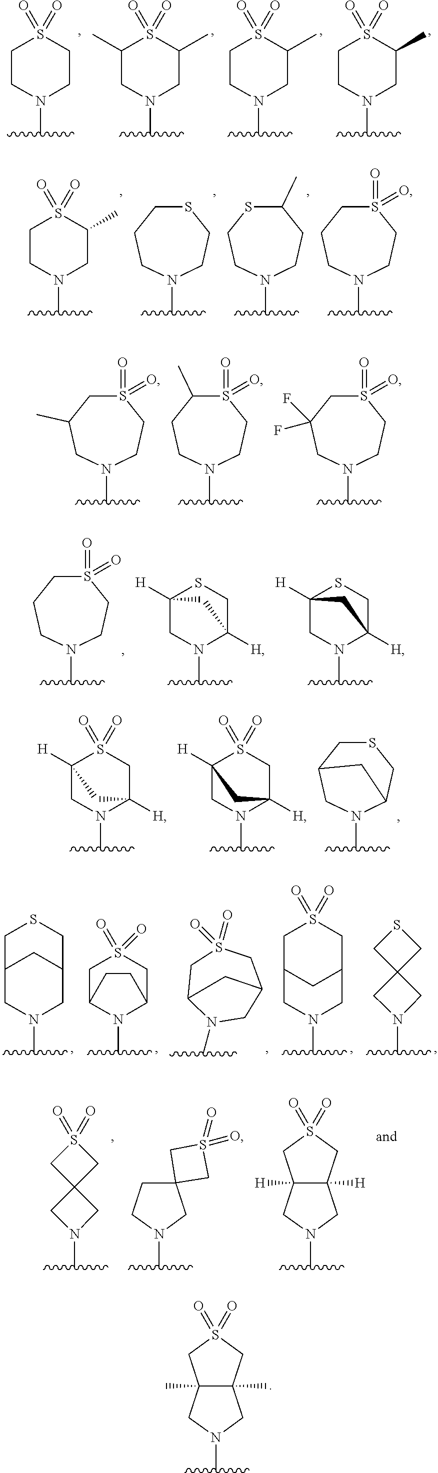

In an embodiment R^{e1} is selected from

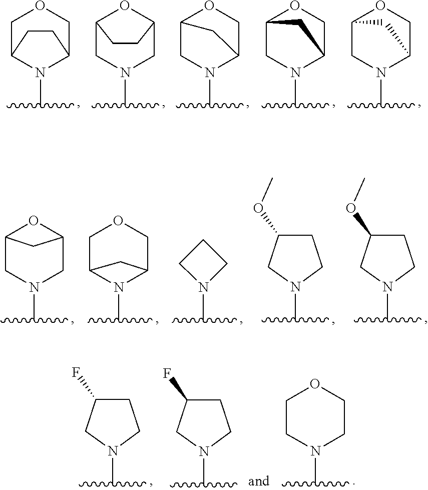

In an embodiment R^{e1} is unsubstituted

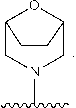 [SMILES: *N1CC2CCC(C1)O2]

In an embodiment R^{e1} is unsubstituted

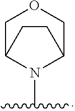 [SMILES: *N1C2CCC1COC2]

In an embodiment R^{e1} is unsubstituted

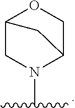 [SMILES: *N1CC2CC1CO2]

In an embodiment R^{e1} is unsubstituted

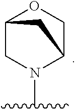 [SMILES: *N1C[C@H]2C[C@@H]1CO2]

In an embodiment R^{e1} is unsubstituted

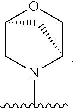 [SMILES: *N1C[C@@H]2C[C@H]1CO2]

In an embodiment R^{e1} is unsubstituted

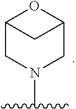 [SMILES: *N1CC2CC(C1)O2]

In an embodiment R^{e1} is unsubstituted

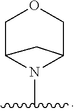 [SMILES: *N1C2COCC1C2]

In an embodiment R^{e1} is unsubstituted

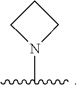 [SMILES: *N1CCC1]

In an embodiment R^{e1} is

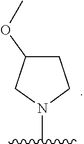 [SMILES: *N1CCC(OC)C1]

In an embodiment R^{e1} is

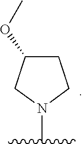 [SMILES: *N1CC[C@@H](OC)C1]

In an embodiment R^{e1} is

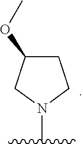 [SMILES: *N1CC[C@H](OC)C1]

In an embodiment R^{e1} is

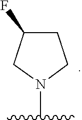 [SMILES: *N1CC[C@H](F)C1]

In an embodiment R^{e1} is

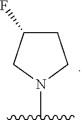 [SMILES: *N1CC[C@@H](F)C1]

In an embodiment R^{e1} is

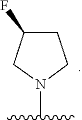 [SMILES: *N1CC[C@H](F)C1]

In an embodiment R^{e1} is unsubstituted

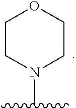 [SMILES: *N1CCOCC1]

As generally defined herein, R^{e2} is a 5-6 membered heteroaryl group optionally substituted with 0, 1, 2 or 3 substituents independently selected from halo, hydroxy, C₁-C₄ alkyl, C₁-C₄ hydroxyalkyl, C₁-C₄ alkoxy, C₁-C₄ haloalkyl, C₁-C₄ haloalkoxy, C₃-C₆ heterocyclyl optionally substituted with one or two substituents independently selected from halo, hydroxy and methyl, and C₃-C₆ cycloalkyl optionally substituted with one or two substituents independently selected from halo, hydroxy and methyl.

In an embodiment, R^{e2} is a 5-6 membered heteroaryl group containing at least one nitrogen atom, wherein the attachment point for the heteroaryl group is a carbon atom group and wherein the heteroaryl is substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is selected from the group consisting of pyrimidinyl, 1,2,4-oxadiazolyl and 1H-1,2,4-triazolyl, each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is selected from the group consisting of:

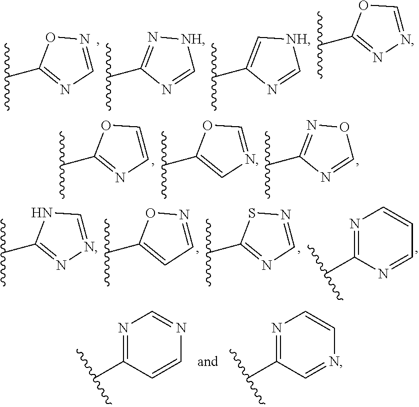

each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is selected from the group consisting of:

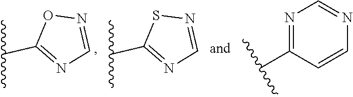

and each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is selected from the group consisting of:

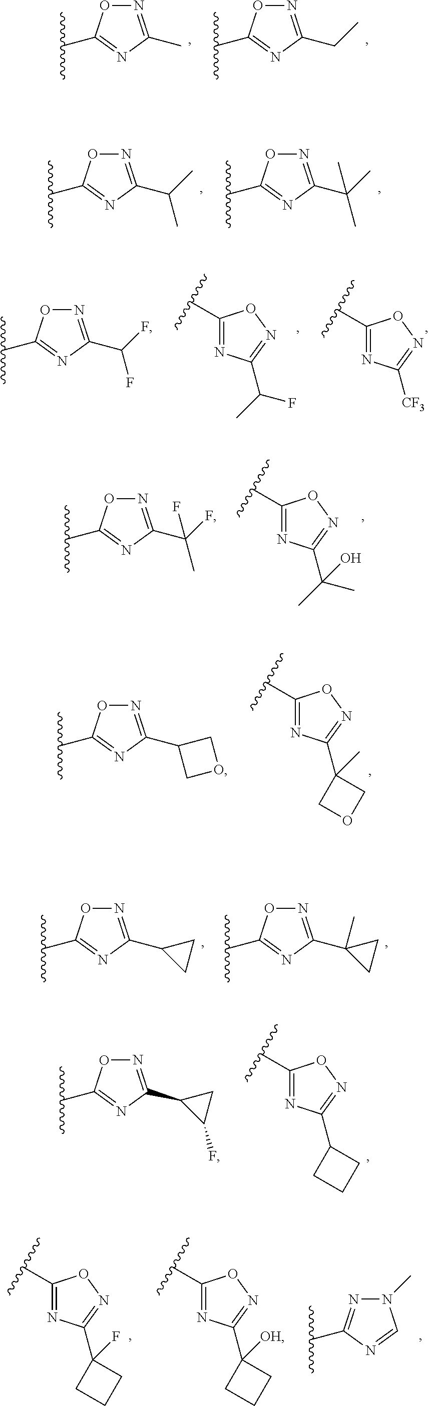

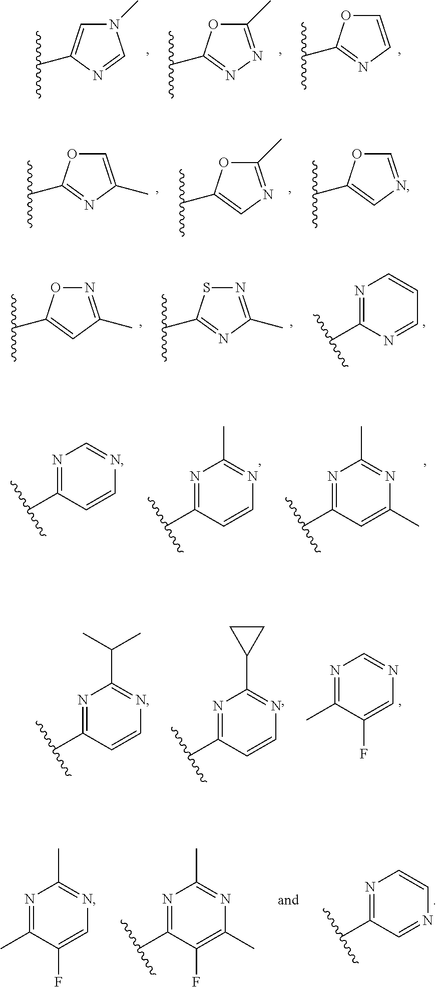

In an embodiment, R^{e2} is selected from the group consisting of:

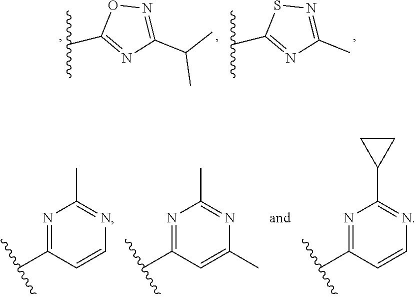

In an embodiment, R^{e2} is a 6 membered heteroaryl group substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is pyrimidinyl or pyridazinyl substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is pyrimidinyl or pyridazinyl substituted with 0, 1 or 2 instances of C₁-C₄ alkyl. In an embodiment, R^{e2} is pyrimidinyl substituted with 0, 1 or 2 instances of C₁-C₄ alkyl. In an embodiment, R^{e2} is pyridazinyl substituted with 0, 1 or 2 instances of C₁-C₄ alkyl.

In an embodiment, R^{e2} is pyrimidinyl or pyridazinyl substituted with 0, 1 or 2 instances of -Me. In an embodiment, R^{e2} is pyrimidinyl substituted with 0, 1 or 2 instances of -Me. In an embodiment, R^{e2} is pyridazinyl substituted with 0, 1 or 2 instances of -Me.

In an embodiment, R^{e2} is selected from the group consisting of:

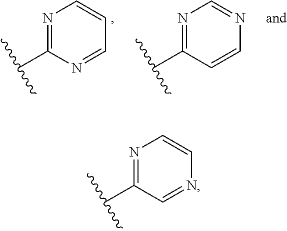 [SMILES: *c1ncccn1 ; *c1ccncn1 ; "and" ; *c1cnccn1]

substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is selected from the group consisting of

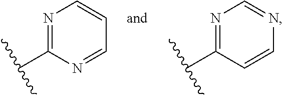 [SMILES: *c1ncccn1 ; "and" ; *c1ccncn1]

substituted with 0, 1 or 2 instances of C₁-C₄ alkyl.

In an embodiment, R^{e2} is

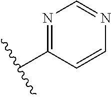 [SMILES: *c1ccncn1]

substituted with 0, 1 or 2 instances of C₁-C₄ alkyl. In an embodiment, R^{e2} is

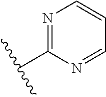 [SMILES: *c1ncccn1]

substituted with 0, 1 or 2 instances of C₁-C₄ alkyl. In an embodiment, R^{e2} is

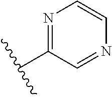 [SMILES: *c1cnccn1]

substituted with 0, 1 or 2 instances of C₁-C₄ alkyl.

In an embodiment, R^{e2} is selected from the group consisting of

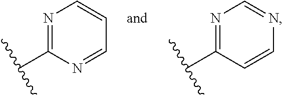 [SMILES: *c1ncccn1 ; "and" ; *c1ccncn1]

substituted with 0, 1 or 2 instances of -Me. In an embodiment, R^{e2} is

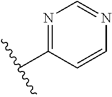 [SMILES: *c1ccncn1]

substituted with 0, 1 or 2 instances of -Me. In an embodiment, R^{e2} is

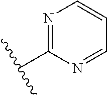 [SMILES: *c1ncccn1]

substituted with 0, 1 or 2 instances of -Me. In an embodiment, R^{e2} is

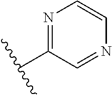 [SMILES: *c1cnccn1]

substituted with 0, 1 or 2 instances of -Me.

In an embodiment, R^{e2} is selected from the group consisting of

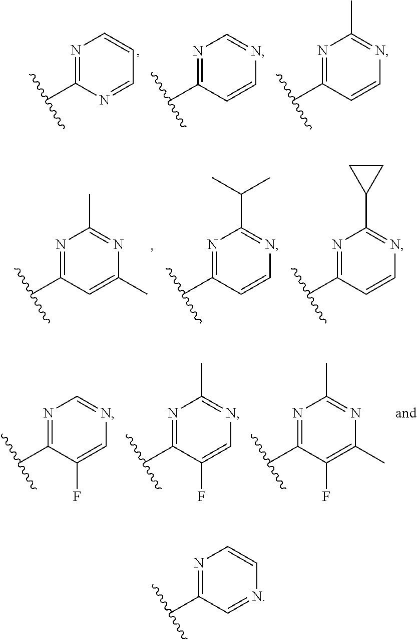

In an embodiment, R^{e2} is selected from the group consisting of

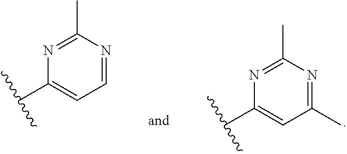

In an embodiment, R^{e2} is

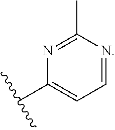 [SMILES: *c1ccnc(C)n1]

In an embodiment, R^{e2} is

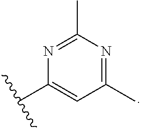 [SMILES: *c1cc(C)nc(C)n1]

In an embodiment, R^{e2} is a 5 membered heteroaryl group containing at least one nitrogen atom, wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo, hydroxy and methyl, and C₃-C₆ cycloalkyl optionally substituted with one or two substituents independently selected from halo, hydroxy and methyl.

In an embodiment, R^{e2} is selected from the group consisting of oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo, hydroxy and methyl, and C₃-C₆ cycloalkyl optionally substituted with one or two substituents independently selected from halo, hydroxy and methyl.

In an embodiment, R^{e2} is selected from the group consisting of

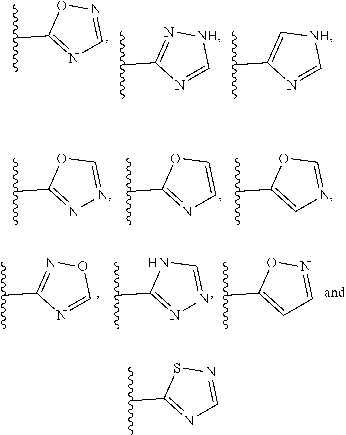

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo, hydroxy and methyl, and C₃-C₆ cycloalkyl optionally substituted with one or two substituents independently selected from halo, hydroxy and methyl.

In an embodiment, R^{e2} is selected from the group consisting of

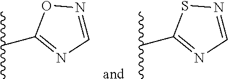 [SMILES: *c1ncno1 ; *c1ncns1 ; "and"]

each substituted with 1 substituent independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo, hydroxy and methyl, and C₃-C₆ cycloalkyl optionally substituted with one or two substituents independently selected from halo, hydroxy and methyl.

In an embodiment, R^{e2} is selected from the group consisting of

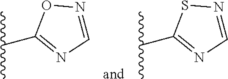 [SMILES: *c1ncno1 ; *c1ncns1 ; "and"]

each substituted with 1 instance of C₁-C₄ alkyl.

In an embodiment, R^{e2} is selected from the group consisting of:

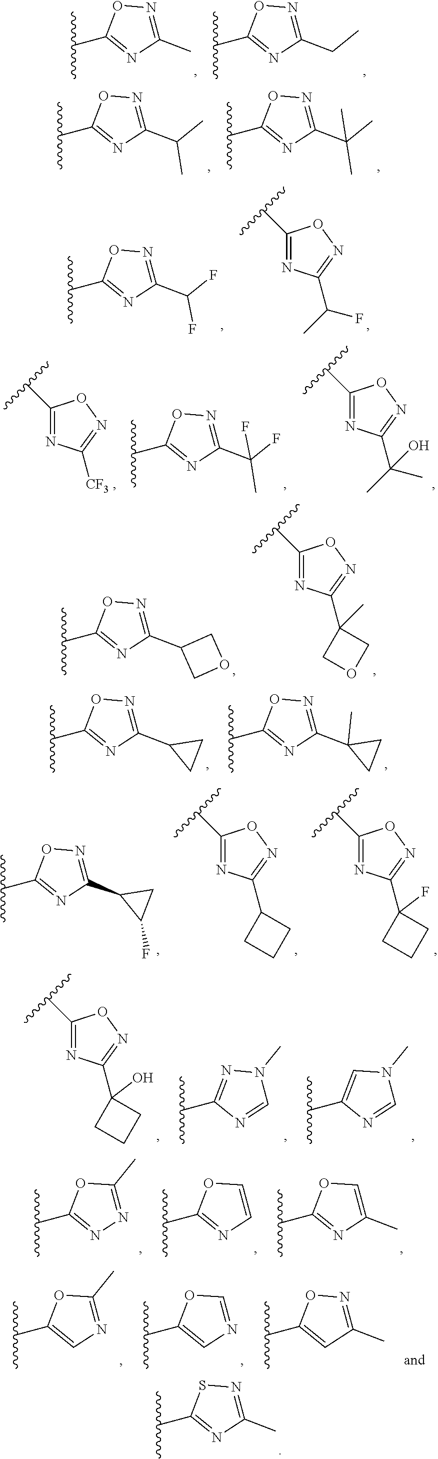

In an embodiment, R^{e2} is selected from the group consisting of

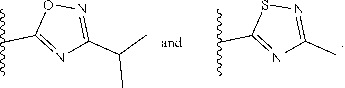

In an embodiment, R^{e2} is

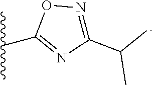 [SMILES: *c1nc(C(C)C)no1]

In an embodiment, R^{e2} is

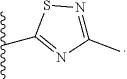 [SMILES: *c1nc(C)ns1]

As generally defined herein, R^{e3} is —NR³¹R³², wherein R³¹ and R³² are as defined in any of the embodiments described herein. In an embodiment, R^{e3} is selected from

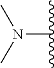 [SMILES: *N(C)C]

and

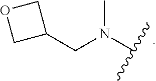 [SMILES: *N(C)CC1COC1]

In an embodiment, R^{e3} is

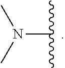 [SMILES: *N(C)C]

In an embodiment, R^{e3} is

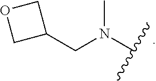 [SMILES: *N(C)CC1COC1]

As generally defined herein, R³¹ is selected from the group consisting of hydrogen, C₁-C₄ alkyl, C₁-C₄ alkoxy, C₁-C₄ haloalkoxy, and C₁-C₄ alkyl substituted with a 3-6 membered heterocycle. In an embodiment, R³¹ is selected from the group consisting of C₁-C₄ alkyl, C₁-C₄ alkoxy, C₁-C₄ haloalkoxy, and C₁-C₄ alkyl substituted with a 3-6 membered heterocycle containing 1-2 atoms independently selected from N, O and S, including S(O)₂.

In an embodiment, R³¹ is C₁-C₄ alkyl. In an embodiment, R³¹ is -Me.

As generally defined herein, R³² is selected from the group consisting of hydrogen, C₁-C₄ alkyl, C₁-C₄ alkoxy, C₁-C₄ haloalkoxy, and C₁-C₄ alkyl substituted with a 3-6 membered heterocycle. In an embodiment, R³² is selected from the group consisting of C₁-C₄ alkyl, C₁-C₄ alkoxy, C₁-C₄ haloalkoxy, and C₁-C₄ alkyl substituted with a 3-6 membered heterocycle containing 1-2 atoms independently selected from N, O and S, including S(O)₂.

In an embodiment, R³² is C₁-C₄ alkyl or C₁-C₄ alkyl substituted with a 3-6 membered heterocycle. In an embodiment, R³² is C₁-C₄ alkyl substituted with a 3-6 membered heterocycle. In an embodiment, R³² is C₁-C₄ alkyl substituted with a heterocycle selected from azetidine, oxetane, pyrrolidine, tetrahydrofuran, piperidine, tetrahydropyran and morpholine. In an embodiment, R³² is C₁-C₄ alkyl substituted with oxetane. In an embodiment, R³² is

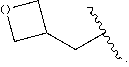 [SMILES: *CC1COC1]

In an embodiment, R³² is C₁-C₄ alkyl. In an embodiment, R³² is Me.

As generally defined herein, n is 0, 1 or 2. In an embodiment, n is 0 or 1. In an embodiment, n is 1 or 2. In an embodiment, n is 0. In an embodiment, n is 0 or 1. In an embodiment, n is 1. In an embodiment, n is 2.

As generally defined herein, R^{x} is selected from -halo and C₁-C₄ alkyl. In an embodiment, R^{x} is selected from —F and -Me. In an embodiment, R^{x} is -Me. In an embodiment, R^{x} is —F.

In an embodiment, the compound is of Formula (I-a), Formula (II-a), Formula (III-a), Formula (IV-a), Formula (V-a) or Formula (VI-a):

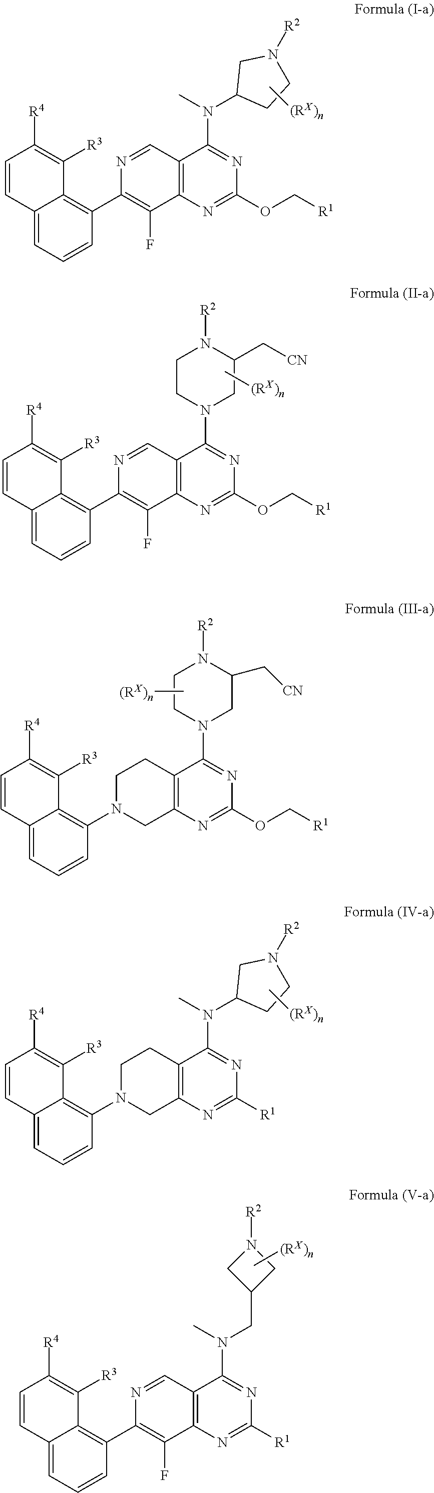

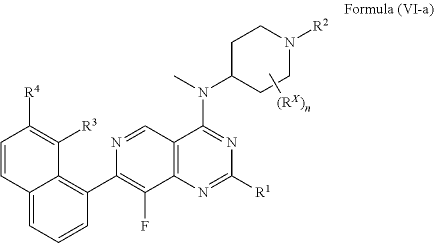

or a salt thereof; and/or an isotopologue thereof; wherein R¹, R², R³, R⁴, R^{x} and n are as defined in any of the embodiments described herein.

In an embodiment, the compound is of Formula (I-a).

In an embodiment, the compound is of Formula (II-a).

In an embodiment, the compound is of Formula (III-a).

In an embodiment, the compound is of Formula (IV-a).

In an embodiment, the compound is of Formula (V-a).

In an embodiment, the compound is of Formula (V-a).

In an embodiment, the stereochemistry of the pyrrolidine is (R) (i.e., the moiety represented as is

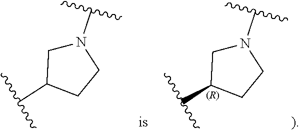

In an embodiment, the stereochemistry of the cyanomethyl groups is (S) (i.e., the moiety represented as is

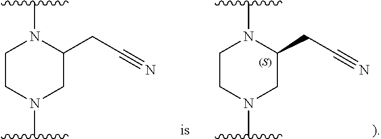

In an embodiment, the compound is of Formula (I-b), Formula (II-b), Formula (III-b), Formula (IV-b), Formula (V-b) or Formula (VI-b):

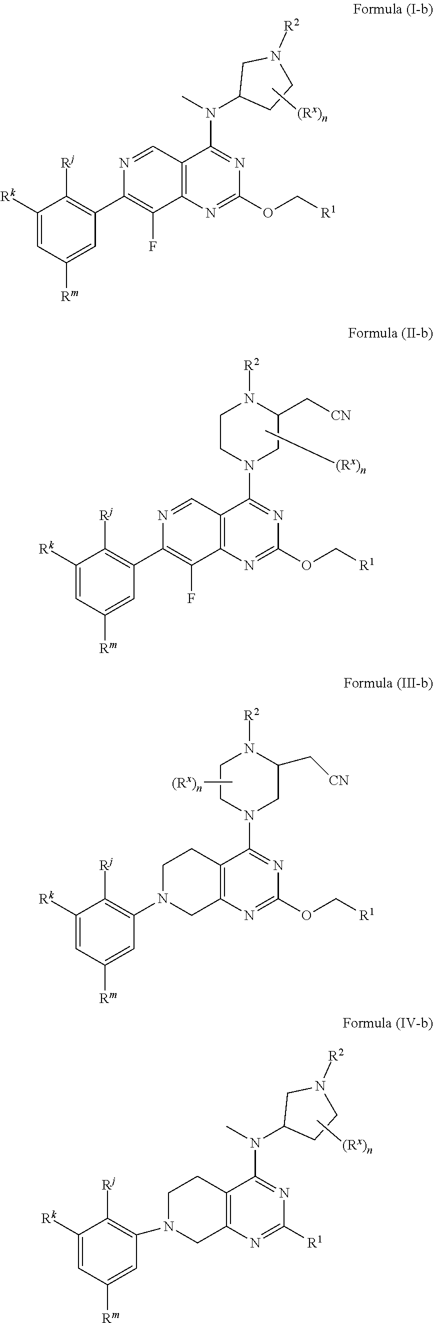

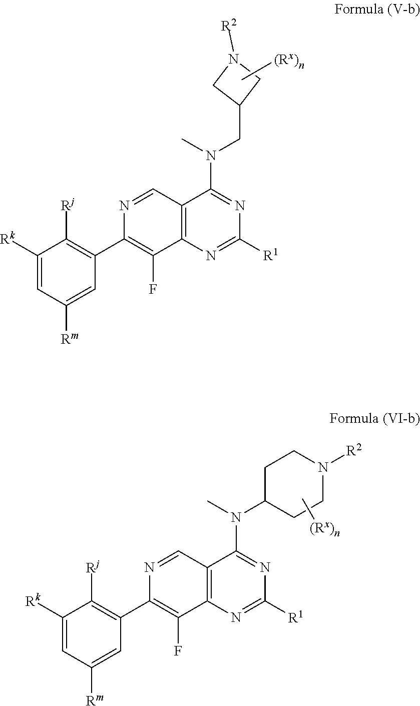

wherein R¹, R², Rⁱ, R^{k}, Rⁿ, R^{x} and n are as defined in any of the embodiments described herein.

In an embodiment, the compound is of Formula (I-b).

In an embodiment, the compound is of Formula (II-b).

In an embodiment, the compound is of Formula (III-b).

In an embodiment, the compound is of Formula (IV-b).

In an embodiment, the compound is of Formula (V-b).

In an embodiment, the compound is of Formula (VI-b).

In an embodiment, the stereochemistry of the pyrrolidine is (R) (i.e., the moiety represented as

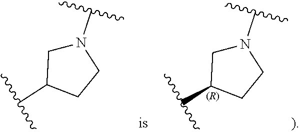

In an embodiment, the stereochemistry of the cyanomethyl groups is (S) (i.e., the moiety represented as is

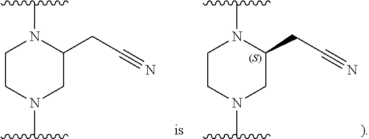

In an embodiment, the compound is selected from the compounds of Table 1 or a salt thereof; and/or an isotopologue thereof. In an embodiment, the compound is not a salt.

In an embodiment, the compound is a salt. In an embodiment, the salt is a formate salt. In an embodiment, the salt is a trifluoroacetate salt. In an embodiment, the salt is a pharmaceutically acceptable salt.

In some variations, any of the compounds described herein, such as a compound of Formula (I), (II), (III), (IV), (V), or (VI), or a compound of Table 1 may be deuterated (e.g., a hydrogen atom is replaced by a deuterium atom). In some of these variations, the compound is deuterated at a single site. In other variations, the compound is deuterated at multiple sites. Deuterated compounds can be prepared from deuterated starting materials in a manner similar to the preparation of the corresponding non-deuterated compounds. Hydrogen atoms may also be replaced with deuterium atoms using other method known in the art.

Any formula given herein, such as Formula (I), (II), (III), (IV), (V), or (VI), is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Representative examples of compounds detailed herein, including intermediates and final compounds, are depicted in the tables and elsewhere herein. It is understood that in one aspect, any of the compounds may be used in the methods detailed herein, including, where applicable, intermediate compounds that may be isolated and administered to an individual.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

In one variation, the compounds herein are synthetic compounds prepared for administration to an individual. In another variation, compositions are provided containing a compound in substantially pure form. In another variation, provided are pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Any variation or embodiment of R¹, R^{1A}, R^{d}, R², R^{Y1}, R^{Y2}, R^{e}, R^{e1}, R^{e2}, R^{e3}, R³¹, R³², R³, R⁴, R⁵, R⁶, R^{h}, Rⁱ, R^{j}, R^{k}, Rⁿ, R^{o}, R^{q}, R^{r}, R^{s}, R^{g}, R^{m}, R^{p}, R^{a}, R^{x}, Ring A, m, n, R^{x}′, R^{x}″, R^{x}′″, R^{y}, R^{y}′, R^{y}″, R^{z}, R^{z}, R^{z}″, R^{a}′, R^{b}′, or R^{c}′ provided herein can be combined with every other variation or embodiment of R¹, R^{1A}, R^{d}, R², R^{Y1}, R^{Y2}, R^{e}, R^{e1}, R^{e2}, R^{e3}, R³¹, R³², R³, R⁴, R⁵, R⁶, R^{h}, Rⁱ, R^{j}, R^{k}, Rⁿ, R^{o}, R^{q}, R^{r}, R^{s}, R^{g}, R^{m}, R^{p}, R^{a}, R^{x}, Ring A, m, n, R^{x}′, R^{x}″, R^{x}′″, R^{y}, R^{y}′, R^{y}″, R^{z}, R^{z}′, R^{z}″, R^{a′}, R^{b′}, or R^{c′} as if each combination had been individually and specifically described.

As used herein, when any variable occurs more than one time in a chemical formula, its definition on each occurrence is independent of its definition at every other occurrence.

### Methods For Treatment of Cancer

The compounds of Formula (I), Formula (II), Formula (III), Formula (IV), Formula (V), Formula (VI), and pharmaceutically acceptable salts and/or isotopologues thereof, including embodiments thereof disclosed herein, are useful for the treatment of cancer, which include but are not limited to, various types of cancer including e.g. lung, colorectal, pancreatic, bile duct, thyroid, gall bladder, uterine, mesothelioma, cervical, and bladder cancers. More particularly, cancers that may be treated by the compounds of Formula (I), Formula (II), Formula (III), Formula (IV), Formula (V), Formula (VI), and pharmaceutically acceptable salts and/or isotopologues thereof, including embodiments thereof disclosed herein, include, but are not limited to cancers such as glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In some embodiments, including any of the foregoing embodiments, the cancer is a KRAS G12C mediated cancer. In some embodiments, including any of the foregoing embodiments, the subject has been diagnosed as having a KRAS G12C mediated cancer. In some embodiments, including any of the foregoing embodiments, the subject has been determined to be at risk of developing a KRAS G12C mediated cancer.

In an aspect, provided is a compound of Formula (I), Formula (II), Formula (III), Formula (IV), Formula (V) or Formula (VI) as described in any of the embodiments described herein or a pharmaceutical formulation as described in any of the embodiments described herein for use as a medicament.

In an aspect, provided is a compound of Formula (I), Formula (II), Formula (III), Formula (IV), Formula (V) or Formula (VI) as described in any of the embodiments described herein or a pharmaceutical formulation as described in any of the embodiments described herein for use in treating or suppressing cancer. In an embodiment, when the compound is a salt, the salt is a pharmaceutically acceptable salt. In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, non-small cell lung cancer (NSCLC), and melanoma. In an embodiment, the cancer is a KRAS G12C mediated cancer. In an embodiment, the subject has been diagnosed as having a KRAS G12C mediated cancer. In an embodiment, the compound or pharmaceutical composition is configured for administration with a therapeutically effective amount of an additional chemotherapeutic agent. In an embodiment, the compound or pharmaceutical composition is configured for administration in a therapeutically effective amount.

In an aspect, provided is a compound of Formula (I), Formula (II), Formula (III), Formula (IV), Formula (V) or Formula (VI) as described in any of the embodiments described herein or a pharmaceutical formulation as described in any of the embodiments described herein for use in the manufacturing of a medicament for treating or suppressing cancer, wherein when the compound is a salt, the salt is a pharmaceutically acceptable salt. In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, non-small cell lung cancer (NSCLC), and melanoma. In an embodiment, the cancer is a KRAS G12C mediated cancer. In an embodiment, the subject has been diagnosed as having a KRAS G12C mediated cancer. In an embodiment, the compound or pharmaceutical composition is configured for administration with a therapeutically effective amount of an additional chemotherapeutic agent. In an embodiment, the medicament comprises a therapeutically effective amount of the compound or pharmaceutical composition.

In an aspect, provided is a use of a compound of Formula (I), Formula (II), Formula (III), Formula (IV), Formula (V) or Formula (VI) as described in any of the embodiments described herein or a pharmaceutical formulation as described in any of the embodiments described herein in the manufacturing of a medicament for treating or suppressing cancer, wherein when the compound is a salt, the salt is a pharmaceutically acceptable salt. In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, non-small cell lung cancer (NSCLC), and melanoma. In an embodiment, the cancer is a KRAS G12C mediated cancer. In an embodiment, the subject has been diagnosed as having a KRAS G12C mediated cancer. In an embodiment, the compound or pharmaceutical composition is configured for administration with a therapeutically effective amount of an additional chemotherapeutic agent. In an embodiment, the medicament comprises a therapeutically effective amount of the compound or pharmaceutical composition.

In an aspect, provided is a use of a compound of Formula (I), Formula (II), Formula (III), Formula (IV), Formula (V) or Formula (VI) as described in any of the embodiments described herein or a pharmaceutical formulation as described in any of the embodiments described herein for treating or suppressing cancer, wherein when the compound is a salt, the salt is a pharmaceutically acceptable salt.

In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, non-small cell lung cancer (NSCLC), and melanoma. In an embodiment, the cancer is a KRAS G12C mediated cancer. In an embodiment, the subject has been diagnosed as having a KRAS G12C mediated cancer. In an embodiment, the compound or pharmaceutical composition is configured for administration with a therapeutically effective amount of an additional chemotherapeutic agent. In an embodiment, use involves a therapeutically effective amount of the compound or composition. In some embodiments, including any of the foregoing embodiments, the subject and/or the cancer is resistant or refractory to treatment with certain KRAS inhibitors (e.g., G12C KRAS inhibitors).

The compounds of Formula (I), Formula (II), Formula (III), Formula (IV), Formula (V) or Formula (VI), and pharmaceutically acceptable salts and/or isotopologues thereof, including embodiments thereof disclosed herein, may be used for methods for inhibiting KRAS G12C in a cell, by contacting the cell in which inhibition of KRAS G12C activity is desired with an amount of the compound effective to inhibit KRAS G12C activity. Inhibition may be partial or total. In some embodiments, the contacting is in vitro. In some embodiments, the contacting is in vivo.

### Testing

The compounds of Formula (I), Formula (II), Formula (III), Formula (IV), Formula (V), Formula (VI), and pharmaceutically acceptable salts and/or isotopologues thereof, including embodiments thereof disclosed herein, may be tested by, for example, methods described in the Examples below, or by known and generally accepted cell and/or animal models.

The ability of compounds of Formula (I), Formula (II), Formula (III), Formula (IV), Formula (V), Formula (VI), and pharmaceutically acceptable salts and/or isotopologues thereof, to inhibit activity of the GTP-bound form of KRAS G12C can be tested using methods such as the in vitro assay described in Examples 16 and 17 below. Example 16 describes determining, for various compounds, the half-maximal inhibition (IC₅₀) of KRAS G12C loaded with GTP analogue GMPPNP from binding to cRaf, as the Ras-binding domain (RBD). Example 17 describes determining, for various compounds, the half-maximal inhibition (IC₅₀) of KRAS G12C loaded with GTP analogue GMPPNP from binding to PI3Kα, as the Ras-binding domain (RBD). Example 18 describes testing compounds for the ability to inhibit cell viability in MCF10A G12C/A59G mutant, which abrogates GTPase activity, thus preventing hydrolysis of GTP to GDP.

### Pharmaceutical Compositions

The terms “pharmaceutical composition” and “pharmaceutical formulation” are used interchangeably.

In general, the compounds of Formula (I), Formula (II), Formula (III), Formula (IV), Formula (V), Formula (VI), and pharmaceutically acceptable salts and/or isotopologues thereof, of this disclosure (also may be referred to herein as “compounds” or “compounds of this disclosure”) will be administered in a therapeutically effective amount by any of the accepted modes of administration for agents that serve similar utilities. Therapeutically effective amounts of compounds of this disclosure may range from about 0.01 to about 500 mg per kg patient body weight per day, which can be administered in single or multiple doses. In some embodiments, a suitable dosage level may be from about 0.1 to about 250 mg/kg per day; or about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of a compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of this disclosure in combination with at least one pharmaceutically acceptable excipient. Acceptable excipients are non-toxic, aid administration, and do not adversely affect the therapeutic benefit of the compound of this disclosure. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds of the disclosure may be administered topically, that is by non-systemic administration. This includes the application of the compounds externally to the epidermis or the buccal cavity and the instillation of such compounds into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients. For example, the compound is present at a level of about 1-80 wt. %.

### Combinations and Combination Therapies

The compounds of this disclosure may be used in combination with one or more other drugs in the treatment of diseases or conditions for which compounds of this disclosure or the other drugs may have utility. Such other drug(s) may be administered contemporaneously or sequentially with a compound of the present disclosure. When a compound of this disclosure is used contemporaneously with one or more other drugs, a pharmaceutical composition in unit dosage form containing such other drugs and the compound of the present disclosure is contemplated. However, the combination therapy may also include therapies in which the compound of this disclosure and one or more other drugs are administered on different overlapping schedules. It is also contemplated that when used in combination with one or more other active ingredients, the compounds of the present disclosure and the other active ingredients may be used in lower doses than when each is used singly.

Accordingly, the pharmaceutical compositions of the present disclosure also include those that contain one or more other drugs, in addition to a compound of the present disclosure.

The above combinations include combinations of a compound of this disclosure not only with one other drug, but also with two or more other active drugs. Likewise, a compound of this disclosure may be used in combination with other drugs that are used in the prevention, treatment, control, amelioration, or reduction of risk of the diseases or conditions for which a compound of this disclosure is useful. Such other drugs may be administered contemporaneously or sequentially with a compound of the present disclosure. When a compound of this disclosure is used contemporaneously with one or more other drugs, a pharmaceutical composition containing such other drugs in addition to the compound of this disclosure can be used. Accordingly, the pharmaceutical compositions of the present disclosure also include those that also contain one or more other active ingredients, in addition to a compound of this disclosure. The weight ratio of the compound of this disclosure to the second active ingredient may be varied and will depend upon the effective dose of each ingredient. Generally, a therapeutically effective dose of each will be used.

Where the subject in need is suffering from or at risk of suffering from cancer, the subject can be treated with a compound of this disclosure in any combination with one or more other anti-cancer agents.

In some embodiments, the compounds of the present disclosure are used in combination with a CDK 4/6 inhibitor. Examples of CDK 4/6 inhibitors suitable for the provided compositions and methods include, but are not limited to, abemaciclib (N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine); palbociclib (6-acetyl-8-cyclopentyl-5-methyl-2-((5-(piperazin-1-yl)pyridin-2-yl)amino)-pyrido[2,3-d]pyrimidin-7(8H)-one) and ribociclib (7-cyclopentyl-N,N-dimethyl-2-((5-(piperazin-1-yl)pyridin-2-yl)amino)-7H-pyrrolo[2,3-d]pyrimidine-6-carboxamide) whereas the CDK 4/6 inhibitor trilaciclib (2′-((5-(piperazin-1-yl)pyridin-2-yl)amino)-7′,8′-dihydro-6′H-spiro-[cyclohexane-1,9′-pyrazino[1′,2′:1,5]pyrrolo[2,3-d]pyrimidin]-6′-one) is in late stage clinical trials. Another CDK 4/6 inhibitor useful in the methods herein is the CDK 2/4/6 inhibitor PF-06873600 (pyrido[2,3-d]pyrimidin-7(8H)-one, 6-(difluoromethyl)-8-[(1R,2R)-2-hydroxy-2-methylcyclopentyl]-2-[[1-(methylsulfonyl)-4-piperidinyl]amino]).

In another embodiment the compounds of the present disclosure are used in combination with Raf family kinase inhibitors. Examples of Raf family kinase inhibitors suitable for the provided compositions and methods include, but are not limited to, encorafenib (LGX818): methyl (S)-(1-((4-(3-(5-chloro-2-fluoro-3-(methylsulfonamido)phenyl)-1-isopropyl-1H-pyrazol-4-yl)pyrimidin-2-yl)amino)propan-2-yl)carbamate; PLX-8394: N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide; Raf-709: N-(2-methyl-5′-morpholino-6′-((tetrahydro-2H-pyran-4-yl)oxy)-[3,3′-bipyridin]-5-yl)-3-(trifluoromethyl)benzamide; LXH254: N-(3-(2-(2-hydroxyethoxy)-6-morpholinopyridin-4-yl)-4-methylphenyl)-2-(trifluoromethyl)isonicotinamide; Sorafenib: 4-(4-(3-(4-chloro-3-(trifluoromethyl)phenyl)ureido)phenoxy)-N-methylpicolinamide; L Y 3009120: 1-(3,3-dimethylbutyl)-3-(2-fluoro-4-methyl-5-(7-methyl-2-(methylamino)pyrido-[2,3-d]pyrimidin-6-yl)phenyl)urea; Lifirafenib (BGB-283); 5-(((1R,1aS,6bS)-1-(6-(trifluoro-methyl)-1H-benzo[d]imidazol-2-yl)-1a,6b-dihydro-1H-cyclopropa[b]benzofuran-5-yl)methyl)-3,4-dihydro-1,8-naphthyridin-2(1H)-one; Tak-632: N-(7-cyano-6-(4-fluoro-3-(2-(3-(trifluoromethyl)-phenyl)acetamido)phenoxy)benzo[d]thiazol-2-yl)cyclopropanecarboxamide; CEP-32496: 1-(3-((6,7-dimethoxyquinazolin-4-yl)oxy)phenyl)-3-(5-(1,1,1-trifluoro-2-methylpropan-2-yl)isoxazol-3-yl)urea; CCT196969: 1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl)urea; and R05126766: N-[3-fluoro-4-[[4-methyl-2-oxo-7-(2-pyrimidinyloxy)-2H-1-benzopyran-3-yl]methyl]-2-pyridinyl]-N′-methylsulfamide.

In another embodiment the compounds of the present disclosure are used in combination with Src family kinases. Examples of Src family kinase inhibitors suitable for the provided compositions and methods include, but are not limited to, Dasatinib (N-(2-chloro-6-methylphenyl)-2-((6-(4-(2-hydroxyethyl)piperazin-1-yl)-2-methylpyrimidin-4-yl)amino)thiazole-5-carboxamide); Ponatinib (3-(imidazo[1,2-b]pyridazin-3-ylethynyl)-4-methyl-N-(4-((4-methylpiperazin-1-yl)methyl)-3-(trifluoromethyl)phenyl)benzamide); Vandetanib (N-(4-bromo-2-fluorophenyl)-6-methoxy-7-((1-methylpiperidin-4-yl)methoxy)quinazolin-4-amine); Bosutinib (4-((2,4-dichloro-5-methoxyphenyl)amino)-6-methoxy-7-(3-(4-methylpiperazin-1-yl)-propoxy)quinoline-3-carbonitrile); Saracatinib (N-(5-chlorobenzo[d][1,3]dioxol-4-yl)-7-(2-(4-methylpiperazin-1-yl)ethoxy)-5-((tetrahydro-2H-pyran-4-yl)oxy)quinazolin-4-amine); KX2-391 (N-benzyl-2-(5-(4-(2-morpholinoethoxy)phenyl)pyridin-2-yl)acetamide); SU6656 ((Z)—N,N-dimethyl-2-oxo-3-((4,5,6,7-tetrahydro-1H-indol-2-yl)methylene)indoline-5-sulfonamide); PP1 (1-(tert-butyl)-3-(p-tolyl)-1H-pyrazolo[3,4-d]pyrimidin-4-amine); WH-4-023 (2,6-dimethylphenyl (2,4-dimethoxyphenyl)(2-((4-(4-methylpiperazin-1-yl)phenyl)amino)pyrimidin-4-yl)carbamate) and KX-01 (N-benzyl-2-(5-(4-(2-morpholinoethoxy)phenyl)pyridin-2-yl)acetamide). In one embodiment, the Src inhibitor is Dasatinib. In one embodiment, the Src inhibitor is Saracatinib. In one embodiment, the Src inhibitor is Ponatinib. In one embodiment, the Src inhibitor is Vandetanib. In one embodiment, the Src inhibitor is KX-01.

In another embodiment the compounds of the present disclosure are used in combination with a SHP-2 inhibitor which include, but are not limited to SHP-099 (6-(4-amino-4-methylpiperidin-1-yl)-3-(2,3-dichlorophenyl)pyrazine-2-amine dihydrochloride), RMC-4550 (3(3S,4S)-(4-amino-3-methyl-2-oxa-8-azaspiro[4.5]decan-8-yl)-6-(2,3-dichlorophenyl)pyrazin-2-yl)methanol), RMC-4360 (Revolution Medicines), TN0155 (Novartis), BBP-398 (BridgeBio), and ERAS-601 (Erasca).

In another embodiment the compounds of the present disclosure are used in combination with an mTOR inhibitor. Examples of mTOR inhibitors suitable for the provided compositions and methods include, but are not limited to, Everolimus, Rapamycin, Zotarolimus (ABT-578), ridaforolimus (Deforolimus; MK-8669), Sapanisertib (INK128; 5-(4-amino-1-isopropyl-1H-pyrazolo[3,4-d]pyrimidin-3-yl)benzo[d]oxazol-2-amine), Torin-1; 1-(4-(4-propionylpiperazin-1-yl)-3-(trifluoromethyl)cyclohexyl)-9-(quinolin-3-yl)benzo[h][1,6]naphthyridin-2(1H)-one, dactolisib (BEZ235); 2-methyl-2-(4-(3-methyl-2-oxo-8-(quinolin-3-yl)-2,3-dihydro-1H-imidazo[4,5-c]quinolin-1-yl)phenyl)propanenitrile, buparlisib (5-(2,6-dimorpholin-4-ylpyrimidin-4-yl)-4-(trifluoromethyl)pyridin-2-amine); GDC-0941 (pictilisib); 4-[2-(1H-indazol-4-yl)-6-[(4-methylsulfonylpiperazin-1-yl)methyl]thieno[3,2-d]pyrimidin-4-yl]morpholine); GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), VS-5584 (SB2343) (5-(8-methyl-2-morpholin-4-yl-9-propan-2-yl-purin-6-yl)pyrimidin-2-amine) and vistusertib (AZD-2014; 3-(2,4-bis((S)-3-methylmorpholino)pyrido-[2,3-d]pyrimidin-7-yl)-N-methylbenzamide).

In another embodiment the compounds of the present disclosure are used in combination with a pan ErbB family inhibitor. In one embodiment the KRAS and pan ErbB family inhibitors are the only active agents in the provided compositions and methods. In one embodiment, the pan ErbB family inhibitor is an irreversible inhibitor. Examples of irreversible pan ErbB family inhibitors suitable for the provided compositions and methods include, but are not limited to, Afatinib; Dacomitinib; Canertinib; Poziotinib, AV 412 (N-4-([3-(chloro-4-fluorophenyl)amino]-7-[3-methyl-3-(4-methyl-1-piperazin-1-butyn-1-yl]-6-quinazolinyl]-2-prepenamide); PF 6274484 N-4-([3-(chloro-4-fluorophenyl)amino]-7-methoxy-6-quinazolinyl]-2-propenamide) and HKI 357 N-(2(E)-N-[[4-[[3-chloro-4-[(fluorophenyl)methoxy]phenyl]amino]-3-cyano-7-ethoxy-6-quinolinyl]-4-(dimethylamino)-2-butenamide). In another embodiment, the pan ErbB family inhibitor is a reversible inhibitor. Examples of reversible pan ErbB family inhibitors suitable for the provided compositions and methods include, but are not limited to erlotinib, gefitinib, sapitinib; varlitinib; TAK-285 (N-[2-[4-[3-chloro-4-[3-(trifluoromethyl)phenoxy]phenylamino]-5H-pyrrolo[3,2-d]pyrimidin-5-yl]ethyl]-3-hydroxy-3-methylbutanamide); AEE788 (S)-(6-(4-((4-ethylpiperazin-1-ylmethyl)phenyl]-N-(1-phenylethyl)-7H-pyrrolo[2,3-d]pyrimidin-4-amine); tarloxotinib 3-[N-[4-(3-bromo-4-chlorophenylamino)-pyrido[3,4-d]pyrimidin-6-yl]carbamoyl]-N,N-dimethyl-N-(1-methyl-4-nitro-1H-imidazol-5-ylmethyl)-2(E)-propen-1-aminium bromide); BMS 599626 ((3S)—3-morpholinylmethyl-[4-[[1-[(3-fluorophenyl)methyl]-1H-indazol-5-yl]amino]-5-methylpurrolo[2,1-f][1,2,4]triazine-6-yl]carbamate dihydrochloride); and GW 583340 (N-[3-chloro-4-(3-fluorobenzyloxy)phenyl]-6-[2-[2-(methylsulfonyl)ethylaminomethyl]thiazol-4-yl]quinazolin-4-amine dihydrochloride).

In one embodiment, the pan ErbB family inhibitor is a combination of an EGFR inhibitor and a HER2 inhibitor, wherein the EGFR inhibitor and the HER2 inhibitor are a combination of two of: AG 1478 (N-(3-chlorophenyl)-6,7-dimethoxyquinazolin-4-amine hydrochloride); AG 555 ((E)-2-cyano-3-(3,4-dihydoxyphenyl)-N-(3-phenylpropyl)-2-propenamide); AG 556 ((E)-2-cyano-3-(3,4-dihydroxyphenyl)-N-(4-phenylbutyl)-2-propenamide; AG 825 (E-3-[3-benzothiazol-2-ylsulfanylmethyl)-4-hydroxy-5-methoxyphenyl]-2-cyano-2-propenamide); CP 724714 (2-methoxy-N-[(2E)-3-[4-[3-methyl-4-(6-methylpyridin-3-yloxy)phenylamino]quinazolin-6-yl]-2-propen-1-yl]acetamide; BIBU 1361 (N-(3-chloro-4-fluorophenyl)-6-[4-(diethylaminomethyl)-piperidin-1-yl]pyrimido[5,4-d]pyrimidin-4-amine dihydrochloride); BIBU 1382; (N⁸-(3-chloro-4-fluorophenyl)-N²-(1-methyl-4-piperidinyl)pyrimidino[5,4-d]pyrimidin-4-amine dihydrochloride), JNJ 28871063 (5E-4-amino-6-[4-(benzyloxy)-3-chlorophenylamino]-pyrimidine-5-carbaldehyde N-[2-(4-morpholinyl)ethyl]oxime hydrochloride); PD 153035 (4-(3-bromophenylamino)-6,7-dimethoxyquinazoline hydrochloride); and PD 158780 (N⁴-(3-bromophenyl)-N⁶-methyl-pyrido[3,4-d]pyrimidine-4,6-diamine).

In one embodiment, the pan ErbB family inhibitor is an anti-EGFR antibody, an anti-HER2 antibody or combination of an anti-EGFR antibody and anti-HER2 antibody. Antibodies, including monoclonal antibodies, antibody conjugates and bispecific antibodies, targeting EGFR and/or HER2 are well known and several antibodies are commercially available for research and human clinical use. Examples of anti-EGFR antibodies suitable for the provided compositions and methods include necitumumab, panitumumab and cetuximab. Examples of anti-HER2 antibodies suitable for the provided compositions and methods include, pertuzumab, trastuzumab, and trastuzumab emtansine.

In some embodiments, the compounds of the present disclosure are used in combination with an immune checkpoint inhibitor. Examples of immune checkpoint inhibitors suitable for the provided compositions and methods include, but are not limited to, PD-1, PD-L1, CTLA-4, and LAG-3 inhibitors, such as Pembrolizumab (Keytruda®), Nivolumab (Opdivo®), Cemiplimab (Libtayo®), Atezolizumab (Tecentriq®), Avelumab (Bavencio®), Durvalumab (Imfinzi™), Ipilimumab (Yervoy®), Relatlimab, Opdualag, and Dostarlimab (Jemperli).

The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

### ENUMERATED EMBODIMENTS

The following enumerated embodiments are representative of some aspects of the invention.

Embodiment 1. A compound of Formula (I), Formula (II), Formula (III), Formula (IV), Formula (V) or Formula (VI):

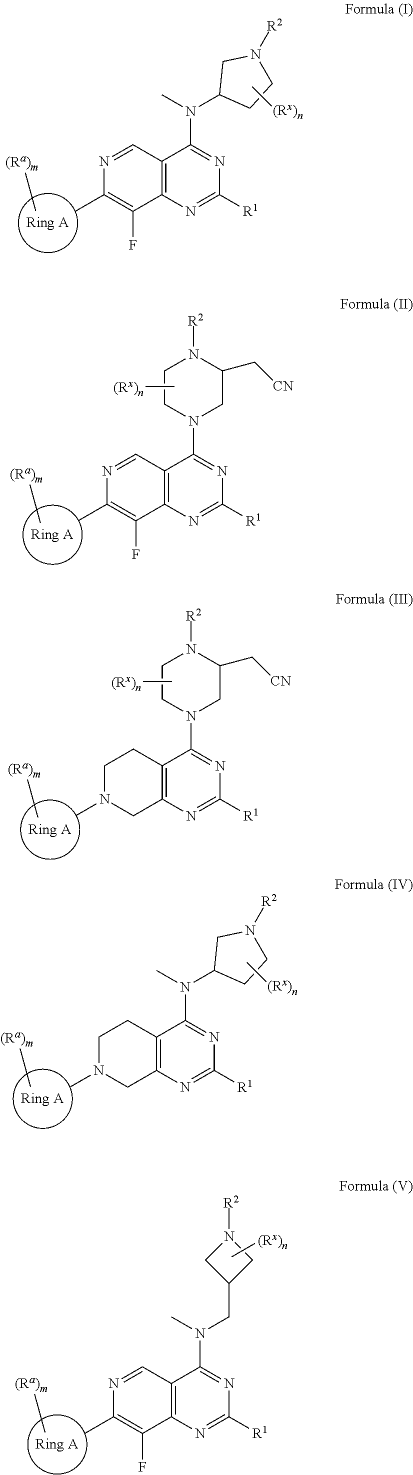

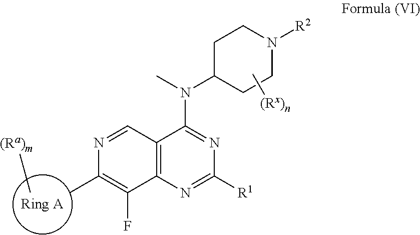

or a salt thereof; and/or an isotopologue thereof; wherein:
Ring A is a 6-10 membered aryl or a 5-10 membered heteroaryl;
each R^{a} is independently selected from halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl;
mis 0, 1, 2 or 3;
R¹ is selected from the group consisting of H, —OCH₂R^{1A}, —OC₁-C₄ alkyl, —C₁-C₄ alkyl and

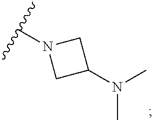 [SMILES: *N1CC(N(C)C)C1]

R^{1A} is selected from the group consisting of

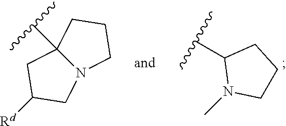

R^{d} is H or F;
R² is

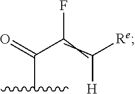

R^{e} is selected from the group consisting of —COOH, —C(O)O—C₁-C₄ alkyl, —C(O)O—C₁-C₄ haloalkyl, —C(O)—C₁-C₄ alkyl, —C(O)—C₁-C₄ haloalkyl, —C(O)N(C₁-C₄ alkyl)₂, —(C₁-C₂ alkyl)-(C₁-C₂ alkoxy), —S(O)₂—C₁-C₄ alkyl, —S(O)₂—C₁-C₄ haloalkyl, R^{e1}, —C(R^{Y}i)(R^{Y2})R^{e}, R^{e2} and —C(R^{Y1})(R^{Y2})R^{e3};
R^{Y1} and R^{Y2} in each occurrence are independently selected from —H and —CH₃;
R^{e1} is a 4-10 membered heterocycle which is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl, and C₁-C₄ haloalkoxy;
R^{e2} is a 5-6 membered heteroaryl group substituted with 0, 1, 2 or 3 substituents independently selected from halo, hydroxy, C₁-C₄ alkyl, C₁-C₄ hydroxyalkyl, C₁-C₄ alkoxy, C₁-C₄ haloalkyl, C₁-C₄ haloalkoxy, C₃-C₆ heterocyclyl substituted with 0, 1 or 2 substituents independently selected from halo, hydroxy and methyl, and C₃-C₆ cycloalkyl substituted with 0, 1 or 2 substituents independently selected from halo, hydroxy and methyl;
R^{e3} is —NR³¹R³²;
R³¹ and R³² are independently selected from the group consisting of hydrogen, C₁-C₄ alkyl, C₁-C₄ alkoxy, C₁-C₄ haloalkoxy, and C₁-C₄ alkyl substituted with a 3-6 membered heterocycle;
R^{x} is selected from —OH, halo and C₁-C₄ alkyl; and
n is 0, 1 or 2.

Embodiment 2. The compound of embodiment 1, wherein the compound is of Formula (I).

Embodiment 3. The compound of embodiment 1, wherein the compound is of Formula (II).

Embodiment 4. The compound of embodiment 1, wherein the compound is of formula (III).

Embodiment 5. The compound of embodiment 1, wherein the compound is of formula (IV).

Embodiment 6. The compound of embodiment 1, wherein the compound is of formula (V).

Embodiment 7. The compound of embodiment 1, wherein the compound is of formula (VI).

Embodiment 8. The compound of any one of embodiments 1, 2 and 5, wherein the stereochemistry of the pyrrolidine is (R) (i.e., the moiety represented as is

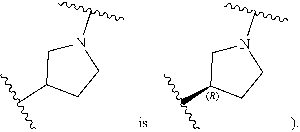

Embodiment 9. The compound of any one of embodiments 1, 3 and 4, wherein the stereochemistry of the cyanomethyl groups is (S) (i.e., the moiety represented as is

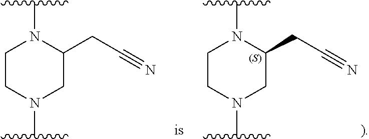

Embodiment 10. The compound of any one of embodiments 1-9, wherein Ring A is selected from a 6-10 membered aryl and a 9-10 membered bicyclic heteroaryl having 1, 2 or 3 heteroatoms independently selected from N, O and S.

Embodiment 11. The compound of any one of embodiments 1-9, wherein Ring A is selected from the group consisting of naphthalenyl, phenyl, isoquinolinyl, indazolyl and pyridinyl.

Embodiment 12. The compound of any one of embodiments 1-9, wherein Ring A is selected from the group consisting of naphthalenyl and phenyl.

Embodiment 13. The compound of any one of embodiments 1-9, wherein Ring A is naphthalenyl.

Embodiment 14. The compound of any one of embodiments 1-9, wherein Ring A is phenyl.

Embodiment 15. The compound of any one of embodiments 1-9, wherein Ring A is selected from the group consisting of naphthalen-1-yl, phenyl, isoquinolin-1-yl, indazol-4-yl and pyridin-1-yl.

Embodiment 16. The compound of any one of embodiments 1-9, wherein Ring A is selected from the group consisting of naphthalen-1-yl and phenyl.

Embodiment 17. The compound of any one of embodiments 1-9, wherein Ring A is naphthalen-1-yl.

Embodiment 18. The compound of any one of embodiments 1-17, wherein each R^{a} is independently selected from halo, —OH, C₁-C₄ alkyl, C₃-C₄ cycloalkyl, C₁-C₄ haloalkyl and C₂-C₃ alkynyl.

Embodiment 19. The compound of any one of embodiments 1-17, wherein each R^{a} is independently selected from halo, C₁-C₄ alkyl, C₃-C₄ cycloalkyl, C₁-C₄ haloalkyl and C₂-C₃ alkynyl.

Embodiment 20. The compound of any one of embodiments 1-17, wherein each R^{a} is independently selected from halo and C₁-C₄ alkyl.

Embodiment 21. The compound of any one of embodiments 1-17, wherein each R^{a} is independently selected from halo and C₃-C₄ cycloalkyl.

Embodiment 22. The compound of any one of embodiments 1-17, wherein each R^{a} is independently selected from halo and C₁-C₄ haloalkyl.

Embodiment 23. The compound of any one of embodiments 1-17, wherein each R^{a} is independently selected from halo and C₂-C₃ alkynyl.

Embodiment 24. The compound of any one of embodiments 1-17, wherein each R^{a} is independently halo.

Embodiment 25. The compound of any one of embodiments 1-17, wherein each R^{a} is independently selected from —F, —Cl, —OH, -Me, -Et, -cyclopropyl, —CF₃ and —C≡CH.

Embodiment 26. The compound of any one of embodiments 1-17, wherein each R^{a} is independently selected from —F, —Cl, -Me, -Et, -cyclopropyl, —CF₃ and —C≡CH.

Embodiment 27. The compound of any one of embodiments 1-17, wherein each R^{a} is independently selected from —F, —Cl, -cyclopropyl, —CF₃ and —C≡CH.

Embodiment 28. The compound of any one of embodiments 1-17, wherein each R^{a} is independently selected from —F, —Cl, -Me and -Et.

Embodiment 29. The compound of any one of embodiments 1-17, wherein each R^{a} is independently selected from —Cl and -cyclopropyl.

Embodiment 30. The compound of any one of embodiments 1-17, wherein each R^{a} is independently selected from —Cl and —CF₃.

Embodiment 31. The compound of any one of embodiments 1-17, wherein each R^{a} is independently selected from —F, —Cl and —C≡CH.

Embodiment 32. The compound of any one of embodiments 1-17, wherein each R^{a} is independently selected from —F and —Cl.

Embodiment 33. The compound of any one of embodiments 1-32, wherein m is 1, 2 or 3.

Embodiment 34. The compound of any one of embodiments 1-32, wherein m is 1 or 2.

Embodiment 35. The compound of any one of embodiments 1-32, wherein m is 2 or 3.

Embodiment 36. The compound of any one of embodiments 1-32, wherein m is 1.

Embodiment 37. The compound of any one of embodiments 1-32, wherein m is 2.

Embodiment 38. The compound of any one of embodiments 1-32, wherein m is 3.

Embodiment 39. The compound of any one of embodiments 1-9, wherein Ring A is selected from the group consisting of:

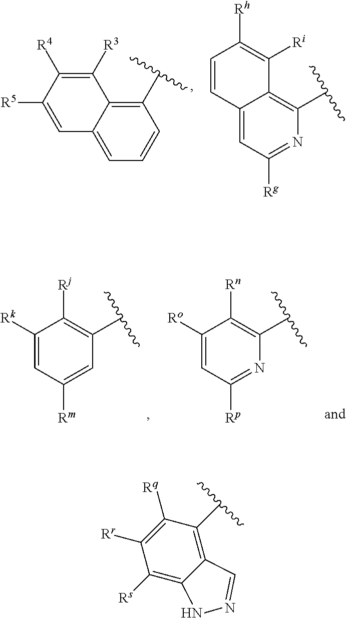

wherein:
each R³, R⁴, R⁵, R^{h}, Rⁱ, R^{j}, R^{k}, Rⁿ, R^{o}, R^{q}, R^{r} and R^{s} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl; and
each R^{g}, R^{m} and R^{p} is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl.

Embodiment 40. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of hydrogen, halo, C₁-C₄ alkyl, C₃-C₄ cycloalkyl, C₁-C₄ haloalkyl and C₂-C₃ alkynyl.

Embodiment 41. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of hydrogen, halo and C₁-C₄ alkyl.

Embodiment 42. The compound of any embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of hydrogen, halo and C₃-C₄ cycloalkyl.

Embodiment 43. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of hydrogen, halo and C₁-C₄ haloalkyl.

Embodiment 44. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of hydrogen, halo and C₂-C₃ alkynyl.

Embodiment 45. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of hydrogen and halo. Embodiment 46. The compound of embodiment 39, wherein each R³, R⁴, R⁵, R^{h}, Rⁱ, R^{j}, R^{k}, Rⁿ, R^{o}, R^{q}, R^{r} and R^{s} is independently halo.

Embodiment 47. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of —H, —F, —Cl, -Me, -Et, -cyclopropyl, —CF₃ and —C≡CH.

Embodiment 48. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of —H, —F, —Cl, -Me and -Et.

Embodiment 49. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of —H, —F, —Cl and -cyclopropyl.

Embodiment 50. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of —H, —F, —Cl and —CF₃.

Embodiment 51. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of —H, —F, —Cl and —C≡CH.

Embodiment 52. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of —H, —F and —Cl.

Embodiment 53. The compound of embodiment 39, wherein each R³, R⁴, R⁵, R^{h}, Rⁱ, R^{j}, R^{k}, Rⁿ, R^{o}, R^{q}, R^{r} and R^{s} is independently selected from the group consisting of —F and —Cl.

Embodiment 54. The compound of any one of embodiments 39 to 53, wherein each R⁹, R^{m} and R^{p} is independently selected from the group consisting of —H and —OH.

Embodiment 55. The compound of any one of embodiments 39 to 53, wherein each R^{g}, R^{m} and R^{p} is independently —H.

Embodiment 56. The compound of any one of embodiments 39 to 53, wherein each R^{g}, R^{m} and R^{p} is independently —OH.

Embodiment 57. The compound of any one of embodiments 39 to 56, wherein Ring A is selected from the group consisting of:

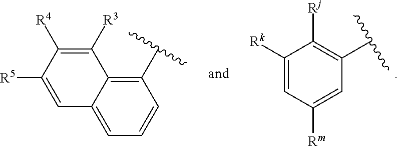

Embodiment 58. The compound of any one of embodiments 39 to 56, wherein Ring A is

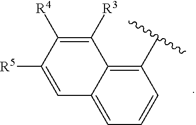

Embodiment 59. The compound of any one of embodiments 39 to 56, wherein Ring A is

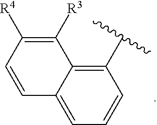

Embodiment 60. The compound of any one of embodiments 39 to 56, wherein Ring A is

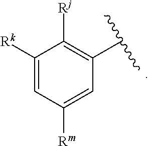

Embodiment 61. The compound of any one of embodiments 1-9, wherein Ring A is selected from:

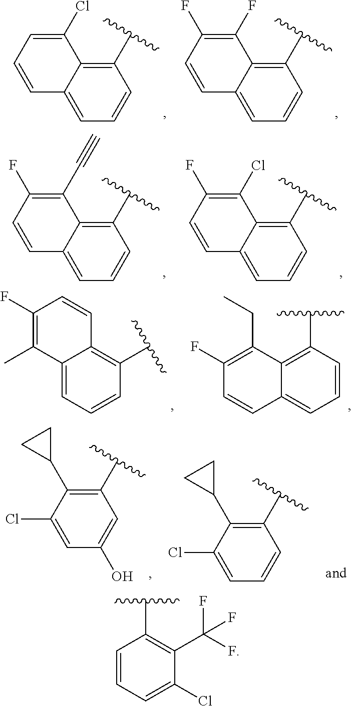

Embodiment 62. The compound of any one of embodiments 1-9, wherein Ring A is

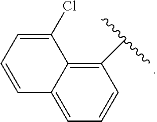 [SMILES: *c1cccc2cccc(Cl)c12]

Embodiment 63. The compound of any one of embodiments 1-9, wherein Ring A is

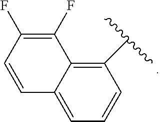 [SMILES: *c1cccc2ccc(F)c(F)c12]

Embodiment 64. The compound of any one of embodiments 1-9, wherein Ring A is

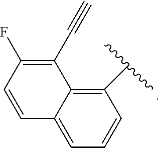 [SMILES: *c1cccc2ccc(F)c(C#C)c12]

Embodiment 65. The compound of any one of embodiments 1-9, wherein Ring A is

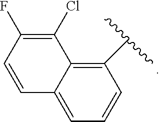 [SMILES: *c1cccc2ccc(F)c(Cl)c12]

Embodiment 66. The compound of any one of embodiments 1-9, wherein Ring A is

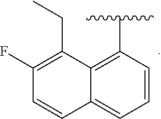 [SMILES: *c1cccc2ccc(F)c(CC)c12 ; "."]

Embodiment 67. The compound of any one of embodiments 1-9, wherein Ring A is

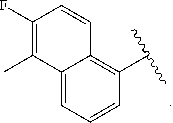 [SMILES: *c1cccc2c(C)c(F)ccc12]

Embodiment 68. The compound of any one of embodiments 1-9, wherein Ring A is

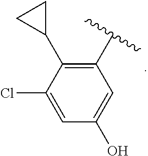 [SMILES: *c1cc(O)cc(Cl)c1C1CC1]

Embodiment 69. The compound of any one of embodiments 1-9, wherein Ring A is

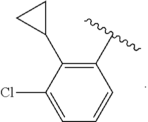 [SMILES: *c1cccc(Cl)c1C1CC1]

Embodiment 70. The compound of any one of embodiments 1-9, wherein Ring A is

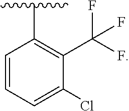 [SMILES: *c1cccc(Cl)c1C(F)(F)F]

Embodiment 71. The compound of embodiment 1, wherein the compound is of Formula (I-a), Formula (II-a), Formula (III-a), Formula (IV-a), Formula (V-a) or Formula (VI-a):

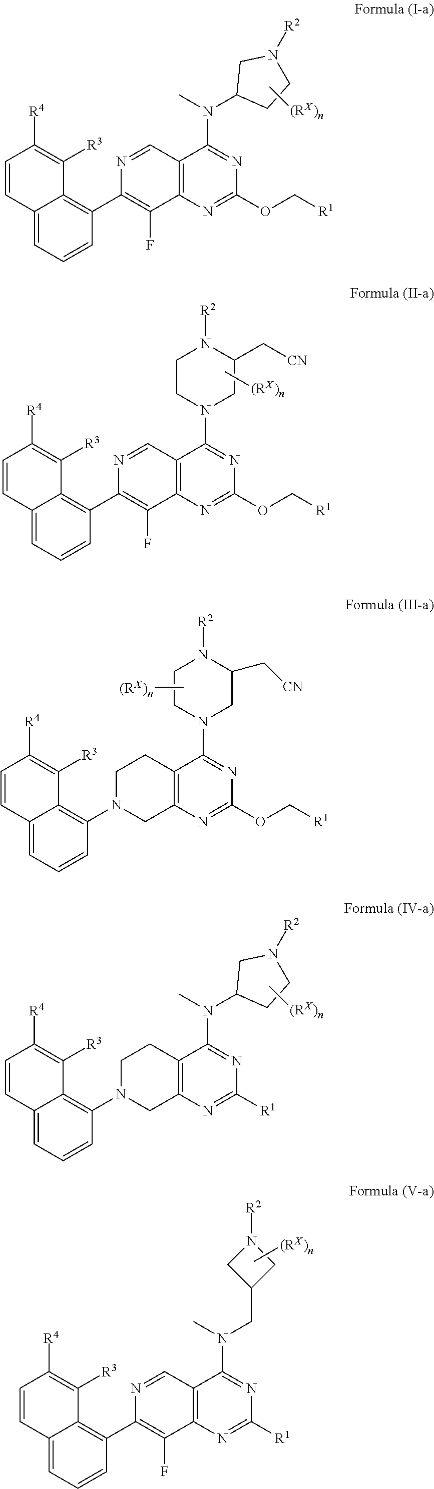

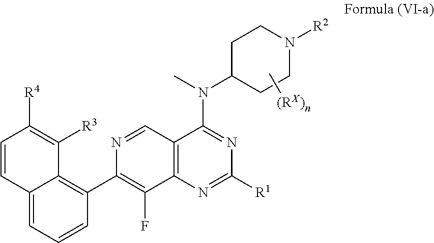

or a salt thereof; and/or an isotopologue thereof; wherein:
R³ is selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl; and
R⁴ is selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl.

Embodiment 72. The compound of embodiment 71, wherein the compound is of Formula (I-a).

Embodiment 73. The compound of embodiment 71, wherein the compound is of Formula (II-a).

Embodiment 74. The compound of embodiment 71, wherein the compound is of Formula (III-a).

Embodiment 75. The compound of embodiment 71, wherein the compound is of Formula (IV-a).

Embodiment 76. The compound of embodiment 71, wherein the compound is of Formula (V-a).

Embodiment 77. The compound embodiment 71, wherein the compound is of Formula (V-a).

Embodiment 78. The compound of any one of embodiments 71, 72 and 75 wherein the stereochemistry of the pyrrolidine is (R) (i.e., the moiety represented as is

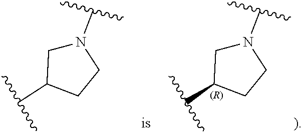

Embodiment 79. The compound of any one of embodiments 71, 73 and 74, wherein the stereochemistry of the cyanomethyl groups is (S) (i.e., the moiety represented as

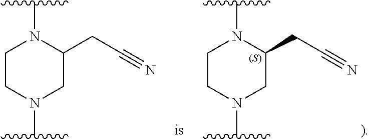

Embodiment 80. The compound of any one of embodiments 39, 57, 59 and 71-79, wherein R³ is selected from halo, C₁-C₄ alkyl, and C₂-C₃ alkynyl.

Embodiment 81. The compound of any one of embodiments 39, 57, 59 and 71-79, wherein R³ is selected from halo and C₁-C₄ alkyl.

Embodiment 82. The compound of any one of embodiments 39, 57, 59 and 71-79, wherein R³ is selected from halo and C₂-C₃ alkynyl.

Embodiment 83. The compound of any one of embodiments 39, 57, 59 and 71-79, wherein R³ is halo.

Embodiment 84. The compound of any one of embodiments 39, 57, 59 and 71-79, wherein R³ is selected from —F, —Cl, -Et, and —C≡CH.

Embodiment 85. The compound of any one of embodiments 39, 57, 59 and 71-79, wherein R³ is selected from —F, —Cl, and -Et.

Embodiment 86. The compound of any one of embodiments 39, 57, 59 and 71-79, wherein R³ is selected from —F, —Cl and —C≡CH.

Embodiment 87. The compound of any one of embodiments 39, 57, 59 and 71-79, wherein R³ is selected from —F and —Cl.

Embodiment 88. The compound of any one of embodiments 39, 57, 59 and 71-79, wherein R³ is —F.

Embodiment 89. The compound of any one of embodiments 39, 57, 59 and 71-79, wherein R³ is —Cl.

Embodiment 90. The compound of any one of embodiments 39, 57, 59 and 71-79, wherein R³ is -Et.

Embodiment 91. The compound of any one of embodiments 39, 39, 57, 59 and 71-79, wherein R³ is —C≡CH.

Embodiment 92. The compound of any one of embodiments 39, 57, 59 and 71-91, wherein R⁴ is selected from hydrogen and halo.

Embodiment 93. The compound of any one of embodiments 39, 57, 59 and 71-91, wherein R⁴ is selected from —H and —F.

Embodiment 94. The compound of any one of embodiments 39, 57, 59 and 71-91, wherein R⁴ is —H.

Embodiment 95. The compound of any one of embodiments 39, 57, 59 and 71-91, wherein R⁴ is —F.

Embodiment 96. The compound of embodiment 1, wherein the compound is of Formula (I-b), Formula (II-b), Formula (III-b), Formula (IV-b), Formula (V-b) or Formula (VI-b):

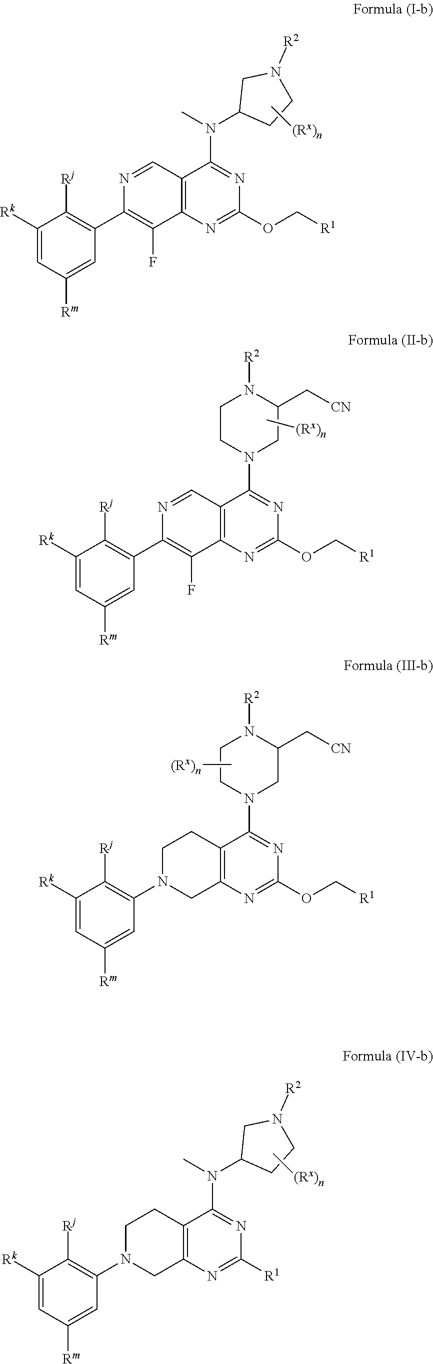

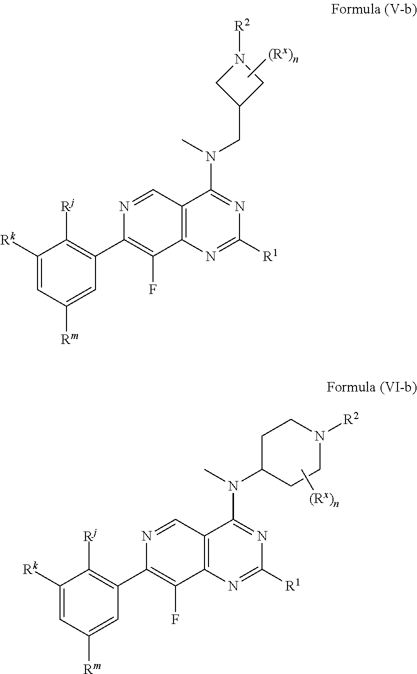

or a salt thereof; and/or an isotopologue thereof; wherein:
R^{j} is selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl;
R^{k} is selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl; and
R^{m} is selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl.

Embodiment 97. The compound of embodiment 96, wherein the compound is of Formula (I-b).

Embodiment 98. The compound of embodiment 96, wherein the compound is of Formula (II-b).

Embodiment 99. The compound of embodiment 96, wherein the compound is of Formula (III-b).

Embodiment 100. The compound of embodiment 96, wherein the compound is of Formula (IV-b).

Embodiment 101. The compound of embodiment 96, wherein the compound is of Formula (V-b).

Embodiment 102. The compound of embodiment 96, wherein the compound is of Formula (VI-b).

Embodiment 103. The compound of any one of embodiments 96, 97 and 100, wherein the stereochemistry of the pyrrolidine is (R) (i.e., the moiety represented as is

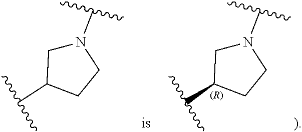

Embodiment 104. The compound of any one of embodiments 96, 98 and 99 wherein the stereochemistry of the cyanomethyl groups is (S) (i.e., the moiety represented as

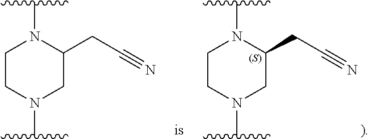

Embodiment 105. The compound of any one of embodiments 39, 57, 60, and 80-104, wherein R^{j} is selected from C₃-C₄ cycloalkyl and C₁-C₄ haloalkyl.

Embodiment 106. The compound of any one of embodiments 39, 57, 60, and 80-104, wherein R^{j} is selected from cyclopropyl, —CHF₂ and —CF₃.

Embodiment 107. The compound of any one of embodiments 39, 57, 60, and 80-104, wherein R^{j} is selected from cyclopropyl and —CF₃.

Embodiment 108. The compound of any one of embodiments 39, 57, 60, and 80-104, wherein R^{j} is cyclopropyl.

Embodiment 109. The compound of any one of embodiments 39, 57, 60, and 80-104, wherein Rⁱ is —CHF₂.

Embodiment 110. The compound of any one of embodiments 39, 57, 60, and 80-104, wherein Rⁱ is —CF₃.

Embodiment 111. The compound of any one of embodiments 39, 57, 60, and 80-110, wherein R^{k} is selected from hydrogen and halo.

Embodiment 112. The compound of any one of embodiments 39, 57, 60, and 80-110, wherein R^{k} is selected from —H and —Cl.

Embodiment 113. The compound of any one of embodiments 39, 57, 60, and 80-110, wherein R^{k} is —H.

Embodiment 114. The compound of any one of embodiments 39, 57, 60, and 80-110, wherein R^{k} is —Cl.

Embodiment 115. The compound of any one of embodiments 39, 57, 60, and 80-114, wherein R^{m} is selected from —H and —OH.

Embodiment 116. The compound of any one of embodiments 39, 57, 60, and 80-114, wherein R^{m} is —H.

Embodiment 117. The compound of any one of embodiments 39, 57, 60, and 80-114, wherein R^{m} is —OH.

Embodiment 118. The compound of any one of embodiments 1-117, wherein R^{d} is H.

Embodiment 119. The compound of any one of embodiments 1-117, wherein R^{d} is F.

Embodiment 120. The compound of any one of embodiments 1-117, wherein R^{1A} is

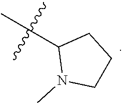 [SMILES: *C1CCCN1C ; "."]

Embodiment 121. The compound of any one of embodiments 1-117, wherein R^{1A} is selected from

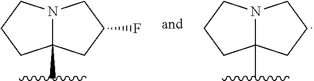 [SMILES: *[C@@]12CCCN1C[C@H](F)C2 ; *C12CCCN1CCC2]

Embodiment 122. The compound of any one of embodiments 1-117, wherein R^{1A} is

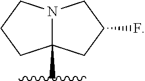 [SMILES: *[C@@]12CCCN1C[C@H](F)C2]

Embodiment 123. The compound of any one of embodiments 1-117, wherein R^{1A} is

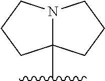 [SMILES: *C12CCCN1CCC2]

Embodiment 124. The compound of any one of embodiments 1-123, wherein R¹ is selected from H, —OCH₂R^{1A}, —OMe, Me and

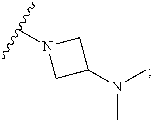 [SMILES: *N1CC(N(C)C)C1]

Embodiment 125. The compound of any one of embodiments 1-123, wherein R¹ is H.

Embodiment 126. The compound of any one of embodiments 1-123, wherein R¹ is —OMe.

Embodiment 127. The compound of any one of embodiments 1-123, wherein R¹ is Me.

Embodiment 128. The compound of any one of embodiments 1-123, wherein R¹ is

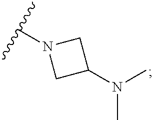 [SMILES: *N1CC(N(C)C)C1]

Embodiment 129. The compound of any one of embodiments 1-123, wherein R¹ is OCH₂R^{1A}.

Embodiment 130. The compound of any one of embodiments 1-123, wherein R¹ is selected from the group consisting of

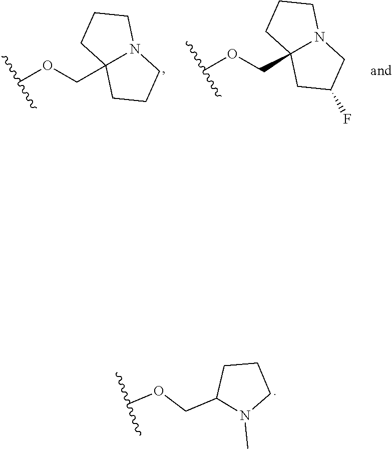

Embodiment 131. The compound of any one of embodiments 1-123, wherein R¹ is

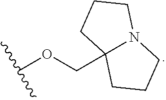 [SMILES: *OCC12CCCN1CCC2]

Embodiment 132. The compound of any one of embodiments 1-123, wherein R¹ is

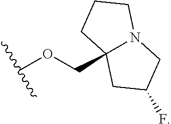 [SMILES: *OC[C@@]12CCCN1C[C@H](F)C2]

Embodiment 133. The compound of any one of embodiments 1-123, wherein R¹ is

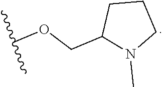 [SMILES: *OCC1CCCN1C]

Embodiment 134. The compound of any one of embodiments 1-133, wherein R² is

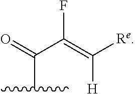

Embodiment 135. The compound of any one of embodiments 1-133, wherein R² is

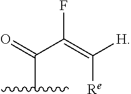

Embodiment 136. The compound of any one of embodiments 1-123, wherein R^{Y}i is -Me.

Embodiment 137. The compound of any one of embodiments 1-123, wherein R^{Y}i is —H.

Embodiment 138. The compound of any one of embodiments 1-137, wherein R^{Y2} is —H.

Embodiment 139. The compound of any one of embodiments 1-137, wherein R^{Y2} is -Me.

Embodiment 140. The compound of any one of embodiments 1-139, wherein R^{e} is selected from the group consisting of —C(O)O—C₁-C₄ alkyl, R^{e1}, —C(R^{Y1})(R^{Y2})R^{e}, R^{e2} and —C(R^{Y1})(R^{Y2})R^{e3}

Embodiment 141. The compound of any one of embodiments 1-139, wherein R^{e} is selected from the group consisting of —C(O)OMe, R^{e1}, —CH₂R^{e1}, R^{e2} and —CH₂R^{e3}.

Embodiment 142. The compound of any one of embodiments 1-139, wherein R^{e} is selected from the group consisting of —C(O)OMe, R^{e1}, —CH₂R^{e1} and R^{e2}.

Embodiment 143. The compound of any one of embodiments 1-139, wherein R^{e} is selected from the group consisting of R^{e1}, —CH₂R^{e1} and R^{e2}.

Embodiment 144. The compound of any one of embodiments 1-139, wherein R^{e} is R^{e1} or —C(R^{Y1})(R^{Y2})R^{e1}.

Embodiment 145. The compound of any one of embodiments 1-144, wherein R^{e1} is a 4-7 membered monocyclic heterocycle containing a nitrogen atom as the only heteroatom or containing one nitrogen atom and one oxygen atom, wherein the monocyclic heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 146. The compound of any one of embodiments 1-144, wherein R^{e1} is a 4-7 membered monocyclic heterocycle containing a nitrogen atom as the only heteroatom, wherein the monocyclic heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 147. The compound of embodiment 145 or 146 wherein the monocyclic heterocycle of R^{e1} is substituted with 0 or 1 instance of methyl, ethyl, isopropyl or methoxyethyl.

Embodiment 148. The compound of embodiment 145 or 146 wherein the monocyclic heterocycle of R^{e1} is substituted with 0 or 1 instance of C₁-C₄ alkyl.

Embodiment 149. The compound of embodiment 145 or 146, wherein the monocyclic heterocycle of R^{e1} is substituted with 0 or 1 instance of methyl.

Embodiment 150. The compound of embodiment 145, wherein R^{e1} is selected from azetidinyl, pyrrolidinyl, tetrahydrofuranyl, and morpholinyl substituted with 0 or 1 instance of methyl, ethyl, isopropyl, or methoxyethyl.

Embodiment 151. The compound of embodiment 145, wherein R^{e1} is selected from azetidinyl, pyrrolidinyl and morpholinyl substituted with 0 or 1 instance of methyl.

Embodiment 152. The compound of embodiment 145 or 146, wherein R^{e1} is azetidinyl substituted with 0 or 1 instance of methyl, ethyl, isopropyl or methoxyethyl.

Embodiment 153. The compound of embodiment 145 or 146, wherein R^{e1} is azetidinyl substituted with 0 or 1 instance of methyl.

Embodiment 154. The compound of embodiment 145 or 146, wherein R^{e1} is N-methyl azetidinyl.

Embodiment 155. The compound of any one of embodiments 145-154, wherein the attachment point for the monocyclic heterocycle is on a carbon atom.

Embodiment 156. The compound of embodiment 155 wherein R^{e1} is selected from the group consisting of:

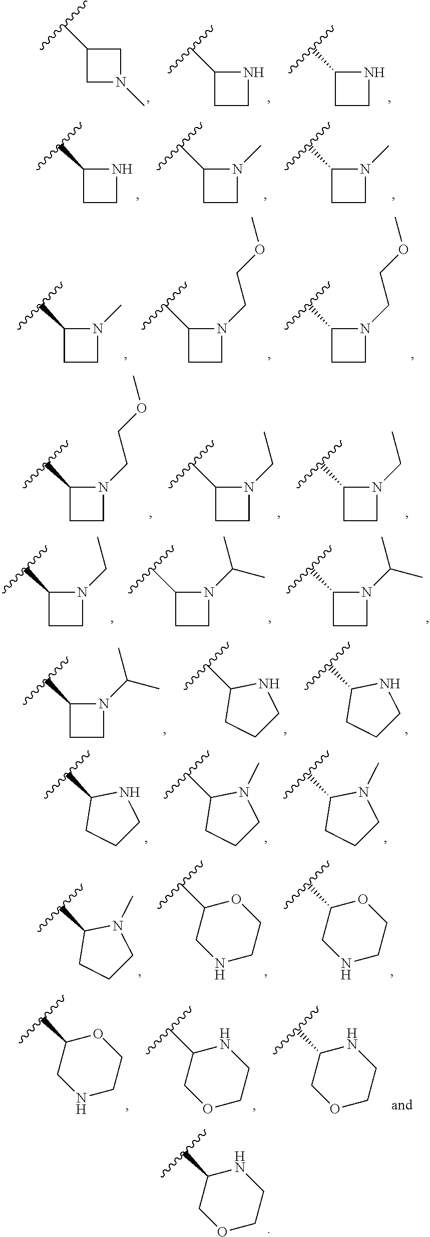

Embodiment 157. The compound of embodiment 155 wherein R^{e} is

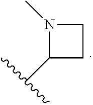 [SMILES: *C1CCN1C]

Embodiment 158. The compound of embodiment 155 wherein R^{e1} is

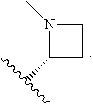 [SMILES: *[C@H]1CCN1C]

Embodiment 159. The compound of any one of embodiments 145-158, wherein R^{e} is R^{e1}.

Embodiment 160. The compound of any one of embodiments 1-144, wherein R^{e1} is a 4-10 membered heterocycle containing a nitrogen atom and one or two additional heteroatoms independently selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 161. The compound of embodiment 160, wherein R^{e1} is a 4-10 membered heterocycle containing a nitrogen atom and one or two additional heteroatoms independently selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 162. The compound of embodiment 160, wherein R^{e1} is a 4-8 member monocyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 163. The compound of embodiment 160, wherein R^{e1} is a 6-10 member fused bicyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 164. The compound of embodiment 160, wherein R^{e1} is a 6-10 member bridged heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 165. The compound of embodiment 160, wherein R^{e1} is a 6-10 member spiro heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 166. The compound of embodiment 160, wherein R^{e1} is selected from azetidine, pyrrolidine, 2-azabicyclo[2.1.1]hexane, morpholine, 2-oxa-5-azabicyclo[4.1.0]heptane, 1, 4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide,4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 167. The compound of embodiment 160, wherein R^{e1} is morpholine substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 168. The compound of any one of embodiments 160-167, wherein the attachment point for R^{e1} is the nitrogen atom of the heterocycle.

Embodiment 169. The compound of embodiment 168, wherein R^{e1} is selected from the group consisting of:

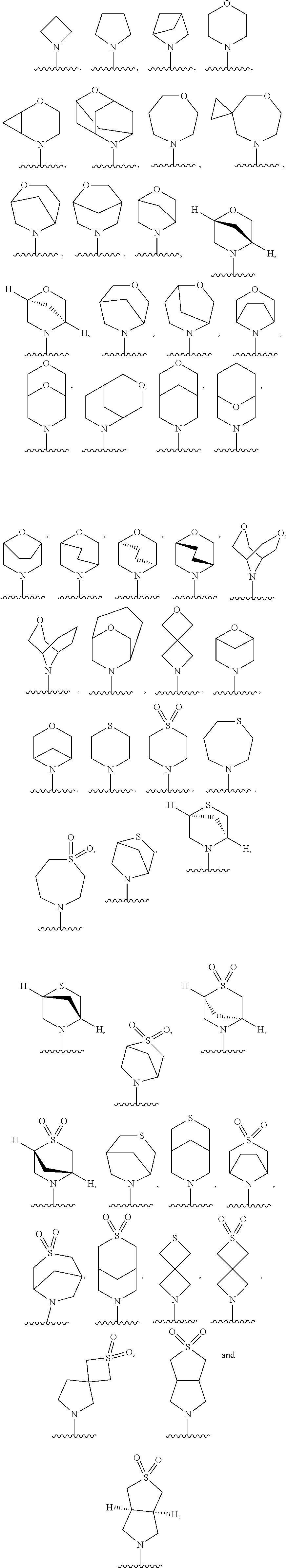

each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 170. The compound of embodiment 168, wherein R^{e1} is selected from

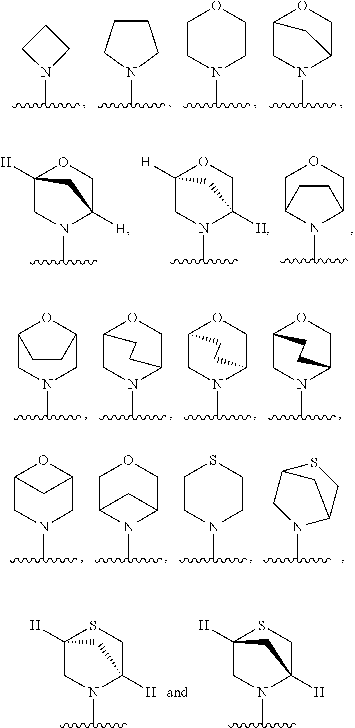

substituted with 0, 1, 2, 3 or 4 substituents independently selected from halo, hydroxy, C₁-C₄ alkyl, C₁-C₆ alkoxy, C₁-C₆ alkoxyalkoxy C₁-C₄ haloalkyl and C₁-C₄ haloalkoxy.

Embodiment 171. The compound of embodiment 168, wherein R^{e1} is selected from

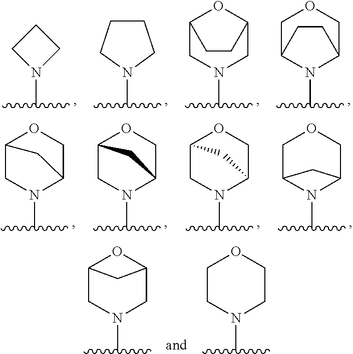

and substituted with 0, 1, 2, 3 or 4 substituents independently selected from halo, hydroxy, C₁-C₄ alkyl, C₁-C₆ alkoxy, C₁-C₄ alkoxyalkoxy, C₁-C₄ haloalkyl and C₁-C₄ haloalkoxy.

Embodiment 172. The compound of embodiment 168, wherein R^{e1} is

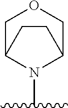 [SMILES: *N1C2CCC1COC2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 173. The compound of embodiment 168, wherein R^{e1} is

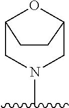 [SMILES: *N1CC2CCC(C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 174. The compound of embodiment 168, wherein R^{e1} is

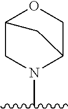 [SMILES: *N1CC2CC1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 175. The compound of embodiment 168, wherein R^{e1} is

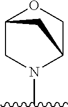 [SMILES: *N1C[C@H]2C[C@@H]1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 176. The compound of embodiment 168, wherein R^{e1} is

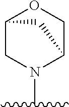 [SMILES: *N1C[C@@H]2C[C@H]1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 177. The compound of embodiment 168, wherein R^{e1} is

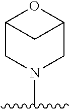 [SMILES: *N1CC2CC(C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 178. The compound of embodiment 168, wherein R^{e1} is

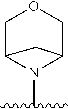 [SMILES: *N1C2COCC1C2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 179. The compound of embodiment 168, wherein R^{e1} is

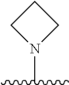 [SMILES: *N1CCC1]

substituted with 0, 1, 2, 3 or 4 substituents independently selected from halo, hydroxy, C₁-C₄ alkyl, C₁-C₆ alkoxy, C₁-C₆alkoxyalkoxy, C₁-C₄ haloalkyl and C₁-C₄ haloalkoxy.

Embodiment 180. The compound of embodiment 168, wherein R^{e1} is

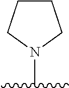 [SMILES: *N1CCCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 181. The compound of embodiment 168, wherein R^{e1} is

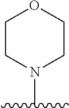 [SMILES: *N1CCOCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

Embodiment 182. The compound of any one of embodiments 160-181, wherein the 4-10 membered heterocycle of R^{e1} is substituted with 0, 1 or 2 substituents independently selected from —F, —OMe and -Me.

Embodiment 183. The compound of any one of embodiments 160-181, wherein the 4-10 membered heterocycle of R^{e1} is substituted with 0, 1 or 2 substituents independently selected from —F and, —OMe.

Embodiment 184. The compound of any one of embodiments 160-181, wherein the 4-10 membered heterocycle of R^{e1} is substituted with 0, 1 or 2 instances of —F.

Embodiment 185. The compound of any one of embodiments 160-181, wherein the 4-10 membered heterocycle of R^{e1} is substituted with 0 or 1 instances of —OMe.

Embodiment 186. The compound of any one of embodiments 160-181, wherein the 4-10 membered heterocycle of R¹ is unsubstituted.

Embodiment 187. The compound of embodiment 168, wherein R^{e1} is selected from the group consisting of:

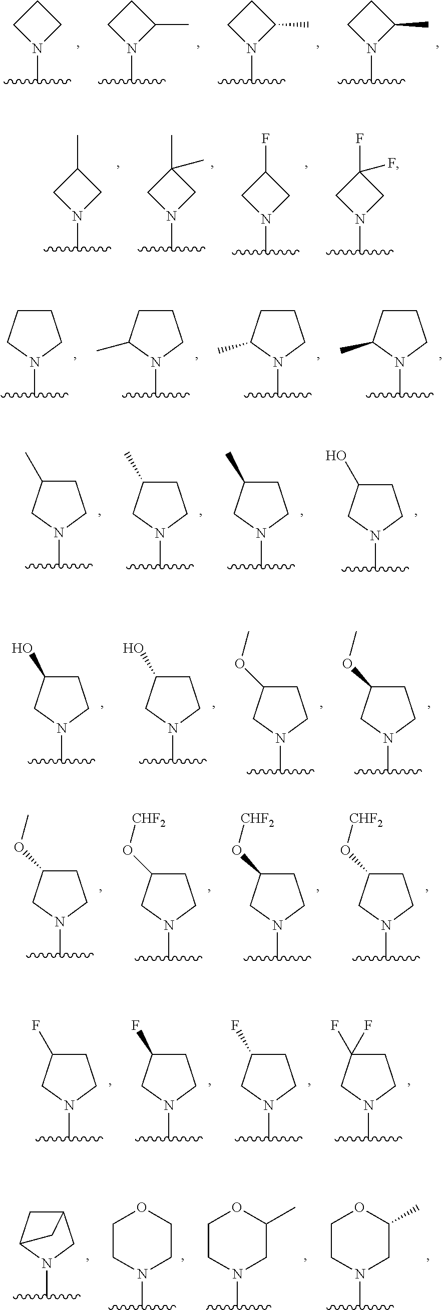

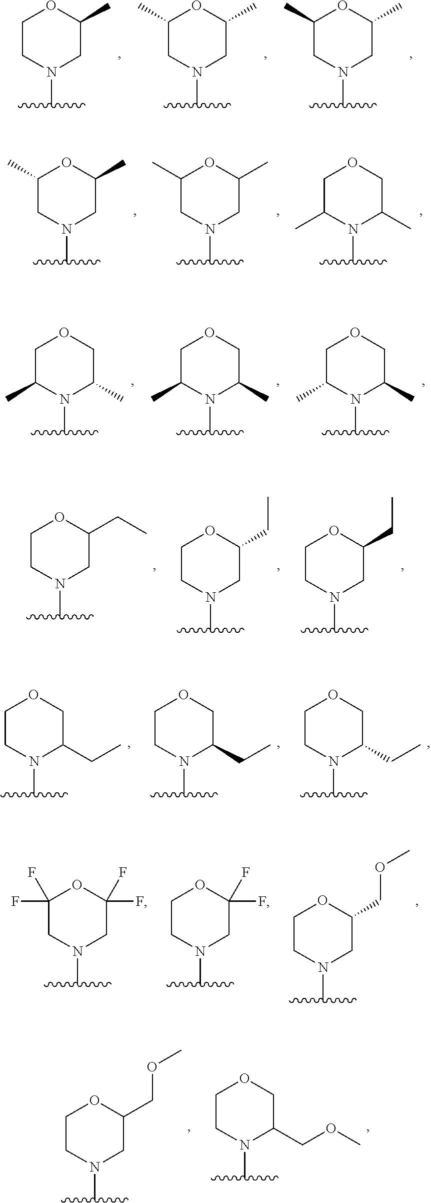

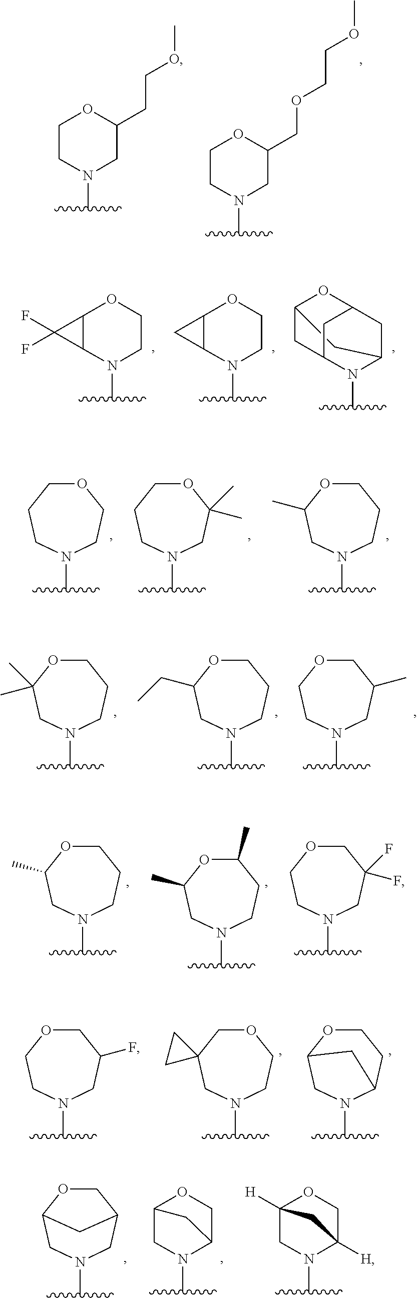

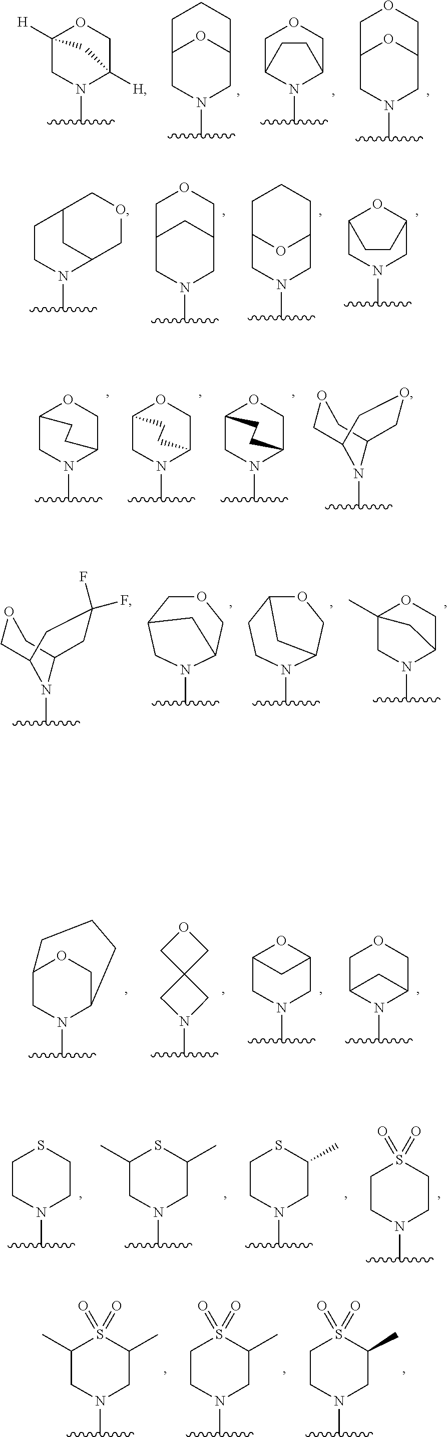

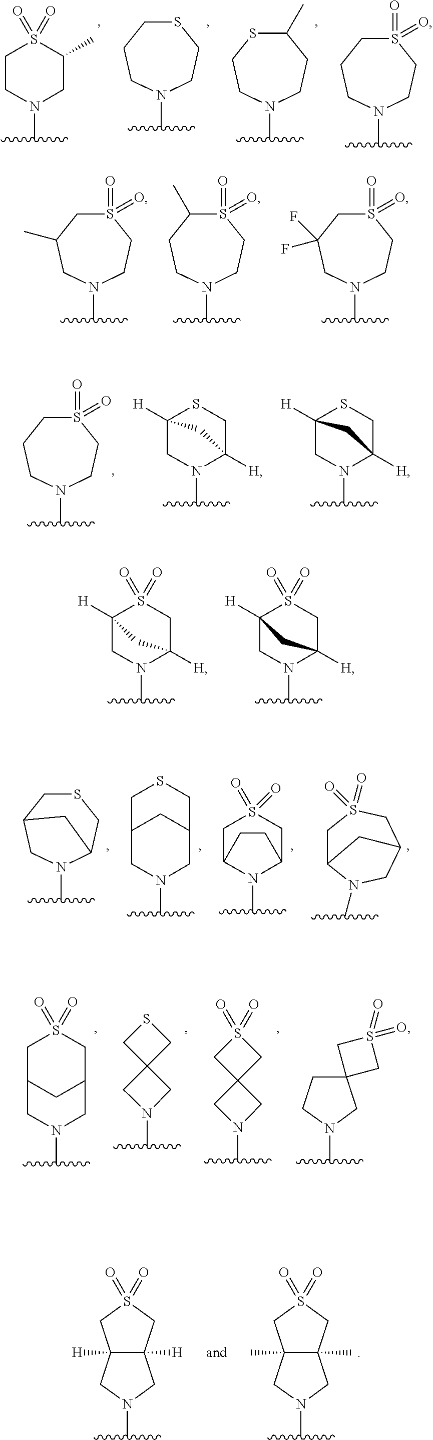

Embodiment 188. The compound of embodiment 168, wherein R^{e1} is selected from

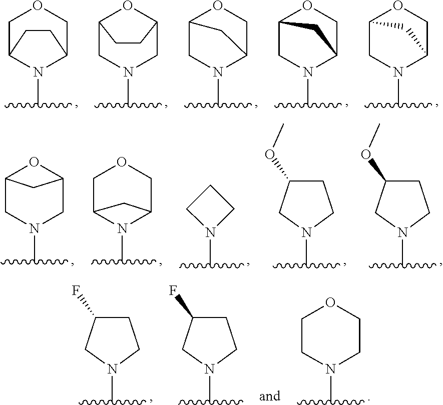

Embodiment 189. The compound of embodiment 168, wherein R^{e1} is selected from and

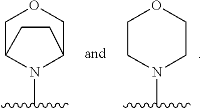 [SMILES: *N1C2CCC1COC2 ; *N1CCOCC1]

Embodiment 190. The compound of embodiment 168, wherein R^{e1} is unsubstituted

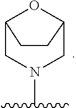 [SMILES: *N1CC2CCC(C1)O2]

Embodiment 191. The compound of embodiment 168, wherein R^{e} is unsubstituted

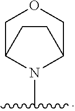 [SMILES: *N1C2CCC1COC2]

Embodiment 192. The compound of embodiment 168, wherein R^{e1} is unsubstituted

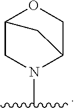 [SMILES: *N1CC2CC1CO2]

Embodiment 193. The compound of embodiment 168, wherein R^{e1} is unsubstituted

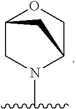 [SMILES: *N1C[C@H]2C[C@@H]1CO2]

Embodiment 194. The compound of embodiment 168, wherein R^{e1} is unsubstituted

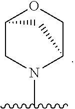 [SMILES: *N1C[C@@H]2C[C@H]1CO2]

Embodiment 195. The compound of embodiment 168, wherein R^{e1} is unsubstituted

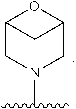 [SMILES: *N1CC2CC(C1)O2]

Embodiment 196. The compound of embodiment 168, wherein R^{e1} is unsubstituted

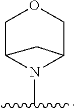 [SMILES: *N1C2COCC1C2]

Embodiment 197. The compound of embodiment 168, wherein R^{e1} is unsubstituted

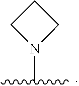 [SMILES: *N1CCC1]

Embodiment 198. The compound of embodiment 168, wherein R^{e1} is

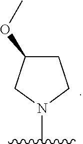 [SMILES: *N1CC[C@H](OC)C1]

Embodiment 199. The compound of embodiment 168, wherein R^{e1} is

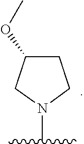 [SMILES: *N1CC[C@@H](OC)C1]

Embodiment 200. The compound of embodiment 168, wherein R^{e1} is

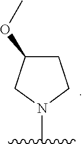 [SMILES: *N1CC[C@H](OC)C1]

Embodiment 201. The compound of embodiment 168, wherein R^{e1} is

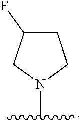 [SMILES: *N1CCC(F)C1]

Embodiment 202. The compound of embodiment 168, wherein R^{e1} is

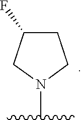 [SMILES: *N1CC[C@@H](F)C1]

Embodiment 203. The compound of embodiment 168, wherein R^{e1} is

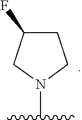 [SMILES: *N1CC[C@H](F)C1]

Embodiment 204. The compound of embodiment 168, wherein R^{e1} is unsubstituted

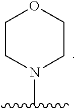 [SMILES: *N1CCOCC1]

Embodiment 205. The compound of any one of embodiments 160-204, wherein R^{e} is —C(R^{Y1})(R^{Y2})R^{e1}.

Embodiment 206. The compound of any one of embodiments 160-204, wherein R^{e} is —CH₂R^{e1}.

Embodiment 207. The compound of any one of embodiments 1-139 wherein R^{e} is R^{e2}.

Embodiment 208. The compound of any one of embodiments 1-143, 145-204 and 207, wherein R^{e2} is a 5-6 membered heteroaryl group containing at least one nitrogen atom, wherein the attachment point for the heteroaryl group is a carbon atom group and wherein the heteroaryl is substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

Embodiment 209. The compound of any one of embodiments 1-143, 145-204 and 207, wherein R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

Embodiment 210. The compound of any one of embodiments 1-143, 145-204 and 207, wherein R^{e2} is selected from the group consisting of pyrimidinyl, 1,2,4-oxadiazolyl and 1H-1,2,4-triazolyl, each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

Embodiment 211. The compound of any one of embodiments 1-143, 145-204 and 207, wherein R^{e2} is selected from the group consisting of:

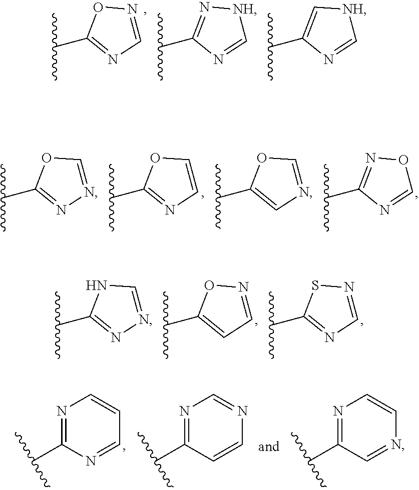

each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

Embodiment 212. The compound of any one of embodiments 1-143, 145-204 and 207, wherein R^{e2} is selected from the group consisting of:

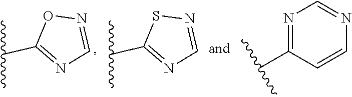

each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

Embodiment 213. The compound of any one of embodiments 1-143, 145-204 and 207, wherein R^{e2} is selected from the group consisting of:

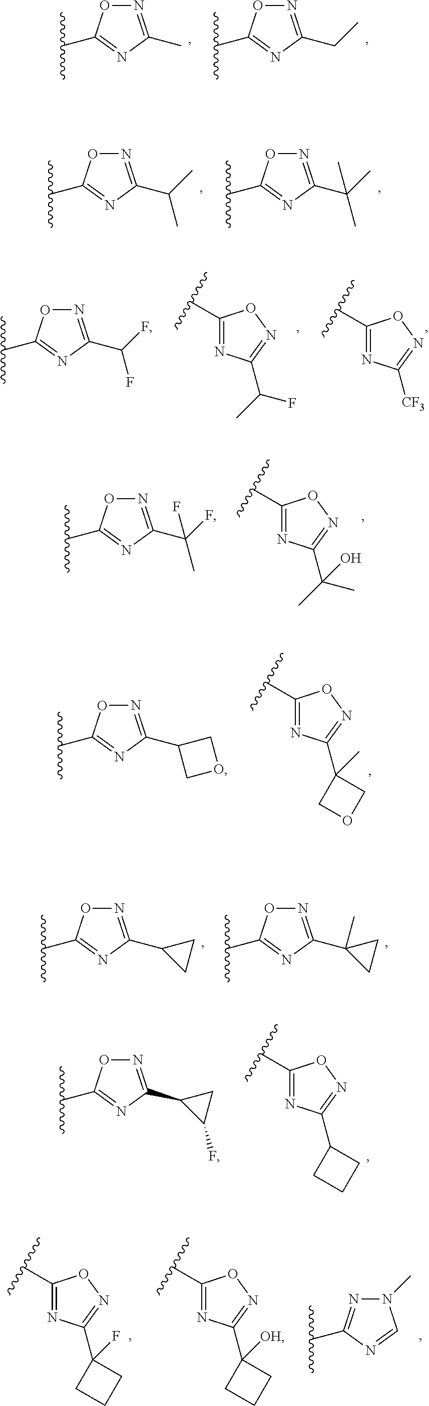

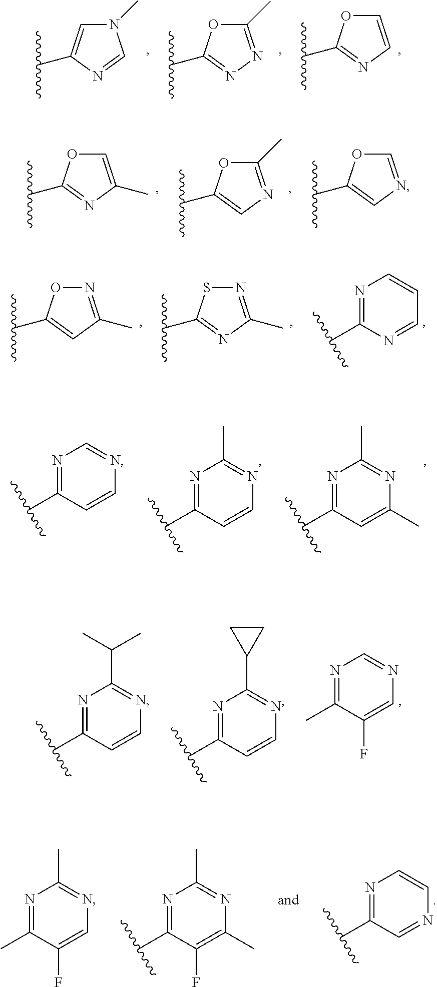

Embodiment 214. The compound of any one of embodiments 1-143, 145-204 and 207, wherein R^{e2} is selected from the group consisting of:

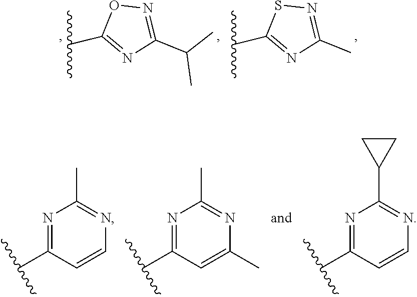

Embodiment 215. The compound of any one of embodiments 1-143, 145-204 and 207, wherein R^{e2} is a 6 membered heteroaryl group substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

Embodiment 216. The compound of embodiment 215, wherein R^{e2} is pyrimidinyl or pyridazinyl substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

Embodiment 217. The compound of embodiment 215, wherein R^{e2} is pyrimidinyl or pyridazinyl substituted with 0, 1 or 2 instances of C₁-C₄ alkyl.

Embodiment 218. The compound of embodiment 215, wherein R^{e2} is pyrimidinyl substituted with 0, 1 or 2 instances of C₁-C₄ alkyl.

Embodiment 219. The compound of embodiment 215, wherein R^{e2} is pyridazinyl substituted with 0, 1 or 2 instances of C₁-C₄ alkyl.

Embodiment 220. The compound of embodiment 215, wherein R^{e2} is pyrimidinyl or pyridazinyl substituted with 0, 1 or 2 instances of -Me.

Embodiment 221. The compound of embodiment 215, wherein R^{e2} is pyrimidinyl substituted with 0, 1 or 2 instances of -Me.

Embodiment 222. The compound of embodiment 215, wherein R^{e2} is pyridazinyl substituted with 0, 1 or 2 instances of -Me.

Embodiment 223. The compound of embodiment 215, wherein R^{e2} is selected from the group consisting of:

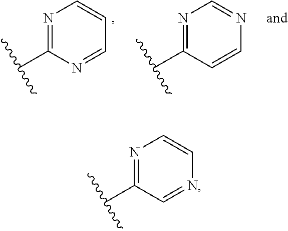 [SMILES: *c1ncccn1 ; *c1ccncn1 ; "and" ; *c1cnccn1]

substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl. Embodiment 224. The compound of embodiment 215, wherein R^{e2} is selected from the group consisting of

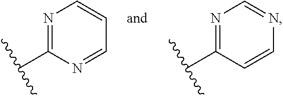 [SMILES: *c1ncccn1 ; "and" ; *c1ccncn1]

substituted with 0, 1 or 2 instances of C₁-C₄ alkyl.

Embodiment 225. The compound of embodiment 215, wherein R^{e2} is

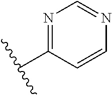 [SMILES: *c1ccncn1]

substituted with 0, 1 or 2 instances of C₁-C₄ alkyl.

Embodiment 226. The compound of embodiment 215, wherein R^{e2} is

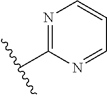 [SMILES: *c1ncccn1]

substituted with 0, 1 or 2 instances of C₁-C₄ alkyl.

Embodiment 227. The compound of embodiment 215, wherein R^{e2} is

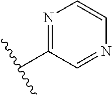 [SMILES: *c1cnccn1]

substituted with 0, 1 or 2 instances of C₁-C₄ alkyl.

Embodiment 228. The compound of embodiment 215, wherein R^{e2} is selected from the group consisting of

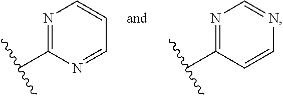 [SMILES: *c1ncccn1 ; "and" ; *c1ccncn1]

substituted with 0, 1 or 2 instances of -Me.

Embodiment 229. The compound of embodiment 215, wherein R^{e2} is

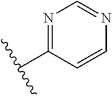 [SMILES: *c1ccncn1]

substituted with 0, 1 or 2 instances of -Me.

Embodiment 230. The compound of embodiment 215, wherein R^{e2} is

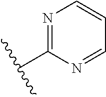 [SMILES: *c1ncccn1]

substituted with 0, 1 or 2 instances of -Me.

Embodiment 231. The compound of embodiment 215, wherein R^{e2} is

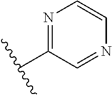 [SMILES: *c1cnccn1]

substituted with 0, 1 or 2 instances of -Me.

Embodiment 232. The compound of embodiment 215, wherein R^{e2} is selected from the group consisting of

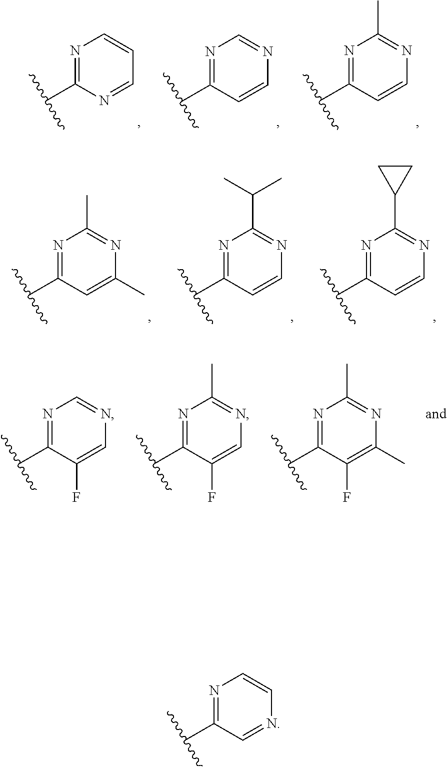

Embodiment 233. The compound of embodiment 215, wherein R^{e2} is selected from the group consisting of

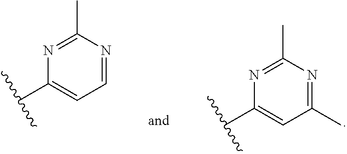

Embodiment 234. The compound of embodiment 215, wherein R^{e2} is

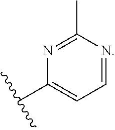 [SMILES: *c1ccnc(C)n1]

Embodiment 235. The compound of embodiment 215, wherein R^{e2} is

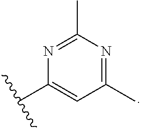 [SMILES: *c1cc(C)nc(C)n1]

Embodiment 236. The compound of any one of embodiments 1-143, 145-204 and 207, wherein R^{e2} is a 5 membered heteroaryl group containing at least one nitrogen atom, wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo, hydroxy and methyl, and C₃-C₆ cycloalkyl optionally substituted with one or two substituents independently selected from halo, hydroxy and methyl.

Embodiment 237. The compound of any one of embodiments 1-143, 145-204 and 207, wherein R^{e2} is selected from the group consisting of oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo, hydroxy and methyl, and C₃-C₆ cycloalkyl optionally substituted with one or two substituents independently selected from halo, hydroxy and methyl.

Embodiment 238. The compound of any one of embodiments 1-143, 145-204 and 207, wherein R^{e2} is selected from the group consisting of

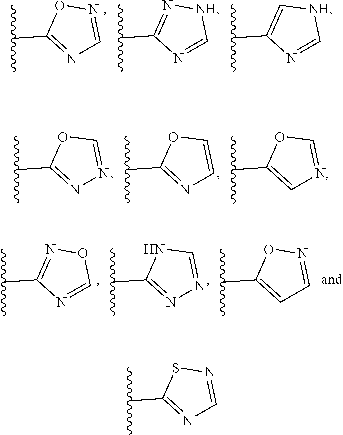

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo, hydroxy and methyl, and C₃-C₆ cycloalkyl optionally substituted with one or two substituents independently selected from halo, hydroxy and methyl.

Embodiment 239. The compound of any one of embodiments 1-143, 145-204 and 207, wherein R^{e2} is selected from the group consisting of

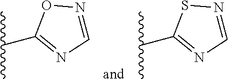 [SMILES: *c1ncno1 ; *c1ncns1 ; "and"]

each substituted with 1 substituent independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo, hydroxy and methyl, and C₃-C₆ cycloalkyl optionally substituted with one or two substituents independently selected from halo, hydroxy and methyl.

Embodiment 240. The compound of any one of embodiments 1-143, 145-204 and 207, wherein R^{e2} is selected from the group consisting of

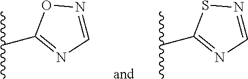 [SMILES: *c1ncno1 ; *c1ncns1 ; "and"]

each substituted with 1 instance of C₁-C₄ alkyl.

Embodiment 241. The compound of any one of embodiments 1-143, 145-204 and 207, wherein R^{e2} is selected from the group consisting of:

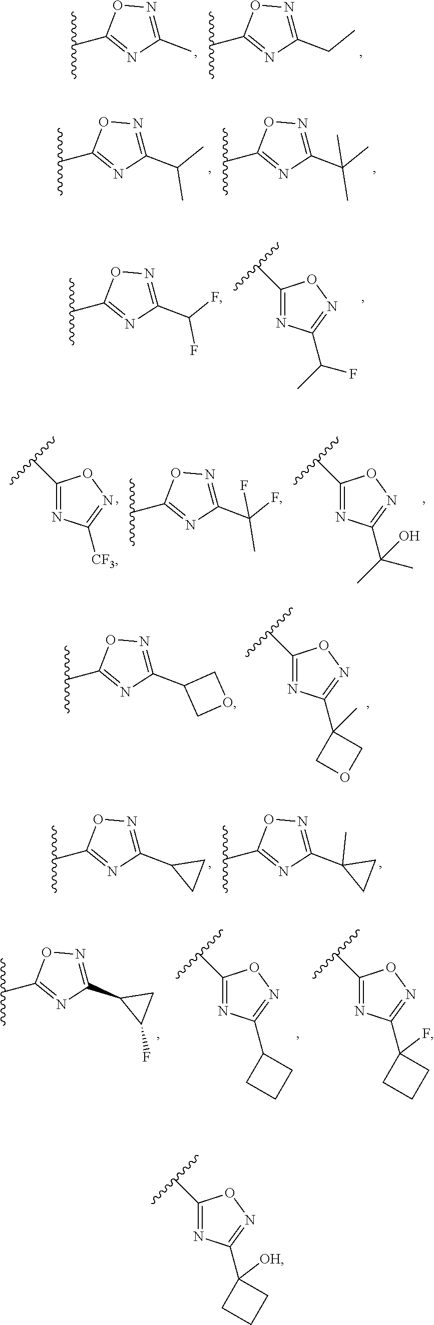

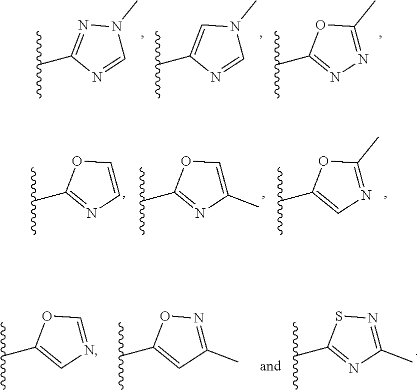

Embodiment 242. The compound of any one of embodiments 1-143, 145-204 and 207,

wherein R^{e2} is selected from the group consisting of

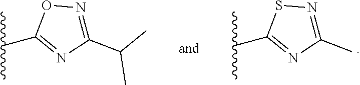

Embodiment 243. The compound of any one of embodiments 1-143, 145-204 and 207, wherein R^{e2} is

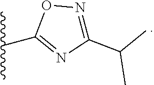 [SMILES: *c1nc(C(C)C)no1]

Embodiment 244. The compound of any one of embodiments 1-143, 145-204 and 207, wherein R^{e2} is

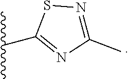 [SMILES: *c1nc(C)ns1]

Embodiment 245. The compound of any one of embodiments 1-139, wherein R^{e} is —C(R^{Y1})(R^{Y2})R^{e3}.

Embodiment 246. The compound of any one of embodiments 1-143, 145-204 and 208-245, wherein R³¹ and R³² are independently selected from the group consisting of C₁-C₄ alkyl, C₁-C₄ alkoxy, C₁-C₄ haloalkoxy, and C₁-C₄ alkyl substituted with a 3-6 membered heterocycle containing 1-2 atoms independently selected from N, O and S, including S(O)₂.

Embodiment 247. The compound of any one of embodiments 1-143, 145-204 and 208-245, wherein R³¹ is C₁-C₄ alkyl.

Embodiment 248. The compound of any one of embodiments 1-143, 145-204 and 208-245, wherein R³¹ is -Me.

Embodiment 249. The compound of any one of embodiments 1-143, 145-204 and 208-248, wherein R³² is C₁-C₄ alkyl or C₁-C₄ alkyl substituted with a 3-6 membered heterocycle.

Embodiment 250. The compound of any one of embodiments 1-143, 145-204 and 208-248, wherein R³² is C₁-C₄ alkyl substituted with a 3-6 membered heterocycle.

Embodiment 251. The compound of any one of embodiments 1-143, 145-204 and 208-248, wherein R³² is C₁-C₄ alkyl substituted with a heterocycle selected from azetidine, oxetane, pyrrolidine, tetrahydrofuran, piperidine, tetrahydropyran and morpholine.

Embodiment 252. The compound of any one of embodiments 1-143, 145-204 and 208-248, wherein R³² is C₁-C₄ alkyl substituted with oxetane.

Embodiment 253. The compound of any one of embodiments 1-143, 145-204 and 208-248, wherein R³² is

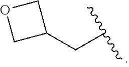 [SMILES: *CC1COC1]

Embodiment 254. The compound of any one of embodiments 1-143, 145-204 and 208-248, wherein R³² is C₁-C₄ alkyl.

Embodiment 255. The compound of any one of embodiments 1-143, 145-204 and 208-248 wherein R³² is Me.

Embodiment 256. The compound of any one of embodiments 1-143, 145-204 and 208-245, wherein R^{e3} is selected from

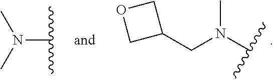

Embodiment 257. The compound of any one of embodiments 1-143, 145-204 and 208-245, wherein R^{e3} is

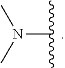 [SMILES: *N(C)C]

Embodiment 258. The compound of any one of embodiments 1-143, 145-204 and 208-245, wherein R^{e3} is

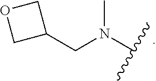 [SMILES: *N(C)CC1COC1]

Embodiment 259. The compound of any one of embodiments 1-139, wherein R^{e} is selected from the group consisting of:

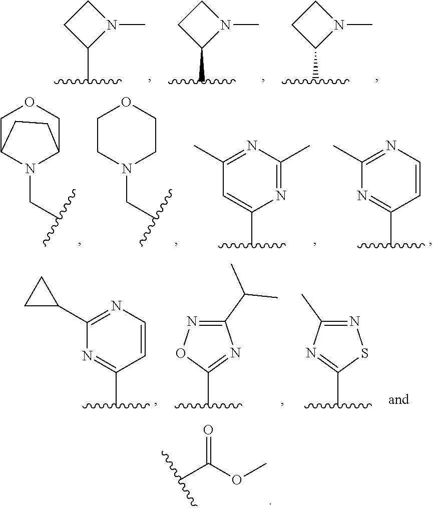

Embodiment 260. The compound of any one of embodiments 1-139, wherein R^{e} is

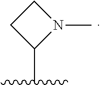 [SMILES: *C1CCN1C]

Embodiment 261. The compound of any one of embodiments 1-139, wherein R^{e} is

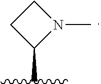 [SMILES: *[C@H]1CCN1C]

Embodiment 262. The compound of any one of embodiments 1-139, wherein R^{e} is

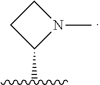 [SMILES: *[C@@H]1CCN1C]

Embodiment 263. The compound of any one of embodiments 1-139, wherein R^{e} is

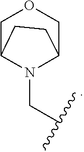 [SMILES: *CN1C2CCC1COC2]

Embodiment 264. The compound of any one of embodiments 1-139, wherein R^{e} is

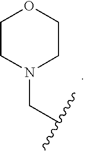 [SMILES: *CN1CCOCC1]

Embodiment 265. The compound of any one of embodiments 1-139, wherein R^{e} is

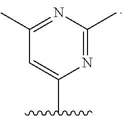

Embodiment 266. The compound of any one of embodiments 1-139, wherein R^{e} is

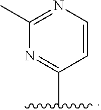 [SMILES: *c1ccnc(C)n1]

Embodiment 267. The compound of any one of embodiments 1-139, wherein R^{e} is

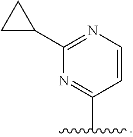 [SMILES: *c1ccnc(C2CC2)n1]

Embodiment 268. The compound of any one of embodiments 1-139, wherein R^{e} is

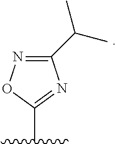 [SMILES: *c1nc(C(C)C)no1]

Embodiment 269. The compound of any one of embodiments 1-139, wherein R^{e} is

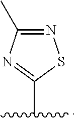 [SMILES: *c1nc(C)ns1]

Embodiment 270. The compound of any one of embodiments 1-139, wherein R^{e} is —OH. C(O)OMe.

Embodiment 271. The compound of any one of embodiments 1-270, wherein R^{x} is selected from —F and -Me.

Embodiment 272. The compound of any one of embodiments 1-270, wherein R^{x} is —OH.

Embodiment 273. The compound of any one of embodiments 1-270, wherein R^{x} is -Me.

Embodiment 274. The compound of any one of embodiments 1-270 wherein R^{x} is —F.

Embodiment 275. The compound of any one of embodiments 1-274, wherein n is 0 or 1.

Embodiment 276. The compound of any one of embodiments 1-274, wherein n is 0.

Embodiment 277. The compound of any one of embodiments 1-274, wherein n is 1.

Embodiment 278. The compound of any one of embodiments 1-274, wherein n is 2.

Embodiment 279. The compound of any one of embodiments 1-278, wherein the compound is selected from the group consisting of:

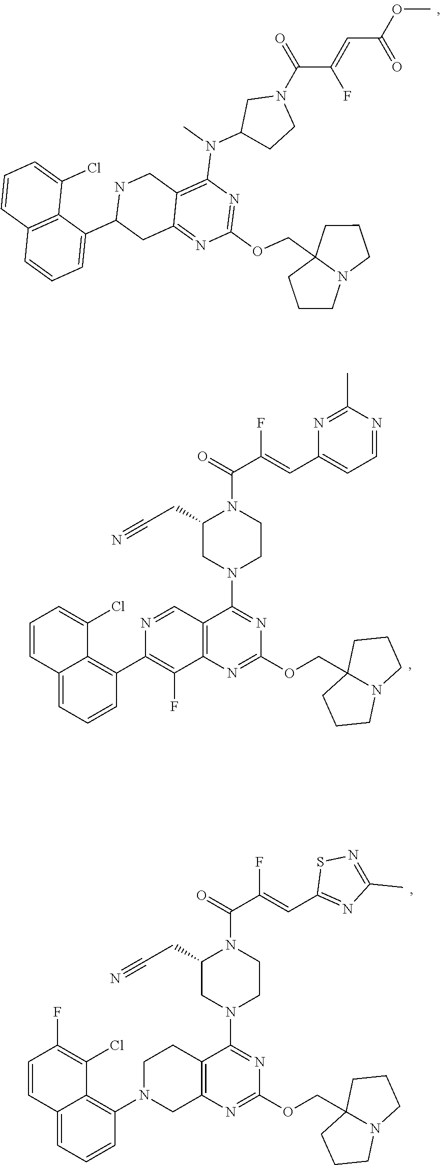 [SMILES: COC(=O)/C=C(\F)C(=O)N1CCC(N(C)c2nc(OCC34CCCN3CCC4)nc3c2CNC(c2cccc4cccc(Cl)c24)C3)C1 ; Cc1nccc(/C=C(\F)C(=O)N2CCN(c3nc(OCC45CCCN4CCC5)nc4c(F)c(-c5cccc6cccc(Cl)c56)ncc34)C[C@@H]2CC#N)n1 ; Cc1nsc(/C=C(\F)C(=O)N2CCN(c3nc(OCC45CCCN4CCC5)nc4c3CCN(c3cccc5ccc(F)c(Cl)c35)C4)C[C@@H]2CC#N)n1]

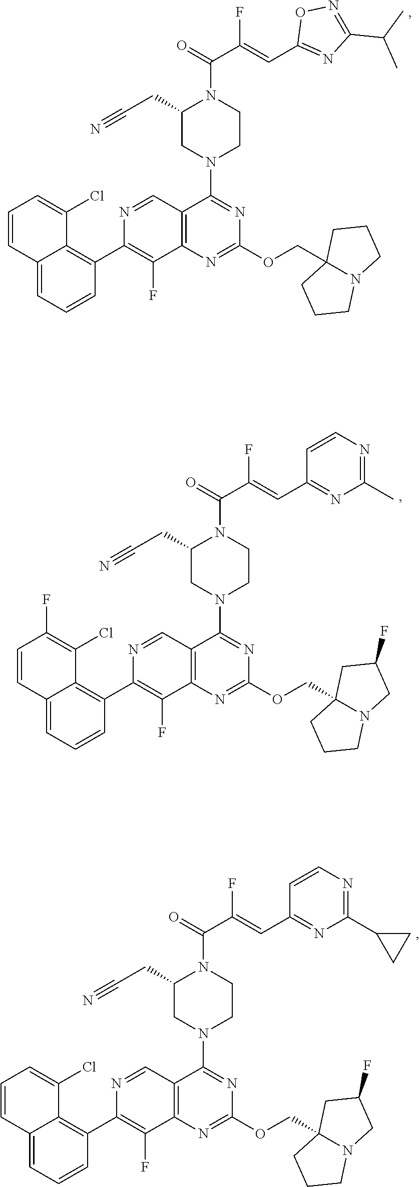 [SMILES: CC(C)c1noc(/C=C(\F)C(=O)N2CCN(c3nc(OCC45CCCN4CCC5)nc4c(F)c(-c5cccc6cccc(Cl)c56)ncc34)C[C@@H]2CC#N)n1 ; Cc1nccc(/C=C(\F)C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1 ; N#CC[C@H]1CN(c2nc(OC[C@@]34CCCN3C[C@H](F)C4)nc3c(F)c(-c4cccc5cccc(Cl)c45)ncc23)CCN1C(=O)/C(F)=C/c1ccnc(C2CC2)n1]

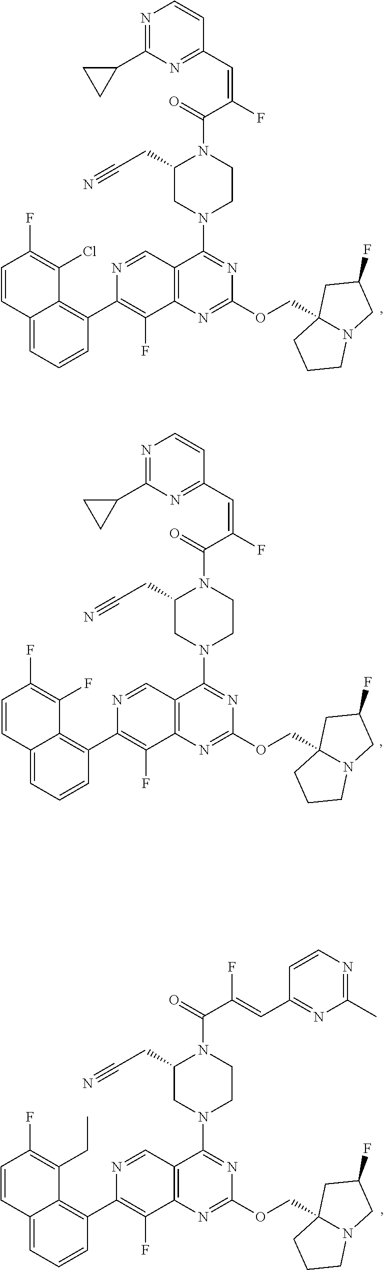 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C(F)=C\c1ccnc(C2CC2)n1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C(F)=C\c1ccnc(C2CC2)n1 ; CCc1c(F)ccc2cccc(-c3ncc4c(N5CCN(C(=O)/C(F)=C/c6ccnc(C)n6)[C@@H](CC#N)C5)nc(OC[C@@]56CCCN5C[C@H](F)C6)nc4c3F)c12]

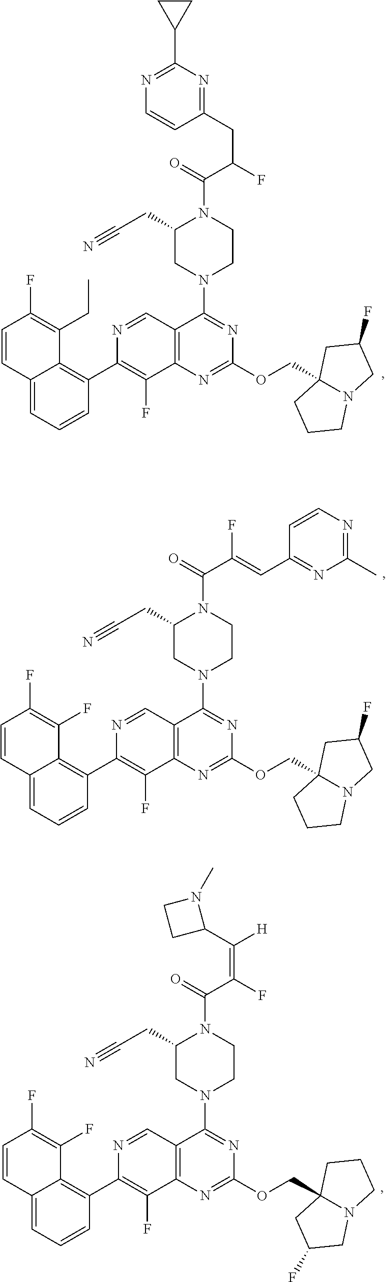 [SMILES: CCc1c(F)ccc2cccc(-c3ncc4c(N5CCN(C(=O)C(F)Cc6ccnc(C7CC7)n6)[C@@H](CC#N)C5)nc(OC[C@@]56CCCN5C[C@H](F)C6)nc4c3F)c12 ; Cc1nccc(/C=C(\F)C(=O)N2CCN(c3nc(OC[C@@]45CCCN4C[C@H](F)C5)nc4c(F)c(-c5cccc6ccc(F)c(F)c56)ncc34)C[C@@H]2CC#N)n1 ; CN1CCC1/C=C(/F)C(=O)N1CCN(c2nc(OC[C@@]34CCCN3C[C@H](F)C4)nc3c(F)c(-c4cccc5ccc(F)c(F)c45)ncc23)C[C@@H]1CC#N]

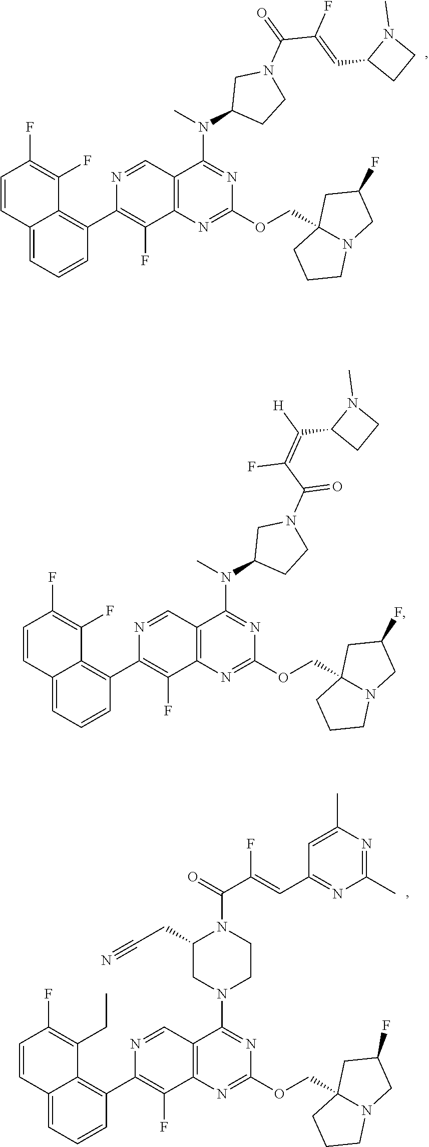 [SMILES: CN1CC[C@@H]1/C=C(\F)C(=O)N1CC[C@@H](N(C)c2nc(OC[C@@]34CCCN3C[C@H](F)C4)nc3c(F)c(-c4cccc5ccc(F)c(F)c45)ncc23)C1 ; CN1CC[C@@H]1/C=C(/F)C(=O)N1CC[C@@H](N(C)c2nc(OC[C@@]34CCCN3C[C@H](F)C4)nc3c(F)c(-c4cccc5ccc(F)c(F)c45)ncc23)C1 ; CCc1c(F)ccc2cccc(-c3ncc4c(N5CCN(C(=O)/C(F)=C/c6cc(C)nc(C)n6)[C@@H](CC#N)C5)nc(OC[C@@]56CCCN5C[C@H](F)C6)nc4c3F)c12]

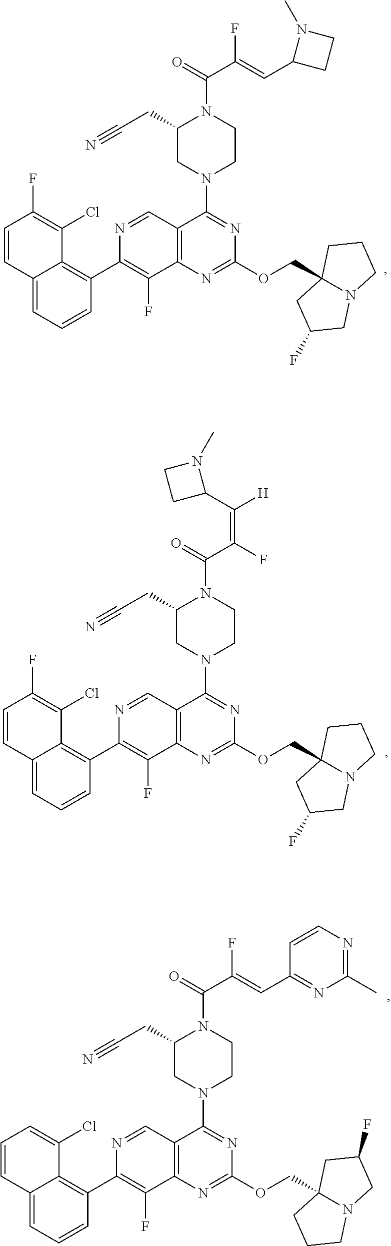 [SMILES: CN1CCC1/C=C(\F)C(=O)N1CCN(c2nc(OC[C@@]34CCCN3C[C@H](F)C4)nc3c(F)c(-c4cccc5ccc(F)c(Cl)c45)ncc23)C[C@@H]1CC#N ; CN1CCC1/C=C(/F)C(=O)N1CCN(c2nc(OC[C@@]34CCCN3C[C@H](F)C4)nc3c(F)c(-c4cccc5ccc(F)c(Cl)c45)ncc23)C[C@@H]1CC#N ; Cc1nccc(/C=C(\F)C(=O)N2CCN(c3nc(OC[C@@]45CCCN4C[C@H](F)C5)nc4c(F)c(-c5cccc6cccc(Cl)c56)ncc34)C[C@@H]2CC#N)n1]

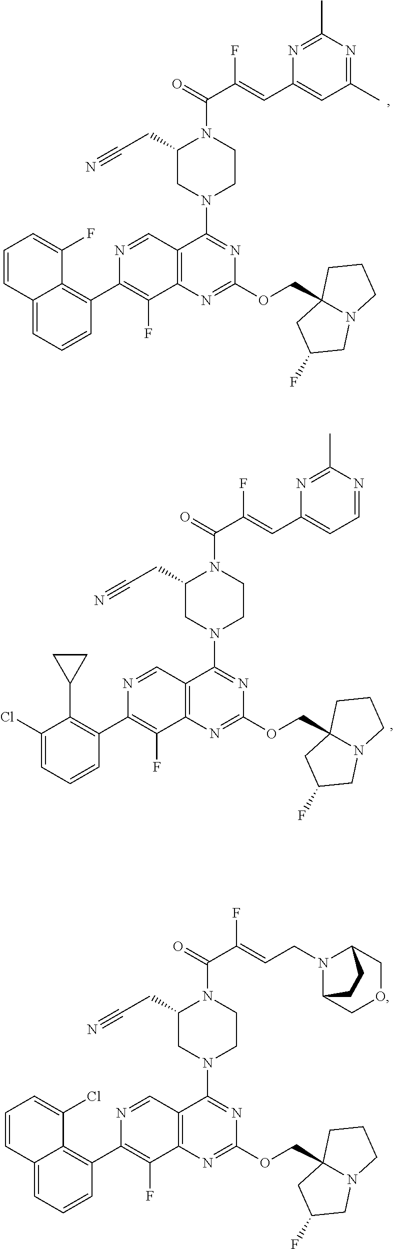

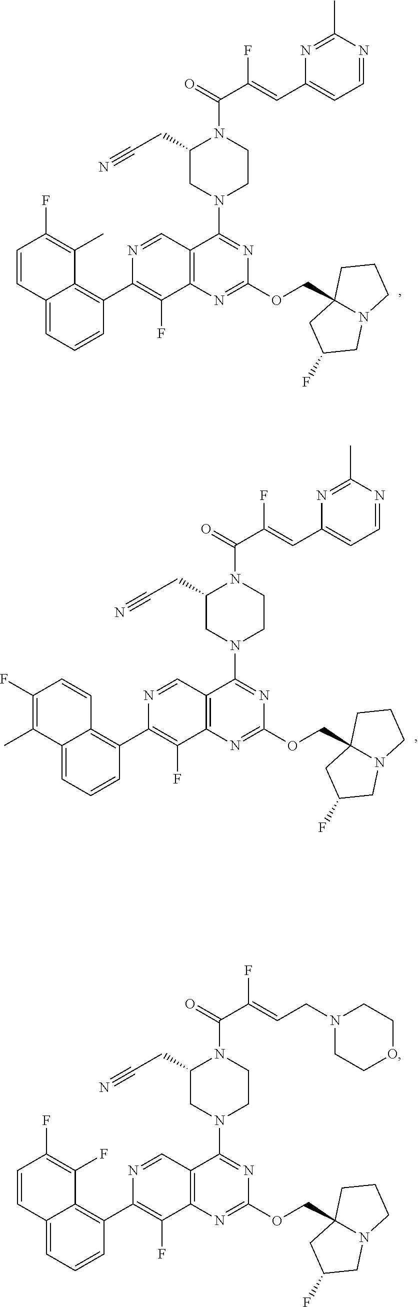 [SMILES: Cc1nccc(/C=C(\F)C(=O)N2CCN(c3nc(OC[C@@]45CCCN4C[C@H](F)C5)nc4c(F)c(-c5cccc6ccc(F)c(C)c56)ncc34)C[C@@H]2CC#N)n1 ; Cc1nccc(/C=C(\F)C(=O)N2CCN(c3nc(OC[C@@]45CCCN4C[C@H](F)C5)nc4c(F)c(-c5cccc6c(C)c(F)ccc56)ncc34)C[C@@H]2CC#N)n1 ; N#CC[C@H]1CN(c2nc(OC[C@@]34CCCN3C[C@H](F)C4)nc3c(F)c(-c4cccc5ccc(F)c(F)c45)ncc23)CCN1C(=O)/C(F)=C/CN1CCOCC1]

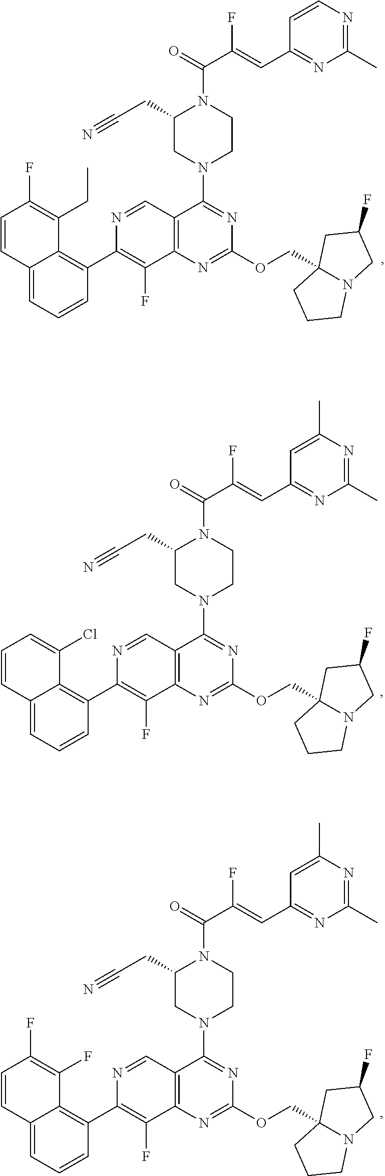 [SMILES: CCc1c(F)ccc2cccc(-c3ncc4c(N5CCN(C(=O)/C(F)=C/c6ccnc(C)n6)[C@@H](CC#N)C5)nc(OC[C@@]56CCCN5C[C@H](F)C6)nc4c3F)c12 ; Cc1cc(/C=C(\F)C(=O)N2CCN(c3nc(OC[C@@]45CCCN4C[C@H](F)C5)nc4c(F)c(-c5cccc6cccc(Cl)c56)ncc34)C[C@@H]2CC#N)nc(C)n1 ; Cc1cc(/C=C(\F)C(=O)N2CCN(c3nc(OC[C@@]45CCCN4C[C@H](F)C5)nc4c(F)c(-c5cccc6ccc(F)c(F)c56)ncc34)C[C@@H]2CC#N)nc(C)n1]

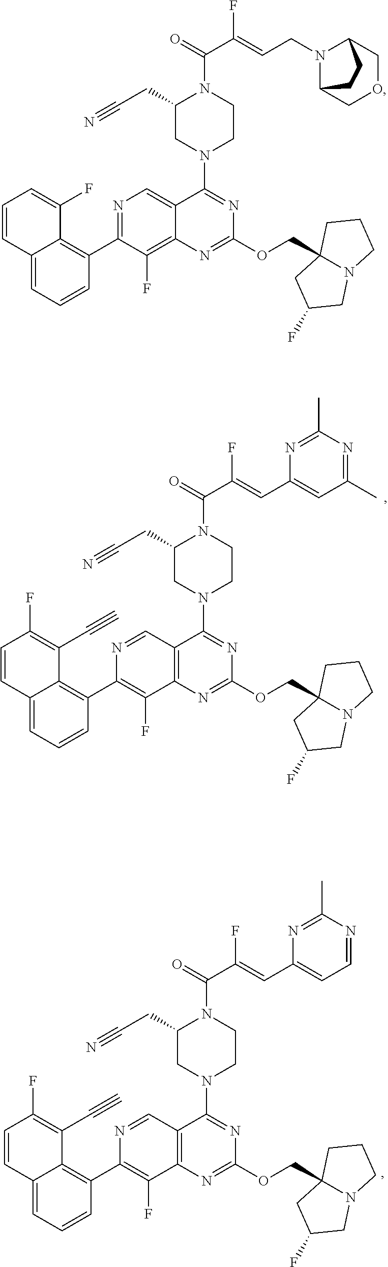

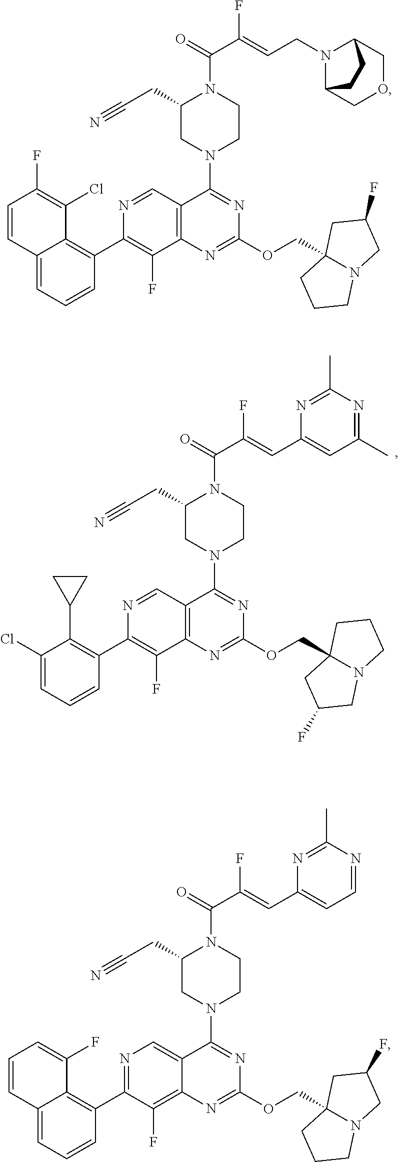

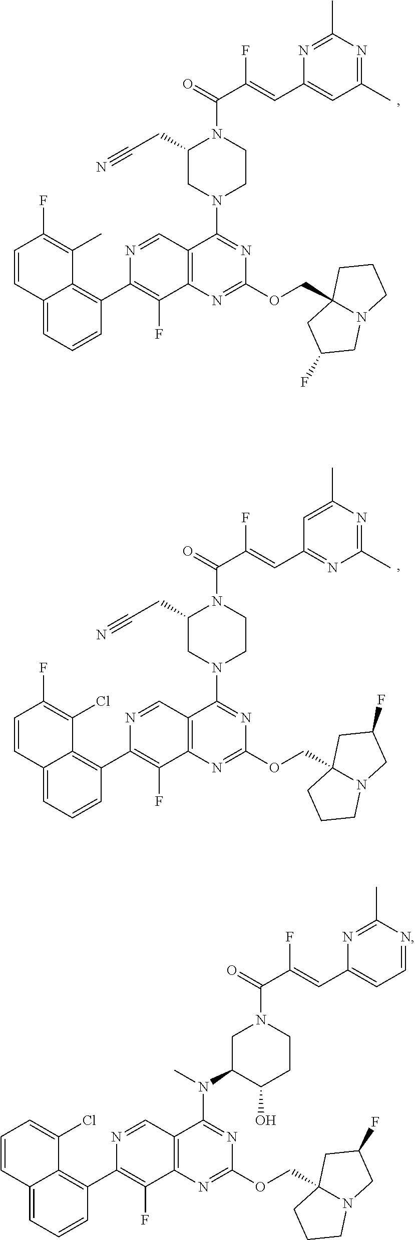 [SMILES: Cc1cc(/C=C(\F)C(=O)N2CCN(c3nc(OC[C@@]45CCCN4C[C@H](F)C5)nc4c(F)c(-c5cccc6ccc(F)c(C)c56)ncc34)C[C@@H]2CC#N)nc(C)n1 ; Cc1cc(/C=C(\F)C(=O)N2CCN(c3nc(OC[C@@]45CCCN4C[C@H](F)C5)nc4c(F)c(-c5cccc6ccc(F)c(Cl)c56)ncc34)C[C@@H]2CC#N)nc(C)n1 ; Cc1nccc(/C=C(\F)C(=O)N2CC[C@H](O)[C@@H](N(C)c3nc(OC[C@@]45CCCN4C[C@H](F)C5)nc4c(F)c(-c5cccc6cccc(Cl)c56)ncc34)C2)n1]

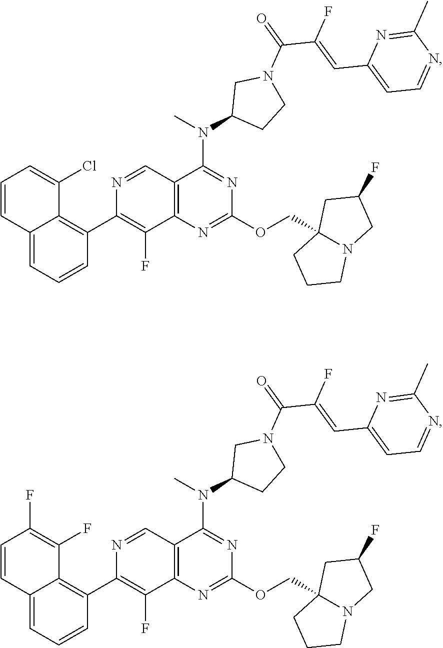 [SMILES: Cc1nccc(/C=C(\F)C(=O)N2CC[C@@H](N(C)c3nc(OC[C@@]45CCCN4C[C@H](F)C5)nc4c(F)c(-c5cccc6cccc(Cl)c56)ncc34)C2)n1 ; Cc1nccc(/C=C(\F)C(=O)N2CC[C@@H](N(C)c3nc(OC[C@@]45CCCN4C[C@H](F)C5)nc4c(F)c(-c5cccc6ccc(F)c(F)c56)ncc34)C2)n1]

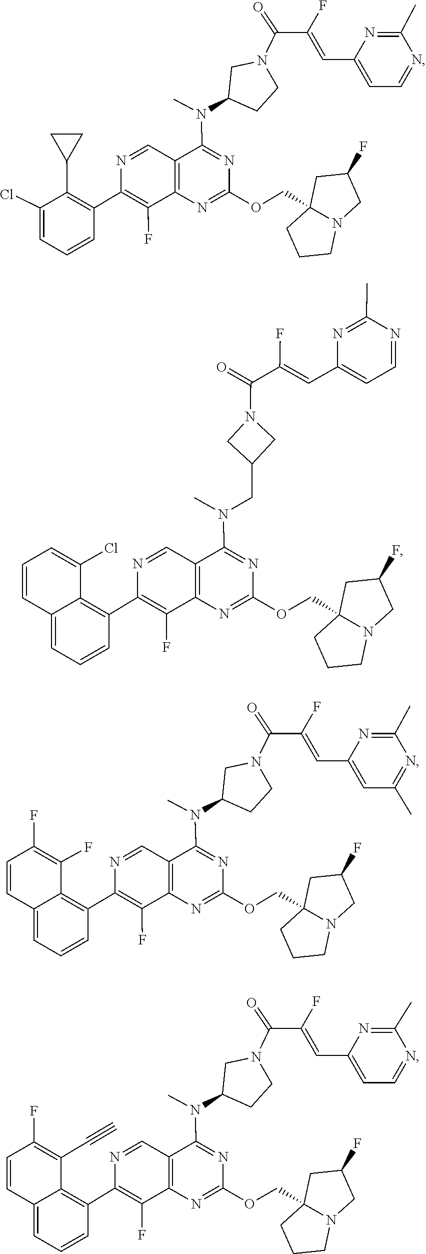

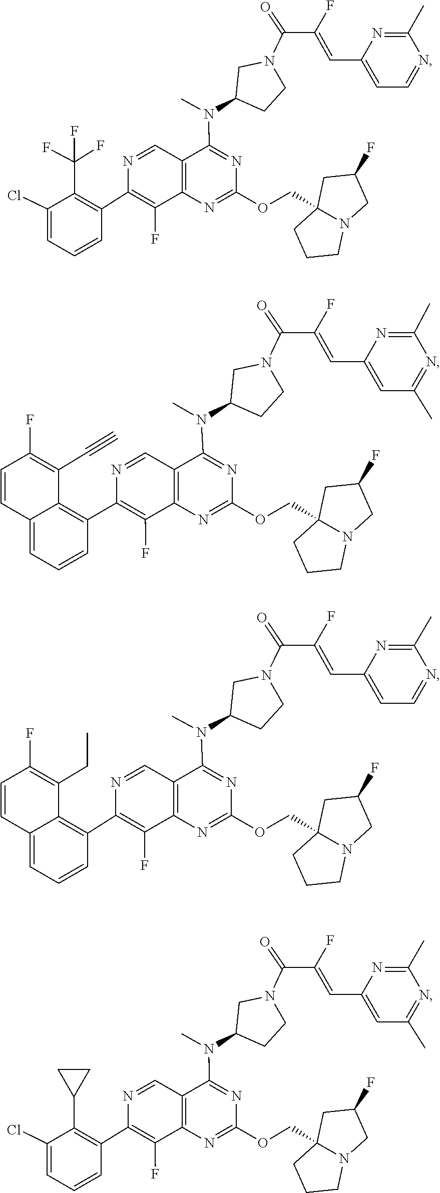 [SMILES: Cc1nccc(/C=C(\F)C(=O)N2CC[C@@H](N(C)c3nc(OC[C@@]45CCCN4C[C@H](F)C5)nc4c(F)c(-c5cccc(Cl)c5C(F)(F)F)ncc34)C2)n1 ; C#Cc1c(F)ccc2cccc(-c3ncc4c(N(C)[C@@H]5CCN(C(=O)/C(F)=C/c6cc(C)nc(C)n6)C5)nc(OC[C@@]56CCCN5C[C@H](F)C6)nc4c3F)c12 ; CCc1c(F)ccc2cccc(-c3ncc4c(N(C)[C@@H]5CCN(C(=O)/C(F)=C/c6ccnc(C)n6)C5)nc(OC[C@@]56CCCN5C[C@H](F)C6)nc4c3F)c12 ; Cc1cc(/C=C(\F)C(=O)N2CC[C@@H](N(C)c3nc(OC[C@@]45CCCN4C[C@H](F)C5)nc4c(F)c(-c5cccc(Cl)c5C5CC5)ncc34)C2)nc(C)n1]

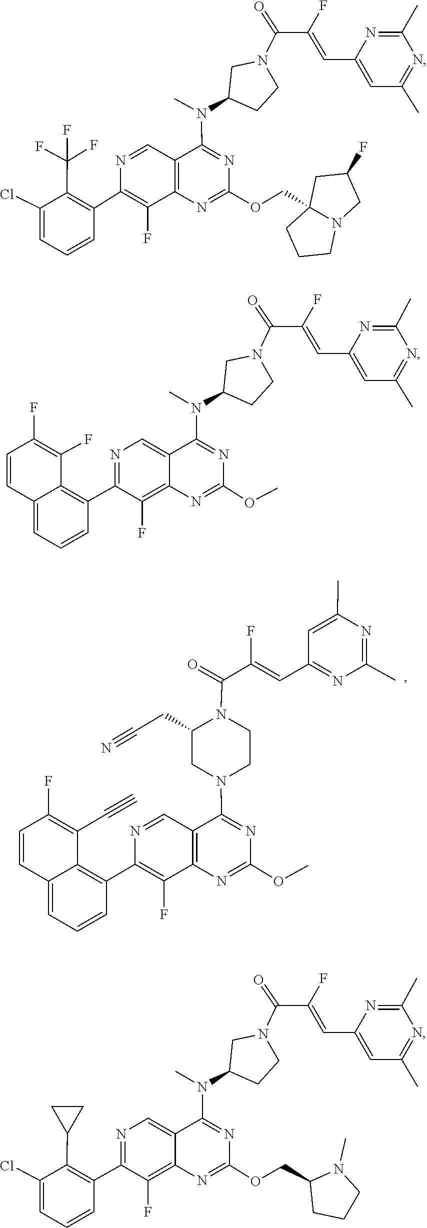 [SMILES: Cc1cc(/C=C(\F)C(=O)N2CC[C@@H](N(C)c3nc(OC[C@@]45CCCN4C[C@H](F)C5)nc4c(F)c(-c5cccc(Cl)c5C(F)(F)F)ncc34)C2)nc(C)n1 ; COc1nc(N(C)[C@@H]2CCN(C(=O)/C(F)=C/c3cc(C)nc(C)n3)C2)c2cnc(-c3cccc4ccc(F)c(F)c34)c(F)c2n1 ; C#Cc1c(F)ccc2cccc(-c3ncc4c(N5CCN(C(=O)/C(F)=C/c6cc(C)nc(C)n6)[C@@H](CC#N)C5)nc(OC)nc4c3F)c12 ; Cc1cc(/C=C(\F)C(=O)N2CC[C@@H](N(C)c3nc(OC[C@@H]4CCCN4C)nc4c(F)c(-c5cccc(Cl)c5C5CC5)ncc34)C2)nc(C)n1]

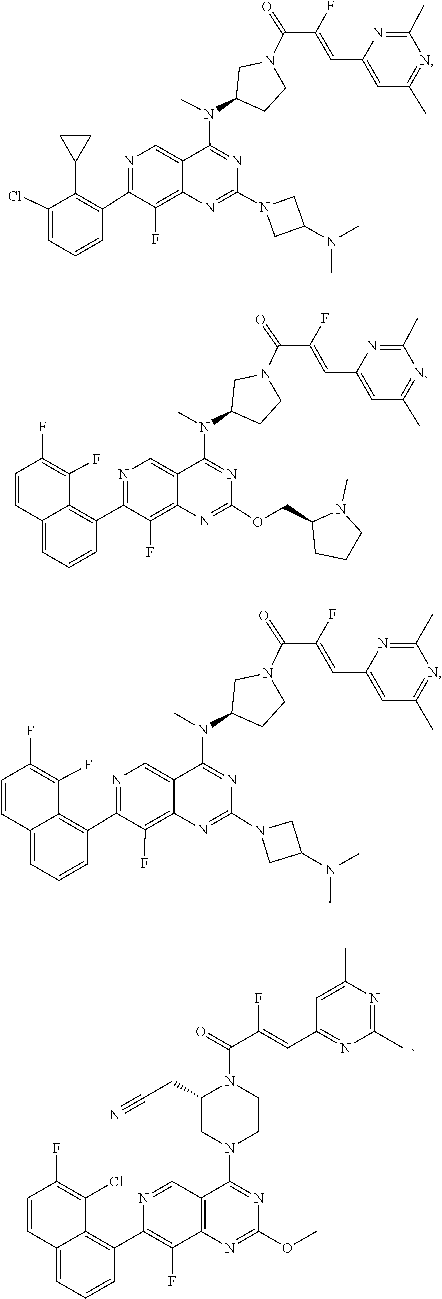 [SMILES: Cc1cc(/C=C(\F)C(=O)N2CC[C@@H](N(C)c3nc(N4CC(N(C)C)C4)nc4c(F)c(-c5cccc(Cl)c5C5CC5)ncc34)C2)nc(C)n1 ; Cc1cc(/C=C(\F)C(=O)N2CC[C@@H](N(C)c3nc(OC[C@@H]4CCCN4C)nc4c(F)c(-c5cccc6ccc(F)c(F)c56)ncc34)C2)nc(C)n1 ; Cc1cc(/C=C(\F)C(=O)N2CC[C@@H](N(C)c3nc(N4CC(N(C)C)C4)nc4c(F)c(-c5cccc6ccc(F)c(F)c56)ncc34)C2)nc(C)n1 ; COc1nc(N2CCN(C(=O)/C(F)=C/c3cc(C)nc(C)n3)[C@@H](CC#N)C2)c2cnc(-c3cccc4ccc(F)c(Cl)c34)c(F)c2n1]

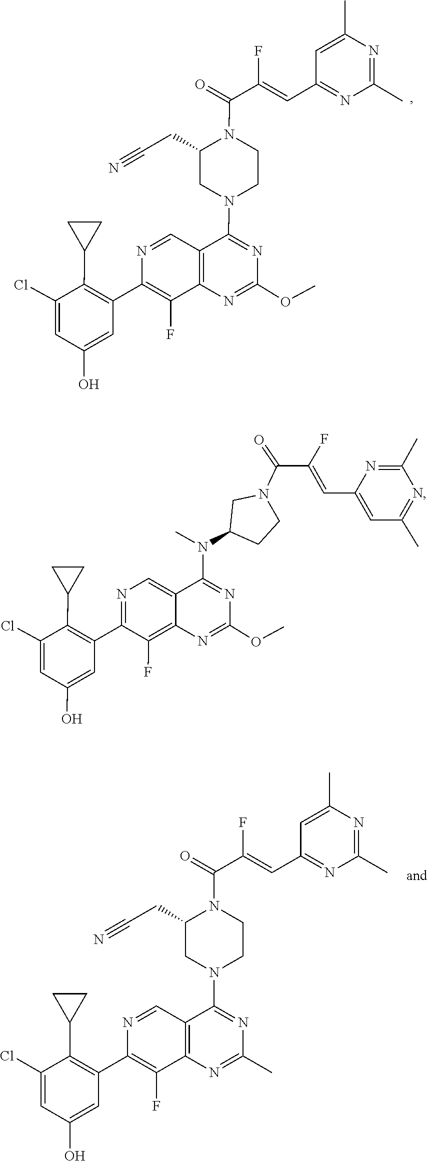 [SMILES: COc1nc(N2CCN(C(=O)/C(F)=C/c3cc(C)nc(C)n3)[C@@H](CC#N)C2)c2cnc(-c3cc(O)cc(Cl)c3C3CC3)c(F)c2n1 ; COc1nc(N(C)[C@@H]2CCN(C(=O)/C(F)=C/c3cc(C)nc(C)n3)C2)c2cnc(-c3cc(O)cc(Cl)c3C3CC3)c(F)c2n1 ; Cc1cc(/C=C(\F)C(=O)N2CCN(c3nc(C)nc4c(F)c(-c5cc(O)cc(Cl)c5C5CC5)ncc34)C[C@@H]2CC#N)nc(C)n1]

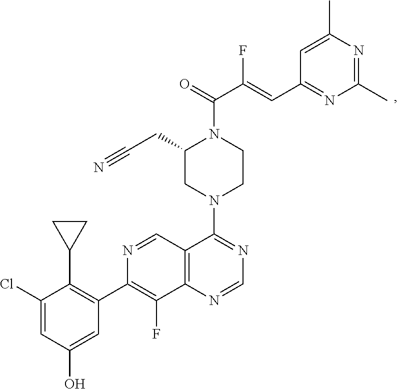 [SMILES: Cc1cc(/C=C(\F)C(=O)N2CCN(c3ncnc4c(F)c(-c5cc(O)cc(Cl)c5C5CC5)ncc34)C[C@@H]2CC#N)nc(C)n1]

or a salt thereof; and/or an isotopologue thereof.

Embodiment 280. The compound of any one of embodiments 1-279, wherein the compound is not a salt.

Embodiment 281. The compound of any one of embodiments 1-279, wherein the compound is a salt.

Embodiment 282. The compound of embodiment 281, wherein the salt is a formate salt.

Embodiment 283. The compound of embodiment 281, wherein the salt is a trifluoroacetate salt.

Embodiment 284. The compound of embodiment 281, wherein the salt is a pharmaceutically acceptable salt.

Embodiment 285. A pharmaceutical formulation comprising the compound of any one of embodiments 1-284, wherein when the compound is a salt, the salt is a pharmaceutically acceptable salt, and a pharmaceutically acceptable carrier.

Embodiment 286. A method of treating or suppressing cancer comprising: administering a therapeutically effective amount of a compound of any one of embodiments 1-284, wherein when the compound is a salt, the salt is a pharmaceutically acceptable salt, or a pharmaceutical formulation according to embodiment 285, to a subject in need thereof.

Embodiment 287. The method of embodiment 286, wherein the cancer is selected from the group consisting of: lung, colorectal, pancreatic, bile duct, thyroid, gall bladder, uterine, mesothelioma, cervical, and bladder cancers.

Embodiment 288. The method of embodiment 286, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cellmyeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cellmalignancies, pediatric neuroblastoma, and melanoma.

Embodiment 289. The method of any one of embodiments 286 to 288, wherein the cancer is a KRAS G12C mediated cancer.

Embodiment 290. The method of any one of embodiments 286 to 288, wherein the subject has been diagnosed as having a KRAS G12C mediated cancer.

Embodiment 291. The method of any one of embodiments 286 to 288, wherein the method further comprises administering to the subject a therapeutically effective amount of an additional chemotherapeutic agent.

Embodiment 292. A compound of any one of embodiments 1-284 or a pharmaceutical formulation according to embodiment 285 for use as a medicament.

Embodiment 293. A compound of any one of embodiments 1-284 or a pharmaceutical formulation according to embodiment 285, for use in treating or suppressing cancer, wherein when the compound is a salt, the salt is a pharmaceutically acceptable salt.

Embodiment 294. The compound or pharmaceutical formulation for use of embodiment 293, wherein the cancer is selected from the group consisting of: lung, colorectal, pancreatic, bile duct, thyroid, gall bladder, uterine, mesothelioma, cervical, and bladder cancers.

Embodiment 295. The compound or pharmaceutical formulation for use of embodiment 293, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

Embodiment 296. The compound or pharmaceutical formulation for use of any one of embodiments 292-295, wherein the cancer is a KRAS G12C mediated cancer.

Embodiment 297. The compound or pharmaceutical formulation for use of any one of embodiments 292-295, wherein the subject has been diagnosed as having a KRAS G12C mediated cancer.

Embodiment 298. The compound or pharmaceutical formulation for use of any one of embodiments 292-297, wherein the compound or pharmaceutical formulation is configured for administration with a therapeutically effective amount of an additional chemotherapeutic agent.

Embodiment 299. The compound or pharmaceutical formulation for use of any one of embodiments 292-298, wherein the compound or pharmaceutical composition is configured for administration in a therapeutically effective amount.

Embodiment 300. A compound of any one of embodiments 1-284 or a pharmaceutical formulation according to embodiment 285 for use in the manufacturing of a medicament for treating or suppressing cancer, wherein when the compound is a salt, the salt is a pharmaceutically acceptable salt.

Embodiment 301. The compound or pharmaceutical formulation for use of embodiment 300, wherein the cancer is selected from the group consisting of: lung, colorectal, pancreatic, bile duct, thyroid, gall bladder, uterine, mesothelioma, cervical, and bladder cancers.

Embodiment 302. The compound or pharmaceutical formulation for use of embodiment 300, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

Embodiment 303. The compound or pharmaceutical formulation for use of any one of embodiments 300-303, wherein the cancer is a KRAS G12C mediated cancer.

Embodiment 304. The compound or pharmaceutical formulation for use of any one of embodiments 300-303, wherein the subject has been diagnosed as having a KRAS G12C mediated cancer.

Embodiment 305. The compound or pharmaceutical formulation for use of any one of embodiments 300-304, wherein the compound or pharmaceutical formulation is configured for administration with a therapeutically effective amount of an additional chemotherapeutic agent.

Embodiment 306. The compound or pharmaceutical formulation for use of any one of embodiments 300-305, wherein the medicament comprises a therapeutically effective amount of the compound or composition.

Embodiment 307. Use of a compound of any one of embodiments 1-284 or a pharmaceutical formulation according to embodiment 285 in the manufacturing of a medicament for treating or suppressing cancer, wherein when the compound is a salt, the salt is a pharmaceutically acceptable salt.

Embodiment 308. The use of embodiment 307, wherein the cancer is selected from the group consisting of: lung, colorectal, pancreatic, bile duct, thyroid, gall bladder, uterine, mesothelioma, cervical, and bladder cancers.

Embodiment 309. The use of embodiment 307, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

Embodiment 310. The use of any one of embodiments 307-309, wherein the cancer is a KRAS G12C mediated cancer.

Embodiment 311. The use of any one of embodiments 307-309, wherein the subject has been diagnosed as having a KRAS G12C mediated cancer.

Embodiment 312. The use of any one of embodiments 307-311, wherein the compound or pharmaceutical composition is configured for administration with a therapeutically effective amount of an additional chemotherapeutic agent.

Embodiment 313. The use of any one of embodiments 307-312, wherein the medicament comprises a therapeutically effective amount of the compound or pharmaceutical composition.

Embodiment 314. Use of a compound of any one of embodiments 1-284 or a pharmaceutical formulation according to embodiment 285 for treating or suppressing cancer, wherein when the compound is a salt, the salt is a pharmaceutically acceptable salt.

Embodiment 315. The use of embodiment 314, wherein the cancer is selected from the group consisting of: lung, colorectal, pancreatic, bile duct, thyroid, gall bladder, uterine, mesothelioma, cervical, and bladder cancers.

Embodiment 316. The use of embodiment 314, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

Embodiment 317. The use of any one of embodiments 314-316, wherein the cancer is a KRAS G12C mediated cancer.

Embodiment 318. The use of any one of embodiments 314-316, wherein the subject has been diagnosed as having a KRAS G12C mediated cancer.

Embodiment 319. The use of any one of embodiments 314-318, wherein the compound or pharmaceutical composition is configured for administration with a therapeutically effective amount of an additional chemotherapeutic agent.

Embodiment 320. The use of any one of embodiments 314-319, wherein use involves a therapeutically effective amount of the compound or composition.

### General Synthetic Methods

Compounds 1-58 in Table 1 of the instant disclosure were prepared according to methods described in the Examples section or variations thereof that would be within the knowledge of one of skill in the art.

The starting materials and reagents used in preparing these compounds are either available from commercial suppliers such as MilliporeSigma., Bachem., etc. or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

### EXAMPLES

The following preparations of compounds of Formula (I), Formula (II), Formula (III), Formula (IV), Formula (V), Formula (VI) and pharmaceutically acceptable salts thereof are given to enable those skilled in the art to more clearly understand and to practice the present disclosure. They should not be considered as limiting the scope of the disclosure, but merely as being illustrative and representative thereof.

The following abbreviations are used in this section:

| | |
|---|---|
| Boc | tert-butyloxycarbonyl |
| PMB | p-methoxybenzyl |
| ACN | acetonitrile |
| TFA | trifluoroacetic acid |
| FA | formic acid |
| mmol | Millimoles |
| g | Grams |
| mg | Milligrams |
| h | Hours |
| mL | Milliliters |
| NMR | Nuclear magnetic resonance |
| MHz | Megahertz |
| Hz | Hertz |
| LCMS | Liquid chromatography-mass spectrometry |
| Rt | Retention time |
| m/z | Mass/charge |
| min | Minutes |
| um | Micrometers |
| mm | Millimeters |
| umol | Micromoles |
| BEH | Ethylene bridged hybrid |
| HPLC | High-performance liquid chromatography |
| ESI | Electrospray ionization |
| mM | Millimolar |
| OBD | Optimum bed density |
| TIPS | Triisopropylsilyl |

All reagents were obtained from commercial suppliers and used without further purification unless otherwise stated.

### SYNTHETIC EXAMPLES

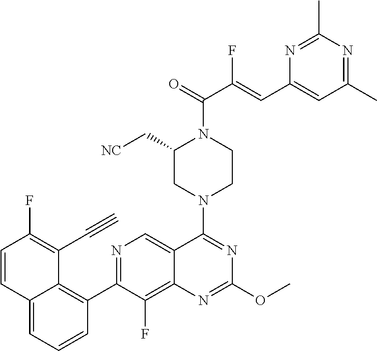 [SMILES: C#Cc1c(F)ccc2cccc(-c3ncc4c(N5CCN(C(=O)/C(F)=C/c6cc(C)nc(C)n6)[C@@H](CC#N)C5)nc(OC)nc4c3F)c12]

### Example 1: Synthesis of Compound 49—(S,Z)-2-(1-(3-(2,6-dimethylpyrimidin-4-yl)-2-fluoroacryloyl)-4-(7-(8-ethynyl-7-fluoronaphthalen-1-yl)-8-fluoro-2-methoxypyrido[4,3-d]pyrimidin-4-yl)piperazin-2-yl)acetonitrile (Method 1)

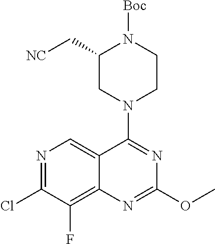 [SMILES: COc1nc(N2CCN(C(=O)OC(C)(C)C)[C@@H](CC#N)C2)c2cnc(Cl)c(F)c2n1]

### Step 1: tert-butyl (2S)-4-(7-chloro-8-fluoro-2-methoxy-pyrido[4,3-d]pyrimidin-4-yl)-2-(cyanomethyl)piperazine-1-carboxylate

To a solution of tert-butyl (2S)-2-(cyanomethyl)-4-(2,7-dichloro-8-fluoro-pyrido[4,3-d]pyrimidin-4-yl)piperazine-1-carboxylate (1.5 g, 3.40 mmol) in methanol (20 mL) was added sodium methoxide (734.54 mg, 4.08 mmol). The mixture was stirred at 25° C. for 0.5 h. The reaction mixture was diluted with water (30 mL) and extracted with dichloromethane (3×20 mL). The combined organic layers were dried over sodium sulphate and concentrated under vacuo affording tert-butyl (2S)-4-(7-chloro-8-fluoro-2-methoxy-pyrido[4,3-d]pyrimidin-4-yl)-2-(cyanomethyl)piperazine-1-carboxylate (950 mg, 63.97%) as a yellow solid and used in the next step without further purification. LCMS Rt=0.511 min, m/z=436.1 [M+H]⁺.

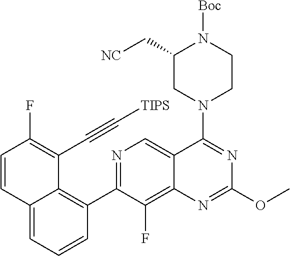

### Step 2: tert-butyl (2S)-2-(cyanomethyl)-4-[8-fluoro-7-[7-fluoro-8-(2-triisopropylsilylethynyl)-1-naphthyl]-2-methoxy-pyrido[4,3-d]pyrimidin-4-yl]piperazine-1-carboxylate

A mixture of tert-butyl (2S)-4-(7-chloro-8-fluoro-2-methoxy-pyrido[4,3-d]pyrimidin-4-yl)-2-(cyanomethyl)piperazine-1-carboxylate (300 mg, 686.71 mol), 2-[2-fluoro-8-(4,4,5,5-tetramethyl-1,3,2-dioxaborolan-2-yl)-1-naphthyl]ethynyl-triisopropyl-silane (683.59 mg, 1.51 mmol), [2-(2-aminophenyl)phenyl]-chloro-palladium;bis(1-adamantyl)-butyl-phosphane (45.92 mg, 68.67 mol), potassium phosphate (437.29 mg, 2.06 mmol) in dioxane (3 mL), water (1 mL) was degassed and purged with nitrogen for 3 times, and then the mixture was stirred at 80° C. for 1 h under nitrogen atmosphere. The reaction mixture was diluted with water (3 mL) and extracted with dichloromethane (3×3 mL). The combined organic layers were dried over sodium sulphate and concentrated under vacuo. The residue was purified by reverse phase HPLC (column: Waters Xbridge BEH C18 100*30 mm*10 um; mobile phase: [water(NH₄HCO₃)-ACN]; B %: 75%-95%,8 min) affording tert-butyl (2S)-2-(cyanomethyl)-4-[8-fluoro-7-[7-fluoro-8-(2-triisopropylsilylethynyl)-1-naphthyl]-2-methoxy-pyrido[4,3-d]pyrimidin-4-yl]piperazine-1-carboxylate (77 mg, 15.43%) as a brown solid. LCMS Rt=2.20 min, m/z=726.4 [M+H]⁺.

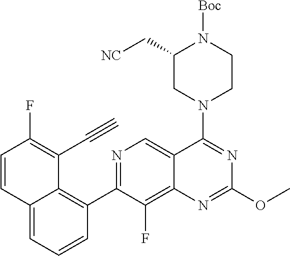 [SMILES: C#Cc1c(F)ccc2cccc(-c3ncc4c(N5CCN(C(=O)OC(C)(C)C)[C@@H](CC#N)C5)nc(OC)nc4c3F)c12]

### Step 3: tert-butyl (2S)-2-(cyanomethyl)-4-[7-(8-ethynyl-7-fluoro-1-naphthyl)-8-fluoro-2-methoxy-pyrido[4,3-d]pyrimidin-4-yl]piperazine-1-carboxylate

To a solution of tert-butyl (2S)-2-(cyanomethyl)-4-[8-fluoro-7-[7-fluoro-8-(2-triisopropylsilylethynyl)-1-naphthyl]-2-methoxy-pyrido[4,3-d]pyrimidin-4-yl]piperazine-1-carboxylate (67 mg, 92.17 mol) in acetonitrile (1.5 mL) was added cesium fluoride (70.00 mg, 460.84 mol). The mixture was stirred at 25° C. for 0.5 h.

The reaction mixture was diluted with water (10 mL) and extracted with dichloromethane (3×10 mL). The combined organic layers were dried over sodium sulphate and concentrated under vacuo affording tert-butyl (2S)-2-(cyanomethyl)-4-[7-(8-ethynyl-7-fluoro-1-naphthyl)-8-fluoro-2-methoxy-pyrido[4,3-d]pyrimidin-4-yl]piperazine-1-carboxylate (53 mg, crude) as a brown oil used into the next step without further purification. LCMS Rt=0.57 min, m/z=570.2 [M+H]⁺.

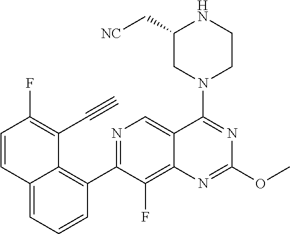 [SMILES: C#Cc1c(F)ccc2cccc(-c3ncc4c(N5CCN[C@@H](CC#N)C5)nc(OC)nc4c3F)c12]

### Step 4: 2—[(2S)-4-[7-(8-ethynyl-7-fluoro-1-naphthyl)-8-fluoro-2-methoxy-pyrido[4,3-d]pyrimidin-4-yl]piperazin-2-yl]acetonitrile

To a solution of tert-butyl (2S)-2-(cyanomethyl)-4-[7-(8-ethynyl-7-fluoro-1-naphthyl)-8-fluoro-2-methoxy-pyrido[4,3-d]pyrimidin-4-yl]piperazine-1-carboxylate (53 mg, 92.89 mol) in dichloromethane (1 mL) was added trifluoroacetic acid (0.3 mL). The mixture was stirred at 25° C. for 0.5 h. The reaction mixture was concentrated under vacuo affording 2-[(2S)-4-[7-(8-ethynyl-7-fluoro-1-naphthyl)-8-fluoro-2-methoxy-pyrido[4,3-d]pyrimidin-4-yl]piperazin-2-yl]acetonitrile (54 mg, crude, trifluoroacetic acid salt) as a yellow oil used into the next step without further purification. LCMS Rt=0.471 min, m/z=470.2 [M+H]⁺.

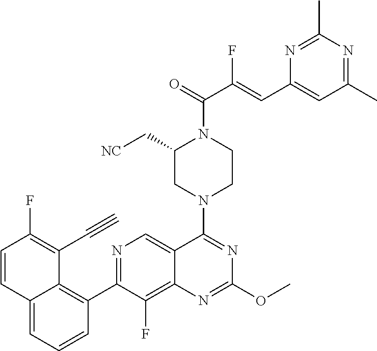 [SMILES: C#Cc1c(F)ccc2cccc(-c3ncc4c(N5CCN(C(=O)/C(F)=C/c6cc(C)nc(C)n6)[C@@H](CC#N)C5)nc(OC)nc4c3F)c12]

### Step 5: (S,Z)-2-(1-(3-(2,6-dimethylpyrimidin-4-yl)-2-fluoroacryloyl)-4-(7-(8-ethynyl-7-fluoronaphthalen-1-yl)-8-fluoro-2-methoxypyrido[4,3-d]pyrimidin-4-yl)piperazin-2-yl)acetonitrile

To a solution of 2-[(2S)-4-[7-(8-ethynyl-7-fluoro-1-naphthyl)-8-fluoro-2-methoxy-pyrido[4,3-d]pyrimidin-4-yl]piperazin-2-yl]acetonitrile (54 mg, 92.39 mol, trifluoroacetic acid salt) in N,N-dimethylformaldehyde (1 mL) was added N,N-diisopropylethylamine (35.82 mg, 277.16 mol), (Z)-3-(2,6-dimethylpyrimidin-4-yl)-2-fluoro-prop-2-enoic acid (36.25 mg, 184.77 mol) and 2-Chloro-1,3-dimethylimidazolinium chloride (39.92 mg, 277.16 mol). The mixture was stirred at 25° C. for 10 min. The reaction mixture was concentrated to dryness in vacuo. The resulting residue was purified by prep-HPLC (column: Waters Xbridge C18 150*50 mm*10 um; mobile phase: [H₂O(10 mM NH₄HCO₃)-ACN]; gradient: 40%-60% B over 8.0 min) affording (S,Z)-2-(1-(3-(2,6-dimethylpyrimidin-4-yl)-2-fluoroacryloyl)-4-(7-(8-ethynyl-7-fluoronaphthalen-1-yl)-8-fluoro-2-methoxypyrido[4,3-d]pyrimidin-4-yl)piperazin-2-yl)acetonitrile (21.68 mg, 35.24%) as a yellow solid: ¹H NMR (400 MHz, Acetonitrile-d3) δ 9.10 (d, J=8.0 Hz, 1H), 8.12 (dd, J=5.8, 8.9 Hz, 2H), 7.71-7.64 (m, 2H), 7.50-7.41 (m, 2H), 6.54-6.38 (m, 1H), 4.92 (dt, J=1.5, 6.3 Hz, 1H), 4.62-4.39 (m, 2H), 4.26-4.10 (m, 1H), 4.04 (d, J=2.5 Hz, 3H), 3.98-3.86 (m, 1H), 3.85-3.67 (m, 2H), 3.30-3.22 (m, 1H), 3.15-3.05 (m, 1H), 3.04-2.90 (m, 1H), 2.59 (s, 3H), 2.47 (s, 3H). LCMS Rt=2.937 min, m/z=648.2 [M+H]⁺. LCMS (5 to 95% acetonitrile in water+0.03% ammonium bicarbonate over 6 min) retention time 2.937 min, ESI+ found [M+H]=648.2.

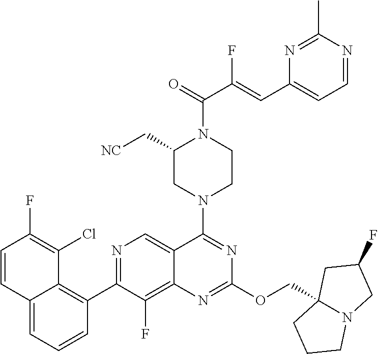 [SMILES: Cc1nccc(/C=C(\F)C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1]

### Example 2: Synthesis of Compound 5—2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(2-methylpyrimidin-4-yl)acryloyl)piperazin-2-yl)acetonitrile (Method 1)

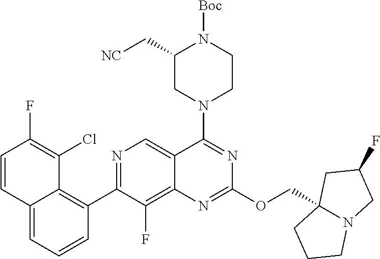 [SMILES: CC(C)(C)OC(=O)N1CCN(c2nc(OC[C@@]34CCCN3C[C@H](F)C4)nc3c(F)c(-c4cccc5ccc(F)c(Cl)c45)ncc23)C[C@@H]1CC#N]

### Step 1: (S)-tert-butyl 4-(7-(8-chloro-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-(cyanomethyl)piperazine-1-carboxylate

The Suzuki reaction was prepared in a similar fashion to Method #1, Step 2. The residue was purified by column chromatography (silica gel, 100-200 mesh, 0-100% ethyl acetate in petroleum ether) affording (S)-tert-butyl 4-(7-(8-chloro-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-(cyanomethyl)piperazine-1-carboxylate (4.8 g, 76.46%) as a brown solid. LCMS Rt=0.685 min, m/z=707.3 [M+H]⁺.

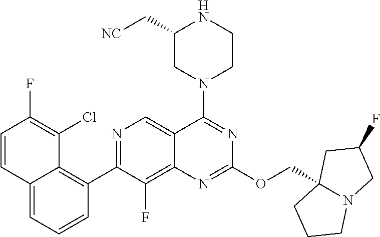 [SMILES: N#CC[C@H]1CN(c2nc(OC[C@@]34CCCN3C[C@H](F)C4)nc3c(F)c(-c4cccc5ccc(F)c(Cl)c45)ncc23)CCN1]

### Step 2: 2—((S)-4-(7-(8-chloro-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The deprotection of Boc group was prepared in a similar fashion to Method #1, Step 4. The reaction mixture was concentrated in vacuo affording 2-((S)-4-(7-(8-chloro-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (600.00 mg, crude) as a brown oil used into the next step without further purification. LCMS Rt=0.519 min, m/z=607.2 [M+H]⁺.

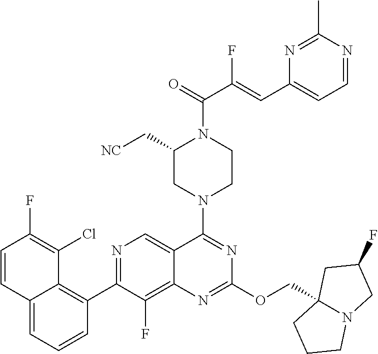 [SMILES: Cc1nccc(/C=C(\F)C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1]

### Step 3: 2—((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(2-methylpyrimidin-4-yl)acryloyl)piperazin-2-yl)acetonitrile

The amide coupling reaction was prepared in a similar fashion to Method #1, Step 5. The residue was purified by reverse phase HPLC (column: Waters Xbridge Prep OBD C18 150*40 mm*10 um; mobile phase: [water(NH₄HCO₃)-ACN]; B %: 35%-65%, 8 min) affording 2-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(2-methylpyrimidin-4-yl)acryloyl)piperazin-2-yl)acetonitrile (163.03 mg, 25.41%) as a amorphous solid: ¹H NMR (400 MHz, Acetonitrile-d3) δ 9.26-8.99 (m, 1H), 8.70 (d, J=5.3 Hz, 1H), 8.18-8.09 (m, 1H), 8.06 (dd, J=5.9, 9.1 Hz, 1H), 7.79-7.61 (m, 2H), 7.59-7.35 (m, 2H), 6.64-6.34 (m, 1H), 5.37-5.16 (m, 1H), 5.04-4.82 (m, 1H), 4.69-4.39 (m, 2H), 4.33-4.09 (m, 3H), 3.96-3.61 (m, 3H), 3.24-3.12 (m, 2H), 3.08 (br s, 1H), 3.02 (br d, J=7.8 Hz, 1H), 2.99-2.86 (m, 2H), 2.66-2.52 (m, 3H), 2.20 (br s, 1H), 2.12 (br s, 1H), 2.09-2.04 (m, 1H), 1.92-1.81 (m, 3H). LCMS Rt=2.236 min, m/z=771.3 [M+H]⁺. LCMS (5 to 95% acetonitrile in water+0.03% ammonium bicarbonate over 6 mins) retention time 2.236 min, ESI+ found [M+H]=771.3.

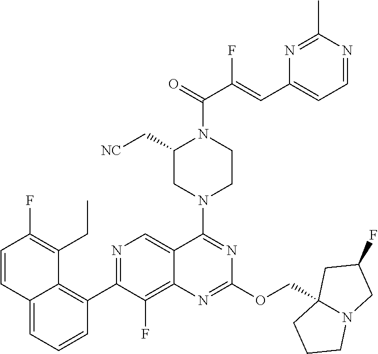 [SMILES: CCc1c(F)ccc2cccc(-c3ncc4c(N5CCN(C(=O)/C(F)=C/c6ccnc(C)n6)[C@@H](CC#N)C5)nc(OC[C@@]56CCCN5C[C@H](F)C6)nc4c3F)c12]

### Example 3: Synthesis of Compound 9—2-((S)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(2-methylpyrimidin-4-yl)acryloyl)piperazin-2-yl)acetonitrile (Method 1)

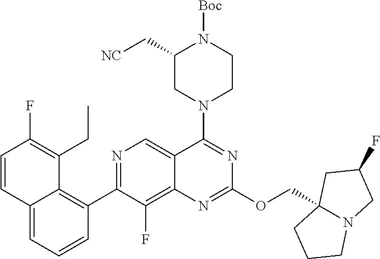 [SMILES: CCc1c(F)ccc2cccc(-c3ncc4c(N5CCN(C(=O)OC(C)(C)C)[C@@H](CC#N)C5)nc(OC[C@@]56CCCN5C[C@H](F)C6)nc4c3F)c12]

### Step 1: (S)-tert-butyl 2-(cyanomethyl)-4-(7-(8-ethyl-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazine-1-carboxylate

The Suzuki reaction was prepared in a similar fashion to Method #1, Step 2. The residue was purified by reverse phase HPLC (column: Phenomenex Luna C18 (250*70 mm,15 um); mobile phase: [water(TFA)-ACN]; B %: 33%-60%, 22 min) affording (S)-tert-butyl 2-(cyanomethyl)-4-(7-(8-ethyl-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazine-1-carboxylate (1.5 g, 20.74%, trifluoroacetic salt) as a yellow oil: ¹H NMR (400 MHz, Acetonitrile-d3) δ 9.20 (d, J=6.8 Hz, 1H), 8.08 (br d, J=8.1 Hz, 1H), 7.95 (dd, J=5.9, 8.9 Hz, 1H), 7.61-7.54 (m, 1H), 7.52-7.47 (m, 1H), 7.41 (t, J=9.4 Hz, 1H), 5.57-5.41 (m, 1H), 4.66-4.61 (m, 3H), 4.45-4.39 (m, 1H), 4.05-3.98 (m, 2H), 3.71-3.62 (m, 2H), 3.36-3.27 (m, 2H), 2.87-2.82 (m, 2H), 2.58 (br d, J=10.0 Hz, 1H), 2.50 (br s, 1H), 2.37 (br d, J=5.3 Hz, 1H), 2.29-2.22 (m, 3H), 2.13-2.07 (m, 1H), 1.49 (s, 9H), 1.47 (br d, J=2.4 Hz, 3H), 1.33-1.25 (m, 1H), 0.85-0.79 (m, 3H) LCMS Rt=0.796 min, m/z=701.3 [M+H]⁺.

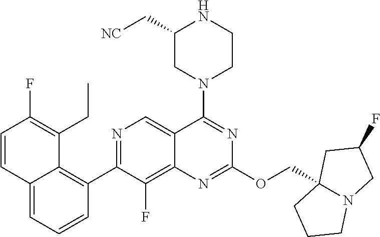 [SMILES: CCc1c(F)ccc2cccc(-c3ncc4c(N5CCN[C@@H](CC#N)C5)nc(OC[C@@]56CCCN5C[C@H](F)C6)nc4c3F)c12]

### Step 2: 2—((S)-4-(7-(8-ethyl-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The deprotection of Boc group was prepared in a similar fashion to Method #1, Step 4. The reaction mixture was concentrated in vacuo affording 2-((S)-4-(7-(8-ethyl-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (400.00 mg, crude, trifluoroacetic salt) as a brown oil used into the next step without further purification. LCMS Rt=0.515 min, m/z=601.3 [M+H]⁺.

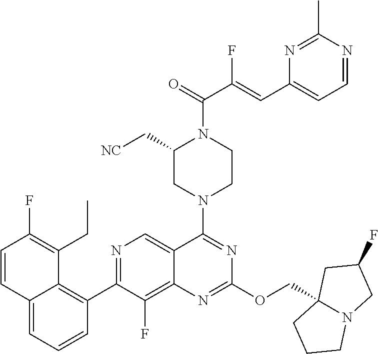 [SMILES: CCc1c(F)ccc2cccc(-c3ncc4c(N5CCN(C(=O)/C(F)=C/c6ccnc(C)n6)[C@@H](CC#N)C5)nc(OC[C@@]56CCCN5C[C@H](F)C6)nc4c3F)c12]

### Step 3: 2—((S)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(2-methylpyrimidin-4-yl)acryloyl)piperazin-2-yl)acetonitrile

The amide coupling reaction was prepared in a similar fashion to Method #1, Step 5. The residue was purified by reverse phase HPLC (column: Waters Xbridge Prep OBD C18 150*40 mm*10 um; mobile phase: [water(NH₄HCO₃)-ACN]; B %: 35%-65%, 8 min) affording 2-((S)-4-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(2-methylpyrimidin-4-yl)acryloyl)piperazin-2-yl)acetonitrile (130.0 mg, 30.22%) as a amorphous solid: ¹H NMR (400 MHz, Dimethylsulfoxide-d6) δ 9.24-9.17 (m, 1H), 8.83-8.75 (m, 1H), 8.13 (br d, J=8.0 Hz, 1H), 8.03 (dd, J=6.1, 9.0 Hz, 1H), 7.63-7.57 (m, 2H), 7.52-7.46 (m, 2H), 6.65-6.47 (m, 1H), 5.38-5.18 (m, 1H), 4.99-4.86 (m, 1H), 4.61-4.39 (m, 2H), 4.22-4.06 (m, 3H), 4.00-3.65 (m, 3H), 3.17-3.00 (m, 5H), 2.89-2.78 (m, 1H), 2.64 (s, 3H), 2.46-2.37 (m, 1H), 2.25-2.12 (m, 2H), 2.08-2.00 (m, 2H), 1.88-1.76 (m, 3H), 0.77 (q, J=7.4 Hz, 3H). LCMS Rt=3.216 min, m/z=765.3 [M+H]⁺. LCMS (5 to 95% acetonitrile in water+0.03% ammonium bicarbonate over 6 mins) retention time 3.216 min, ESI+ found [M+H]=765.3.

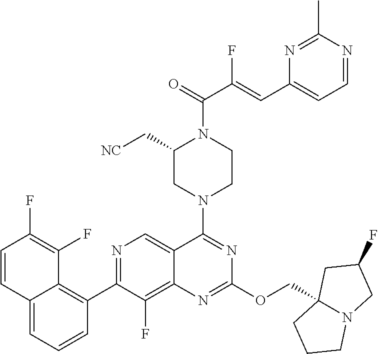 [SMILES: Cc1nccc(/C=C(\F)C(=O)N2CCN(c3nc(OC[C@@]45CCCN4C[C@H](F)C5)nc4c(F)c(-c5cccc6ccc(F)c(F)c56)ncc34)C[C@@H]2CC#N)n1]

### Example 4: Synthesis of Compound 11—2-((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(2-methylpyrimidin-4-yl)acryloyl)piperazin-2-yl)acetonitrile (Method Boc

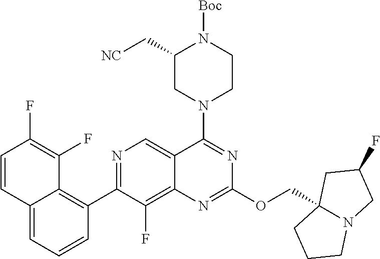 [SMILES: CC(C)(C)OC(=O)N1CCN(c2nc(OC[C@@]34CCCN3C[C@H](F)C4)nc3c(F)c(-c4cccc5ccc(F)c(F)c45)ncc23)C[C@@H]1CC#N]

### Step 1: (S)-tert-butyl 2-(cyanomethyl)-4-(7-(7,8-difluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazine-1-carboxylate

The Suzuki reaction was prepared in a similar fashion to Method #, Step 2. The residue was purified by flash column (ISCO 25 g silica, 0-30% ethyl acetate in petroleum ether, gradient over 30 min) affording (S)-tert-butyl 2-(cyanomethyl)-4-(7-(7,8-difluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazine-1-carboxylate (4.5 g, 87.42%) as a yellow gum: ¹H NMR (400 MHz, Chloroform-d) δ 9.00 (s, 1H), 7.89 (br d, J=7.9 Hz, 1H), 7.67-7.61 (m, 1H), 7.58-7.50 (m, 2H), 7.36-7.28 (m, 1H), 5.30-5.12 (m, 1H), 4.62-4.35 (m, 3H), 4.25 (br dd, J=5.1, 10.3 Hz, 1H), 4.16 (br d, J=10.4 Hz, 1H), 3.79-3.59 (m, 2H), 3.38-3.27 (m, 1H), 3.24-3.16 (m, 2H), 3.14-3.09 (m, 1H), 2.96-2.88 (m, 1H), 2.75 (br d, J=6.8 Hz, 1H), 2.71-2.63 (m, 1H), 2.39 (br dd, J=6.6, 14.1 Hz, 1H), 2.21-2.07 (m, 3H), 1.90-1.80 (m, 3H), 1.45 (s, 9H). LCMS Rt=0.638 min, m/z=691.3 [M+H]⁺.

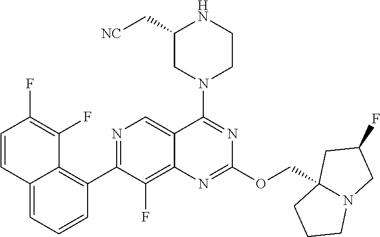 [SMILES: N#CC[C@H]1CN(c2nc(OC[C@@]34CCCN3C[C@H](F)C4)nc3c(F)c(-c4cccc5ccc(F)c(F)c45)ncc23)CCN1]

### Step 2: ((S)-4-(7-(7,8-difluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The deprotection of Boc group was prepared in a similar fashion to Method #1, Step 4. The reaction mixture was concentrated in vacuo affording 2-((S)-4-(7-(7,8-difluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (181.00 mg, crude, trifluoroacetic salt) as a brown gum used into the next step without further purification. LCMS Rt=0.539 min, m/z=591.2 [M+H]⁺.

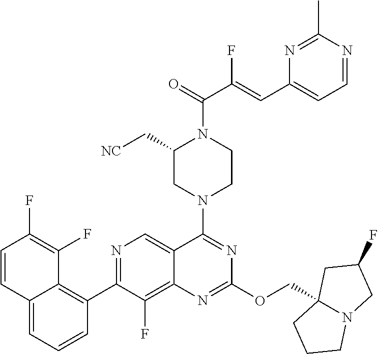 [SMILES: Cc1nccc(/C=C(\F)C(=O)N2CCN(c3nc(OC[C@@]45CCCN4C[C@H](F)C5)nc4c(F)c(-c5cccc6ccc(F)c(F)c56)ncc34)C[C@@H]2CC#N)n1]

### Step 3: 2—((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(2-methylpyrimidin-4-yl)acryloyl)piperazin-2-yl)acetonitrile

The amide coupling reaction was prepared in a similar fashion to Method #1, Step 5. The residue was purified by reverse phase HPLC (column: Waters Xbridge Prep OBD C18 150*40 mm*10 um; mobile phase: [water(NH₄HCO₃)-ACN]; B %: 30%-60%, 8 min) affording 2-((S)-4-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(2-methylpyrimidin-4-yl)acryloyl)piperazin-2-yl)acetonitrile (82.53 mg, 32.30%) as a yellow oil: ¹H NMR (400 MHz, Chloroform-d) δ 9.12-9.05 (m, 1H), 8.72 (d, J=5.3 Hz, 1H), 7.99 (br d, J=7.8 Hz, 1H), 7.76-7.69 (m, 1H), 7.67-7.58 (m, 2H), 7.51 (d, J=5.1 Hz, 1H), 7.45-7.37 (m, 1H), 6.88-6.63 (m, 1H), 5.40-5.22 (m, 1H), 5.08-4.92 (m, 1H), 4.85 (br s, 1H), 4.80-4.72 (m, 1H), 4.61-4.46 (m, 2H), 4.39-4.30 (m, 2H), 4.13-3.96 (m, 1H), 3.96-3.76 (m, 2H), 3.37-3.32 (m, 1H), 3.27-3.21 (m, 1H), 3.14-2.97 (m, 2H), 2.95-2.87 (m, 1H), 2.76 (s, 3H), 2.33-2.16 (m, 3H), 2.03-1.93 (m, 3H). LCMS Rt=3.013 min, m/z=755.3 [M+H]⁺. LCMS (5 to 95% acetonitrile in water+0.03% ammonium bicarbonate over 6 mins) retention time 3.013 min, ESI+ found [M+H]=755.3.

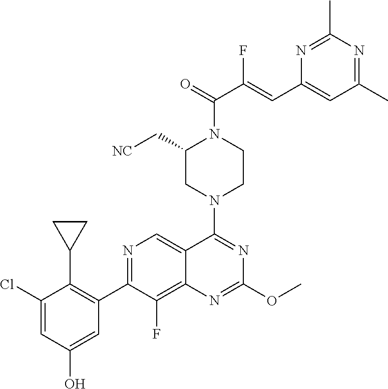 [SMILES: COc1nc(N2CCN(C(=O)/C(F)=C/c3cc(C)nc(C)n3)[C@@H](CC#N)C2)c2cnc(-c3cc(O)cc(Cl)c3C3CC3)c(F)c2n1]

### Example 5: Synthesis of Compound 55—(S,Z)-2-(4-(7-(3-chloro-2-cyclopropyl-5-hydroxyphenyl)-8-fluoro-2-methoxypyrido[4,3-d]pyrimidin-4-yl)-1-(3-(2,6-dimethylpyrimidin-4-yl)-2-fluoroacryloyl)piperazin-2-yl)acetonitrile (Method 1)

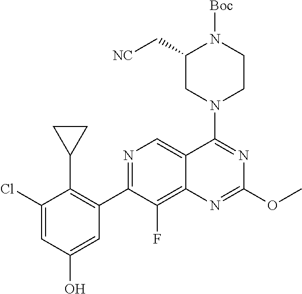 [SMILES: COc1nc(N2CCN(C(=O)OC(C)(C)C)[C@@H](CC#N)C2)c2cnc(-c3cc(O)cc(Cl)c3C3CC3)c(F)c2n1]

### Step 1: tert-butyl (2S)-4-[7-(3-chloro-2-cyclopropyl-5-hydroxy-phenyl)-8-fluoro-2-methoxy-pyrido[4,3-d]pyrimidin-4-yl]-2-(cyanomethyl)piperazine-1-carboxylate

The Suzuki reaction was prepared in a similar fashion to Method #1, Step 2. The residue was purified by prep-HPLC (column: Waters Xbridge Prep OBD C18 150*40 mm*10 um; mobile phase: [H₂O(10 mM NH₄HCO₃)-ACN]; gradient: 35%-70% B over 8.0 min) affording tert-butyl (2S)-4-[7-(3-chloro-2-cyclopropyl-5-hydroxy-phenyl)-8-fluoro-2-methoxy-pyrido[4,3-d]pyrimidin-4-yl]-2-(cyanomethyl)piperazine-1-carboxylate (65 mg, 19.96%) as a white solid. LCMS Rt=0.547 min, m/z=568.2 [M+H]⁺.

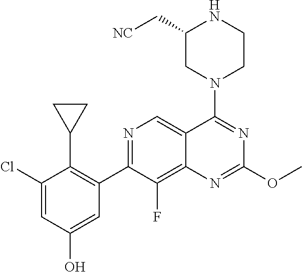 [SMILES: COc1nc(N2CCN[C@@H](CC#N)C2)c2cnc(-c3cc(O)cc(Cl)c3C3CC3)c(F)c2n1]

### Step 2: 2—[(2S)-4-[7-(3-chloro-2-cyclopropyl-5-hydroxy-phenyl)-8-fluoro-2-methoxy-pyrido[4,3-d]pyrimidin-4-yl]piperazin-2-yl]acetonitrile

The deprotection of Boc group was prepared in a similar fashion to Method #1, Step 4. The reaction mixture was concentrated to dryness in vacuo affording 2-[(2S)-4-[7-(3-chloro-2-cyclopropyl-5-hydroxy-phenyl)-8-fluoro-2-methoxy-pyrido[4,3-d]pyrimidin-4-yl]piperazin-2-yl]acetonitrile (51 mg, crude, trifluoroacetic acid salt) as a yellow oil used into the next step without further purification. LCMS Rt=0.438 min, m/z=468.2 [M+H]⁺.

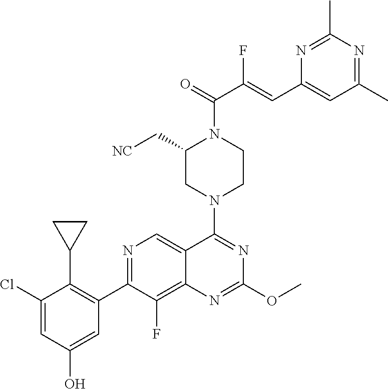 [SMILES: COc1nc(N2CCN(C(=O)/C(F)=C/c3cc(C)nc(C)n3)[C@@H](CC#N)C2)c2cnc(-c3cc(O)cc(Cl)c3C3CC3)c(F)c2n1]

### Step 3: (S,Z)-2-(4-(7-(3-chloro-2-cyclopropyl-5-hydroxyphenyl)-8-fluoro-2-methoxypyrido[4,3-d]pyrimidin-4-yl)-1-(3-(2,6-dimethylpyrimidin-4-yl)-2-fluoroacryloyl)piperazin-2-yl)acetonitrile

The amide coupling reaction was prepared in a similar fashion to Method #1, Step 5. The residue was purified by prep-HPLC (column: Waters Xbridge C18 150*50 mm*10 um; mobile phase: [H₂O(10 mM NH₄HCO₃)-ACN]; gradient: 30%-60% B over 8.0 min) affording (S,Z)-2-(4-(7-(3-chloro-2-cyclopropyl-5-hydroxyphenyl)-8-fluoro-2-methoxypyrido[4,3-d]pyrimidin-4-yl)-1-(3-(2,6-dimethylpyrimidin-4-yl)-2-fluoroacryloyl)piperazin-2-yl)acetonitrile (11.97 mg, 20.73%) as a white solid: ¹H NMR (400 MHz, Acetonitrile-d3) δ 9.10 (s, 1H), 7.44 (s, 1H), 6.99 (d, J=2.6 Hz, 1H), 6.84 (d, J=2.5 Hz, 1H), 6.52-6.39 (m, 1H), 4.91 (br s, 1H), 4.55-4.39 (m, 2H), 4.26-4.08 (m, 1H), 4.03 (s, 3H), 4.02-3.86 (m, 1H), 3.85-3.67 (m, 2H), 3.10-3.01 (m, 1H), 2.97-2.90 (m, 1H), 2.59 (s, 3H), 2.47 (s, 3H), 1.88-1.79 (m, 1H), 0.60 (br d, J=6.6 Hz, 2H), 0.02 (br s, 2H). LCMS Rt=2.864 min, m/z=646.2 [M+H]⁺. LCMS (5 to 95% acetonitrile in water+0.03% ammonium bicarbonate over 6 min) retention time 2.864 min, ESI+ found [M+H]=646.2.

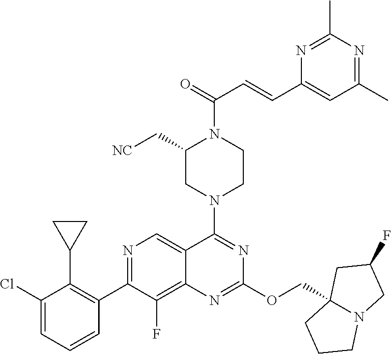 [SMILES: Cc1cc(/C=C/C(=O)N2CCN(c3nc(OC[C@@]45CCCN4C[C@H](F)C5)nc4c(F)c(-c5cccc(Cl)c5C5CC5)ncc34)C[C@@H]2CC#N)nc(C)n1]

### Example 6: Synthesis of Compound 32—2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-3-(2,6-dimethylpyrimidin-4-yl)-2-fluoroacryloyl)piperazin-2-yl)acetonitrile (Method 1)

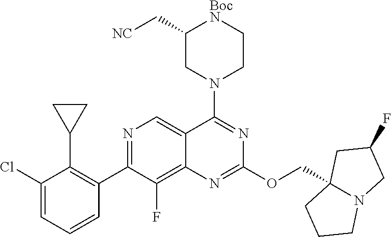 [SMILES: CC(C)(C)OC(=O)N1CCN(c2nc(OC[C@@]34CCCN3C[C@H](F)C4)nc3c(F)c(-c4cccc(Cl)c4C4CC4)ncc23)C[C@@H]1CC#N]

### Step 1: tert-butyl (S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate

The Suzuki reaction was prepared in a similar fashion to Method #1, Step 2. The resulting residue was purified by column chromatography (silica gel, 100-200 mesh, 0-40% ethyl acetate in petroleum ether) affording tert-butyl (S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (500 mg, 62.19%) as a yellow solid. LCMS Rt=0.446 min, m/z=679.3 [M+H]⁺.

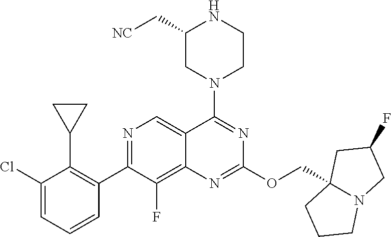 [SMILES: N#CC[C@H]1CN(c2nc(OC[C@@]34CCCN3C[C@H](F)C4)nc3c(F)c(-c4cccc(Cl)c4C4CC4)ncc23)CCN1]

### Step 2: 2—((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The deprotection of Boc group was prepared in a similar fashion to Method #1, Step 4. The reaction mixture was concentrated in vacuo affording 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (407 mg, crude, hydrochloride salt) as a yellow oil used in next step without further purification. LCMS Rt=0.353 min, m/z=579.2 [M+H]⁺.

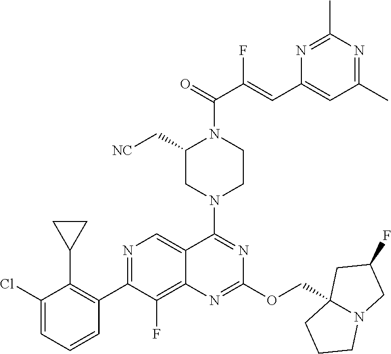 [SMILES: Cc1cc(/C=C(\F)C(=O)N2CCN(c3nc(OC[C@@]45CCCN4C[C@H](F)C5)nc4c(F)c(-c5cccc(Cl)c5C5CC5)ncc34)C[C@@H]2CC#N)nc(C)n1]

### Step 3: 2—((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-3-(2,6-dimethylpyrimidin-4-yl)-2-fluoroacryloyl)piperazin-2-yl)acetonitrile

The amide coupling reaction was prepared in a similar fashion to Method #1, Step 5. The reaction mixture was concentrated in vacuo and purified by reverse phase HPLC (column: Waters Xbridge Prep OBD C18 150*40 mm*Oum; mobile phase: [H₂O (10 mM NH₄HCO₃)-ACN]; gradient: 40%-70% B over 8.0 min) affording 2-((S)-4-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-3-(2,6-dimethylpyrimidin-4-yl)-2-fluoroacryloyl)piperazin-2-yl)acetonitrile (118.11 mg, 23.60%) as a yellow solid: ¹H NMR (400 MHz, Chloroform-d) δ 9.14-9.04 (m, 1H), 7.50 (d, J=7.8 Hz, 1H), 7.41-7.35 (m, 2H), 7.34-7.29 (m, 1H), 6.82-6.63 (m, 1H), 5.43-5.18 (m, 1H), 5.03-4.81 (m, 1H), 4.61-4.44 (m, 2H), 4.41-4.13 (m, 3H), 3.93-3.68 (m, 2H), 3.40-3.16 (m, 3H), 3.12-2.98 (m, 2H), 2.89 (br dd, J=4.9, 16.6 Hz, 1H), 2.72 (s, 3H), 2.56 (s, 3H), 2.36-2.15 (m, 3H), 2.14-1.87 (m, 5H), 0.72 (br d, J=7.1 Hz, 2H), 0.23-0.07 (m, 2H). LCMS Rt=3.175 min, m/z=757.3 [M+H]⁺. LCMS (5 to 95% acetonitrile in water+0.03% ammonium bicarbonate over 6 mins) retention time 3.175 min, ESI+ found [M+H]=757.3.

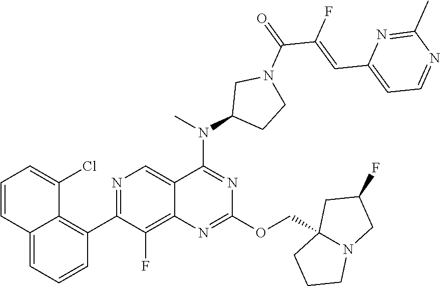 [SMILES: Cc1nccc(/C=C(\F)C(=O)N2CC[C@@H](N(C)c3nc(OC[C@@]45CCCN4C[C@H](F)C5)nc4c(F)c(-c5cccc6cccc(Cl)c56)ncc34)C2)n1]

### Example 7: Synthesis of Compound 37—(Z)-1-((R)-3-((7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(2-methylpyrimidin-4-yl)prop-2-en-1-one (Method 1)

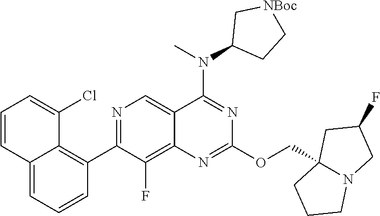 [SMILES: CN(c1nc(OC[C@@]23CCCN2C[C@H](F)C3)nc2c(F)c(-c3cccc4cccc(Cl)c34)ncc12)[C@@H]1CCN(C(=O)OC(C)(C)C)C1]

### Step 1: tert-butyl (R)-3-((7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Suzuki reaction was prepared in a similar fashion to Method #1, Step 2. The resulting residue was purified by column chromatography (silica gel, 100-200 mesh, 0-40% ethyl acetate in petroleum ether) affording tert-butyl (R)-3-((7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (1.96 g, 79.41%) as a yellow solid. LCMS Rt=0.749 min, m/z=664.3 [M+H]⁺.

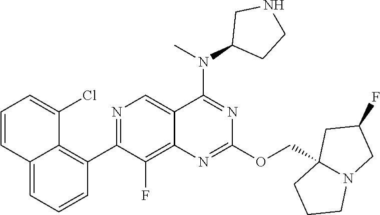 [SMILES: CN(c1nc(OC[C@@]23CCCN2C[C@H](F)C3)nc2c(F)c(-c3cccc4cccc(Cl)c34)ncc12)[C@@H]1CCNC1]

### Step 2: 7-(8-chloronaphthalen-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc group was prepared in a similar fashion to Method #1, Step 4. The reaction mixture was concentrated in vacuo affording 7-(8-chloronaphthalen-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (40 mg, crude, trifluoroacetate salt) as a yellow oil used in next step without further purification. LCMS Rt=0.483 min, m/z=564.2 [M+H]⁺.

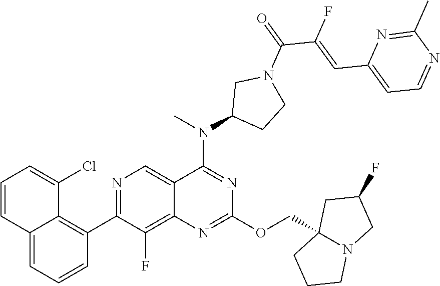 [SMILES: Cc1nccc(/C=C(\F)C(=O)N2CC[C@@H](N(C)c3nc(OC[C@@]45CCCN4C[C@H](F)C5)nc4c(F)c(-c5cccc6cccc(Cl)c56)ncc34)C2)n1]

### Step 3: (Z)-1-((R)-3-((7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(2-methylpyrimidin-4-yl)prop-2-en-1-one

The amide coupling reaction was prepared in a similar fashion to Method #1, Step 5. The reaction mixture was concentrated in vacuo and purified by reverse phase HPLC (column: Waters Xbridge BEH C18 100*30 mm*10 um; mobile phase: [water(NH₄HCO₃)-ACN]; B %: 40%-70%, 8 min) affording (Z)-1-((R)-3-((7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(2-methylpyrimidin-4-yl)prop-2-en-1-one (14.11 mg, 31.36%) as a yellow solid: ¹H NMR (400 MHz, Acetonitrile-d3) δ 9.34-9.16 (m, 1H), 8.76-8.66 (m, 1H), 8.21-8.13 (m, 1H), 8.10-8.01 (m, 1H), 7.74-7.68 (m, 1H), 7.68-7.61 (m, 2H), 7.60-7.51 (m, 2H), 6.79-6.61 (m, 1H), 5.45-5.16 (m, 2H), 4.35-4.18 (m, 2H), 4.16 (br d, J=3.8 Hz, 2H), 3.90-3.80 (m, 1H), 3.73-3.59 (m, 1H), 3.52-3.44 (m, 3H), 3.20-3.14 (m, 1H), 3.13-3.02 (m, 2H), 2.96-2.84 (m, 1H), 2.67 (d, J=3.9 Hz, 3H), 2.46-2.31 (m, 2H), 2.11-1.99 (m, 2H), 1.94 (br s, 4H). LCMS Rt=3.022 min, m/z=728.3 [M+H]⁺. LCMS (5 to 95% acetonitrile in water+0.03% ammonium bicarbonate over 6 mins) retention time 3.022 min, ESI+ found [M+H]=728.3.

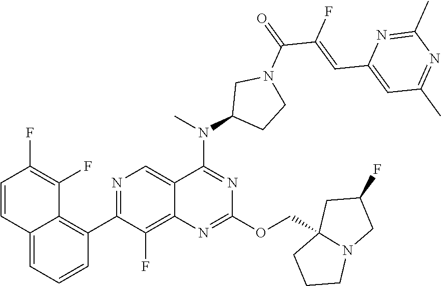 [SMILES: Cc1cc(/C=C(\F)C(=O)N2CC[C@@H](N(C)c3nc(OC[C@@]45CCCN4C[C@H](F)C5)nc4c(F)c(-c5cccc6ccc(F)c(F)c56)ncc34)C2)nc(C)n1]

### Example 8: Synthesis of Compound 41—(Z)-1-((R)-3-((7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(2,6-dimethylpyrimidin-4-yl)-2-fluoroprop-2-en-1-one (Method 1)

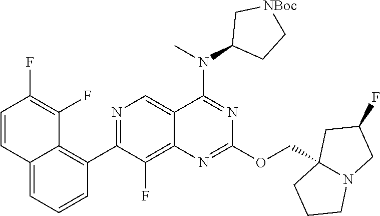 [SMILES: CN(c1nc(OC[C@@]23CCCN2C[C@H](F)C3)nc2c(F)c(-c3cccc4ccc(F)c(F)c34)ncc12)[C@@H]1CCN(C(=O)OC(C)(C)C)C1]

### Step 1: tert-butyl (R)-3-((7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Suzuki reaction was prepared in a similar fashion to Method #1, Step 2. The residue was purified by column chromatography (silica gel, 100-200 mesh, 0-50% ethyl acetate in petroleum ether) affording tert-butyl (R)-3-((7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (642 mg, 86.51%) as a yellow solid. LCMS Rt=0.431 min, m/z=666.3 [M+H]⁺.

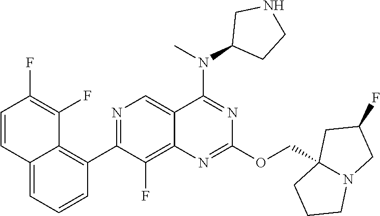 [SMILES: CN(c1nc(OC[C@@]23CCCN2C[C@H](F)C3)nc2c(F)c(-c3cccc4ccc(F)c(F)c34)ncc12)[C@@H]1CCNC1]

### Step 2: 7-(7,8-difluoronaphthalen-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc group was prepared in a similar fashion to Method #1, Step 4. The reaction mixture was concentrated in vacuo affording 7-(7,8-difluoronaphthalen-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (150 mg, crude, trifluoroacetate salt) as a yellow oil used in next step without further purification. LCMS Rt=0.474 min, m/z=566.2 [M+H]⁺.

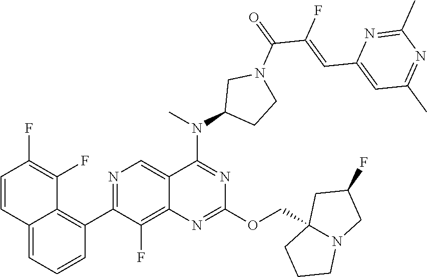 [SMILES: Cc1cc(/C=C(\F)C(=O)N2CC[C@@H](N(C)c3nc(OC[C@@]45CCCN4C[C@H](F)C5)nc4c(F)c(-c5cccc6ccc(F)c(F)c56)ncc34)C2)nc(C)n1]

### Step 3: (Z)-1-((R)-3-((7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(2,6-dimethylpyrimidin-4-yl)-2-fluoroprop-2-en-1-one

The amide coupling reaction was prepared in a similar fashion to Method #1, Step 5. The reaction mixture was concentrated in vacuo and purified by reverse phase HPLC (column: Waters Xbridge Prep OBD C18 150*40 mm*Oum; mobile phase: [water(NH₄HCO₃)-ACN]; B %: 30%-60%, 8 min) affording (Z)-1-((R)-3-((7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(2,6-dimethylpyrimidin-4-yl)-2-fluoroprop-2-en-1-one (8.25 mg, 10.05%) as a yellow solid: ¹H NMR (400 MHz, Acetonitrile-d3) δ 9.25 (s, 1H), 8.16-8.10 (m, 1H), 7.92 (ddd, J=1.6, 5.0, 9.2 Hz, 1H), 7.75-7.68 (m, 2H), 7.56 (dt, J=7.6, 9.6 Hz, 1H), 7.46 (br d, J=9.6 Hz, 1H), 6.78-6.56 (m, 1H), 5.47-5.14 (m, 2H), 4.26-4.13 (m, 2H), 4.12-3.94 (m, 1H), 3.93-3.72 (m, 2H), 3.71-3.56 (m, 1H), 3.47 (s, 3H), 3.13-3.04 (m, 2H), 3.02-2.80 (m, 2H), 2.61 (br s, 3H), 2.49 (br d, J=6.9 Hz, 3H), 2.44-2.32 (m, 2H), 2.14-2.01 (m, 3H), 1.92-1.76 (m, 3H). LCMS Rt=2.806 min, m/z=744.3 [M+H]⁺. LCMS (5 to 95% acetonitrile in water+0.03% ammonium bicarbonate over 6 mins) retention time 2.806 min, ESI+ found [M+H]=744.3.

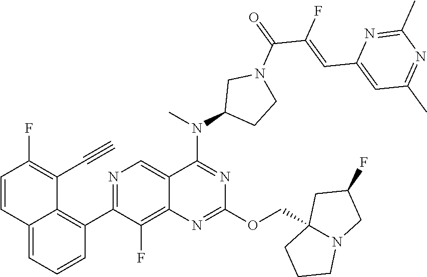 [SMILES: C#Cc1c(F)ccc2cccc(-c3ncc4c(N(C)[C@@H]5CCN(C(=O)/C(F)=C/c6cc(C)nc(C)n6)C5)nc(OC[C@@]56CCCN5C[C@H](F)C6)nc4c3F)c12]

### Example 9: Synthesis of Compound 44—(Z)-3-(2,6-dimethylpyrimidin-4-yl)-1-((R)-3-((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoroprop-2-en-1-one (Method 1)

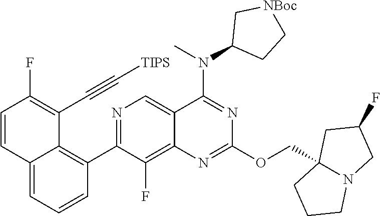

### Step 1: tert-butyl (R)-3-((8-fluoro-7-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Suzuki reaction was prepared in a similar fashion to Method #1, Step 2. The resulting residue was purified by column chromatography (silica gel, 100-200 mesh, 0-70% ethyl acetate in petroleum ether) affording tert-butyl (R)-3-((8-fluoro-7-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (480 mg, 52.01%) as a yellow solid. LCMS Rt=0.584 min, m/z=828.4 [M+H]⁺.

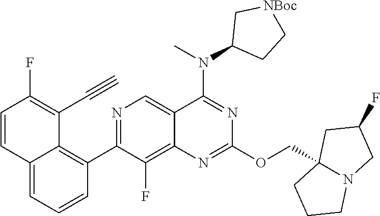 [SMILES: C#Cc1c(F)ccc2cccc(-c3ncc4c(N(C)[C@@H]5CCN(C(=O)OC(C)(C)C)C5)nc(OC[C@@]56CCCN5C[C@H](F)C6)nc4c3F)c12]

### Step 2: tert-butyl (R)-3-((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The deprotection of TIPS group was prepared in a similar fashion to Method #1, Step 3. The reaction mixture was concentrated in vacuo affording tert-butyl (R)-3-((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (600 mg, crude) as a white solid used in next step without further purification. LCMS Rt=0.663 min, m/z=672.3 [M+H]⁺.

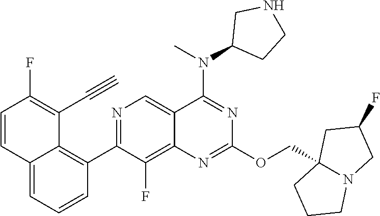 [SMILES: C#Cc1c(F)ccc2cccc(-c3ncc4c(N(C)[C@@H]5CCNC5)nc(OC[C@@]56CCCN5C[C@H](F)C6)nc4c3F)c12]

### Step 3: 7-(8-ethynyl-7-fluoronaphthalen-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc group was prepared in a similar fashion to Method #1, Step 4. The reaction mixture was concentrated in vacuo affording 7-(8-ethynyl-7-fluoronaphthalen-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (153 mg, crude, hydrochloride salt) as a yellow oil used in next step without further purification. LCMS Rt=0.328 min, m/z=572.3 [M+H]⁺.

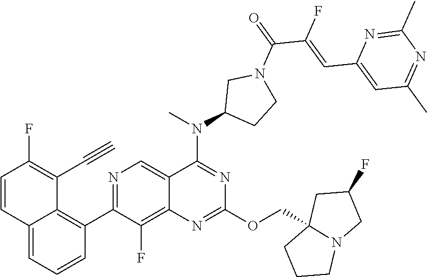 [SMILES: C#Cc1c(F)ccc2cccc(-c3ncc4c(N(C)[C@@H]5CCN(C(=O)/C(F)=C/c6cc(C)nc(C)n6)C5)nc(OC[C@@]56CCCN5C[C@H](F)C6)nc4c3F)c12]

### Step 4: (Z)-3-(2,6-dimethylpyrimidin-4-yl)-1-((R)-3-((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoroprop-2-en-1-one

The amide coupling reaction was prepared in a similar fashion to Method #1, Step 5. The reaction mixture was concentrated in vacuo and purified by reverse phase HPLC (column: Phenomenex Luna C18 200*40 mm*10 um; mobile phase: [water(FA)-ACN]; B %: 25%-60%, 8 min) affording (Z)-3-(2,6-dimethylpyrimidin-4-yl)-1-((R)-3-((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoroprop-2-en-1-one (25.43 mg, 12.18%, formate salt) as a yellow solid: ¹H NMR (400 MHz, Chloroform-d) δ 9.15 (s, 1H), 8.03-7.92 (m, 2H), 7.66-7.57 (m, 2H), 7.41-7.31 (m, 2H), 6.81 (s, 1H), 5.49-5.38 (m, 1H), 4.51-4.27 (m, 2H), 4.19-3.95 (m, 2H), 3.85-3.63 (m, 2H), 3.49 (s, 3H), 2.93 (br s, 2H), 2.72 (s, 3H), 2.56-2.52 (m, 3H), 2.44-2.28 (m, 3H), 2.12-1.89 (m, 3H), 1.73-1.50 (m, 6H). LCMS Rt=2.214 min, m/z=750.3 [M+H]⁺. LCMS (5 to 95% acetonitrile in water+0.03% ammonium bicarbonate over 6 mins) retention time 2.214 min, ESI+ found [M+H]=750.3.

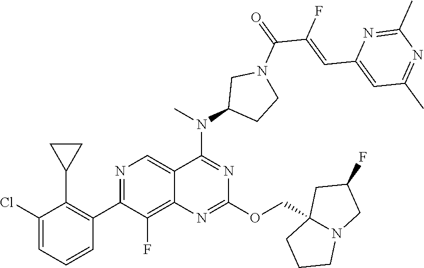 [SMILES: Cc1cc(/C=C(\F)C(=O)N2CC[C@@H](N(C)c3nc(OC[C@@]45CCCN4C[C@H](F)C5)nc4c(F)c(-c5cccc(Cl)c5C5CC5)ncc34)C2)nc(C)n1]

### Example 10: Synthesis of Compound 46—(Z)-1-((R)-3-((7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(2,6-dimethylpyrimidin-4-yl)-2-fluoroprop-2-en-1-one (Method 1)

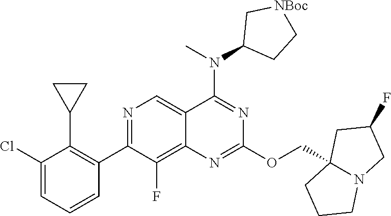 [SMILES: CN(c1nc(OC[C@@]23CCCN2C[C@H](F)C3)nc2c(F)c(-c3cccc(Cl)c3C3CC3)ncc12)[C@@H]1CCN(C(=O)OC(C)(C)C)C1]

### Step 1: tert-butyl (R)-3-((7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Suzuki reaction was prepared in a similar fashion to Method #1, Step 2. The resulting residue was purified by column chromatography (silica gel, 100-200 mesh, 0-50% ethyl acetate in petroleum ether) affording tert-butyl (R)-3-((7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (330 mg, 60.63%) as a yellow solid. LCMS Rt=0.497 min, m/z=654.3 [M+H]⁺.

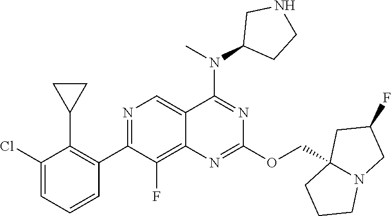 [SMILES: CN(c1nc(OC[C@@]23CCCN2C[C@H](F)C3)nc2c(F)c(-c3cccc(Cl)c3C3CC3)ncc12)[C@@H]1CCNC1]

### Step 2: 7-(3-chloro-2-cyclopropylphenyl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc group was prepared in a similar fashion to Method #1, Step 4. The reaction mixture was concentrated in vacuo affording 7-(3-chloro-2-cyclopropylphenyl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (130 mg, crude, hydrochloride salt) as a yellow oil used in next step without further purification. LCMS Rt=0.377 min, m/z=554.2 [M+H]⁺.

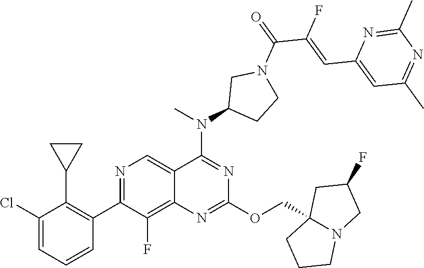 [SMILES: Cc1cc(/C=C(\F)C(=O)N2CC[C@@H](N(C)c3nc(OC[C@@]45CCCN4C[C@H](F)C5)nc4c(F)c(-c5cccc(Cl)c5C5CC5)ncc34)C2)nc(C)n1]

### Step 3: (Z)-1-((R)-3-((7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(2,6-dimethylpyrimidin-4-yl)-2-fluoroprop-2-en-1-one

The amide coupling reaction was prepared in a similar fashion to Method #1, Step 5. The reaction mixture was concentrated in vacuo and purified by reverse phase HPLC (column: Waters Xbridge Prep OBD C18 150*40 mm*Oum; mobile phase: [A: H₂O(10 mM NH₄HCO₃); B: ACN]; B %: 40.00%-70.00%, 8.00 min) affording (Z)-1-((R)-3-((7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(2,6-dimethylpyrimidin-4-yl)-2-fluoroprop-2-en-1-one (39.79 mg, 24.42%) as a yellow solid: ¹H NMR (400 MHz, Chloroform-d) δ 9.17 (s, 1H), 7.49 (dd, J=1.3, 7.9 Hz, 1H), 7.42-7.35 (m, 2H), 7.34-7.28 (m, 1H), 6.98-6.83 (m, 1H), 5.53-5.15 (m, 2H), 4.42-4.21 (m, 2H), 4.18-4.08 (m, 1H), 4.08-3.87 (m, 1H), 3.86-3.61 (m, 2H), 3.53-3.44 (m, 3H), 3.42-3.08 (m, 3H), 3.08-2.91 (m, 1H), 2.72 (s, 3H), 2.60-2.50 (m, 3H), 2.49-2.11 (m, 5H), 2.09-1.84 (m, 4H), 0.83-0.61 (m, 2H), 0.26-0.04 (m, 2H). LCMS Rt=3.195 min, m/z=732.3 [M+H]⁺. LCMS (5 to 95% acetonitrile in water+0.03% ammonium bicarbonate over 6 mins) retention time 3.195 min, ESI+ found [M+H]=732.3.

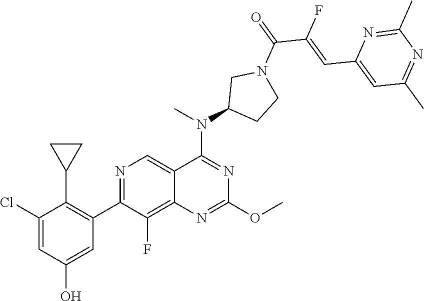 [SMILES: COc1nc(N(C)[C@@H]2CCN(C(=O)/C(F)=C/c3cc(C)nc(C)n3)C2)c2cnc(-c3cc(O)cc(Cl)c3C3CC3)c(F)c2n1]

### Example 11: Synthesis of Compound 56—(R,Z)-1-(3-((7-(3-chloro-2-cyclopropyl-5-hydroxyphenyl)-8-fluoro-2-methoxypyrido[4,3-d]pyrimidin-4-yl)(methyl)amino)pyrrolidin-1-yl)-3-(2,6-dimethylpyrimidin-4-yl)-2-fluoroprop-2-en-1-one (Method 1)

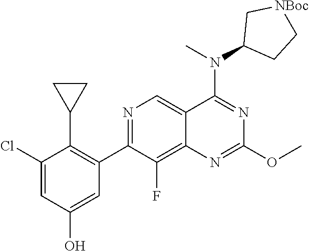 [SMILES: COc1nc(N(C)[C@@H]2CCN(C(=O)OC(C)(C)C)C2)c2cnc(-c3cc(O)cc(Cl)c3C3CC3)c(F)c2n1]

### Step 1: tert-butyl (R)-3-((7-(3-chloro-2-cyclopropyl-5-hydroxyphenyl)-8-fluoro-2-methoxypyrido[4,3-d]pyrimidin-4-yl)(methyl)amino)pyrrolidine-1-carboxylate

The Suzuki reaction was prepared in a similar fashion to Method #1, Step 2. The resulting residue was purified by column chromatography (silica gel, 100-200 mesh, 0-50% ethyl acetate in petroleum ether) affording tert-butyl (R)-3-((7-(3-chloro-2-cyclopropyl-5-hydroxyphenyl)-8-fluoro-2-methoxypyrido[4,3-d]pyrimidin-4-yl)(methyl)amino)pyrrolidine-1-carboxylate (150 mg, 56.78%) as a yellow solid. LCMS Rt=0.574 min, m/z=543.2 [M+H]⁺.

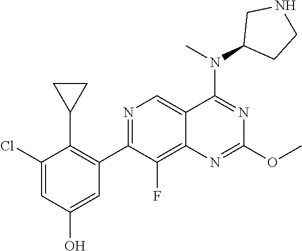 [SMILES: COc1nc(N(C)[C@@H]2CCNC2)c2cnc(-c3cc(O)cc(Cl)c3C3CC3)c(F)c2n1]

### Step 2: (R)-3-chloro-4-cyclopropyl-5-(8-fluoro-2-methoxy-4-(methyl(pyrrolidin-3-yl)amino)pyrido[4,3-d]pyrimidin-7-yl)phenol

The deprotection of Boc group was prepared in a similar fashion to Method #1, Step 4. The reaction mixture was concentrated in vacuo affording (R)-3-chloro-4-cyclopropyl-5-(8-fluoro-2-methoxy-4-(methyl(pyrrolidin-3-yl)amino)pyrido[4,3-d]pyrimidin-7-yl)phenol (130 mg, crude, hydrochloride salt) as a yellow oil used in next step without further purification. LCMS Rt=0.364 min, m/z=443.2 [M+H]⁺.

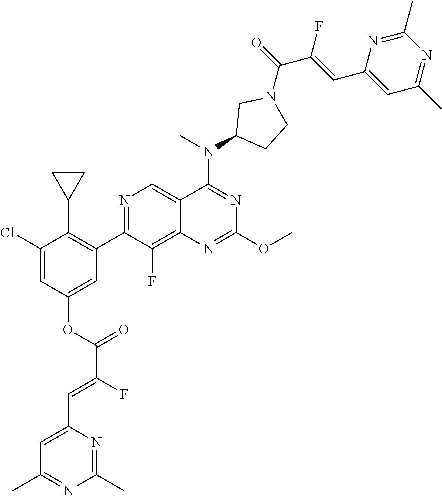

### Step 3: 3-chloro-4-cyclopropyl-5-(4-(((R)-1-((Z)-3-(2,6-dimethylpyrimidin-4-yl)-2-fluoroacryloyl)pyrrolidin-3-yl)(methyl)amino)-8-fluoro-2-methoxypyrido[4,3-d]pyrimidin-7-yl)phenyl (Z)-3-(2,6-dimethylpyrimidin-4-yl)-2-fluoroacrylate

The amide coupling reaction was prepared in a similar fashion to Method #1, Step 5. The reaction mixture was concentrated in vacuo affording 3-chloro-4-cyclopropyl-5-(4-(((R)-1-((Z)-3-(2,6-dimethylpyrimidin-4-yl)-2-fluoroacryloyl)pyrrolidin-3-yl)(methyl)amino)-8-fluoro-2-methoxypyrido[4,3-d]pyrimidin-7-yl)phenyl (Z)-3-(2,6-dimethylpyrimidin-4-yl)-2-fluoroacrylate (150 mg, crude) as a brown oil used in next step without further purification. LCMS Rt=2.017 min, m/z=799.3 [M+H]⁺.

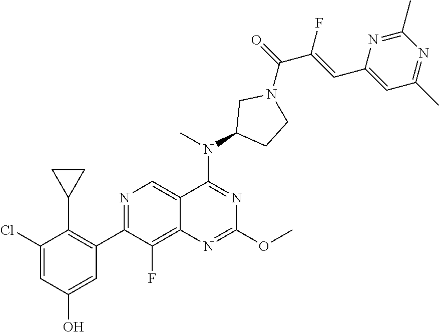 [SMILES: COc1nc(N(C)[C@@H]2CCN(C(=O)/C(F)=C/c3cc(C)nc(C)n3)C2)c2cnc(-c3cc(O)cc(Cl)c3C3CC3)c(F)c2n1]

### Step 4: (R,Z)-1-(3-((7-(3-chloro-2-cyclopropyl-5-hydroxyphenyl)-8-fluoro-2-methoxypyrido[4,3-d]pyrimidin-4-yl)(methyl)amino)pyrrolidin-1-yl)-3-(2,6-dimethylpyrimidin-4-yl)-2-fluoroprop-2-en-1-one

To a solution of 3-chloro-4-cyclopropyl-5-(4-(((R)-1-((Z)-3-(2,6-dimethylpyrimidin-4-yl)-2-fluoroacryloyl)pyrrolidin-3-yl)(methyl)amino)-8-fluoro-2-methoxypyrido[4,3-d]pyrimidin-7-yl)phenyl (Z)-3-(2,6-dimethylpyrimidin-4-yl)-2-fluoroacrylate (200 mg, 249.93 umol) in water (2 mL) was added lithium hydrate (2M, 374.89 L), the mixture was stirred at 20° C. for 1 h. The reaction mixture was concentrated in vacuo and purified by reverse phase HPLC (column: Waters Xbridge Prep OBD C18 150*40 mm*Oum; mobile phase: [H₂O(10 mM NH₄HCO₃)-ACN]; gradient:20%-50% B over 8.0 min) affording (R,Z)-1-(3-((7-(3-chloro-2-cyclopropyl-5-hydroxyphenyl)-8-fluoro-2-methoxypyrido[4,3-d]pyrimidin-4-yl)(methyl)amino)pyrrolidin-1-yl)-3-(2,6-dimethylpyrimidin-4-yl)-2-fluoroprop-2-en-1-one (5.72 mg, 3.36%) as a yellow solid: ¹H NMR (400 MHz, Chloroform-d) δ 9.17 (s, 1H), 7.41-7.32 (m, 1H), 6.86 (br d, J=2.5 Hz, 3H), 5.56-5.31 (m, 1H), 4.12 (s, 3H), 4.07-3.92 (m, 1H), 3.89-3.60 (m, 2H), 3.42 (br s, 3H), 2.77-2.62 (m, 3H), 2.54 (br d, J=8.5 Hz, 3H), 2.42-2.13 (m, 2H), 1.97-1.90 (m, 1H), 1.38-1.23 (m, 2H), 0.72-0.57 (m, 2H), 0.17-0.02 (m, 2H). LCMS Rt=2.869 min, m/z=621.2 [M+H]⁺. LCMS (5 to 95% acetonitrile in water+0.03% ammonium bicarbonate over 6 mins) retention time 2.869 min, ESI+ found [M+H]=621.2.

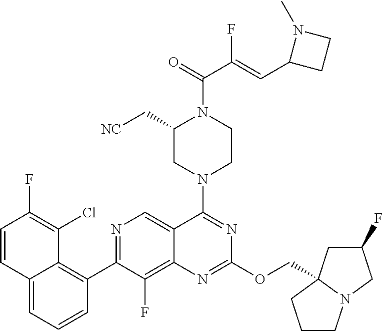 [SMILES: CN1CCC1/C=C(\F)C(=O)N1CCN(c2nc(OC[C@@]34CCCN3C[C@H](F)C4)nc3c(F)c(-c4cccc5ccc(F)c(Cl)c45)ncc23)C[C@@H]1CC#N]

### Example 12: Synthesis of Compound 16—2-((2S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(1-methylazetidin-2-yl)acryloyl)piperazin-2-yl)acetonitrile (Method 2)

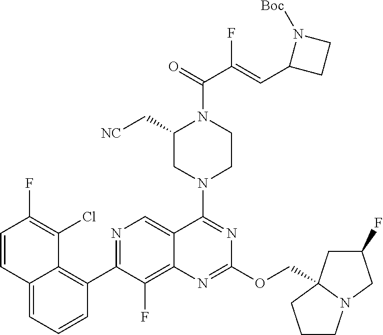 [SMILES: CC(C)(C)OC(=O)N1CCC1/C=C(\F)C(=O)N1CCN(c2nc(OC[C@@]34CCCN3C[C@H](F)C4)nc3c(F)c(-c4cccc5ccc(F)c(Cl)c45)ncc23)C[C@@H]1CC#N]

### Step 1: tert-butyl 2-((Z)-3-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazin-1-yl)-2-fluoro-3-oxoprop-1-en-1-yl)azetidine-1-carboxylate

The amide coupling reaction was prepared in a similar fashion to Method #1, Step 5. The reaction mixture was purified by reverse phase HPLC (column: Phenomenex Luna C18 250*50 mm*10 um; mobile phase: [water(TFA)-ACN]; B %: 25%-55%,10 min) affording tert-butyl 2-((Z)-3-((S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(cyanomethyl)piperazin-1-yl)-2-fluoro-3-oxoprop-1-en-1-yl)azetidine-1-carboxylate (280 mg, 53.43%, trifluoroacetic acid salt) as a white solid. LCMS Rt=0.812 min, m/z=834.3 [M+H]⁺.

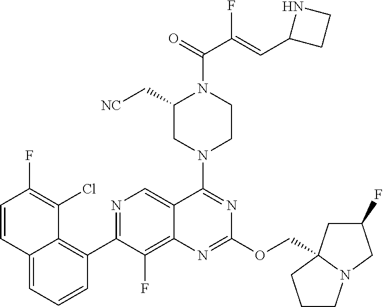 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C(F)=C/C1CCN1]

### Step 2: 2-((2S)-1-((Z)-3-(azetidin-2-yl)-2-fluoroacryloyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The deprotection of Boc group was prepared in a similar fashion to Method #1, Step 4. The mixture was stirred at 25° C. for 0.5 h. The reaction mixture was concentrated in vacuo affording 2-((2S)-1-((Z)-3-(azetidin-2-yl)-2-fluoroacryloyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (70 mg, crude, trifluoroacetic acid salt) as a brown oil, used in the next step without further purification. LCMS Rt=0.639 min, m/z=734.3 [M+H]⁺.

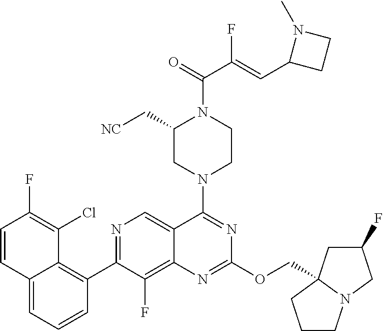 [SMILES: CN1CCC1/C=C(\F)C(=O)N1CCN(c2nc(OC[C@@]34CCCN3C[C@H](F)C4)nc3c(F)c(-c4cccc5ccc(F)c(Cl)c45)ncc23)C[C@@H]1CC#N]

### Step 3: 2-((2S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(1-methylazetidin-2-yl)acryloyl)piperazin-2-yl)acetonitrile

To a solution of 2-((2S)-1-((Z)-3-(azetidin-2-yl)-2-fluoroacryloyl)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (80 mg, 94.21 umol) in methanol (5 mL) was added N,N-Diisopropylethylamine (36.53 mg, 282.62 umol), sodium cyanoborohydride (17.76 mg, 282.62 umol), acetic acid (565.7 ug, 9.42 umol) and formaldehyde (15.29 mg, 188.41 umol) at 0° C. The mixture was stirred at 25° C. for 1 h. The reaction mixture was concentrated in vacuo and the resulting residue was purified by reverse phase HPLC (column: Waters Xbridge Prep OBD C18 150*40 mm*10 um; mobile phase: [water(NH₄HCO₃)-ACN]; B %: 35%-65%, 8 min) affording 2-((2S)-4-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((Z)-2-fluoro-3-(1-methylazetidin-2-yl)acryloyl)piperazin-2-yl)acetonitrile (9.98 mg, 13.98%) as a white solid: ¹H NMR (400 MHz, Acetonitrile-d3) δ 9.13-9.06 (m, 1H), 8.16-8.11 (m, 1H), 8.07 (dd, J=5.7, 9.1 Hz, 1H), 7.70-7.64 (m, 2H), 7.52 (dt, J=1.8, 8.9 Hz, 1H), 5.85-5.63 (m, 1H), 5.39-5.16 (m, 1H), 4.85 (br d, J=0.9 Hz, 1H), 4.61-4.38 (m, 2H), 4.26-4.06 (m, 3H), 3.89 (q, J=8.0 Hz, 1H), 3.83-3.50 (m, 3H), 3.35-3.27 (m, 1H), 3.20-3.11 (m, 2H), 3.07 (s, 1H), 3.03-2.96 (m, 1H), 2.95-2.87 (m, 2H), 2.85-2.69 (m, 1H), 2.24 (d, J=2.8 Hz, 3H), 2.11 (br d, J=2.9 Hz, 3H), 2.08-2.00 (m, 2H), 1.92-1.83 (m, 3H). LCMS Rt=2.021 min, m/z=748.3 [M+H]⁺. LCMS (5 to 95% acetonitrile in water+0.1% trifluoroacetic acid over 6 mins) retention time 2.021 min, ESI+ found [M+H]=748.3.

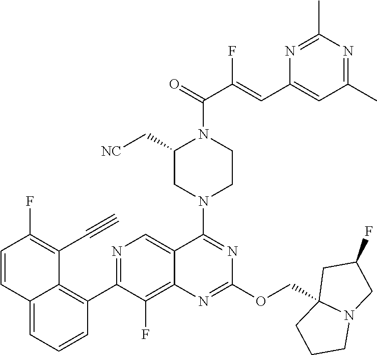 [SMILES: C#Cc1c(F)ccc2cccc(-c3ncc4c(N5CCN(C(=O)/C(F)=C/c6cc(C)nc(C)n6)[C@@H](CC#N)C5)nc(OC[C@@]56CCCN5C[C@H](F)C6)nc4c3F)c12]

### Example 13: Synthesis of Compound 29—2-((S)-1-((Z)-3-(2,6-dimethylpyrimidin-4-yl)-2-fluoroacryloyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (Method 1)

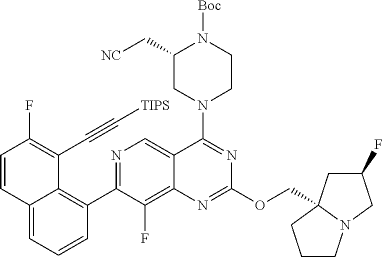

### Step 1: tert-butyl (S)-2-(cyanomethyl)-4-(8-fluoro-7-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperazine-1-carboxylate

The Suzuki reaction was prepared in a similar fashion to Method #1, Step 2. The mixture was purified by column chromatography (silica gel, 100-200 mesh, 0-10% methanol in dichloromethane) affording tert-butyl (S)-2-(cyanomethyl)-4-(8-fluoro-7-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperazine-1-carboxylate (180 mg, 71.52%) as a brown solid. LCMS Rt=0.919 min, m/z=853.4 [M+H]⁺.

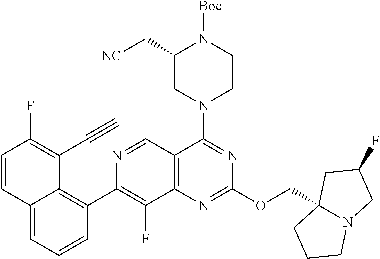 [SMILES: C#Cc1c(F)ccc2cccc(-c3ncc4c(N5CCN(C(=O)OC(C)(C)C)[C@@H](CC#N)C5)nc(OC[C@@]56CCCN5C[C@H](F)C6)nc4c3F)c12]

### Step 2: tert-butyl (S)-2-(cyanomethyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazine-1-carboxylate

The deprotection of TIPS group was prepared in a similar fashion to Method #1, Step 3. The resulting residue was purified by column chromatography (silica gel, 100-200 mesh, 0-10% methanol in dichloromethane) affording tert-butyl (S)-2-(cyanomethyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazine-1-carboxylate (200 mg, 98%) as a brown oil. LCMS Rt=0.727 min, m/z=697.3 [M+H]⁺.

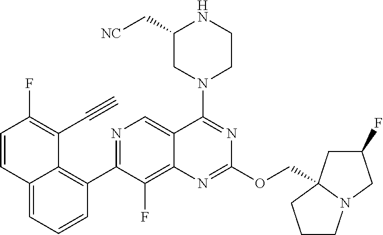 [SMILES: C#Cc1c(F)ccc2cccc(-c3ncc4c(N5CCN[C@@H](CC#N)C5)nc(OC[C@@]56CCCN5C[C@H](F)C6)nc4c3F)c12]

### Step 3: 2—((S)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The deprotection of Boc group was prepared in a similar fashion to Method #1, Step 4. The reaction mixture was concentrated in vacuo affording 2-((S)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (80 mg, crude, hydrochloric acid salt) as a yellow solid, used in the next step without further purification. LCMS Rt=0.712 min, m/z=597.3 [M+H]⁺.

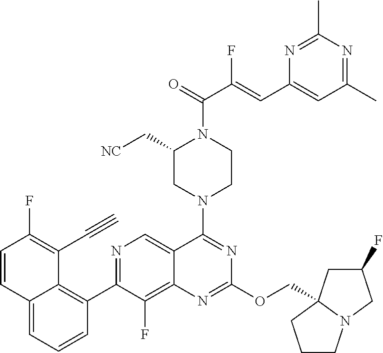 [SMILES: C#Cc1c(F)ccc2cccc(-c3ncc4c(N5CCN(C(=O)/C(F)=C/c6cc(C)nc(C)n6)[C@@H](CC#N)C5)nc(OC[C@@]56CCCN5C[C@H](F)C6)nc4c3F)c12]

### Step 4: 2—((S)-1-((Z)-3-(2,6-dimethylpyrimidin-4-yl)-2-fluoroacryloyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile

The amide coupling reaction was prepared in a similar fashion to Method #1, Step 5. The reaction mixture was purified by reverse phase HPLC (column: Waters Xbridge BEH C18 100*30 mm*10 um; mobile phase: [water(NH₄HCO₃)-ACN]; B %: 35%-65%, 8 min) affording 2-((S)-1-((Z)-3-(2,6-dimethylpyrimidin-4-yl)-2-fluoroacryloyl)-4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-2-yl)acetonitrile (16.83 mg, 16.56%) as a yellow solid: ¹H NMR (400 MHz, Chloroform-d) δ 9.09-9.03 (m, 1H), 8.03-7.93 (m, 2H), 7.68-7.56 (m, 2H), 7.42-7.33 (m, 2H), 6.90-6.64 (m, 1H), 5.46-5.20 (m, 1H), 5.05-4.81 (m, 1H), 4.60-4.44 (m, 2H), 4.42-4.31 (m, 2H), 4.24-4.12 (m, 1H), 4.00-3.77 (m, 2H), 3.46-3.28 (m, 2H), 3.27-3.10 (m, 2H), 3.08-2.99 (m, 2H), 2.94-2.89 (m, 1H), 2.77-2.68 (m, 3H), 2.60-2.53 (m, 3H), 2.32 (br s, 1H), 2.27-2.16 (m, 2H), 2.07-1.88 (m, 4H). LCMS Rt=3.028 min, m/z=775.3 [M+H]⁺. LCMS (5 to 95% acetonitrile in water+0.03% ammonium bicarbonate over 6 mins) retention time 3.028 min, ESI+ found [M+H]=775.3.

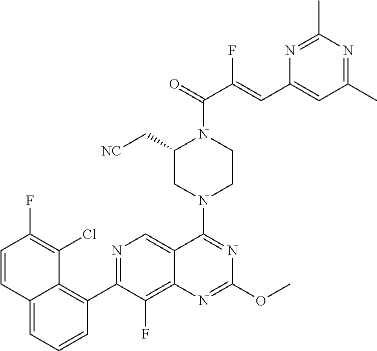 [SMILES: COc1nc(N2CCN(C(=O)/C(F)=C/c3cc(C)nc(C)n3)[C@@H](CC#N)C2)c2cnc(-c3cccc4ccc(F)c(Cl)c34)c(F)c2n1]

### Example 14: Synthesis of Compound 54—(S,Z)-2-(4-(7-(8-chloro-7-fluoronaphthalen-1-yl)-8-fluoro-2-methoxypyrido[4,3-d]pyrimidin-4-yl)-1-(3-(2,6-dimethylpyrimidin-4-yl)-2-fluoroacryloyl)piperazin-2-yl)acetonitrile (Method 1)

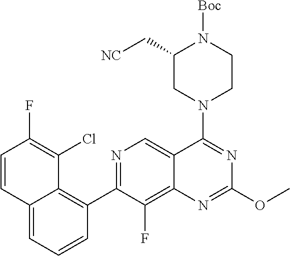 [SMILES: COc1nc(N2CCN(C(=O)OC(C)(C)C)[C@@H](CC#N)C2)c2cnc(-c3cccc4ccc(F)c(Cl)c34)c(F)c2n1]

### Step 1: tert-butyl (S)-4-(7-(8-chloro-7-fluoronaphthalen-1-yl)-8-fluoro-2-methoxypyrido[4,3-d]pyrimidin-4-yl)-2-(cyanomethyl)piperazine-1-carboxylate

The Suzuki reaction was prepared in a similar fashion to Method #1, Step 2. The mixture was purified by reverse phase HPLC (column: Waters Xbridge C18 150*50 mm*10 um; mobile phase: [H₂O(10 mM NH₄HCO₃)-ACN]; gradient:55%-75% B over 8.0 min) affording tert-butyl (S)-4-(7-(8-chloro-7-fluoronaphthalen-1-yl)-8-fluoro-2-methoxypyrido[4,3-d]pyrimidin-4-yl)-2-(cyanomethyl)piperazine-1-carboxylate (75 mg, 28.20%) as a yellow oil. LCMS Rt=0.581 min, m/z=580.2 [M+H]⁺.

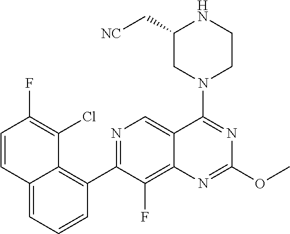 [SMILES: COc1nc(N2CCN[C@@H](CC#N)C2)c2cnc(-c3cccc4ccc(F)c(Cl)c34)c(F)c2n1]

### Step 2: (S)-2-(4-(7-(8-chloro-7-fluoronaphthalen-1-yl)-8-fluoro-2-methoxypyrido[4,3-d]pyrimidin-4-yl)piperazin-2-yl)acetonitrile

The deprotection of Boc group was prepared in a similar fashion to Method #1, Step 4. The reaction mixture was concentrated in vacuo affording (S)-2-(4-(7-(8-chloro-7-fluoronaphthalen-1-yl)-8-fluoro-2-methoxypyrido[4,3-d]pyrimidin-4-yl)piperazin-2-yl)acetonitrile (55 mg, crude, trifluoroacetic acid salt) as a yellow oil used in the next step without further purification. LCMS Rt=0.389 min, m/z=480.1 [M+H]⁺.

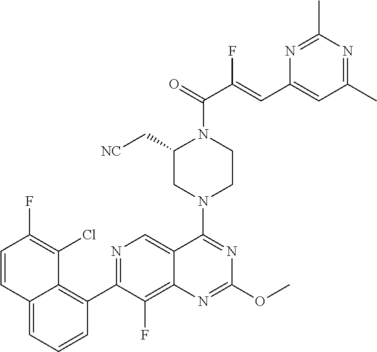 [SMILES: COc1nc(N2CCN(C(=O)/C(F)=C/c3cc(C)nc(C)n3)[C@@H](CC#N)C2)c2cnc(-c3cccc4ccc(F)c(Cl)c34)c(F)c2n1]

### Step 3: (S,Z)-2-(4-(7-(8-chloro-7-fluoronaphthalen-1-yl)-8-fluoro-2-methoxypyrido[4,3-d]pyrimidin-4-yl)-1-(3-(2,6-dimethylpyrimidin-4-yl)-2-fluoroacryloyl)piperazin-2-yl)acetonitrile

The amide coupling reaction was prepared in a similar fashion to Method #1, Step 5. The reaction mixture was purified by reverse phase HPLC (column: Waters Xbridge BEH C18 100*30 mm*10 um; mobile phase: [H₂O(10 mM NH₄HCO₃)-ACN]; gradient: 35%-65% B over 8.0 min) affording (S,Z)-2-(4-(7-(8-chloro-7-fluoronaphthalen-1-yl)-8-fluoro-2-methoxypyrido[4,3-d]pyrimidin-4-yl)-1-(3-(2,6-dimethylpyrimidin-4-yl)-2-fluoroacryloyl)piperazin-2-yl)acetonitrile (25.46 mg, 41.52%) as a white solid: ¹H NMR (400 MHz, Acetonitrile-d3) δ 9.12 (d, J=4.9 Hz, 1H), 8.17-8.11 (m, 1H), 8.10-8.05 (m, 1H), 7.77-7.61 (m, 2H), 7.55-7.48 (m, 1H), 7.45 (s, 1H), 6.54-6.40 (m, 1H), 4.99-4.87 (m, 1H), 4.61-4.41 (m, 2H), 4.27-4.09 (m, 1H), 4.06-4.03 (m, 3H), 3.96-3.66 (m, 3H), 3.12-3.02 (m, 1H), 3.00-2.89 (m, 1H), 2.59 (s, 3H), 2.47 (s, 3H). LCMS Rt=2.994 min, m/z=658.2 [M+H]⁺. LCMS (5 to 95% acetonitrile in water+0.03% ammonium bicarbonate over 6 mins) retention time 2.994 min, ESI+ found [M+H]=658.2.

### Example 15: Other Results

| | Synthetic | | LCMS rt, |
|---|---|---|---|
| Cmpd. | Method | | m/z |
| No. | Used | ¹H NMR Data | [M + H]⁺ |
| 13 | Method | ¹H NMR (400 MHz, CD₃CN) δ 9.21 (s, 1H), 8.15-8.04 (m, | LCMS Rt = |
| | 2 | 1H), 7.89 (ddd, J = 1.4, 5.1, 9.2 Hz, 1H), 7.68 (d, J = 6.6 | 3.059 min, |
| | | Hz, 2H), 7.58-7.49 (m, 1H), 5.95-5.76 (m, 1H), 5.39- | m/z = |
| | | 5.14 (m, 2H), 4.22-4.11 (m, 2H), 3.99-3.74 (m, 3H), 3.69- | 708.4 |
| | | 3.50 (m, 2H), 3.42 (s, 3H), 3.30 (t, J = 6.3 Hz, 1H), 3.17- | [M + H]⁺. |
| | | 3.04 (m, 3H), 2.91-2.77 (m, 2H), 2.23 (s, 4H), 2.12-1.99 | |
| | | (m, 6H), 1.90-1.79 (m, 3H). | |
| 14 | Method | ¹H NMR (400 MHz, CD₃CN) δ 9.21 (d, J = 7.9 Hz, 1H), | LCMS Rt = |
| | 2 | 8.13-8.06 (m, 1H), 7.89 (dd, J = 5.4, 8.4 Hz, 1H), 7.74- | 2.028 min, |
| | | 7.65 (m, 2H), 7.59-7.49 (m, 1H), 5.73-5.61 (m, 1H), 5.42- | m/z = |
| | | 5.15 (m, 2H), 4.26-4.11 (m, 2H), 4.05-3.75 (m, 3H), | 708.3 |
| | | 3.65-3.48 (m, 2H), 3.42 (d, J = 5.3 Hz, 3H), 3.36-3.26 (m, | [M + H]⁺. |
| | | 1H), 3.17-3.05 (m, 3H), 2.93-2.75 (m, 2H), 2.42-2.29 | |
| | | (m, 3H), 2.23 (d, J = 5.8 Hz, 3H), 2.11 (d, J = 2.6 Hz, 2H), | |
| | | 2.09-2.02 (m, 2H), 1.91-1.74 (m, 3H). | |
| 37 | Method | ¹H NMR (400 MHz, CD₃CN) δ 9.34-9.16 (m, 1H), 8.76- | LCMS Rt = |
| | #1 | 8.66 (m, 1H), 8.21-8.13 (m, 1H), 8.10-8.01 (m, 1H), | 3.022 min, |
| | | 7.74-7.68 (m, 1H), 7.68-7.61 (m, 2H), 7.60-7.51 (m, 2H), | m/z = |
| | | 6.79-6.61 (m, 1H), 5.45-5.16 (m, 2H), 4.35-4.18 (m, | 728.3 |
| | | 2H), 4.16 (d, J = 3.8 Hz, 2H), 3.90-3.80 (m, 1H), 3.73- | [M + H]⁺. |
| | | 3.59 (m, 1H), 3.52-3.44 (m, 3H), 3.20-3.14 (m, 1H), 3.13- | |
| | | 3.02 (m, 2H), 2.96-2.84 (m, 1H), 2.67 (d, J = 3.9 Hz, | |
| | | 3H), 2.46-2.31 (m, 2H), 2.11-1.99 (m, 2H), 1.94 (s, 4H). | |
| 38 | Method | ¹H NMR (400 MHz, CD₃CN) δ 9.26 (s, 1H), 8.73 (t, J = 5.6 | LCMS Rt = |
| | 1 | Hz, 1H), 8.18-8.11 (m, 1H), 7.93 (dd, J = 5.2, 7.9 Hz, 1H), | 2.771 min, |
| | | 7.73 (d, J = 5.9 Hz, 2H), 7.62-7.52 (m, 2H), 6.83-6.59 | m/z = |
| | | (m, 1H), 5.47-5.12 (m, 2H), 4.27-4.21 (m, 1H), 4.17 (d, | 731.3 |
| | | J = 4.5 Hz, 1H), 4.08 (s, 1H), 3.97-3.55 (m, 3H), 3.49 (s, | [M + H]⁺. |
| | | 3H), 3.20-3.04 (m, 3H), 2.95-2.86 (m, 1H), 2.68 (d, J = | |
| | | 4.4 Hz, 3H), 2.47-2.32 (m, 2H), 2.12 (d, J = 16.0 Hz, 2H), | |
| | | 2.17-2.02 (m, 1H), 1.95-1.76 (m, 3H). | |
| 39 | Method | ¹H NMR (400 MHz, CD₃CN) δ 9.23 (s, 1H), 8.73 (t, J = 6.2 | LCMS Rt = |
| | 1 | Hz, 1H), 7.61-7.54 (m, 2H), 7.48-7.37 (m, 2H), 6.78- | 3.165 min, |
| | | 6.65 (m, 1H), 5.45-5.15 (m, 2H), 4.35-4.20 (m, 2H), | m/z = |
| | | 4.19-4.11 (m, 2H), 3.95-3.78 (m, 1H), 3.75-3.56 (m, 1H), | 719.3 |
| | | 3.50-3.44 (m, 3H), 3.22-3.03 (m, 3H), 2.99-2.82 (m, | [M + H]⁺. |
| | | 1H), 2.68 (d, J = 5.4 Hz, 3H), 2.10 (s, 2H), 2.07-2.02 (m, | |
| | | 4H), 1.95-1.76 (m, 3H), 0.73 (d, J = 3.3 Hz, 2H), 0.14 | |
| | | (d, J = 1.6 Hz, 2H). | |
| 41 | Method | ¹H NMR (400 MHz, CD₃CN) δ 9.25 (s, 1H), 8.16-8.10 (m, | LCMS Rt = |
| | #1 | 1H), 7.92 (ddd, J = 1.6, 5.0, 9.2 Hz, 1H), 7.75-7.68 (m, | 2.806 min, |
| | | 2H), 7.56 (dt, J = 7.6, 9.6 Hz, 1H), 7.46 (d, J = 9.6 Hz, 1H), | m/z = |
| | | 6.78-6.56 (m, 1H), 5.47-5.14 (m, 2H), 4.26-4.13 (m, | 744.3 |
| | | 2H), 4.12-3.94 (m, 1H), 3.93-3.72 (m, 2H), 3.71-3.56 | [M + H]⁺. |
| | | (m, 1H), 3.47 (s, 3H), 3.13-3.04 (m, 2H), 3.02-2.80 (m, | |
| | | 2H), 2.61 (s, 3H), 2.49 (d, J = 6.9 Hz, 3H), 2.44-2.32 (m, | |
| | | 2H), 2.14-2.01 (m, 3H), 1.92-1.76 (m, 3H). | |
| 42 | Method | ¹H NMR (400 MHz, CDCl₃) δ 9.20-9.09 (m, 1H), 8.69 (s, | LCMS Rt = |
| | 1 | 1H), 8.01-7.94 (m, 2H), 7.64-7.57 (m, 2H), 7.50 (d, J = | 3.425 min, |
| | | 5.8 Hz, 1H), 7.37-7.32 (m, 1H), 7.01-6.86 (m, 1H), 5.53- | m/z = |
| | | 5.19 (m, 2H), 4.34-4.21 (m, 2H), 4.18-3.97 (m, 2H), 3.81- | 737.3 |
| | | 3.64 (m, 2H), 3.48 (s, 3H), 3.27-3.10 (m, 4H), 2.99-2.83 | [M + H]⁺. |
| | | (m, 1H), 2.76 (s, 3H), 2.42-2.11 (m, 4H), 2.00-1.58 (m, | |
| | | 4H). | |
| 43 | Method | ¹H NMR (400 MHz, CD₃CN) δ 9.15 (s, 1H), 8.66 (dd, J = | LCMS Rt = |
| | 1 | 5.4, 8.2 Hz, 1H), 7.78-7.74 (m, 1H), 7.72-7.66 (m, 1H), | 3.111 min, |
| | | 7.51 (dd, J = 5.0, 14.1 Hz, 1H), 7.45 (d, J = 7.4 Hz, 1H), | m/z = |
| | | 6.73-6.60 (m, 1H), 5.38-5.11 (m, 2H), 4.21-4.15 (m, | 747.2 |
| | | 1H), 4.14-4.08 (m, 1H), 4.07-3.94 (m, 1H), 3.88-3.70 | [M + H]⁺. |
| | | (m, 2H), 3.68-3.52 (m, 1H), 3.41 (s, 3H), 3.14-3.08 (m, | |
| | | 1H), 3.06-2.98 (m, 2H), 2.91-2.80 (m, 1H), 2.68-2.58 | |
| | | (m, 3H), 2.40-2.29 (m, 2H), 2.18-1.99 (m, 3H), 1.89- | |
| | | 1.75 (m, 3H). | |
| 44 | Method | ¹H NMR (400 MHz, CDCl₃) δ 9.15 (s, 1H), 8.03-7.92 (m, | LCMS Rt = |
| | #1 | 2H), 7.66-7.57 (m, 2H), 7.41-7.31 (m, 2H), 6.81 (s, 1H), | 2.214 min, |
| | | 5.49-5.38 (m, 1H), 4.51-4.27 (m, 2H), 4.19-3.95 (m, | m/z = |
| | | 2H), 3.85-3.63 (m, 2H), 3.49 (s, 3H), 2.93 (s, 2H), 2.72 (s, | 750.3 |
| | | 3H), 2.56-2.52 (m, 3H), 2.44-2.28 (m, 3H), 2.12-1.89 | [M + H]⁺. |
| | | (m, 3H), 1.73-1.50 (m, 6H). | |
| 45 | Method | ¹H NMR (400 MHz, CD₃CN) δ 9.19 (s, 1H), 8.66 (dd, J = | LCMS Rt = |
| | 1 | 4.7, 10.6 Hz, 1H), 8.03 (d, J = 7.9 Hz, 1H), 7.94-7.88 (m, | 3.148 min, |
| | | 1H), 7.51 (dd, J = 7.1, 15.9 Hz, 3H), 7.36 (t, J = 9.4 Hz, | m/z = |
| | | 1H), 6.73-6.60 (m, 1H), 5.37-5.12 (m, 2H), 4.31-4.15 | 741.2 |
| | | (m, 2H), 4.14-4.08 (m, 1H), 4.06-3.94 (m, 1H), 3.88- | [M + H]⁺. |
| | | 3.72 (m, 2H), 3.69-3.54 (m, 1H), 3.42 (s, 3H), 3.17-2.98 | |
| | | (m, 4H), 2.90-2.79 (m, 1H), 2.63 (d, J = 6.6 Hz, 3H), 2.46 | |
| | | (d, J = 0.6 Hz, 1H), 2.36 (d, J = 3.8 Hz, 2H), 2.07 (d, J = | |
| | | 16.7 Hz, 2H), 1.82 (s, 3H), 0.81-0.76 (m, 3H). | |
| 46 | Method | ¹H NMR (400 MHz, CDC₃) δ 9.17 (s, 1H), 7.49 (dd, J = 1.3, | LCMS Rt = |
| | #1 | 7.9 Hz, 1H), 7.42-7.35 (m, 2H), 7.34-7.28 (m, 1H), 6.98- | 3.195 min, |
| | | 6.83 (m, 1H), 5.53-5.15 (m, 2H), 4.42-4.21 (m, 2H), 4.18- | m/z = |
| | | 4.08 (m, 1H), 4.08-3.87 (m, 1H), 3.86-3.61 (m, 2H), 3.53- | 732.3 |
| | | 3.44 (m, 3H), 3.42-3.08 (m, 3H), 3.08-2.91 (m, 1H), 2.72 | [M + H]⁺. |
| | | (s, 3H), 2.60-2.50 (m, 3H), 2.49-2.11 (m, 5H), 2.09-1.84 | |
| | | (m, 4H), 0.83-0.61 (m, 2H), 0.26-0.04 (m, 2H). | |
| 47 | Method | ¹H NMR (400 MHz, CDCl₃) δ 9.16-9.09 (m, 1H), 7.70- | LCMS Rt = |
| | 1 | 7.64 (m, 1H), 7.61-7.53 (m, 1H), 7.40-7.32 (m, 2H), | 2.314 min, |
| | | 6.98-6.82 (m, 1H), 5.50-5.18 (m, 2H), 4.42-4.22 (m, 2H), | m/z = |
| | | 4.17-3.92 (m, 2H), 3.81-3.62 (m, 2H), 3.51-3.43 (m, 3H), | 761.2 |
| | | 3.36-3.16 (m, 2H), 3.13 (s, 1H), 3.08-2.94 (m, 1H), 2.74- | [M + H]⁺. |
| | | 2.70 (m, 3H), 2.57-2.52 (m, 3H), 2.45-2.08 (m, 5H), | |
| | | 2.07-1.83 (m, 3H). | |
| 48 | Method | ¹H NMR (400 MHz, CDCl₃) δ 9.20 (s, 1H), 7.99 (d, | LCMS Rt = |
| | 1 | J = 8.0 Hz, 1H), 7.77-7.70 (m, 1H), 7.69-7.58 | 2.500 min, |
| | | (m, 2H), 7.46-7.35 (m, 2H), 7.01-6.82 (m, 1H), 5.52- | m/z = |
| | | 5.36 (m, 1H), 4.43-4.16 (m, 1H), 4.15-4.12 (m, 3H), | 618.2 |
| | | 4.11-3.92 (m, 1H), 3.90-3.60 (m, 2H), 3.53-3.44 (m, 3H), | [M + H]⁺. |
| | | 2.77-2.68 (m, 3H), 2.57-2.52 (m, 3H), 2.50-2.20 (m, | |
| | | 2H). | |
| 50 | Method | ¹H NMR (400 MHz, CDCl₃) δ 9.18 (s, 1H), 7.49 (d, J = 7.5 | LCMS Rt = |
| | 1 | Hz, 1H), 7.38 (t, J = 6.5 Hz, 2H), 7.31 | 2.106 min, |
| | | (d, J = 7.7 Hz, 1H), 6.97-6.84 (m, 1H), 5.55-5.36 (m, | m/z = |
| | | 1H), 4.63-4.52 (m, 1H), 4.36 (dd, J = 6.7, 9.8 Hz, 2H), | 689.3 |
| | | 4.18-3.97 (m, 2H), 3.93-3.61 (m, 3H), 3.48 (d, J = 4.5 | [M + H]⁺. |
| | | Hz, 3H), 3.17-3.05 (m, 2H), 2.72 (s, 3H), 2.56-2.47 (m, | |
| | | 6H), 2.31-2.26 (m, 1H), 2.11-2.01 (m, 2H), 1.84-1.74 | |
| | | (m, 3H), 0.73 (d, J = 2.6 Hz, 2H), 0.17 (d, J = 4.5Hz, 2H). | |
| 51 | Method | ¹H NMR (400 MHz, CDCl₃) δ 8.98-8.92 (m, 1H), 7.49- | LCMS Rt = |
| | 1 | 7.42 (m, 1H), 7.41-7.32 (m, 2H), 7.31-7.28 (m, 1H), | 3.062 min, |
| | | 6.99-6.81 (m, 1H), 5.24-5.08 (m, 1H), 4.36-4.22 (m, 2H), | m/z = |
| | | 4.18-4.03 (m, 3H), 4.03-3.95 (m, 1H), 3.91- | 674.3 |
| | | 3.80 (m, 1H), 3.78-3.57 (m, 2H), 3.43-3.30 (m, 3H), | [M + H]⁺. |
| | | 3.26-3.14 (m, 1H), 2.77-2.68 (m, 3H), 2.59-2.50 (m, 3H), | |
| | | 2.43-2.34 (m, 1H), 2.28-2.22 (m, 3H), 2.22-2.15 (m, | |
| | | 3H), 2.05 (s, 1H), 0.81-0.64 (m, 2H), 0.28-0.07 (m, 2H). | |
| 52 | Method | ¹H NMR (400 MHz, CDCl₃) δ 9.18 (s, 1H), 7.98 (d, J = 8.0 | LCMS Rt = |
| | 1 | Hz, 1H), 7.73 (td, J = 3.8, 5.1 Hz, 1H), 7.67-7.58 (m, 2H), | 2.031 min, |
| | | 7.46-7.37 (m, 2H), 7.01-6.85 (m, 1H), 5.54-5.41 (m, | m/z = |
| | | 1H), 4.59 (dd, J = 3.9, 7.1 Hz, 1H), 4.38 (dd, J = 3.6, 5.1 | 701.3 |
| | | Hz, 1H), 4.20-3.96 (m, 2H), 3.91-3.67 (m, 2H), 3.49 (s, | [M + H]⁺. |
| | | 3H), 3.15-3.07 (m, 1H), 2.72 (s, 3H), 2.54 (d, J = 6.9 Hz, | |
| | | 7H), 2.46-2.25 (m, 3H), 2.16-2.00 (m, 1H), 1.88-1.74 | |
| | | (m, 3H). | |
| 53 | Method | ¹H NMR (400 MHz, CD₃CN) δ 9.06-8.96 (m, 1H), 8.10- | LCMS Rt = |
| | 1 | 8.05 (m, 1H), 7.87 (ddd, J = 1.6, 5.1, 9.1 Hz, 1H), 7.66 | 2.911 min, |
| | | (d, J = 4.8 Hz, 2H), 7.52 (dt, J = 7.6, 9.6 Hz, 1H), 7.43 | m/z = |
| | | (s, 1H), 6.70-6.54 (m, 1H), 5.26-5.10 (m, 1H), 4.41-4.11 (m, | 686.3 |
| | | 3H), 4.05-3.89 (m, 3H), 3.84-3.77 (m, 1H), 3.68-3.53 | [M + H]⁺. |
| | | (m, 1H), 3.37 (s, 3H), 3.19-3.06 (m, 1H), 2.59 (s, 3H), 2.46 | |
| | | (s, 3H), 2.39-2.30 (m, 2H), 2.16 (s, 3H), 2.10 (s, 3H). | |
| 56 | Method | ¹H NMR (400 MHz, CDCl₃) δ 9.17 (s, 1H), 7.41-7.32 (m, | LCMS Rt = |
| | 1 | 1H), 6.86 (d, J = 2.5 Hz, 3H), 5.56-5.31 (m, 1H), 4.12 (s, | 2.869 min, |
| | | 3H), 4.07-3.92 (m, 1H), 3.89-3.60 (m, 2H), 3.42 (s, 3H), | m/z = |
| | | 2.77-2.62 (m, 3H), 2.54 (d, J = 8.5 Hz, 3H), 2.42-2.13 | 621.2 |
| | | (m, 2H), 1.97-1.90 (m, 1H), 1.38-1.23 (m, 2H), 0.72- | [M + H]⁺. |
| | | 0.57 (m, 2H), 0.17-0.02 (m, 2H). | |
| 2 | Method | 1H NMR (400 MHz, CD3CN) δ 9.19-9.08 (m, 1H), 8.78- | LCMS Rt = |
| | 1 | 8.64 (m, 1H), 8.22-8.01 (m, 2H), 7.78-7.50 (m, 5H), 6.66- | 2.725 min, |
| | | 6.44 (m, 1H), 5.10-4.84 (m, 1H), 4.65-4.33 (m, 3H), | m/z = |
| | | 4.28-4.12 (m, 3H), 3.98-3.68 (m, 3H), 3.13-2.97 (m, 4H), | 736.3 |
| | | 2.69-2.62 (m, 4H), 2.17-2.12 (m, 2H), 1.92-1.80 (m, | [M + H]+. |
| | | 4H), 1.73-1.62 (m, 2H). | |
| 4 | Method | 1H NMR (400 MHz, DMSO-d6) δ 9.24-9.10 (m, 1H), 8.27- | LCMS Rt = |
| | 1 | 8.18 (m, 1H), 8.12 (d, J = 8.3 Hz, 1H), 7.74 (dt, J = 3.3, | 2.424 min, |
| | | 7.7 Hz, 1H), 7.69-7.54 (m, 3H), 7.09-6.85 (m, 1H), 4.90 | m/z = |
| | | (d, J = 14.0 Hz, 1H), 4.59-4.40 (m, 2H), 4.27-4.06 (m, | 754.3 |
| | | 3H), 4.05-3.68 (m, 3H), 3.19-3.03 (m, 3H), 3.03-2.95 | [M + H]+. |
| | | (m, 2H), 2.63-2.57 (m, 2H), 1.91 (dd, J = 6.2, 11.4 Hz, | |
| | | 2H), 1.85-1.74 (m, 4H), 1.65-1.55 (m, 2H), 1.38-1.23 | |
| | | (m, 6H). | |
| 5 | Method | 1H NMR (400 MHz, CD3CN) δ 9.26-8.99 (m, 1H), 8.70 | LCMS Rt = |
| | 1 | (d, J = 5.3 Hz, 1H), 8.18-8.09 (m, 1H), 8.06 (dd, J = 5.9, | 2.236 min, |
| | | 9.1 Hz, 1H), 7.79-7.61 (m, 2H), 7.59-7.35 (m, 2H), 6.64- | m/z = |
| | | 6.34 (m, 1H), 5.37-5.16 (m, 1H), 5.04-4.82 (m, 1H), | 771.3 |
| | | 4.69-4.39 (m, 2H), 4.33-4.09 (m, 3H), 3.96-3.61 (m, 3H), | [M + H]+. |
| | | 3.24-3.12 (m, 2H), 3.08 (s, 1H), 3.02 (d, J = 7.8 Hz, 1H), | |
| | | 2.99-2.86 (m, 2H), 2.66-2.52 (m, 3H), 2.20 (s, 1H), 2.12 | |
| | | (s, 1H), 2.09-2.04 (m, 1H), 1.92-1.81 (m, 3H). | |
| 6 | Method | 1H NMR (400 MHz, CDCl3) δ 9.11-9.02 (m, 1H), 8.69- | LCMS Rt = |
| | 1 | 8.56 (m, 1H), 8.02 (d, J = 7.6 Hz, 1H), 7.90 (d, J = 8.3 Hz, | 3.239 min, |
| | | 1H), 7.68-7.50 (m, 3H), 7.48-7.38 (m, 2H), 6.82-6.62 | m/z = |
| | | (m, 1H), 5.39-5.18 (m, 1H), 5.06-4.82 (m, 1H), 4.64- | 780.3 |
| | | 4.44 (m, 2H), 4.42-4.13 (m, 3H), 4.11-3.62 (m, 3H), | [M + H]+. |
| | | 3.36-3.15 (m, 3H), 3.10-2.83 (m, 3H), 2.33-2.07 (m, 4H), | |
| | | 2.02-1.84 (m, 3H), 1.20-1.04 (m, 4H). | |
| 7 | Method | 1H NMR (400 MHz, CDCl3) δ 9.14-9.03 (m, 1H), 8.63- | LCMS Rt = |
| | 1 | 8.51 (m, 1H), 8.06-7.99 (m, 1H), 7.95-7.88(m, 1H), 7.68- | 3.201 min, |
| | | 7.57 (m, 2H), 7.47-7.38 (m, 1H), 7.03-6.91 (m, 1H), 6.54- | m/z = |
| | | 6.38 (m, 1H), 5.45-5.22 (m, 1H), 5.11-5.02 (m, 1H), | 798.3 |
| | | 4.79-4.76 (m, 1H), 4.79-4.55 (m, 1H), 4.53-4.29 (m, 3H), | [M + H]+. |
| | | 4.22-4.12 (m, 1H), 3.98 (d, J = 2.5Hz, 1H), 3.87 (d, J = 8.1 | |
| | | Hz, 1H), 3.43-3.27 (m, 2H), 3.25-3.09 (m, 2H), 3.07- | |
| | | 2.98 (m, 1H), 2.95-2.84 (m, 1H), 2.38-2.25 (m, 2H), | |
| | | 2.22-2.12 (m, 2H), 2.02 (td, J = 4.2, 14.8 Hz, 3H), | |
| | | 1.11-0.99 (m, 4H). | |
| 8 | Method | 1H NMR (400 MHz, CDCl3) δ 9.13-9.05 (m, 1H), 8.60- | LCMS Rt = |
| | 1 | 8.51 (m, 1H), 8.02-7.95 (m, 1H), 7.78-7.69 (m, 1H), 7.68- | 3.186 min, |
| | | 7.56 (m, 2H), 7.46-7.34 (m, 1H), 7.00-6.88 (m, 1H), | m/z = |
| | | 6.54-6.37 (m, 1H), 5.42-5.19 (m, 1H), 5.12-4.97 (m, | 782.3 |
| | | 1H), 4.90-4.55 (m, 2H), 4.52-4.40 (m, 1H), 4.38-4.31 | [M + H]+. |
| | | (m, 1H), 4.30-4.22 (m, 1H), 4.21-4.07 (m, 1H), 4.01- | |
| | | 3.93 (m, 1H), 3.89-3.81 (m, 1H), 3.79-3.61 (m, 1H), 3.32- | |
| | | 3.25 (m, 1H), 3.21-3.16 (m, 1H), 3.15-3.05 (m, 1H), | |
| | | 3.04-2.95 (m, 1H), 2.93-2.82 (m, 1H), 2.33-2.25 (m, | |
| | | 1H), 2.24-2.03 (m, 3H), 2.00-1.84 (m, 3H), 1.11-0.96 | |
| | | (m, 4H). | |
| 9 | Method | 1H NMR (400 MHz, DMSO-d6) δ 9.24-9.17 (m, 1H), 8.83- | LCMS Rt = |
| | 1 | 8.75 (m, 1H), 8.13 (d, J = 8.0 Hz, 1H), 8.03 (dd, J = 6.1, | 3.216 min, |
| | | 9.0 Hz, 1H), 7.63-7.57 (m, 2H), 7.52-7.46 (m, 2H), 6.65- | m/z = |
| | | 6.47 (m, 1H), 5.38-5.18 (m, 1H), 4.99-4.86 (m, 1H), | 765.3 |
| | | 4.61-4.39 (m, 2H), 4.22-4.06 (m, 3H), 4.00-3.65 (m, 3H), | [M + H]+. |
| | | 3.17-3.00 (m, 5H), 2.89-2.78 (m, 1H), 2.64 (s, 3H), 2.46- | |
| | | 2.37 (m, 1H), 2.25-2.12 (m, 2H), 2.08-2.00 (m, 2H), | |
| | | 1.88-1.76 (m, 3H), 0.77 (q, J = 7.4 Hz, 3H). | |
| 10 | Method | 1H NMR (400 MHz, CD3CN) δ 9.17-9.07 (m, 1H), 8.60- | LCMS Rt = |
| | 1 | 8.50 (m, 1H), 8.07 (d, J = 8.0 Hz, 1H), 7.95 (dd, J = 5.9, | 3.338 min, |
| | | 8.8 Hz, 1H), 7.61-7.54 (m, 1H), 7.52-7.46 (m, 1H), 7.40 | m/z = |
| | | (t, J = 8.9 Hz, 1H), 7.12-7.05 (m, 1H), 6.66-6.53 (m, 1H), | 792.4 |
| | | 5.40-5.18 (m, 1H), 5.02 (d, J = 3.8 Hz, 1H), 4.65-4.45 | [M + H]+. |
| | | (m, 2H), 4.41-4.23 (m, 2H), 4.22-4.03 (m, 2H), 3.96-3.61 (m, | |
| | | 3H), 3.59-3.34 (m, 1H), 3.24-3.13 (m, 2H), 3.12-2.97 | |
| | | (m, 3H), 2.94 (dd, J = 6.0, 9.1 Hz, 1H), 2.53-2.42 (m, 1H), | |
| | | 2.15 (d, J = 2.5 Hz, 1H), 2.11-2.02 (m, 2H), 1.93-1.90 (m, | |
| | | 1H), 1.89-1.83 (m, 1H), 1.10-1.03 (m, 1H), 1.03-0.90 | |
| | | (m, 4H), 0.87-0.75 (m, 3H). | |
| 11 | Method | 1H NMR (400 MHz, CDCl3) δ 9.08 (s, 1H), 8.72 (d, J = 5.3 | LCMS Rt = |
| | 1 | Hz, 1H), 7.99 (d, J = 7.8 Hz, 1H), 7.76-7.69 (m, 1H), 7.67- | 3.013 min, |
| | | 7.58 (m, 2H), 7.51 (d, J = 5.1 Hz, 1H), 7.45-7.37 (m, 1H), | m/z = |
| | | 6.88-6.63 (d, J = 20 Hz, 1H), 5.40-5.22 (m, 1H), 5.08- | 756.3 |
| | | 4.92 (m, 1H), 4.85 (s, 1H), 4.80-4.72 (m, 1H), 4.61-4.46 | [M + H]+. |
| | | (m, 2H), 4.39-4.30 (m, 2H), 4.13-3.96 (m, 1H), 3.96- | |
| | | 3.76 (m, 2H), 3.37-3.32 (m, 1H), 3.27-3.21 (m, 1H), 3.14- | |
| | | 2.97 (m, 2H), 2.95-2.87 (m, 1H), 2.76 (s, 3H), 2.33-2.16 | |
| | | (m, 3H), 2.03-1.93 (m, 3H) | |
| 12 | Method | 1H NMR (400 MHz, CD3CN) δ 9.19-9.07 (m, 1H), 8.14- | LCMS Rt = |
| | 2 | 8.06 (m, 1H), 7.94-7.86 (m, 1H), 7.68 (dd, J = 3.1, 5.9 Hz, | 2.872 min, |
| | | 2H), 7.59-7.47 (m, 1H), 5.75-5.61 (m, 1H), 5.37-5.16 | m/z = |
| | | (m, 1H), 5.01 (dd, J = 1.6, 5.4 Hz, 1H), 4.60-4.36 (m, 2H), | 733.3 |
| | | 4.29-4.08 (m, 2H), 3.96-3.48 (m, 5H), 3.36-3.21 (m, | [M + H]+. |
| | | 1H), 3.18-3.04 (m, 3H), 3.02-2.85 (m, 3H), 2.82-2.70 | |
| | | (m, 1H), 2.38-2.19 (m, 4H), 2.13-2.03 (m, 4H), 1.89- | |
| | | 1.78 (m, 3H). | |
| 15 | Method | 1H NMR (400 MHz, CD3CN) δ 9.12 (d, J = 6.3 Hz, 1H), | LCMS Rt = |
| | 1 | 8.06 (dd, J = 1.1, 8.1 Hz, 1H), 7.93 (dd, J =6.1, 8.9 Hz, 1H), | 3.212 min, |
| | | 7.59-7.52 (m, 1H), 7.51-7.43 (m, 2H), 7.42-7.35 (m, | m/z = |
| | | 1H), 6.54-6.39 (m, 1H), 5.42-5.16 (m, 1H), 5.04-4.78 | 780.4 |
| | | (m, 1H), 4.66-4.38 (m, 2H), 4.28-4.22 (m, 1H), 4.18- | [M + H]+. |
| | | 4.12 (m, 1H), 3.87-3.69 (m, 2H), 3.22-3.12 (m, 2H), 3.11- | |
| | | 3.02 (m, 2H), 2.98 (dd, J = 7.0, 16.9 Hz, 1H), 2.94-2.85 | |
| | | (m, 1H), 2.59 (s, 3H), 2.47 (s, 3H), 2.36-2.19 (m, 4H), | |
| | | 2.14-2.01 (m, 3H), 1.92-1.75 (m, 3H), 0.86-0.73 (m, 3H). | |
| 16 | Method | 1H NMR (400 MHz, CD3CN) δ 9.13-9.06 (m, 1H), 8.16- | LCMS Rt = |
| | 2 | 8.11 (m, 1H), 8.07 (dd, J = 5.7, 9.1 Hz, 1H), 7.70-7.64 (m, | 2.021 min, |
| | | 2H), 7.52 (dt, J = 1.8, 8.9 Hz, 1H), 5.85-5.63 (m, 1H), 5.39- | m/z = |
| | | 5.16 (m, 1H), 4.85 (d, J = 0.9 Hz, 1H), 4.61-4.38 (m, 2H), | 749.2 |
| | | 4.26-4.06 (m, 3H), 3.89 (q, J = 8.0 Hz, 1H), 3.83-3.50 (m, | [M + H]+. |
| | | 3H), 3.35-3.27 (m, 1H), 3.20-3.11 (m, 2H), 3.07 (s, 1H), | |
| | | 3.03-2.96 (m, 1H), 2.95-2.87 (m, 2H), 2.85-2.69 (m, | |
| | | 1H), 2.24 (d, J = 2.8 Hz, 3H), 2.11 (d, J = 2.9 Hz, 3H), | |
| | | 2.08-2.00 (m, 2H), 1.92-1.83 (m, 3H). | |
| 17 | Method | 1H NMR (400 MHz, CD3CN) δ 9.15-9.03 (m, 1H), 8.17- | LCMS Rt = |
| | 2 | 8.01 (m, 2H), 7.71-7.60 (m, 2H), 7.52 (dt, J = 1.3, 8.9 Hz, | 2.029 min, |
| | | 1H), 5.93-5.65 (m, 1H), 5.35-5.16 (m, 1H), 5.00 (dd, J = | m/z = |
| | | 3.3, 6.8 Hz, 1H), 4.61-4.40 (m, 2H), 4.26-4.12 (m, 2H), | 749.3 |
| | | 3.90 (s, 1H), 3.86-3.50 (m, 4H), 3.31 (d, J = 6.6 Hz, 1H), | [M + H]+. |
| | | 3.23-3.06 (m, 3H), 3.04-2.86 (m, 3H), 2.84-2.69 (m, | |
| | | 1H), 2.36 (s, 3H), 2.20-2.09 (m, 3H), 2.09-1.97 (m, 2H), | |
| | | 1.91-1.80 (m, 3H). | |
| 18 | Method | 1H NMR (400 MHz, CDCl3) δ 9.06 (s, 1H), 8.72 (d, J = 5.1 | LCMS Rt = |
| | 1 | Hz, 1H), 8.02 (dd, J = 1.3, 7.9 Hz, 1H), 7.90 (d, J = 8.3 Hz, | 3.007 min, |
| | | 1H), 7.65-7.53 (m, 3H), 7.50 (d, J = 5.3 Hz, 1H), 7.44 (dt, | m/z = |
| | | J = 2.1, 7.8 Hz, 1H), 6.82-6.69 (m, 1H), 5.39-5.18 (m, | 754.0 |
| | | 1H), 5.04-4.84 (m, 1H), 4.63-4.44 (m, 2H), 4.39-4.21 | [M + H]+. |
| | | (m, 3H), 4.08-3.67 (m, 3H), 3.26 (d, J = 11.4 Hz, 2H), 3.18 | |
| | | (s, 1H), 3.10-2.96 (m, 2H), 2.94-2.85 (m, 1H), 2.76 (s, | |
| | | 3H), 2.32-2.25 (m, 1H), 2.23-2.10 (m, 2H), 2.02-1.86 | |
| | | (m, 3H). | |
| 19 | Method | 1H NMR (400 MHz, CD3CN) δ 9.14 (d, J = 3.4 Hz, 1H), | LCMS Rt = |
| | 1 | 8.12 (d, J = 8.3 Hz, 1H), 7.87 (d, J = 8.1Hz, 1H), 7.74-7.68 | 2.284 min, |
| | | (m, 1H), 7.66-7.60 (m, 1H), 7.55 (dt, J = 5.1, 7.9 Hz, 1H), | m/z = |
| | | 7.45 (s, 1H), 7.22 (dd, J = 7.7, 13.2 Hz, 1H), 6.57-6.31 (m, | 752.2 |
| | | 1H), 5.39-5.14 (m, 1H), 5.01-4.84 (m, 1H), 4.65-4.43 | [M + H]+. |
| | | (m, 2H), 4.27-4.08 (m, 3H), 3.92-3.66 (m, 3H), 3.22-3.13 | |
| | | (m, 2H), 3.11-3.04 (m, 2H), 3.01-2.96 (m, 1H), 2.94- | |
| | | 2.87 (m, 1H), 2.59 (s, 3H), 2.47 (s, 3H), 2.10-2.02 (m, 3H), | |
| | | 1.92-1.82 (m, 3H). | |
| 20 | Method | 1H NMR (400 MHz, CDCl3) δ 9.08 (s, 1H), 8.73 (d, J = 5.3 | LCMS Rt = |
| | 1 | Hz, 1H), 7.50 (d, J = 6.3 Hz, 2H), 7.40-7.36 (m, 1H), | 3.151 min, |
| | | 7.34-7.28 (m, 1H), 6.83-6.69 (m, 1H), 5.41-5.18 (m, 1H), | m/z = |
| | | 5.02-4.83 (m, 1H), 4.62-4.43 (m, 2H), 4.41-4.17 (m, | 744.0 |
| | | 3H), 4.15-3.75 (m, 3H), 3.36-3.12 (m, 3H), 3.10-2.96 | [M + H]+. |
| | | (m, 2H), 2.93-2.85 (m, 1H), 2.77 (s, 3H), 2.33-2.11 (m, | |
| | | 3H), 2.09-1.87 (m, 4H), 0.77-0.67 (m, 2H), 0.19-0.10 | |
| | | (m, 2H). | |
| 21 | Method | 1H NMR (400 MHz, CDCl3) δ 9.09-9.00 (m, 1H), 8.06- | LCMS Rt = |
| | 1 | 7.98 (m, 1H), 7.93-7.86 (m, 1H), 7.67-7.51 (m, 3H), | 3.042 min, |
| | | 7.48-7.39 (m, 1H), 6.17-5.69 (m, 1H), 5.40-5.20 (m, 1H), | m/z = |
| | | 5.19-4.88 (m, 1H), 4.88-4.57 (m, 1H), 4.56-4.39 (m, | 787.0 |
| | | 2H), 4.37-4.19 (m, 3H), 4.08-3.92 (m, 1H), 3.91-3.52 | [M + H]+. |
| | | (m, 3H), 3.63-3.47 (m, 2H), 3.25 (d, J = 9.9 Hz, 2H), 3.21- | |
| | | 3.03 (m, 4H), 3.03-2.93 (m, 2H), 2.92-2.75 (m, 1H), | |
| | | 2.32-2.24 (m, 1H), 2.23-2.09 (m, 2H), 2.01 (s, 6H), | |
| | | 1.56-1.19 (m, 2H). | |
| 22 | Method | 1H NMR (400 MHz, CD3CN) δ 9.13 (d, J = 4.6 Hz, 1H), | LCMS Rt = |
| | 1 | 8.70 (d, J = 5.3 Hz, 1H), 8.05 (dd, J = 2.1, 7.4 Hz, 1H), 7.93 | 2.247 min, |
| | | (dd, J = 6.0, 8.9 Hz, 1H), 7.63-7.50 (m, 3H), 7.38 (t, J = | m/z = |
| | | 9.2 Hz, 1H), 6.60-6.39 (m, 1H), 5.39-5.14 (m, 1H), 5.02- | 752.2 |
| | | 4.87 (m, 1H), 4.61-4.43 (m, 2H), 4.29-4.10 (m, 3H), 3.97- | [M + H]+. |
| | | 3.64 (m, 3H), 3.20-3.13 (m, 2H), 3.10-3.02 (m, 2H), | |
| | | 3.00-2.88 (m, 2H), 2.65 (s, 3H), 2.20-2.00 (m, 4H), 1.91- | |
| | | 1.82 (m, 5H). | |
| 23 | Method | 1H NMR (400 MHz, CD3CN) δ 9.19 (s, 1H), 8.78-8.61 | LCMS Rt = |
| | 1 | (m, 1H), 8.21 (d, J = 8.5 Hz, 1H), 7.78-7.69 (m, 1H), 7.68- | 2.304 min, |
| | | 7.59 (m, 2H), 7.56 (d, J = 5.0 Hz, 1H), 7.28 (t, J = 9.4 Hz, | m/z = |
| | | 1H), 6.61-6.39 (m, 1H), 5.45-5.16 (m, 1H), 5.02-4.82 (m, | 752.3 |
| | | 1H), 4.56 (d, J = 14.0 Hz, 2H), 4.32-4.11 (m, 3H), 3.96- | [M + H]+. |
| | | 3.62 (m, 3H), 3.24-3.13 (m, 2H), 3.13-3.03 (m, 2H), 3.00- | |
| | | 2.86 (m, 2H), 2.70-2.57 (m, 6H), 2.21 (s, 1H), 2.15-2.04 | |
| | | (m, 2H), 1.92-1.83 (m, 3H). | |
| 24 | Method | 1H NMR (400 MHz, CDCl3) δ 9.10-9.03 (m, 1H), 8.02- | LCMS Rt = |
| | 1 | 7.94 (m, 1H), 7.73 (dd, J = 4.6, 8.6 Hz, 1H), 7.68-7.56 (m, | 2.977 min, |
| | | 2H), 7.47-7.34 (m, 1H), 6.13-5.75 (m, 1H), 5.39-5.21 | m/z = |
| | | (m, 1H), 5.11-4.70 (m, 1H), 4.59-4.43 (m, 2H), 4.38- | 763.4 |
| | | 4.24 (m, 2H), 4.01 (s, 1H), 3.87-3.77 (m, 1H), 3.76-3.63 | [M + H]+. |
| | | (m, 5H), 3.41-3.12 (m, 6H), 3.09-2.95 (m, 2H), 2.91- | |
| | | 2.78 (m, 1H), 2.51 (d, J = 2.8 Hz, 4H), 2.31-2.12 (m, 3H), | |
| | | 2.01-1.88 (m, 3H). | |
| 25 | Method | 1H NMR (400 MHz, DMSO-d6) δ 9.24-9.17 (m, 1H), 8.84- | LCMS Rt = |
| | 1 | 8.66 (m, 1H), 8.14 (d, J = 8.0 Hz, 1H), 8.04 (dd, J = 6.3, | 3.177 min, |
| | | 8.8 Hz, 1H), 7.65-7.55 (m, 2H), 7.54-7.43 (m, 2H), 6.97- | m/z = |
| | | 6.40 (m, 1H), 5.39-5.19 (m, 1H), 4.99-4.83 (m, 1H), | 766.1 |
| | | 4.63-4.36 (m, 2H), 4.23-4.06 (m, 3H), 4.00-3.66 (m, 3H), | [M + H]+. |
| | | 3.16-3.06 (m, 3H), 3.02 (s, 1H), 2.88-2.79 (m, 1H), 2.64 | |
| | | (s, 2H), 2.60-2.52 (m, 1H), 2.47-2.38 (m, 1H), 2.29-2.12 | |
| | | (m, 2H), 2.09-1.98 (m, 2H), 1.90-1.74 (m, 3H), 1.33- | |
| | | 1.18 (m, 1H), 0.81-0.72 (m, 3H). | |
| 26 | Method | 1H NMR (400 MHz, CDCl3) δ 9.10-9.04 (m, 1H), 8.07- | LCMS Rt = |
| | 1 | 7.97 (m, 1H), 7.90 (d, J = 8.1 Hz, 1H), 7.67-7.52 (m, 3H), | 3.058 min, |
| | | 7.47-7.35 (m, 2H), 6.83-6.61 (m, 1H), 5.45-5.27 (m, | m/z = |
| | | 1H), 5.09-4.80 (m, 1H), 4.77-4.61 (m, 1H), 4.57-4.33 | 768.3 |
| | | (m, 3H), 4.21-4.01 (m, 1H), 4.00-3.61 (m, 3H), 3.57- | [M + H]+. |
| | | 3.23 (m, 3H), 3.20-3.01 (m, 2H), 3.00-2.85 (m, 1H), 2.72 | |
| | | (s, 3H), 2.59-2.53 (m, 3H), 2.46-2.20 (m, 3H), 2.12-1.94 | |
| | | (m, 3H). | |
| 27 | Method | 1H NMR (400 MHz, CDCl3) δ 9.08 (s, 1H), 7.99 (d, J = 7.9 | LCMS Rt = |
| | 1 | Hz, 1H), 7.77-7.69 (m, 1H), 7.68-7.57 (m, 2H), 7.45- | 3.050 min, |
| | | 7.35 (m, 2H), 6.82-6.63 (m, 1H), 5.42-5.20 (m, 1H), | m/z = |
| | | 5.06-4.78 (m, 1H), 4.66-4.45 (m, 2H), 4.41-4.16 (m, 3H), | 770.3 |
| | | 4.13-3.70 (m, 3H), 3.46-3.19 (m, 3H), 3.15-2.97 (m, | [M + H]+. |
| | | 2H), 2.90 (dd, J = 4.4, 16.5 Hz, 1H), 2.72 (s, 3H), | |
| | | 2.56 (s, 3H), 2.34-2.14 (m, 3H), 2.02-1.90 (m, 3H). | |
| 28 | Method | 1H NMR (400 MHz, CDCl3) δ 9.09-9.03 (m, 1H), 8.02 (d, | LCMS Rt = |
| | 1 | J = 8.1 Hz, 1H), 7.76 (d, J = 8.1 Hz, 1H), 7.68-7.56 (m, | 3.005 min, |
| | | 2H), 7.46 (dt, J = 5.0, 7.8 Hz, 1H), 7.13 (dd, J = 7.6, 12.7 | m/z = |
| | | Hz, 1H), 6.15-5.73 (m, 1H), 5.40-5.20 (m, 1H), 5.06- | 771.3 |
| | | 4.78 (m, 1H), 4.73 (s, 3H), 4.62-4.44 (m, 2H), 4.38-4.25 | [M + H]+. |
| | | (m, 2H), 4.00 (d, J = 3.1 Hz, 1H), 3.74-3.66 (m, 2H), 3.54 | |
| | | (d, J = 10.5 Hz, 2H), 3.38-3.18 (m, 3H), 3.12-3.06 (m, | |
| | | 3H), 2.97 (s, 2H), 2.87 (d, J = 5.5 Hz, 1H), 2.29 (s, 1H), | |
| | | 2.25-2.11 (m, 2H), 2.02 (d, J = 3.9 Hz, 2H), 1.99-1.89 (m, | |
| | | 6H). | |
| 29 | Method | 1H NMR (400 MHz, CDCl3) δ 9.09-9.03 (m, 1H), 8.03- | LCMS Rt = |
| | #1 | 7.93 (m, 2H), 7.68-7.56 (m, 2H), 7.42-7.33 (m, 2H), | 3.028 min, |
| | | 6.90-6.64 (m, 1H), 5.46-5.20 (m, 1H), 5.05-4.81 (m, 1H), | m/z = |
| | | 4.60-4.44 (m, 2H), 4.42-4.31 (m, 2H), 4.24-4.12 (m, | 775.3 |
| | | 1H), 4.00-3.77 (m, 2H), 3.46-3.28 (m, 2H), 3.27-3.10 | [M + H]+. |
| | | (m, 2H), 3.08-2.99 (m, 2H), 2.94-2.89 (m, 1H), 2.77- | |
| | | 2.68 (m, 3H), 2.60-2.53 (m, 3H), 2.32 (s, 1H), 2.27-2.16 | |
| | | (m, 2H), 2.07-1.88 (m, 4H). | |
| 30 | Method | 1H NMR (400 MHz, CDCl3) δ 9.06 (d, J = 6.5 Hz, 1H), | LCMS Rt = |
| | 1 | 8.73 (d, J = 5.0 Hz, 1H), 7.99 (dt, J = 2.3, 7.9 Hz, 2H), | 2.988 min, |
| | | 7.64-7.58 (m, 2H), 7.50 (d, J = 5.0 Hz, 1H), 7.38-7.33 (m, 1H), | m/z = |
| | | 6.84-6.69 (m, 1H), 5.44-5.20 (m, 1H), 5.02-4.84 (m, 1H), | 762.3 |
| | | 4.58-4.43 (m, 2H), 4.41-4.21 (m, 3H), 4.02-3.79 (m, | [M + H]+. |
| | | 2H), 3.44-3.11 (m, 4H), 3.07-2.86 (m, 4H), 2.77 (s, 3H), | |
| | | 2.32-2.26 (m, 1H), 2.23-2.12 (m, 2H), 2.01-1.92 (m, | |
| | | 3H). | |
| 31 | Method | 1H NMR (400 MHz, CDCl3) δ 9.12-8.98 (m, 1H), 8.01 (d, | LCMS Rt = |
| | 1 | J = 9.5 Hz, 1H), 7.94-7.86 (m, 1H), 7.65-7.58 (m, 2H), | 3.058 min, |
| | | 7.41 (d, J = 2.0 Hz, 1H), 6.07-5.75 (m, 1H), 5.40-5.21 (m, | m/z = |
| | | 1H), 5.06-4.78 (m, 1H), 4.57-4.44 (m, 2H), 4.39-4.26 | 805.3 |
| | | (m, 2H), 4.04-3.98 (m, 1H), 3.81-3.76 (m, 1H), 3.72- | [M + H]+. |
| | | 3.67 (m, 2H), 3.57-3.51 (m, 2H), 3.37-3.25 (m, 2H), 3.21- | |
| | | 3.17 (m, 1H), 3.13-3.06 (m, 4H), 3.03-2.94 (m, 2H), | |
| | | 2.91-2.76 (m, 1H), 2.40-2.08 (m, 4H), 2.02-1.87 (m, | |
| | | 8H). | |
| 32 | Method | 1H NMR (400 MHz, CDCl3) δ 9.14-9.04 (m, 1H), 7.50 (d, | LCMS Rt = |
| | 1 | J = 7.8 Hz, 1H), 7.41-7.35 (m, 2H), 7.34-7.29 (m, 1H), | 3.175 min, |
| | | 6.82-6.63 (m, 1H), 5.43-5.18 (m, 1H), 5.03-4.81 (m, | m/z = |
| | | 1H), 4.61-4.44 (m, 2H), 4.41-4.13 (m, 3H), 3.93-3.68 | 757.3 |
| | | (m, 2H), 3.40-3.16 (m, 3H), 3.12-2.98 (m, 2H), 2.89 (dd, | [M + H]+. |
| | | J = 4.9, 16.6 Hz, 1H), 2.72 (s, 3H), 2.56 (s, 3H), 2.36-2.15 | |
| | | (m, 3H), 2.14-1.87 (m, 5H), 0.72 (d, J = 7.1 Hz, 2H), 0.23- | |
| | | 0.07 (m, 2H). | |
| 33 | Method | 1H NMR (400 MHz, CD3CN) δ 9.13 (d, J = 4.3 Hz, 1H), | LCMS Rt = |
| | 1 | 8.70 (d, J = 5.3 Hz, 1H), 8.11 (d, J = 8.1 Hz, 1H), 7.86 (d, | 2.924 min, |
| | | J = 8.1 Hz, 1H), 7.73-7.67 (m, 1H), 7.65-7.59 (m, 1H), 7.57- | m/z = |
| | | 7.50 (m, 2H), 7.21 (dd, J = 7.7, 13.2 Hz, 1H), 6.63-6.36 | 738.3 |
| | | (m, 1H), 5.38-5.14 (m, 1H), 5.03-4.81 (m, 1H), 4.63- | [M + H]+. |
| | | 4.39 (m, 2H), 4.28-4.03 (m, 3H), 4.00-3.53 (m, 3H), 3.13 | |
| | | (d, J = 7.3 Hz, 2H), 3.09-3.02 (m, 2H), 3.00-2.84 (m, 2H), | |
| | | 2.64 (s, 3H), 2.10 (d, J = 2.7 Hz, 1H), 2.08-2.01 (m, 1H), | |
| | | 1.97-1.95 (m, 1H), 1.92-1.79 (m, 3H). | |
| 34 | Method | 1H NMR (400 MHz, CD3CN) δ 9.16 (d, J = 4.0 Hz, 1H), | LCMS Rt = |
| | 1 | 8.08 (dd, J = 1.9, 7.5 Hz, 1H), 7.96 (dd, J = 6.2, 9.1 Hz, 1H), | 3.143 min, |
| | | 7.59 (d, J = 5.3 Hz, 2H), 7.48 (s, 1H), 7.41 (t, J = 9.1 Hz, | m/z = |
| | | 1H), 6.60-6.40 (m, 1H), 5.39-5.20 (m, 1H), 4.66-4.46 | 766.4 |
| | | (m, 2H), 4.32-4.15 (m, 3H), 3.96-3.72 (m, 3H), 3.23- | [M + H]+. |
| | | 3.10 (m, 4H), 3.04-2.91 (m, 3H), 2.62 (s, 3H), 2.50 (s, 3H), | |
| | | 2.15 (s, 4H), 1.90 (d, J = 6.5 Hz, 5H). | |
| 35 | Method | 1H NMR (400 MHz, CD3CN) δ 9.14 (d, J = 6.0 Hz, 1H), | LCMS Rt = |
| | 1 | 8.19-8.13 (m, 1H), 8.09 (dd, J = 5.9, 9.0 Hz, 1H), 7.79- | 2.237 min, |
| | | 7.38 (m, 4H), 6.62-6.39 (m, 1H), 5.42-5.17 (m, 1H), | m/z = |
| | | 4.69-4.43 (m, 2H), 4.33-4.06 (m, 3H), 3.98-3.68 (m, 3H), | 786.2 |
| | | 3.16 (s, 2H), 3.12-2.86 (m, 4H), 2.62 (s, 3H), 2.54-2.38 | [M + H]+. |
| | | (m, 4H), 2.17-2.02 (m, 3H), 1.95-1.83 (m, 3H). | |
| 49 | Method | 1H NMR (400 MHz, CD3CN) δ 9.10 (d, J = 8.0 Hz, 1H), | LCMS Rt = |
| | #1 | 8.12 (dd, J = 5.8, 8.9 Hz, 2H), 7.71-7.64 (m, 2H), 7.50- | 2.937 min, |
| | | 7.41 (m, 2H), 6.54-6.38 (m, 1H), 4.92 (dt, J = 1.5, 6.3 Hz, | m/z = |
| | | 1H), 4.62-4.39 (m, 2H), 4.26-4.10 (m, 1H), 4.04 (d, J = | 648.2 |
| | | 2.5 Hz, 3H), 3.98-3.86 (m, 1H), 3.85-3.67 (m, 2H), 3.30- | [M + H]+. |
| | | 3.22 (m, 1H), 3.15-3.05 (m, 1H), 3.04-2.90 (m, 1H), 2.59 | |
| | | (s, 3H), 2.47 (s, 3H). | |
| 54 | Method | 1H NMR (400 MHz, CD3CN) δ 9.12 (d, J = 4.9 Hz, 1H), | LCMS Rt = |
| | 1 | 8.17-8.11 (m, 1H), 8.10-8.05 (m, 1H), 7.77-7.61 (m, | 2.994 min, |
| | | 2H), 7.55-7.48 (m, 1H), 7.45 (s, 1H), 6.54-6.40 (m, 1H), | m/z = |
| | | 4.99-4.87 (m, 1H), 4.61-4.41 (m, 2H), 4.27-4.09 (m, | 658.2 |
| | | 1H), 4.06-4.03 (m, 3H), 3.96-3.66 (m, 3H), 3.12-3.02 | [M + H]+. |
| | | (m, 1H), 3.00-2.89 (m, 1H), 2.59 (s, 3H), 2.47 (s, 3H). | |
| 55 | Method | 1H NMR (400 MHz, CD3CN) δ 9.10 (s, 1H), 7.44 (s, 1H), | LCMS Rt = |
| | #1 | 6.99 (d, J = 2.6 Hz, 1H), 6.84 (d, J = 2.5 Hz, 1H), | 2.864 min, |
| | | 6.52-6.39 (m, 1H), 4.91 (s, 1H), 4.55-4.39 (m, 2H), 4.26-4.08 | m/z = |
| | | (m, 1H), 4.03 (s, 3H), 4.02-3.86 (m, 1H), 3.85-3.67 (m, | 646.2 |
| | | 2H), 3.10-3.01 (m, 1H), 2.97-2.90 (m, 1H), 2.59 (s, 3H), | [M + H]+. |
| | | 2.47 (s, 3H), 1.88-1.79 (m, 1H), 0.60 (d, J = 6.6 Hz, 2H), | |
| | | 0.02 (s, 2H). | |
| 57 | Method | 1H NMR (400 MHz, CDCl3) δ 9.19-9.13 (m, 1H), 7.38 (s, | LCMS Rt = |
| | 1 | 1H), 6.99 (d, J = 2.4 Hz, 1H), 6.82 (s, 1H), 6.77-6.61 (m, | 2.817 min, |
| | | 1H), 5.05-4.79 (m, 1H), 4.75-4.56 (m, 1H), 4.46 (d, J = | m/z = |
| | | 10.5 Hz, 1H), 4.38-3.89 (m, 2H), 3.82 (s, 2H), 3.12-3.00 | 631.2 |
| | | (m, 1H), 2.85 (dd, J = 4.5, 16.5 Hz, 1H), 2.79-2.74 (m, | [M + H]+. |
| | | 3H), 2.73 (s, 3H), 2.56 (s, 3H), 1.93-1.85 (m, 1H), 0.60 (d, | |
| | | J = 6.6 Hz, 2H), 0.06 (d, J = 15.9 Hz, 2H). | |
| 58 | Method | 1H NMR (400 MHz, CDCl3) δ 9.23 (s, 1H), 8.89 (s, 1H), | LCMS Rt = |
| | 1 | 7.40 (s, 1H), 7.05-6.99 (m, 1H), 6.90-6.83 (m, 1H), 6.82- | 2.745 min, |
| | | 6.64 (m, 1H), 5.07-4.76 (m, 1H), 4.73-4.42 (m, 2H), | m/z = |
| | | 4.41-3.96 (m, 2H), 3.95-3.46 (m, 2H), 3.15-2.98 (m, 1H), | 617.2 |
| | | 2.93-2.81 (m, 1H), 2.75 (s, 3H), 2.59 (s, 3H), 1.94-1.86 | [M + H]+. |
| | | (m, 1H), 0.70-0.48 (m, 2H), 0.17-0.12 (m, 2H). | |
| 3 | Method | 1H NMR (400 MHz, DMSO-d6) δ 8.03 (dd, J = 6.0, 8.8 Hz, | LCMS Rt = |
| | 1 | 1H), 7.80 (dd, J = 4.1, 7.7 Hz, 1H), 7.64-7.49 (m, 2H), | 2.450 min, |
| | | 7.48-7.37 (m, 1H), 7.32-7.16 (m, 1H), 5.07-4.64 (m, | m/z = |
| | | 1H), 4.42-4.19 (m, 1H), 4.18-4.11 (m, 1H), 4.08 (d, J = | 746.2 |
| | | 12.8 Hz, 2H), 3.93 (d, J = 6.0 Hz, 2H), 3.86-3.69 (m, 2H), | [M + H]+. |
| | | 3.47 (d, J = 3.5 Hz, 2H), 3.25 (s, 2H), 3.20-3.03 (m, 4H), | |
| | | 3.01-2.77 (m, 4H), 2.65 (s, 3H), 1.93-1.66 (m, 6H), 1.57 | |
| | | (d, J = 4.0 Hz, 2H). | |
| 1 | Method | 1H NMR (400 MHz, CD3CN) δ 7.88-7.82 (m, 1H), 7.72- | LCMS Rt = |
| | 1 | 7.66 (m, 1H), 7.58-7.53 (m, 1H), 7.51 (s, 1H), 7.44-7.37 | 2.278 min, |
| | | (m, 1H), 7.37-7.30 (m, 1H), 6.68-6.53 (m, 1H), 4.88- | m/z = |
| | | 4.76 (m, 1H), 4.29-4.22 (m, 2H), 3.97-3.87 (m, 1H), 3.86- | 663.2 |
| | | 3.78 (m, 3H), 3.77-3.68 (m, 3H), 3.52 (dd, J = 5.4, 11.4 | [M + H]+. |
| | | Hz, 2H), 3.44-3.38 (m, 1H), 3.36-3.24 (m, 3H), 3.08 (s, | |
| | | 1H), 3.03-2.94 (m, 3H), 2.92-2.84 (m, 2H), 2.63-2.58 | |
| | | (m, 1H), 2.11-2.03 (m, 5H), 2.01-1.96 (m, 3H), 1.88- | |
| | | 1.76 (m, 2H). | |
| 40 | Method | 1H NMR (400 MHz, CD3CN) δ 9.28 (s, 1H), 8.71 (d, J = | LCMS Rt = |
| | 1 | 5.0 Hz, 1H), 8.16 (d, J = 8.2 Hz, 1H), 8.05 (d, J = 8.2 Hz, | 2.974 min, |
| | | 1H), 7.75-7.70 (m, 1H), 7.65 (d, J =7.6 Hz, 2H), 7.58- | m/z = |
| | | 7.52 (m, 2H), 6.83-6.68 (m, 1H), 5.36-5.17 (m, 1H), | 729.3 |
| | | 4.69-4.60 (m, 1H), 4.54-4.42 (m, 1H), 4.28-4.14 (m, 4H), | [M + H]+. |
| | | 4.06-3.94 (m, 1H), 3.62 (s, 3H), 3.30 (d, J = 7.1 Hz, 1H), | |
| | | 3.21-3.12 (m, 2H), 3.09 (s, 1H), 2.95-2.86 (m, 1H), 2.67 | |
| | | (s, 3H), 2.14-2.04 (m, 4H), 1.94-1.80 (m, 3H). | |
| 36 | Method | 1H NMR (400 MHz, CD3CN) δ 9.33 (d, J = 11.4 Hz, 1H), | LCMS Rt = |
| | 1 | 8.69 (s, 1H), 8.15 (d, J = 8.1 Hz, 1H), 8.05 (d, J = 8.1 Hz, | 2.828 min, |
| | | 1H), 7.76-7.62 (m, 3H), 7.59-7.48 (m, 2H), 6.56-6.36 | m/z = |
| | | (m, 1H), 5.37-5.16 (m, 1H), 4.86-4.56 (m, 2H), 4.32- | 759.3 |
| | | 4.11 (m, 4H), 3.69-3.59 (m, 1H), 3.51 (d, J = 8.9 Hz, 3H), | [M + H]+. |
| | | 3.24-3.05 (m, 4H), 2.96-2.86 (m, 1H), 2.65 (s, 3H), 2.14- | |
| | | 2.00 (m, 4H), 1.93-1.80 (m, 3H), 1.74-1.54 (m, 1H). | |

### BIOLOGICAL EXAMPLES

### Example 16: Inhibition of KRAS^{G12C} and cRAF Binding

The AlphaScreen technology was used to determine IC₅₀s for compound inhibition of KRAS G12C (present as the Cys-light (C51S, C80L and C118S), truncated version comprising amino acids 1-169) and cRAF interaction. Compounds were diluted in 100% DMSO and each compound concentration was spotted at 200 nl/well onto low volume, white 384 well plates. The KRAS G12C contained a biotin-AviTag and the cRaf, as Ras-binding domain (amino acids 50-131, RBD), was GST-tagged. KRAS G12C was preloaded with the GTP analogue Guanosine 5′-[0,7-imido]triphosphate (GMPPNP). The KRAS G12C was diluted in 25 mM Hepes, pH 7.4, 150 mM NaCl, 5 mM MgCl₂, 0.01% TritonX-100 and 10 μM GMPPNP and added at 10 ul/well to compound-spotted plates resulting in a DMSO concentration of 2%. Plates were incubated for 2 hours. A mixture of RBD and the AlphaScreen streptavidin donor and glutathione acceptor beads diluted in 25 mM Hepes, pH 7.4, 150 mM NaCl, 5 mM MgCl₂, 0.01% TritonX-100 and 2% DMSO was then added at 10 ul/well and incubated for 60-90 minutes before the samples were read for emission at 570 nm after excitation of the donor beads at 680 nm. All incubations were performed at room temperature. The final top compound concentration was 50 μM with 1:3 titrations for 10-point dose response curves. Final assay conditions were 0.5 nM KRAS G12C, 0.75 nM RBD and 5 μg/ml each of AlphaScreen donor and acceptor beads. IC₅₀s were determined using nonlinear regression fit of [inhibitor] vs. response (4 parameters).

A counter assay was also set up to rule out inhibitors of the AlphaScreen technology itself. Compound plates were incubated for 2 hours as above with buffer only. The AlphaScreen beads were added as above except biotin-AviTag-GST was substituted for the RBD. Samples were read and analyzed as above.

Results for compounds are shown in Table 1.

TABLE 1
Inhibition of KRAS G12C and cRAF Binding (IC₅₀)

| Compound | | |
|---|---|---|
| Nr | Structure | IC50 |
| 1 | 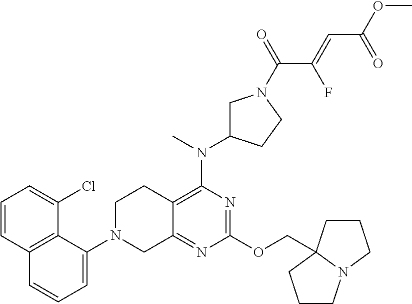 [SMILES: COC(=O)/C=C(\F)C(=O)N1CCC(N(C)c2nc(OCC34CCCN3CCC4)nc3c2CCN(c2cccc4cccc(Cl)c24)C3)C1] | +++ |
| 2 | 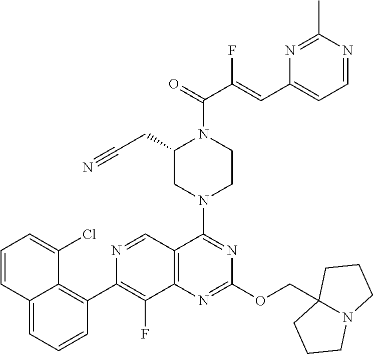 [SMILES: Cc1nccc(/C=C(\F)C(=O)N2CCN(c3nc(OCC45CCCN4CCC5)nc4c(F)c(-c5cccc6cccc(Cl)c56)ncc34)C[C@@H]2CC#N)n1] | ++++ |
| 3 | 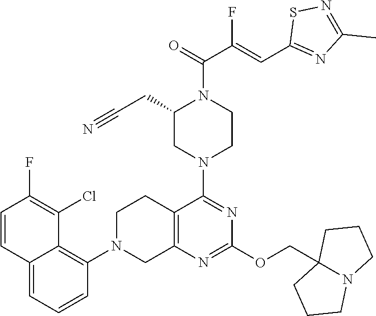 [SMILES: Cc1nsc(/C=C(\F)C(=O)N2CCN(c3nc(OCC45CCCN4CCC5)nc4c3CCN(c3cccc5ccc(F)c(Cl)c35)C4)C[C@@H]2CC#N)n1] | ++++ |
| 4 | 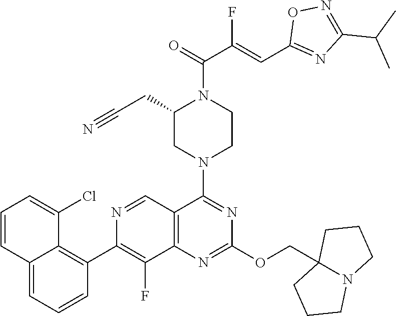 [SMILES: CC(C)c1noc(/C=C(\F)C(=O)N2CCN(c3nc(OCC45CCCN4CCC5)nc4c(F)c(-c5cccc6cccc(Cl)c56)ncc34)C[C@@H]2CC#N)n1] | ++++ |
| 5 | 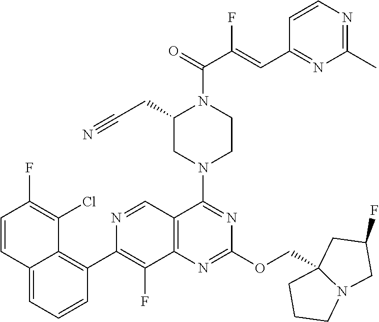 [SMILES: Cc1nccc(/C=C(\F)C(=O)N2CCN(c3nc(OC[C@@]45CCCN4C[C@H](F)C5)nc4c(F)c(-c5cccc6ccc(F)c(Cl)c56)ncc34)C[C@@H]2CC#N)n1] | ++++ |
| 6 | 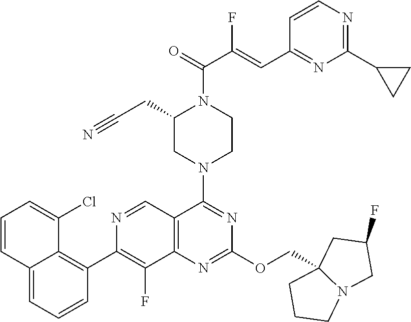 [SMILES: N#CC[C@H]1CN(c2nc(OC[C@@]34CCCN3C[C@H](F)C4)nc3c(F)c(-c4cccc5cccc(Cl)c45)ncc23)CCN1C(=O)/C(F)=C/c1ccnc(C2CC2)n1] | ++++ |
| 7 | 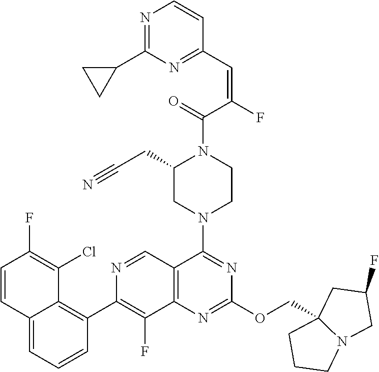 [SMILES: N#CC[C@H]1CN(c2nc(OC[C@@]34CCCN3C[C@H](F)C4)nc3c(F)c(-c4cccc5ccc(F)c(Cl)c45)ncc23)CCN1C(=O)/C(F)=C\c1ccnc(C2CC2)n1] | +++ |
| 8 | 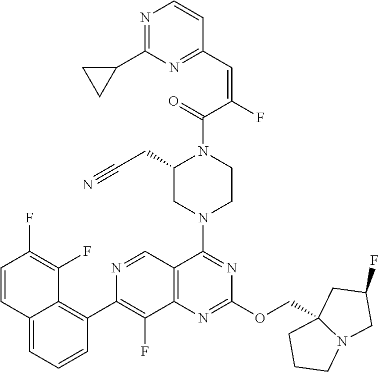 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C(F)=C\c1ccnc(C2CC2)n1] | +++ |
| 9 | 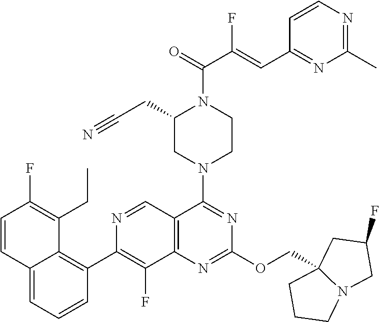 [SMILES: CCc1c(F)ccc2cccc(-c3ncc4c(N5CCN(C(=O)/C(F)=C/c6ccnc(C)n6)[C@@H](CC#N)C5)nc(OC[C@@]56CCCN5C[C@H](F)C6)nc4c3F)c12] | ++++ |
| 10 | 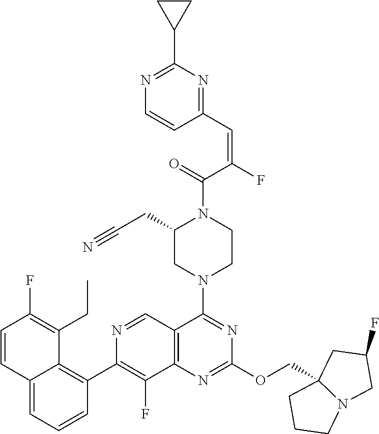 [SMILES: CCc1c(F)ccc2cccc(-c3ncc4c(N5CCN(C(=O)/C(F)=C\c6ccnc(C7CC7)n6)[C@@H](CC#N)C5)nc(OC[C@@]56CCCN5C[C@H](F)C6)nc4c3F)c12] | ++ |
| 11 | 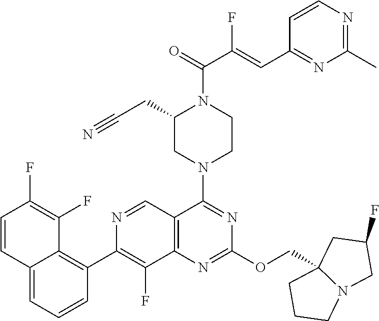 [SMILES: Cc1nccc(/C=C(\F)C(=O)N2CCN(c3nc(OC[C@@]45CCCN4C[C@H](F)C5)nc4c(F)c(-c5cccc6ccc(F)c(F)c56)ncc34)C[C@@H]2CC#N)n1] | ++++ |
| 12 | 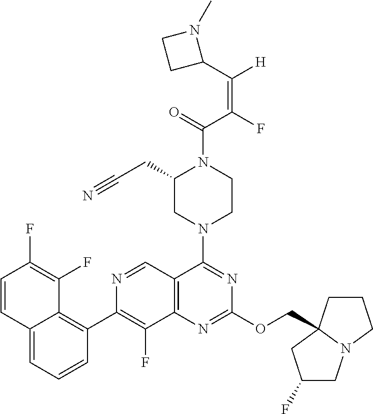 [SMILES: CN1CCC1/C=C(/F)C(=O)N1CCN(c2nc(OC[C@@]34CCCN3C[C@H](F)C4)nc3c(F)c(-c4cccc5ccc(F)c(F)c45)ncc23)C[C@@H]1CC#N] | + |
| 13 | 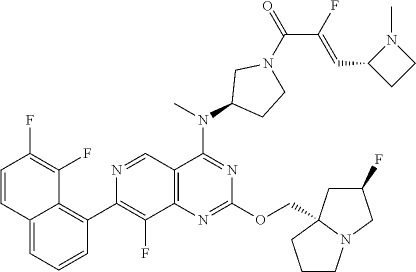 [SMILES: CN1CC[C@@H]1/C=C(\F)C(=O)N1CC[C@@H](N(C)c2nc(OC[C@@]34CCCN3C[C@H](F)C4)nc3c(F)c(-c4cccc5ccc(F)c(F)c45)ncc23)C1] | ++ |
| 14 | 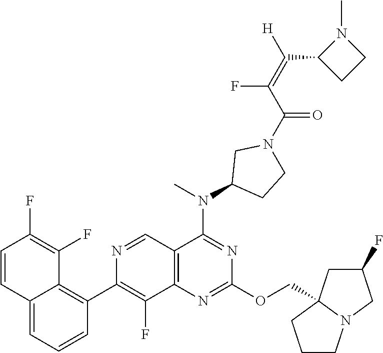 [SMILES: CN1CC[C@@H]1/C=C(/F)C(=O)N1CC[C@@H](N(C)c2nc(OC[C@@]34CCCN3C[C@H](F)C4)nc3c(F)c(-c4cccc5ccc(F)c(F)c45)ncc23)C1] | +++ |
| 15 | 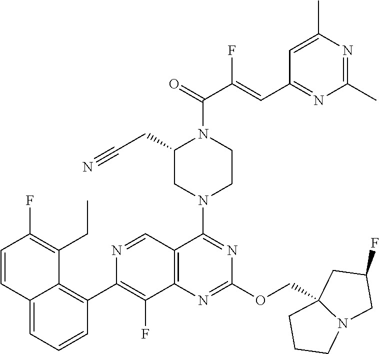 [SMILES: CCc1c(F)ccc2cccc(-c3ncc4c(N5CCN(C(=O)/C(F)=C/c6cc(C)nc(C)n6)[C@@H](CC#N)C5)nc(OC[C@@]56CCCN5C[C@H](F)C6)nc4c3F)c12] | ++++ |
| 16 | 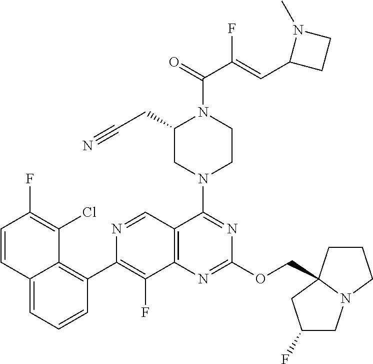 [SMILES: CN1CCC1/C=C(\F)C(=O)N1CCN(c2nc(OC[C@@]34CCCN3C[C@H](F)C4)nc3c(F)c(-c4cccc5ccc(F)c(Cl)c45)ncc23)C[C@@H]1CC#N] | ++ |
| 17 | 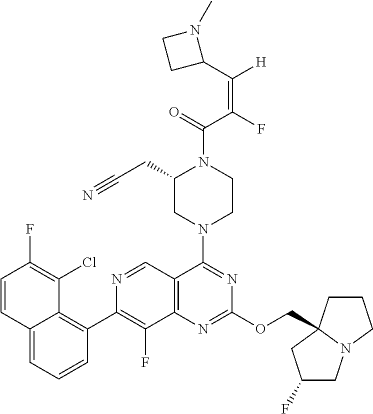 [SMILES: CN1CCC1/C=C(/F)C(=O)N1CCN(c2nc(OC[C@@]34CCCN3C[C@H](F)C4)nc3c(F)c(-c4cccc5ccc(F)c(Cl)c45)ncc23)C[C@@H]1CC#N] | + |
| 18 | 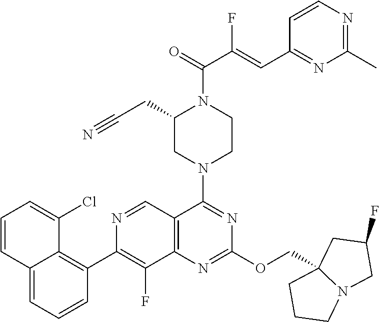 [SMILES: Cc1nccc(/C=C(\F)C(=O)N2CCN(c3nc(OC[C@@]45CCCN4C[C@H](F)C5)nc4c(F)c(-c5cccc6cccc(Cl)c56)ncc34)C[C@@H]2CC#N)n1] | ++++ |
| 19 | 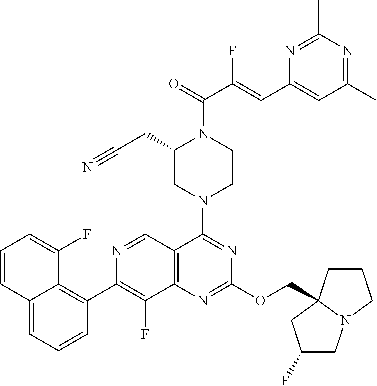 [SMILES: Cc1cc(/C=C(\F)C(=O)N2CCN(c3nc(OC[C@@]45CCCN4C[C@H](F)C5)nc4c(F)c(-c5cccc6cccc(F)c56)ncc34)C[C@@H]2CC#N)nc(C)n1] | ++++ |
| 20 | 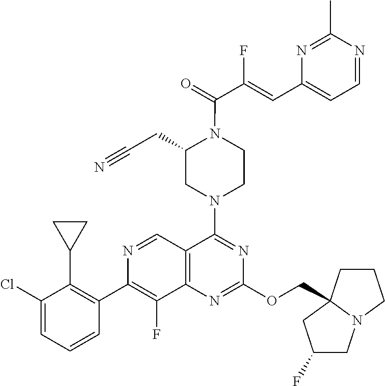 [SMILES: Cc1nccc(/C=C(\F)C(=O)N2CCN(c3nc(OC[C@@]45CCCN4C[C@H](F)C5)nc4c(F)c(-c5cccc(Cl)c5C5CC5)ncc34)C[C@@H]2CC#N)n1] | ++++ |
| 21 | 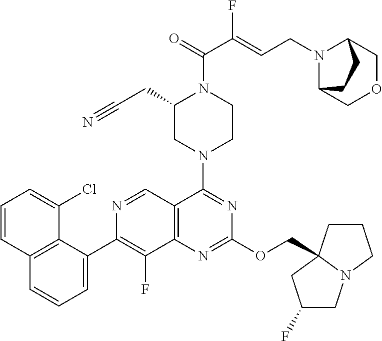 | + |
| 22 | 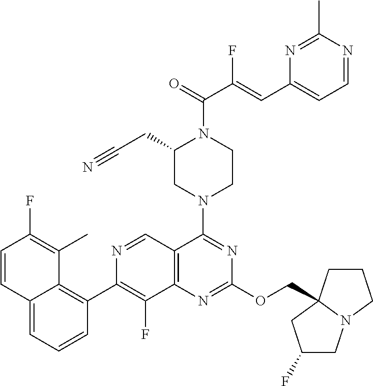 [SMILES: Cc1nccc(/C=C(\F)C(=O)N2CCN(c3nc(OC[C@@]45CCCN4C[C@H](F)C5)nc4c(F)c(-c5cccc6ccc(F)c(C)c56)ncc34)C[C@@H]2CC#N)n1] | ++++ |
| 23 | 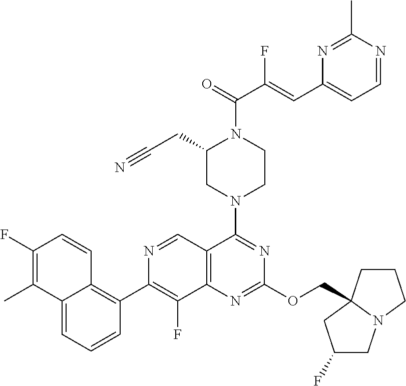 [SMILES: Cc1nccc(/C=C(\F)C(=O)N2CCN(c3nc(OC[C@@]45CCCN4C[C@H](F)C5)nc4c(F)c(-c5cccc6c(C)c(F)ccc56)ncc34)C[C@@H]2CC#N)n1] | +++ |
| 24 | 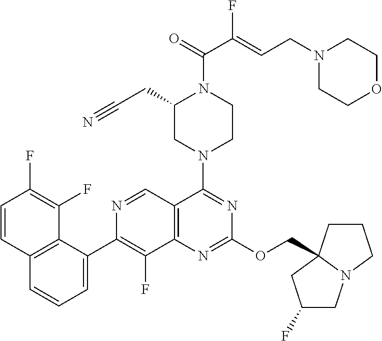 [SMILES: N#CC[C@H]1CN(c2nc(OC[C@@]34CCCN3C[C@H](F)C4)nc3c(F)c(-c4cccc5ccc(F)c(F)c45)ncc23)CCN1C(=O)/C(F)=C/CN1CCOCC1] | ++ |
| 25 | 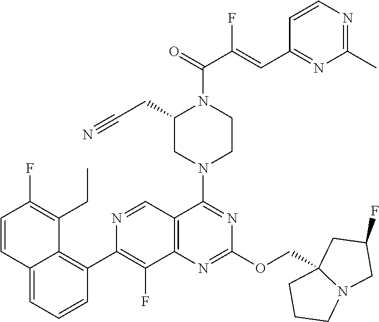 [SMILES: CCc1c(F)ccc2cccc(-c3ncc4c(N5CCN(C(=O)/C(F)=C/c6ccnc(C)n6)[C@@H](CC#N)C5)nc(OC[C@@]56CCCN5C[C@H](F)C6)nc4c3F)c12] | ++++ |
| 26 | 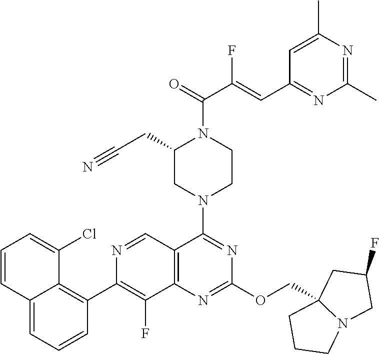 [SMILES: Cc1cc(/C=C(\F)C(=O)N2CCN(c3nc(OC[C@@]45CCCN4C[C@H](F)C5)nc4c(F)c(-c5cccc6cccc(Cl)c56)ncc34)C[C@@H]2CC#N)nc(C)n1] | ++++ |
| 27 | 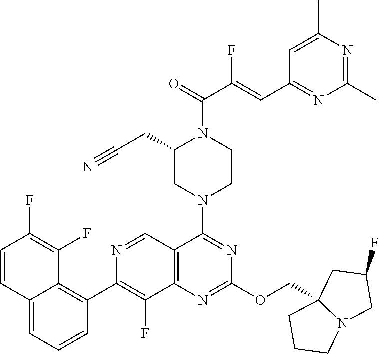 [SMILES: Cc1cc(/C=C(\F)C(=O)N2CCN(c3nc(OC[C@@]45CCCN4C[C@H](F)C5)nc4c(F)c(-c5cccc6ccc(F)c(F)c56)ncc34)C[C@@H]2CC#N)nc(C)n1] | ++++ |
| 28 | 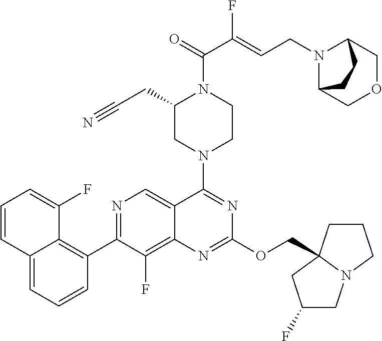 | + |
| 29 | 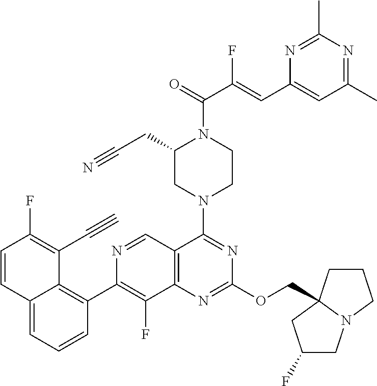 [SMILES: C#Cc1c(F)ccc2cccc(-c3ncc4c(N5CCN(C(=O)/C(F)=C/c6cc(C)nc(C)n6)[C@@H](CC#N)C5)nc(OC[C@@]56CCCN5C[C@H](F)C6)nc4c3F)c12] | ++++ |
| 30 | 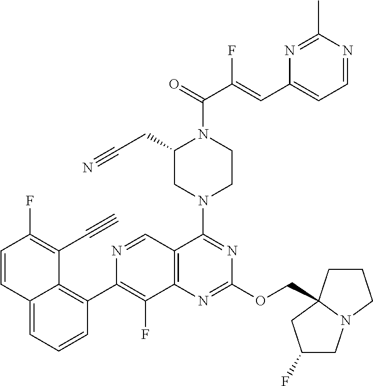 [SMILES: C#Cc1c(F)ccc2cccc(-c3ncc4c(N5CCN(C(=O)/C(F)=C/c6ccnc(C)n6)[C@@H](CC#N)C5)nc(OC[C@@]56CCCN5C[C@H](F)C6)nc4c3F)c12] | ++++ |
| 31 | 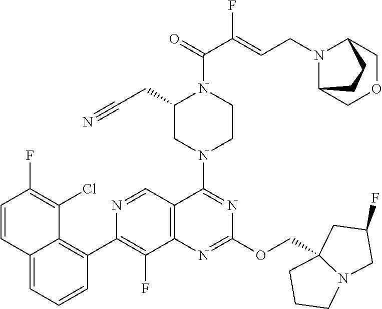 | + |
| 32 | 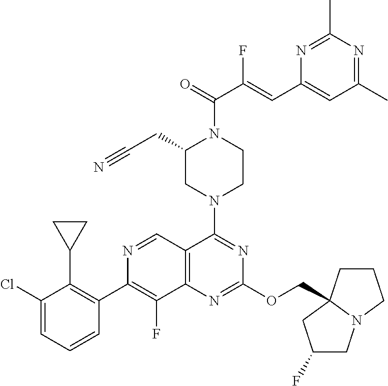 [SMILES: Cc1cc(/C=C(\F)C(=O)N2CCN(c3nc(OC[C@@]45CCCN4C[C@H](F)C5)nc4c(F)c(-c5cccc(Cl)c5C5CC5)ncc34)C[C@@H]2CC#N)nc(C)n1] | ++++ |
| 33 | 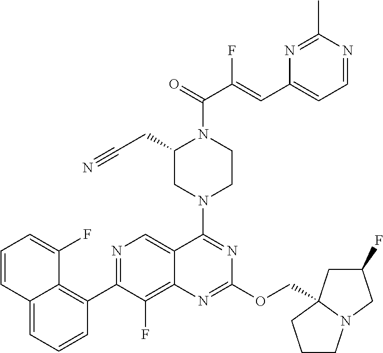 [SMILES: Cc1nccc(/C=C(\F)C(=O)N2CCN(c3nc(OC[C@@]45CCCN4C[C@H](F)C5)nc4c(F)c(-c5cccc6cccc(F)c56)ncc34)C[C@@H]2CC#N)n1] | ++++ |
| 34 | 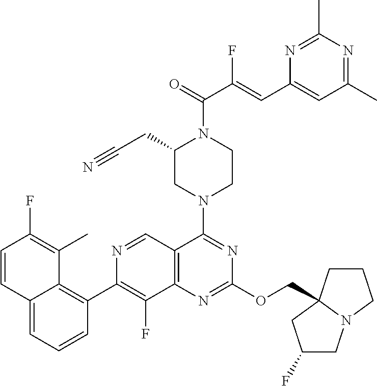 [SMILES: Cc1cc(/C=C(\F)C(=O)N2CCN(c3nc(OC[C@@]45CCCN4C[C@H](F)C5)nc4c(F)c(-c5cccc6ccc(F)c(C)c56)ncc34)C[C@@H]2CC#N)nc(C)n1] | ++++ |
| 35 | 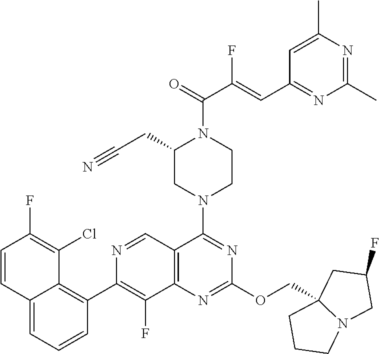 [SMILES: Cc1cc(/C=C(\F)C(=O)N2CCN(c3nc(OC[C@@]45CCCN4C[C@H](F)C5)nc4c(F)c(-c5cccc6ccc(F)c(Cl)c56)ncc34)C[C@@H]2CC#N)nc(C)n1] | ++++ |
| 36 | 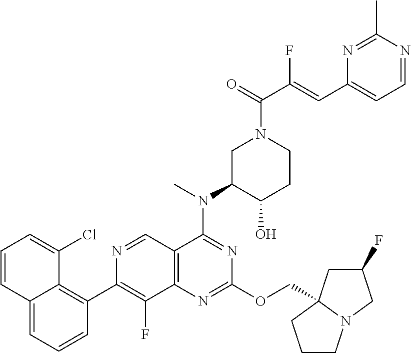 [SMILES: Cc1nccc(/C=C(\F)C(=O)N2CC[C@H](O)[C@@H](N(C)c3nc(OC[C@@]45CCCN4C[C@H](F)C5)nc4c(F)c(-c5cccc6cccc(Cl)c56)ncc34)C2)n1] | ++ |
| 37 | 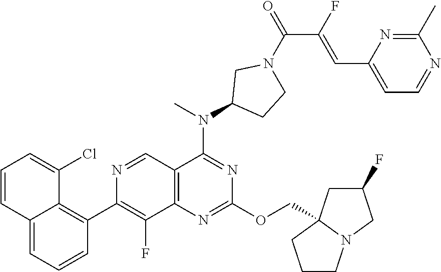 [SMILES: Cc1nccc(/C=C(\F)C(=O)N2CC[C@@H](N(C)c3nc(OC[C@@]45CCCN4C[C@H](F)C5)nc4c(F)c(-c5cccc6cccc(Cl)c56)ncc34)C2)n1] | ++++ |
| 38 | 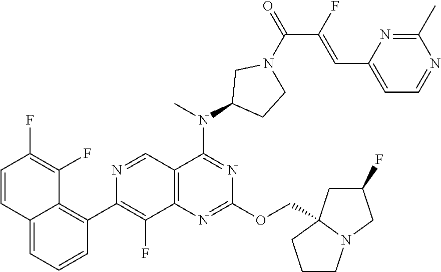 [SMILES: Cc1nccc(/C=C(\F)C(=O)N2CC[C@@H](N(C)c3nc(OC[C@@]45CCCN4C[C@H](F)C5)nc4c(F)c(-c5cccc6ccc(F)c(F)c56)ncc34)C2)n1] | ++++ |
| 39 | 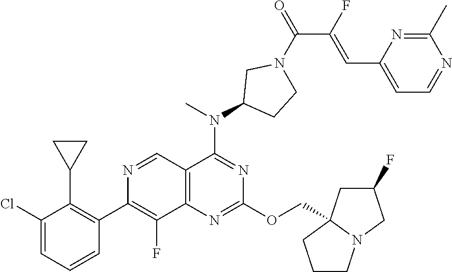 [SMILES: Cc1nccc(/C=C(\F)C(=O)N2CC[C@@H](N(C)c3nc(OC[C@@]45CCCN4C[C@H](F)C5)nc4c(F)c(-c5cccc(Cl)c5C5CC5)ncc34)C2)n1] | ++++ |
| 40 | 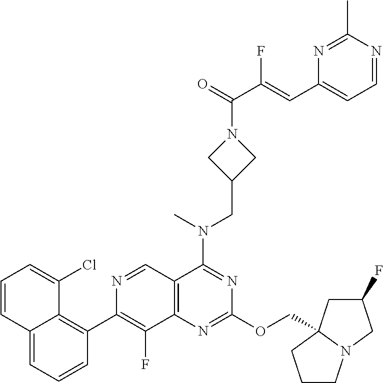 [SMILES: Cc1nccc(/C=C(\F)C(=O)N2CC(CN(C)c3nc(OC[C@@]45CCCN4C[C@H](F)C5)nc4c(F)c(-c5cccc6cccc(Cl)c56)ncc34)C2)n1] | ++++ |
| 41 | 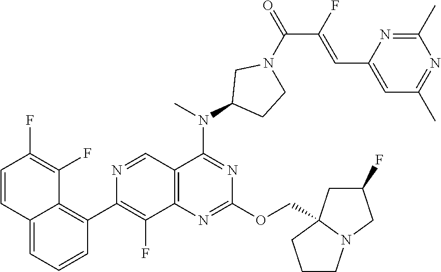 [SMILES: Cc1cc(/C=C(\F)C(=O)N2CC[C@@H](N(C)c3nc(OC[C@@]45CCCN4C[C@H](F)C5)nc4c(F)c(-c5cccc6ccc(F)c(F)c56)ncc34)C2)nc(C)n1] | ++++ |
| 42 | 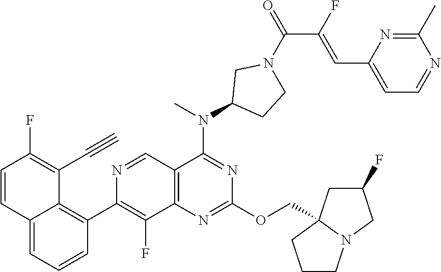 [SMILES: C#Cc1c(F)ccc2cccc(-c3ncc4c(N(C)[C@@H]5CCN(C(=O)/C(F)=C/c6ccnc(C)n6)C5)nc(OC[C@@]56CCCN5C[C@H](F)C6)nc4c3F)c12] | ++++ |
| 43 | 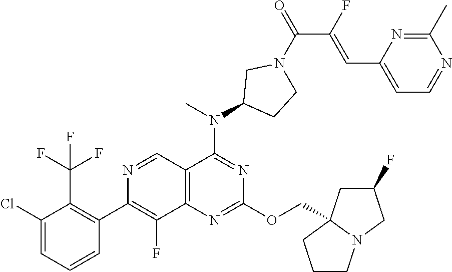 [SMILES: Cc1nccc(/C=C(\F)C(=O)N2CC[C@@H](N(C)c3nc(OC[C@@]45CCCN4C[C@H](F)C5)nc4c(F)c(-c5cccc(Cl)c5C(F)(F)F)ncc34)C2)n1] | ++++ |
| 44 | 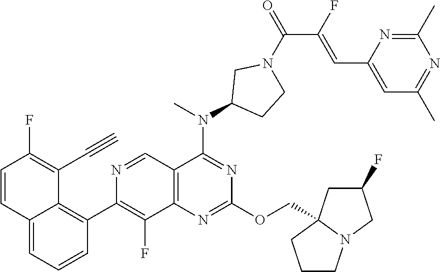 [SMILES: C#Cc1c(F)ccc2cccc(-c3ncc4c(N(C)[C@@H]5CCN(C(=O)/C(F)=C/c6cc(C)nc(C)n6)C5)nc(OC[C@@]56CCCN5C[C@H](F)C6)nc4c3F)c12] | ++++ |
| | Formate salt | |
| 45 | 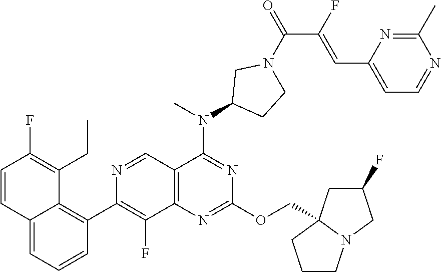 [SMILES: CCc1c(F)ccc2cccc(-c3ncc4c(N(C)[C@@H]5CCN(C(=O)/C(F)=C/c6ccnc(C)n6)C5)nc(OC[C@@]56CCCN5C[C@H](F)C6)nc4c3F)c12] | ++++ |
| 46 | 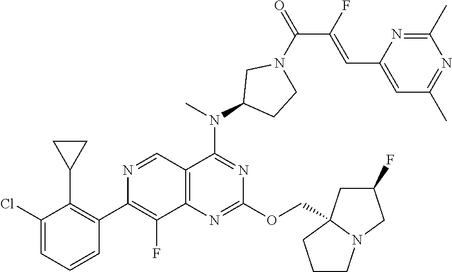 [SMILES: Cc1cc(/C=C(\F)C(=O)N2CC[C@@H](N(C)c3nc(OC[C@@]45CCCN4C[C@H](F)C5)nc4c(F)c(-c5cccc(Cl)c5C5CC5)ncc34)C2)nc(C)n1] | ++++ |
| 47 | 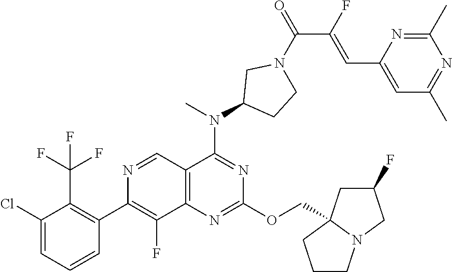 [SMILES: Cc1cc(/C=C(\F)C(=O)N2CC[C@@H](N(C)c3nc(OC[C@@]45CCCN4C[C@H](F)C5)nc4c(F)c(-c5cccc(Cl)c5C(F)(F)F)ncc34)C2)nc(C)n1] | ++++ |
| 48 | 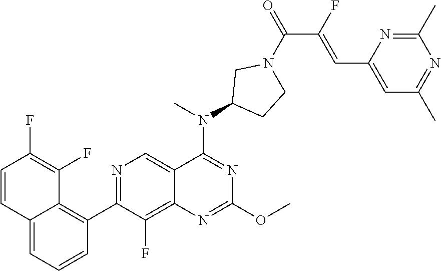 [SMILES: COc1nc(N(C)[C@@H]2CCN(C(=O)/C(F)=C/c3cc(C)nc(C)n3)C2)c2cnc(-c3cccc4ccc(F)c(F)c34)c(F)c2n1] | + |
| 49 | 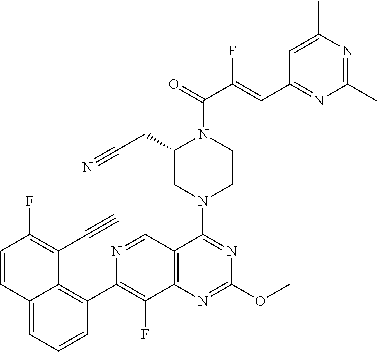 [SMILES: C#Cc1c(F)ccc2cccc(-c3ncc4c(N5CCN(C(=O)/C(F)=C/c6cc(C)nc(C)n6)[C@@H](CC#N)C5)nc(OC)nc4c3F)c12] | ++ |
| 50 | 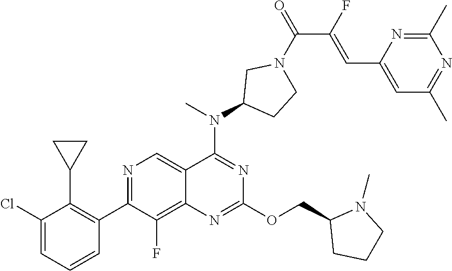 [SMILES: Cc1cc(/C=C(\F)C(=O)N2CC[C@@H](N(C)c3nc(OC[C@@H]4CCCN4C)nc4c(F)c(-c5cccc(Cl)c5C5CC5)ncc34)C2)nc(C)n1] | +++ |
| 51 | 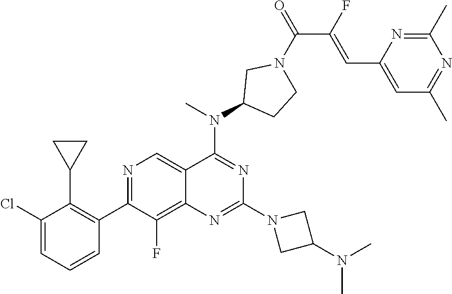 [SMILES: Cc1cc(/C=C(\F)C(=O)N2CC[C@@H](N(C)c3nc(N4CC(N(C)C)C4)nc4c(F)c(-c5cccc(Cl)c5C5CC5)ncc34)C2)nc(C)n1] | ++ |
| 52 | 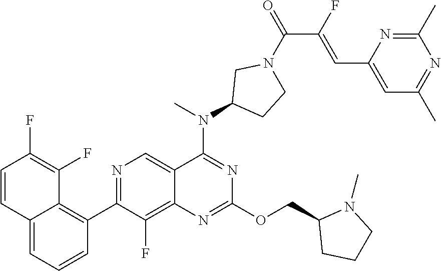 [SMILES: Cc1cc(/C=C(\F)C(=O)N2CC[C@@H](N(C)c3nc(OC[C@@H]4CCCN4C)nc4c(F)c(-c5cccc6ccc(F)c(F)c56)ncc34)C2)nc(C)n1] | ++++ |
| 53 | 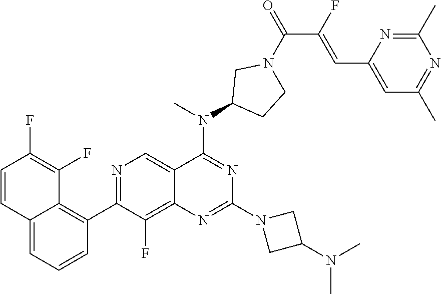 [SMILES: Cc1cc(/C=C(\F)C(=O)N2CC[C@@H](N(C)c3nc(N4CC(N(C)C)C4)nc4c(F)c(-c5cccc6ccc(F)c(F)c56)ncc34)C2)nc(C)n1] | +++ |
| 54 | 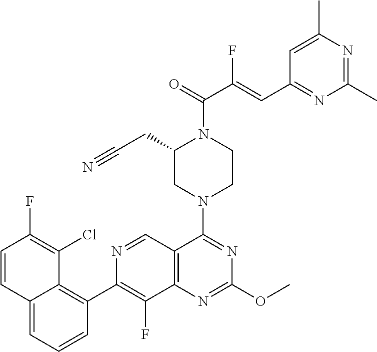 [SMILES: COc1nc(N2CCN(C(=O)/C(F)=C/c3cc(C)nc(C)n3)[C@@H](CC#N)C2)c2cnc(-c3cccc4ccc(F)c(Cl)c34)c(F)c2n1] | + |
| 55 | 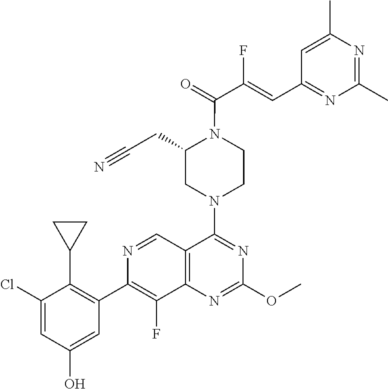 [SMILES: COc1nc(N2CCN(C(=O)/C(F)=C/c3cc(C)nc(C)n3)[C@@H](CC#N)C2)c2cnc(-c3cc(O)cc(Cl)c3C3CC3)c(F)c2n1] | ++++ |
| 56 | 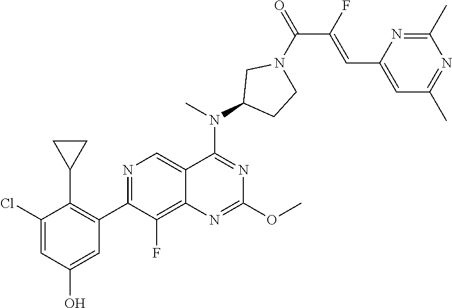 [SMILES: COc1nc(N(C)[C@@H]2CCN(C(=O)/C(F)=C/c3cc(C)nc(C)n3)C2)c2cnc(-c3cc(O)cc(Cl)c3C3CC3)c(F)c2n1] | ++ |
| 57 | 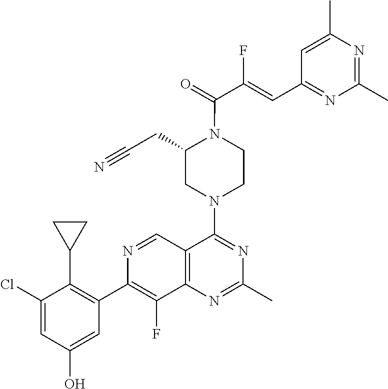 [SMILES: Cc1cc(/C=C(\F)C(=O)N2CCN(c3nc(C)nc4c(F)c(-c5cc(O)cc(Cl)c5C5CC5)ncc34)C[C@@H]2CC#N)nc(C)n1] | ++++ |
| 58 | 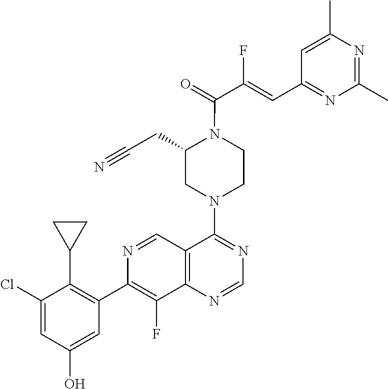 [SMILES: Cc1cc(/C=C(\F)C(=O)N2CCN(c3ncnc4c(F)c(-c5cc(O)cc(Cl)c5C5CC5)ncc34)C[C@@H]2CC#N)nc(C)n1] | ++++ |

IC₅₀ ++++ is less than 10 nM,
+++ is 10 to less than 100 nM,
++ is 100 to less than 500 nM,
+ is greater or equal to 500 nM.

### Example 17: Inhibition of KRAS^{G12C} and PI3Ka Binding

The AlphaScreen technology is used to determine IC₅₀s for compound inhibition of KRAS G12C (present as the Cys-light (C51S, C80L and C118S), truncated version comprising amino acids 1-169) and PJ3Ka interaction. Compounds are diluted in 100% DMSO and each compound concentration is spotted at 200 nl/well onto low volume, white 384 well plates. The KRAS G12C contains a biotin-AviTag and the PI3Ka, as Ras-binding domain (amino acids 157-300, RBD), is His-tagged. KRAS G12C is preloaded with the GTP analogue Guanosine 5′-[0,γ-imido]triphosphate (GMPPNP). The KRAS G12C is diluted in 25 mM Hepes, pH 7.4, 150 mM NaCl, 5 mM MgCl₂, 0.01% TritonX-100 and 10 μM GMPPNP and added at 10 ul/well to compound-spotted plates resulting in a DMSO concentration of 2%. Plates are incubated for 2 hours. A mixture of RBD and the AlphaScreen streptavidin donor and nickel chelate acceptor beads diluted in 25 mM Hepes, pH 7.4, 150 mM NaCl, 5 mM MgCl₂, 0.01% TritonX-100 and 2% DMSO are then added at 10 ul/well and incubated for 60-90 minutes before the samples are read for emission at 570 nm after excitation of the donor beads at 680 nm. All incubations are performed at room temperature. The final top compound concentration is 50 μM with 1:3 titrations for 10-point dose response curves. Final assay conditions are 1.5 nM KRAS G12C, 100 nM RBD, 1.25 ug/ml of AlphaScreen donor beads and 10 μg/ml AlphaLISA acceptor beads. IC₅₀s are determined using nonlinear regression fit of [inhibitor] vs. response (4 parameters).

A counter assay is also set up to rule out inhibitors of the AlphaScreen technology itself. Compound plates are incubated for about 2 hours as above with buffer only. The AlphaScreen beads are added as above except an unrelated biotinylated His-tagged peptide is substituted for the RBD. Samples are read and analyzed as above.

### Example 18: MCF10A (G12C or G12C-A59G)-KRAS Cell Viability Assay

MCF10A (ATCC, cat. CRL-10317) cells are maintained in MEBM (Lonza, cat. CC-3151) with 1% horse serum (Sigma, cat. H1270), MEGM mammary epithelial cell growth medium SingleQuotsKit (Lonza, cat. CC-4146) and 25 ng/ml Cholera toxin (Sigma, cat. C8052). These cells are transduced with either KRAS G12C or G12C/A59G followed by puromycin selection to generate stably expressing cells. For the cell viability assay, 1000 cells of either MCF10A KRAS G12C or MCF10A G12C/A59G are plated in 384-well spheroid microplate (Corning, cat. 3830). The following day, cells are treated with compounds (10 uM top concentration, 3-fold dilution, and 11 doses). 10 uM Tremetinib (MCE, cat. HY-10999/CS-0060) is used as control. The Tecan: HP D300E is used to dispense the compounds. After five days of incubation, celltiter-glo luminescent assay kit (Promega, cat. G7573) is used according to manufacturer's protocol to measure cellular viability using a BioTek plate reader. The data is then imported to and processed in Dotmatics where EC50s were calculated using the Lavenberg-Marquardt 4 parameters fitting procedure, with difference gradients.

### Example 19: Treatment of Human Patients

A human patient suffering from a cancer, (e.g., a KRAS mediated cancer, as disclosed herein) can be administered a therapeutically effective dose of a compound disclosed herein (e.g., a compound of Table 1). The treatment can slow down or halt the growth of a tumor, reduce a tumor volume or mass, or eradicate the tumor in the patient.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A compound of Formula (I), Formula (II), Formula (III), Formula (IV), Formula (V), or Formula (VI): 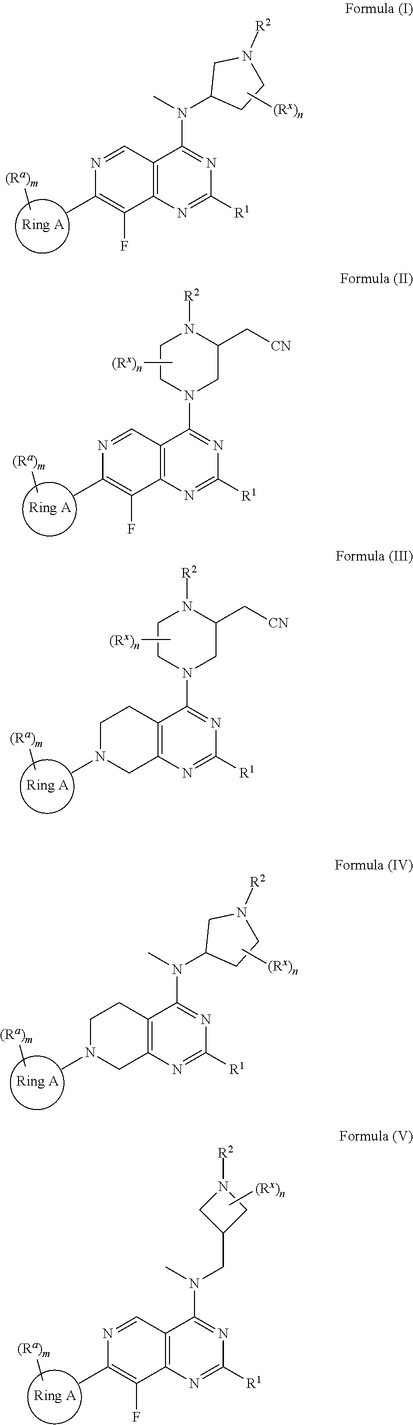 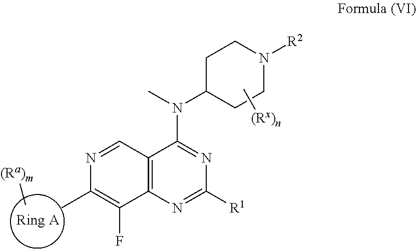
or a salt thereof; and/or an isotopologue thereof; wherein:
Ring A is a 6-10 membered aryl or a 5-10 membered heteroaryl;
each R^{a} is independently selected from the group consisting of halo, —OH, —NH₂, C₁-C₄ alkyl, C₃-C₄ cycloalkyl, C₁-C₄ alkoxy, C₁-C₄ haloalkyl, C₁-C₄ haloalkoxy, and C₂-C₃ alkynyl;
m is 0, 1, 2, or 3;
R¹ is selected from the group consisting of H, —OCH₂R^{1A}, —OC₁-C₄ alkyl, —C₁-C₄ alkyl, and 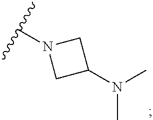 [SMILES: *N1CC(N(C)C)C1]
R^{1A} is selected from the group consisting of 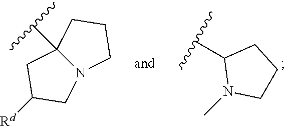
R^{d} is H or F;
R² is 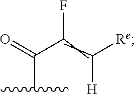
R^{e} is selected from the group consisting of —COOH, —C(O)O—C₁-C₄ alkyl, —C(O)O—C₁-C₄ haloalkyl, —C(O)—C₁-C₄ alkyl, —C(O)—C₁-C₄ haloalkyl, —C(O)N(C₁-C₄ alkyl)₂, —(C₁-C₂ alkyl)-(C₁-C₂ alkoxy), —S(O)₂—C₁-C₄ alkyl, —S(O)₂—C₁-C₄ haloalkyl, R^{e1}, —C(R^{Y1})(R^{Y2})R^{e1}, R^{e2}, and —C(R^{Y1})(R^{Y2})R^{e3};
R^{Y1} and R^{Y2} in each occurrence are independently —H or —CH₃;
R^{e1} is a 4-10 membered heterocycle which is substituted with 0, 1, 2, 3, or 4 substituents independently selected from the group consisting of halo, hydroxy, C₁-C₄ alkyl, C₁-C₆ alkoxy, C₁-C₆alkoxyalkoxy, C₁-C₄ haloalkyl, and C₁-C₄ haloalkoxy;
R^{e2} is a 5-6 membered heteroaryl group substituted with 0, 1, 2, or 3 substituents independently selected from the group consisting of halo, hydroxy, C₁-C₄ alkyl, C₁-C₄ hydroxyalkyl, C₁-C₄ alkoxy, C₁-C₄ haloalkyl, C₁-C₄ haloalkoxy, C₃-C₆ heterocyclyl substituted with 0, 1, or 2 substituents independently selected from the group consisting of halo, hydroxy, and methyl, and C₃-C₆ cycloalkyl substituted with 0, 1, or 2 substituents independently selected from the group consisting of halo, hydroxy, and methyl;
R^{e3} is —NR³¹R³²;
R³¹ and R³² are independently selected from the group consisting of hydrogen, C₁-C₄ alkyl, C₁-C₄ alkoxy, C₁-C₄ haloalkoxy, and C₁-C₄ alkyl substituted with a 3-6 membered heterocycle;
each R^{x} is independently selected from the group consisting of —OH, halo, and C₁-C₄ alkyl; and
n is 0, 1, or 2;
with the proviso that if R^{e} of Formula (II) is R^{e2}, then the 5-6 membered heteroaryl group of R^{e2} is substituted with 1, 2, or 3 substituents; and if R¹ of Formula (III) is 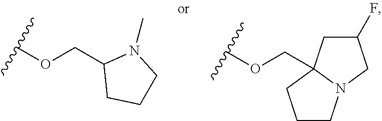
then 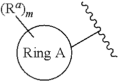
is not

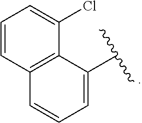 [SMILES: *c1cccc2cccc(Cl)c12]

2. The compound of claim 1, or a salt thereof, and/or an isotopologue thereof, wherein the compound is of Formula (I).

3. The compound of claim 1, or a salt thereof, and/or an isotopologue thereof, wherein the compound is of Formula (II).

4. The compound of claim 1, or a salt thereof, and/or an isotopologue thereof, wherein the compound is of formula (III).

5. The compound of claim 1, or a salt thereof, and/or an isotopologue thereof, wherein the compound is of formula (IV).

6. The compound of claim 1, or a salt thereof, and/or an isotopologue thereof, wherein the compound is of formula (V).

7. The compound of claim 1, or a salt thereof, and/or an isotopologue thereof, wherein the compound is of formula (VI).

8. The compound of any one of claims 1, 2, and 5, or a salt thereof, and/or an isotopologue thereof, wherein the stereochemistry of the pyrrolidine is (R) (i.e., the moiety represented as

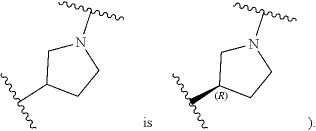

9. The compound of any one of claims 1, 3, and 4, or a salt thereof, and/or an isotopologue thereof, wherein the stereochemistry of the cyanomethyl groups is (S) (i.e., the moiety represented as

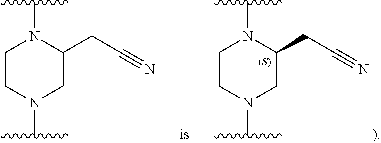

10. The compound of any one of claims 1-9, or a salt thereof, and/or an isotopologue thereof, wherein Ring A is selected from the group consisting of naphthalenyl, phenyl, isoquinolinyl, indazolyl and pyridinyl.

11. The compound of any one of claims 1-9, or a salt thereof, and/or an isotopologue thereof, wherein Ring A is selected from the group consisting of naphthalen-1-yl and phenyl.

12. The compound of any one of claims 1-11, or a salt thereof, and/or an isotopologue thereof, wherein each R^{a} is independently selected from halo, —OH, C₁-C₄ alkyl, C₃-C₄ cycloalkyl, C₁-C₄ haloalkyl, and C₂-C₃ alkynyl.

13. The compound of any one of claims 1-11, or a salt thereof, and/or an isotopologue thereof, wherein each R^{a} is independently selected from —F, —Cl, —OH, -Me, -Et, -cyclopropyl, —CF₃, and —C≡CH.

14. The compound of any one of claims 1-9, or a salt thereof, and/or an isotopologue thereof, wherein Ring A is selected from the group consisting of: 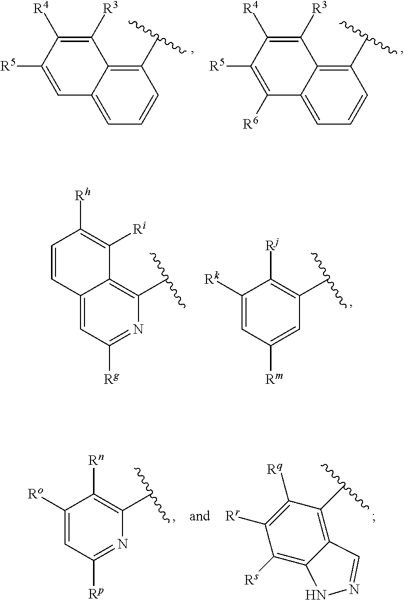
wherein:
each R³, R⁴, R⁵, R⁶, R^{h}, Rⁱ, R^{j}, R^{k}, Rⁿ, R^{o}, R^{q}, R^{r}, and R^{s} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl; and
each R^{g}, R^{m} and R^{p} is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl.

15. The compound of claim 14, wherein each R³, R⁴, R⁵, R⁶, R^{h}, Rⁱ, R^{j}, R^{k}, Rⁿ, R^{o}, R^{q}, R^{r}, and R^{s} is independently selected from the group consisting of —H, —F, —Cl, -Me, -Et, -cyclopropyl, —CF₃, and —C≡CH; and each R⁹, R^{m}, and R^{p} is independently —H or —OH.

16. The compound of claim 14 or 15, or a salt thereof, and/or an isotopologue thereof, wherein Ring A is

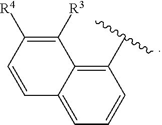

17. The compound of claim 14 or 15, or a salt thereof, and/or an isotopologue thereof, wherein Ring A is

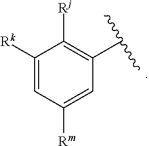

18. The compound of any one of claims 1-9, or a salt thereof, and/or an isotopologue thereof, wherein Ring A is

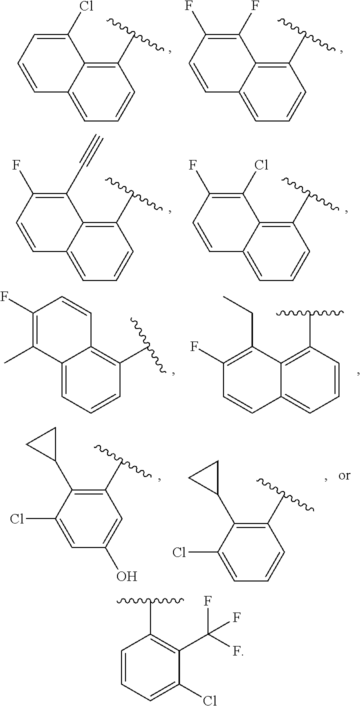

19. The compound of claim 14 or 15, or a salt thereof, and/or an isotopologue thereof, wherein R³ is H, halo, C₁-C₄ alkyl, or C₂-C₃ alkynyl; and R⁴ is H or halo.

20. The compound of claim 14 or 15, or a salt thereof, and/or an isotopologue thereof, wherein R³ is H, —F, —Cl, -Me, -Et, or —C≡CH; and R⁴ is H or —F.

21. The compound of claim 14 or 16, or a salt thereof, and/or an isotopologue thereof, wherein R^{j} is C₃-C₄ cycloalkyl or C₁-C₄ haloalkyl; R^{k} is H or halo; and R^{m} is H or —OH.

22. The compound of claim 14 or 16, or a salt thereof, and/or an isotopologue thereof, wherein R^{j} is cyclopropyl or —CF₃; R^{k} is H or —Cl; and R^{m} is H or —OH.

23. The compound of any one of claims 1-22, or a salt thereof, and/or an isotopologue thereof, wherein R^{d} is H.

24. The compound of any one of claims 1-22, or a salt thereof, and/or an isotopologue thereof, wherein R^{d} is F.

25. The compound of any one of claims 1-22, or a salt thereof, and/or an isotopologue thereof, wherein R^{1A} is

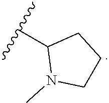 [SMILES: *C1CCCN1C ; "."]

26. The compound of any one of claims 1-22, or a salt thereof, and/or an isotopologue thereof, wherein R^{1A} is

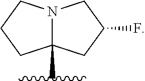 [SMILES: *[C@@]12CCCN1C[C@H](F)C2]

27. The compound of any one of claims 1-22, or a salt thereof, and/or an isotopologue thereof, wherein R¹ is H, —OCH₂R^{1A}, —OMe, Me, or

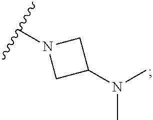 [SMILES: *N1CC(N(C)C)C1]

28. The compound of any one of claims 1-22, or a salt thereof, and/or an isotopologue thereof, wherein R¹ is selected from the group consisting of

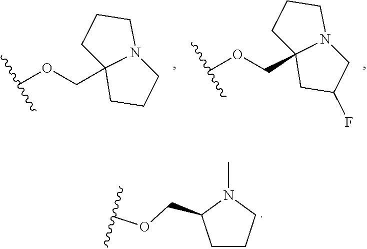

29. The compound of any one of claims 1-28, or a salt thereof, and/or an isotopologue thereof, wherein R² is

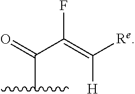

30. The compound of any one of claims 1-28, or a salt thereof, and/or an isotopologue thereof, wherein R² is

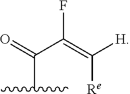

31. The compound of any one of claims 1-30, or a salt thereof, and/or an isotopologue thereof, wherein R^{Y}i is —H and R^{Y2} is —H.

32. The compound of any one of claims 1-31, or a salt thereof, and/or an isotopologue thereof, wherein R^{e} is selected from the group consisting of —C(O)O—C₁-C₄ alkyl, R^{e1}, —C(R^{Y1})(R^{Y2})R^{e}, R^{e2}, and —C(R^{Y1})(R^{Y2})R^{e3}.

33. The compound of any one of claims 1-31, or a salt thereof, and/or an isotopologue thereof, wherein R^{e1} is a 4-7 membered monocyclic heterocycle containing a nitrogen atom as the only heteroatom or containing one nitrogen atom and one oxygen atom; or a 6-10 member bridged heterocycle; wherein the 4-7 membered monocyclic heterocycle or the 6-10 membered bridged heterocycle is substituted with 0, 1, 2, 3, or 4 substituents independently selected from the group consisting of halo, hydroxy, C₁-C₄ alkyl, C₁-C₆ alkoxy, C₁-C₆ alkoxyalkoxy, C₁-C₄ haloalkyl, and C₁-C₄ haloalkoxy.

34. The compound of claim 1-31, or a salt thereof, and/or an isotopologue thereof, wherein R^{e1} is selected from the group consisting of azetidinyl, pyrrolidinyl, tetrahydrofuranyl, and morpholinyl, each substituted with 0 or 1 instance of methyl, ethyl, isopropyl, or methoxyethyl.

35. The compound of any one of claims 1-34, or a salt thereof, and/or an isotopologue thereof, wherein R^{e} is R^{e1} or —C(R^{yl})(R^{Y2})R^{e}.

36. The compound of claim 35, or a salt thereof, and/or an isotopologue thereof, wherein R^{e1} is 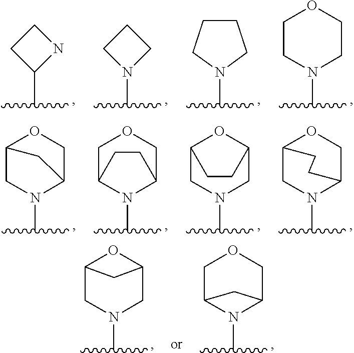
each substituted with 0, 1, 2, 3 or 4 substituents independently selected from halo, hydroxy, C₁-C₄ alkyl, C₁-C₆ alkoxy, C₁-C₆alkoxyalkoxy, C₁-C₄ haloalkyl and C₁-C₄ haloalkoxy.

37. The compound of any one of claims 1-30, or a salt thereof, and/or an isotopologue thereof, wherein R^{e} is R^{e2}.

38. The compound of claim 37, or a salt thereof, and/or an isotopologue thereof, wherein R^{e2} is a 5-6 membered heteroaryl group containing at least one nitrogen atom, wherein the attachment point for the heteroaryl group is a carbon atom group and wherein the heteroaryl is substituted with 0, 1, or 2 substituents independently selected from the group consisting of halo, C₁-C₄ alkyl, C₁-C₄ hydroxyalkyl, C₁-C₆ alkoxy, C₁-C₄ haloalkyl, C₁-C₄ haloalkoxy, and C₃-C₆ cycloalkyl optionally substituted with one or two substituents independently selected from the group consisting of fluoro and methyl.

39. The compound of claim 37, or a salt thereof, and/or an isotopologue thereof, wherein R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1, or 2 substituents independently selected from the group consisting of halo, C₁-C₄ alkyl, C₁-C₄ hydroxyalkyl, C₁-C₆ alkoxy, C₁-C₄ haloalkyl, C₁-C₄ haloalkoxy, and C₃-C₆ cycloalkyl optionally substituted with one or two substituents independently selected from fluoro and methyl.

40. The compound of claim 37, or a salt thereof, and/or an isotopologue thereof, wherein R^{e2} is selected from the group consisting of: 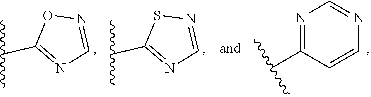
each substituted with 0, 1, or 2 substituents independently selected from the group consisting of halo, C₁-C₄ alkyl, C₁-C₄ hydroxyalkyl, C₁-C₆ alkoxy, C₁-C₄ haloalkyl, C₁-C₄ haloalkoxy, and C₃-C₆ cycloalkyl optionally substituted with one or two substituents independently selected from the group consisting of fluoro and methyl.

41. The compound of any one of claims 1-30, or a salt thereof, and/or an isotopologue thereof, wherein R^{e} is selected from the group consisting of:

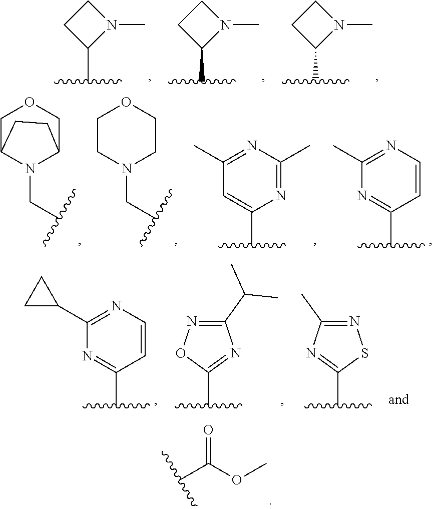

42. The compound of any one of claims 1-41, or a salt thereof, and/or an isotopologue thereof, wherein R^{x} is —OH.

43. The compound of any one of claims 1-41, or a salt thereof, and/or an isotopologue thereof, wherein n is 0.

44. The compound of claim 1, or a salt thereof, and/or an isotopologue thereof, wherein the compound is selected from the group consisting of:

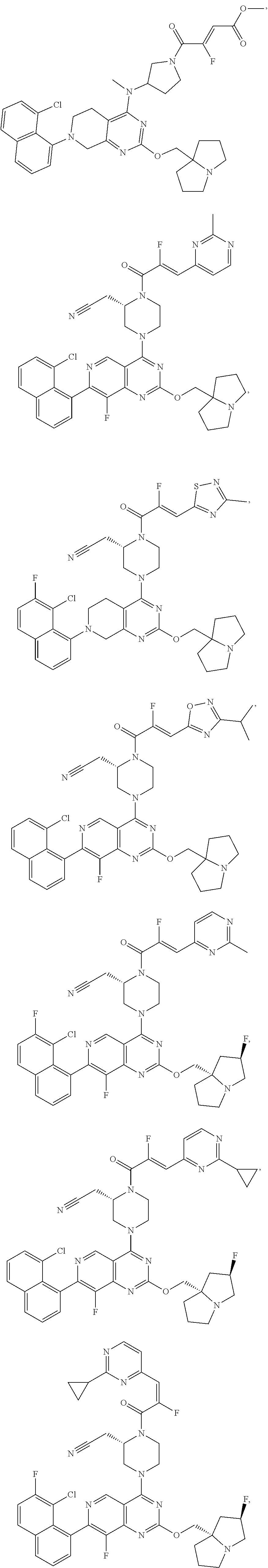

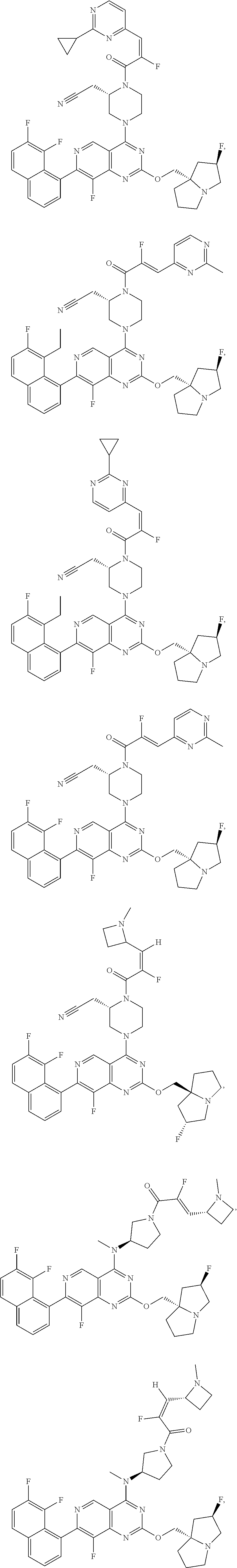

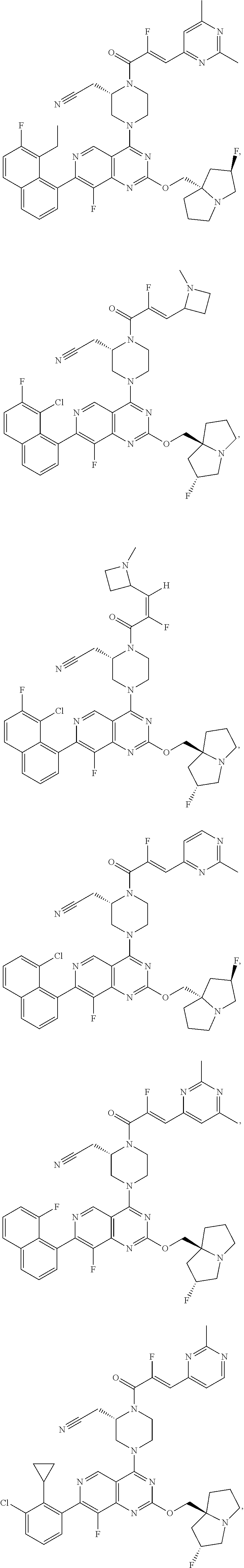

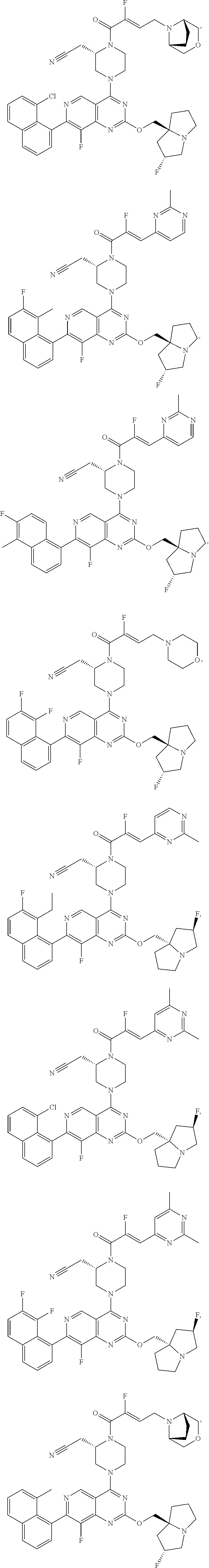

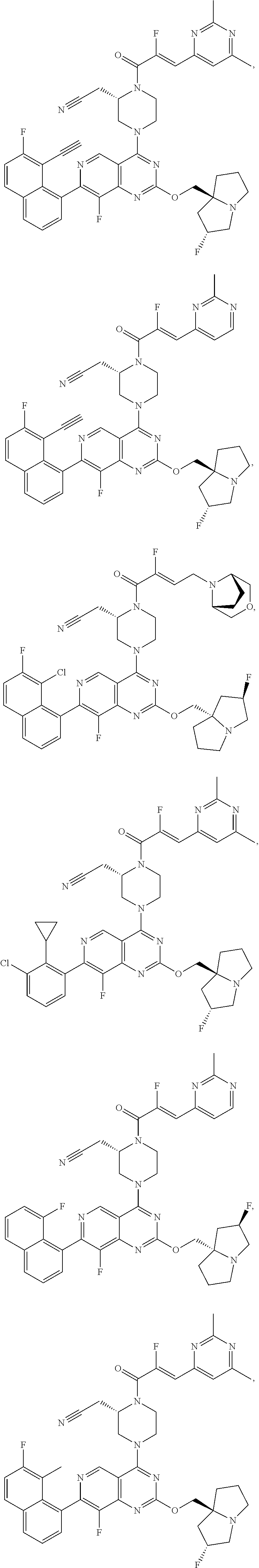

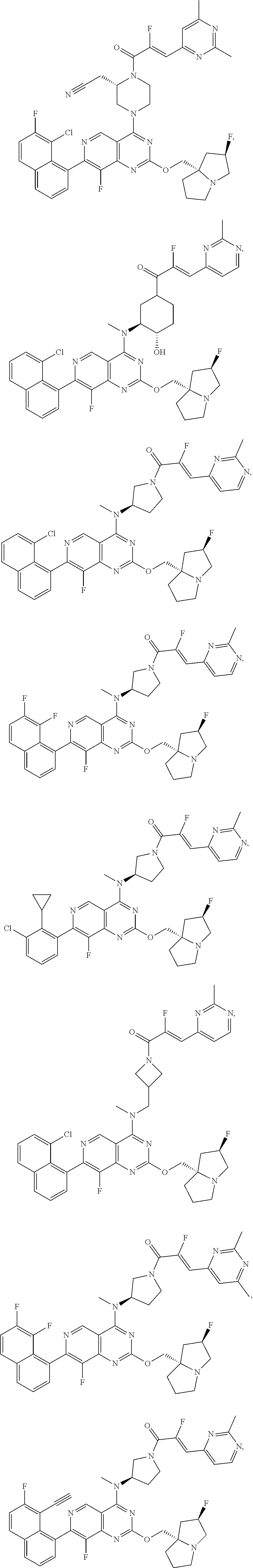

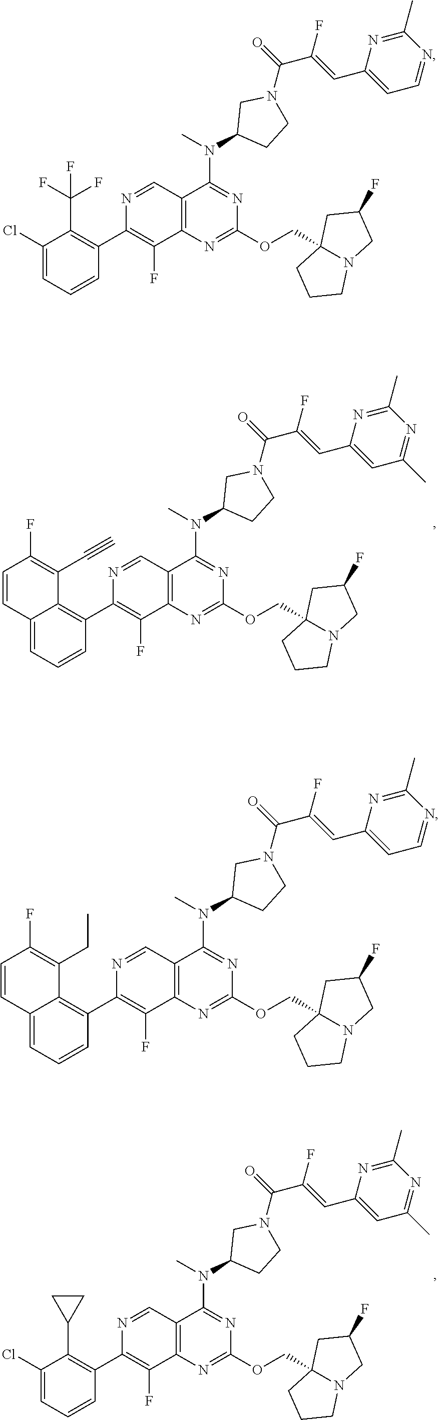 [SMILES: Cc1nccc(/C=C(\F)C(=O)N2CC[C@@H](N(C)c3nc(OC[C@@]45CCCN4C[C@H](F)C5)nc4c(F)c(-c5cccc(Cl)c5C(F)(F)F)ncc34)C2)n1 ; C#Cc1c(F)ccc2cccc(-c3ncc4c(N(C)[C@@H]5CCN(C(=O)/C(F)=C/c6cc(C)nc(C)n6)C5)nc(OC[C@@]56CCCN5C[C@H](F)C6)nc4c3F)c12 ; CCc1c(F)ccc2cccc(-c3ncc4c(N(C)[C@@H]5CCN(C(=O)/C(F)=C/c6ccnc(C)n6)C5)nc(OC[C@@]56CCCN5C[C@H](F)C6)nc4c3F)c12 ; Cc1cc(/C=C(\F)C(=O)N2CC[C@@H](N(C)c3nc(OC[C@@]45CCCN4C[C@H](F)C5)nc4c(F)c(-c5cccc(Cl)c5C5CC5)ncc34)C2)nc(C)n1]

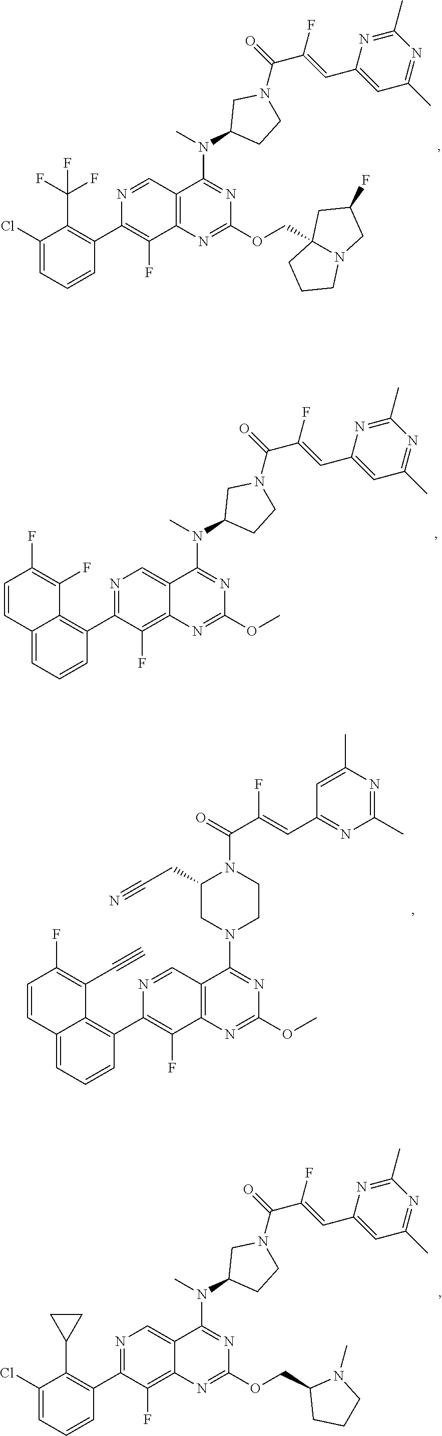 [SMILES: Cc1cc(/C=C(\F)C(=O)N2CC[C@@H](N(C)c3nc(OC[C@@]45CCCN4C[C@H](F)C5)nc4c(F)c(-c5cccc(Cl)c5C(F)(F)F)ncc34)C2)nc(C)n1 ; COc1nc(N(C)[C@@H]2CCN(C(=O)/C(F)=C/c3cc(C)nc(C)n3)C2)c2cnc(-c3cccc4ccc(F)c(F)c34)c(F)c2n1 ; C#Cc1c(F)ccc2cccc(-c3ncc4c(N5CCN(C(=O)/C(F)=C/c6cc(C)nc(C)n6)[C@@H](CC#N)C5)nc(OC)nc4c3F)c12 ; Cc1cc(/C=C(\F)C(=O)N2CC[C@@H](N(C)c3nc(OC[C@@H]4CCCN4C)nc4c(F)c(-c5cccc(Cl)c5C5CC5)ncc34)C2)nc(C)n1]

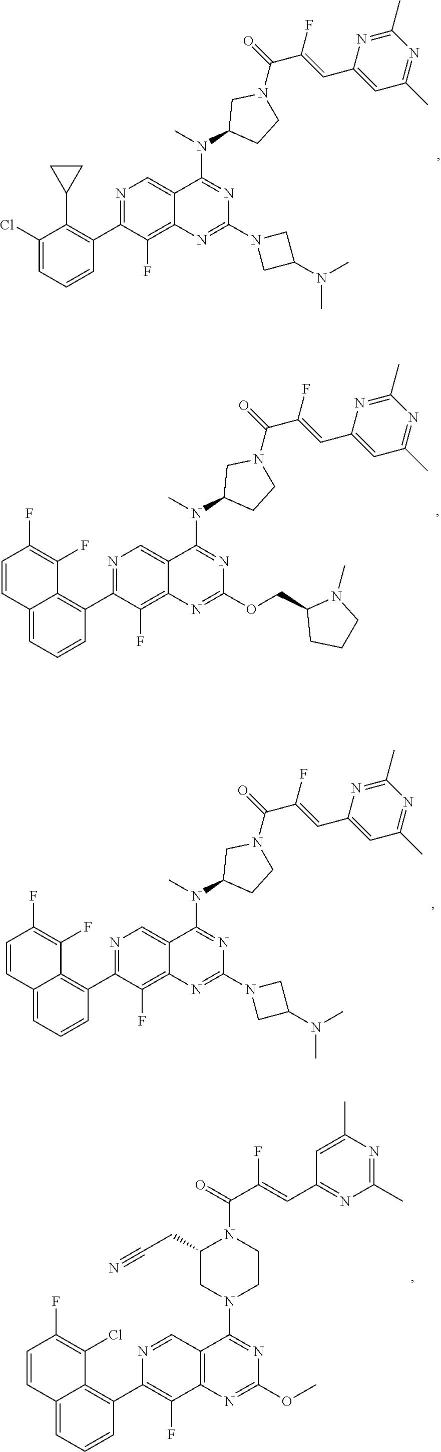 [SMILES: Cc1cc(/C=C(\F)C(=O)N2CC[C@@H](N(C)c3nc(N4CC(N(C)C)C4)nc4c(F)c(-c5cccc(Cl)c5C5CC5)ncc34)C2)nc(C)n1 ; Cc1cc(/C=C(\F)C(=O)N2CC[C@@H](N(C)c3nc(OC[C@@H]4CCCN4C)nc4c(F)c(-c5cccc6ccc(F)c(F)c56)ncc34)C2)nc(C)n1 ; Cc1cc(/C=C(\F)C(=O)N2CC[C@@H](N(C)c3nc(N4CC(N(C)C)C4)nc4c(F)c(-c5cccc6ccc(F)c(F)c56)ncc34)C2)nc(C)n1 ; COc1nc(N2CCN(C(=O)/C(F)=C/c3cc(C)nc(C)n3)[C@@H](CC#N)C2)c2cnc(-c3cccc4ccc(F)c(Cl)c34)c(F)c2n1]

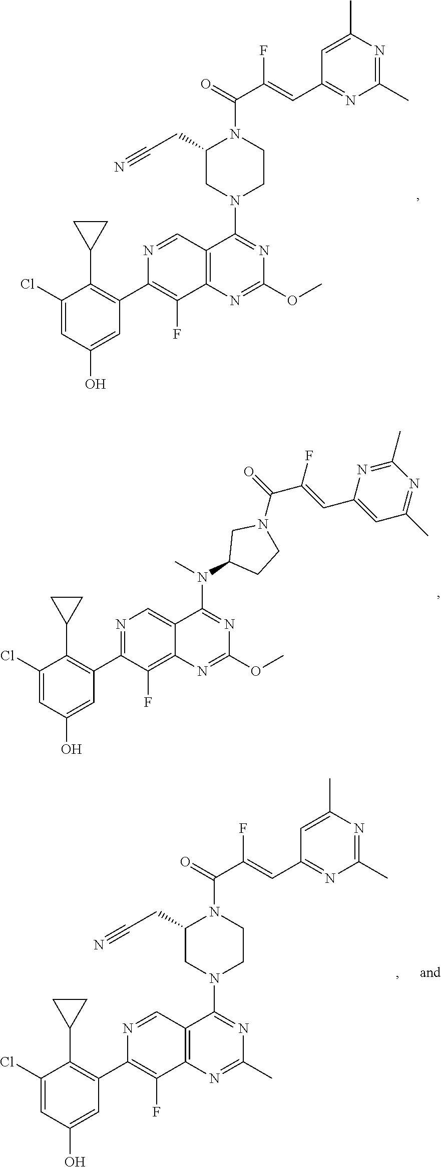 [SMILES: COc1nc(N2CCN(C(=O)/C(F)=C/c3cc(C)nc(C)n3)[C@@H](CC#N)C2)c2cnc(-c3cc(O)cc(Cl)c3C3CC3)c(F)c2n1 ; COc1nc(N(C)[C@@H]2CCN(C(=O)/C(F)=C/c3cc(C)nc(C)n3)C2)c2cnc(-c3cc(O)cc(Cl)c3C3CC3)c(F)c2n1 ; Cc1cc(/C=C(\F)C(=O)N2CCN(c3nc(C)nc4c(F)c(-c5cc(O)cc(Cl)c5C5CC5)ncc34)C[C@@H]2CC#N)nc(C)n1]

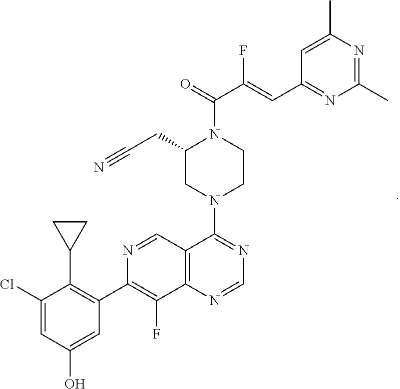 [SMILES: Cc1cc(/C=C(\F)C(=O)N2CCN(c3ncnc4c(F)c(-c5cc(O)cc(Cl)c5C5CC5)ncc34)C[C@@H]2CC#N)nc(C)n1]

45. The compound of any one of claims 1-44, or a salt thereof, and/or an isotopologue thereof, wherein the salt is a formate salt.

46. The compound of any one of claims 1-45, or a salt thereof, and/or an isotopologue thereof, wherein the salt is a pharmaceutically acceptable salt.

47. A pharmaceutical formulation comprising the compound of any one of claims 1-46, or pharmaceutically acceptable salt thereof, and/or an isotopologue thereof, and a pharmaceutically acceptable carrier.

48. A method of treating or suppressing cancer comprising: administering a therapeutically effective amount of a compound of any one of claims 1-46, or pharmaceutically acceptable salt thereof, and/or an isotopologue thereof, or a pharmaceutical formulation according to claim 47, to a subject in need thereof.

49. The method of claim 48, wherein the cancer is selected from the group consisting of: lung, colorectal, pancreatic, bile duct, thyroid, gall bladder, uterine, mesothelioma, cervical, and bladder cancers.

50. The method of claim 48, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cellmyeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, non-small cell lung cancer (NSCLC), and melanoma.

51. The method of any one of claims 48 to 50, wherein the cancer is a KRAS G12C mediated cancer, or wherein the subject has been diagnosed as having a KRAS G12C mediated cancer, or both.

52. The method of any one of claims 48 to 51, wherein the method further comprises administering to the subject a therapeutically effective amount of an additional chemotherapeutic agent.
